(19) United States
(12) Patent Application Publication
(10) Pub. No.: US 2026/0034126 A1
(43) Pub. Date: Feb. 5, 2026

(54) KRAS INHIBITORS

(71) Applicant: Incyte Corporation, Wilmington, DE (US)

(72) Inventors: Alexander Sokolsky, Wilmington, DE (US); Suman Chakrabarty, Wilmington, DE (US); Bin Hu, Garnet Valley, PA (US); Rocco Policarpo, III, Wilmington, DE (US); Brandon R. Smith, Chadds Ford, PA (US); Xiaozhao Wang, Mt. Laurel, NJ (US); Michael R. Witten, Wilmington, DE (US); Wenhao Zhang, Wilmington, DE (US); Wenyu Zhu, Wilmington, DE (US); Chunyin Marshall Law, Wilmington, DE (US); Haolin Yin, Wilmington, DE (US)

(21) Appl. No.: 19/285,011

(22) Filed: Jul. 30, 2025

Related U.S. Application Data
(60) Provisional application No. 63/678,682, filed on Aug. 2, 2024.

(51) Int. Cl. A61K 31/501; A61K 31/4725; A61K 31/497; A61K 31/506; C07D 401/06; C07D 409/14; C07D 417/14

(52) U.S. Cl. CPC A61K 31/501; A61K 31/4725; A61K 31/497; A61K 31/506; C07D 401/06; C07D 409/14; C07D 417/14

(57) ABSTRACT

Disclosed are compounds of Formula (I), methods of using the compounds for inhibiting KRAS activity and pharmaceutical compositions comprising such compounds. The compounds are useful in treating, preventing or ameliorating diseases or disorders associated with KRAS activity such as cancer.

### RELATED APPLICATIONS

This application is related to U.S. Provisional Application No. 63/678,682, filed Aug. 2, 2024, the content of which is incorporated in its entirety.

### FIELD OF THE INVENTION

This disclosure provides compounds as well as their compositions and methods of use. The compounds modulate KRAS activity and are useful in the treatment of various diseases including cancer.

### BACKGROUND OF THE INVENTION

Ras proteins are part of the family of small GTPases that are activated by growth factors and various extracellular stimuli. The Ras family regulates intracellular signaling pathways responsible for growth, migration, survival and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al., Nat. Rev. Drug. Discov., 2014, 13(11), 828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%), and lung adenocarcinomas (23%) (A. D. Cox, et al., Nat. Rev. Drug. Discov., 2014, 13(11), 828-51). In contrast, KRAS G12C mutations predominate in non-small cell lung cancer (NSCLC) comprising 11-16% of lung adenocarcinomas, and 2-5% of pancreatic and colorectal adenocarcinomas (A. D. Cox, et al., Nat. Rev. Drug. Discov., 2014, 13(11), 828-51). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al., Nat. Rev. Drug. Discov., 2014, 13(11), 828-51).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

### SUMMARY

The present disclosure provides, inter alia, a compound of Formula (I):

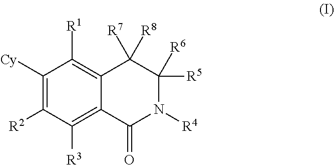

or a pharmaceutically acceptable salt thereof, wherein constituent variables are defined herein.

The present disclosure further provides a pharmaceutical composition comprising a compound of the disclosure, or a pharmaceutically acceptable salt thereof, and at least one pharmaceutically acceptable carrier or excipient.

The present disclosure further provides methods of inhibiting KRAS activity, which comprises administering to an individual a compound of the disclosure, or a pharmaceutically acceptable salt thereof. The present disclosure also provides uses of the compounds described herein in the manufacture of a medicament for use in therapy. The present disclosure also provides the compounds described herein for use in therapy.

The present disclosure further provides methods of treating a disease or disorder in a patient comprising administering to the patient a therapeutically effective amount of a compound of the disclosure, or a pharmaceutically acceptable salt thereof.

The details of one or more embodiments are set forth in the description below. Other features, objects, and advantages will be apparent from the description and from the claims.

### DETAILED DESCRIPTION

For the terms “e.g.” and “such as,” and grammatical equivalents thereof, the phrase “and without limitation” is understood to follow unless explicitly stated otherwise.

The singular forms “a,” “an,” and “the” include plural referents unless the context clearly dictates otherwise.

The term “about” means “approximately” (e.g., plus or minus approximately 10% of the indicated value).

### I. Compounds

In an aspect, provided herein is a compound having Formula (I):

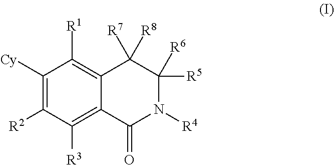

or a pharmaceutically acceptable salt thereof, wherein:
Cy is selected from C₃₋₇ cycloalkyl, 4-10 membered heterocycloalkyl, C₆₋₁₀ aryl, and 5-10 membered heteroaryl; wherein the C₃₋₇ cycloalkyl, 4-10 membered heterocycloalkyl, C₆₋₁₀ aryl, 5-10 membered heteroaryl, C₆₋₁₀ aryl, and 5-10 membered heteroaryl forming Cy is substituted with 1, 2, 3, or 4 substituents each independently selected from R^{Cy};
each R^{Cy} is independently selected from D, C₁₋₃ alkyl, C₁₋₃ haloalkyl, C₂₋₃ alkenyl, C₂₋₃ alkynyl, C₃₋₆ cycloalkyl, 4-10 membered heterocycloalkyl, C₆₋₁₀ aryl, 5-10 membered heteroaryl, halo, CN, OR^{aCy21}, SR^{aCy21}, NR^{cCy21}R^{dCy21}, S(O)R^{bCy21}, S(O)NR^{cCy21}R^{dCy21}, S(O)₂R^{bCy21}, NR^{cCy21}S(O)₂R^{bCy21}, and S(O)₂NR^{cCy21}R^{dCy21}; wherein the C₃₋₆ cycloalkyl, 4-10 membered heterocycloalkyl, C₆₋₁₀ aryl, and 5-10 membered heteroaryl forming R^{Cy} are each optionally substituted by 1, 2, 3, or 4 substituents independently selected from R^{Cy2A}; and wherein the C₁₋₃ alkyl, C₂₋₃ alkenyl, and C₂₋₃ alkynyl forming R^{Cy} are each optionally substituted by 1, 2, or 3 substituents independently selected from R^{Cy2B};
each R^{Cy2A} is independently selected from C₁₋₃ alkyl, C₁₋₃ haloalkyl, C₂₋₃ alkenyl, C₂₋₃ alkynyl, and R^{Cy2B}; wherein the C₁₋₃ alkyl, C₂₋₃ alkenyl, and C₂₋₃ alkynyl forming R^{Cy2A} are each optionally substituted by 1, 2, or 3 substituents independently selected from R^{Cy2B},
each R^{Cy2B} is independently selected from D, halo, CN, OR^{aCy21}, SR^{aCy21}, C(O)R^{bCy21}, C(O)NR^{Cy21}R^{dCy21}, C(O)OR^{aCy21}, OC(O)R^{bCy21}, OC(O)NR^{Cy21}R^{dCy21}, NR^{Cy21}R^{aCy21}, NR^{cCy21}C(O)R^{bCy21}, NR^{cCy21}C(O)NR^{cCy21}R^{aCy21}, NR^{cCy21}C(O)OR^{aCy21}, C(═NR^{cCy21})NR^{cCy21}R^{dCy21}, NR^{cCy21}C(═NR^{eCy21})NR^{cCy21}R^{dCy21}, S(O)R^{bCy21}, S(O)NR^{cCy21}R^{dCy21}, S(O)₂Rb^{Cy21}, NR^{cCy21}S(O)₂R^{bCy21}, and S(O)₂NR^{cCy21}R^{dCy21};
R^{aCy21}, R^{bCy21}, R_{cCy21}, and R^{dCy21} are each independently selected from H, C₁₋₃ alkyl, C₁₋₃ haloalkyl, C₂₋₃ alkenyl, C₂₋₃ alkynyl, C₆₋₁₀ aryl, C₃₋₇cycloalkyl, 5-10 membered heteroaryl, 4-10 membered heterocycloalkyl, C₆₋₁₀ aryl-C₁₋₃ alkylene, 5-10 membered heteroaryl-C₁₋₃ alkylene, C₃₋₇ cycloalkyl-C₁₋₃ alkylene, and 4-10 membered heterocycloalkyl-C₁₋₃ alkylene; wherein the C₆₋₁₀ aryl-C₁₋₃ alkylene, 5-10 membered heteroaryl-C₁₋₃ alkylene, C₃₋₇ cycloalkyl-C₁₋₃ alkylene, and 4-10 membered heterocycloalkyl-C₁₋₃ alkylene forming R^{aCy21}, R^{bCy21}, R^{cCy21}, and R_{eCy21} are each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)R^{bCy22}, OC(O)NR^{cCy22}R^{dCy22}, NR^{cCy22}R^{dCy22}, NR^{cCy22}C(O)R^{bCy22}, NR^{cCy22}C(O)NR^{dCy22}R^{dCy22}, NR^{cCy22}C(O)OR^{aCy22}, C(═NR^{eCy22})NR^{dCy22}R^{dCy22}, NR^{cCy22}C(═NR^{eCy22})NR^{cCy22}R^{dCy22}, S(O)R^{bCy22}, S(O)NR^{cCy22}R^{dCy22}, S(O)₂Rb^{cCy22}, NR^{cCy22}S(O)₂R^{bCy22}, and S(O)₂NR^{Cy22}R^{dCy22};
or R^{cCy21} and R^{dCy21} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)R^{bCy22}, OC(O)NR^{cCy22}R^{dCy22}, NR^{cCy22}R^{dCy22}, NR^{cCy22}C(O)R^{bCy22}, NR^{cCy22}C(O)NR^{Cy22}R^{dCy22}, NR^{cCy22}C(O)OR^{aCy22}, C(═NR^{eCy22})NR^{cCy22}R^{dCy22}, NR^{cCy22}C(═NR^{eCy22})NR^{cCy22}R^{dCy22}, S(O)R^{bCy22}, S(O)NR^{cCy22}R^{aCy22}, S(O)₂R^{bCy22}, NR^{cCy22}S(O)₂R^{bCy22}, and S(O)₂NR^{cCy22}R^{aCy22};
R^{aCy22}, R^{bCy22}, R^{cCy22}, and R^{eCy22} are each independently selected from H, C₁₋₃ alkyl, C₁₋₃ haloalkyl, C₂₋₃ alkenyl, C₂₋₃ alkynyl, C₆₋₁₀ aryl, C₆₋₁₀ aryl-C₁₋₃ alkylene, 5-10 membered heteroaryl-C₁₋₃ alkylene, C₃₋₇ cycloalkyl-C₁₋₃ alkylene, and 4-10 membered heterocycloalkyl-C₁₋₃ alkylene; wherein the C₁₋₃ alkyl, C₂₋₃ alkenyl, C₂₋₃ alkynyl, C₆₋₁₀ aryl-C₁₋₃ alkylene, 5-10 membered heteroaryl-C₁₋₃ alkylene, C₃₋₇ cycloalkyl-C₁₋₃ alkylene, and 4-10 membered heterocycloalkyl-C₁₋₃ alkylene forming R^{aCy22}, R^{bCy22}, R^{cCy22}, and R^{dCy22} are each optionally substituted with 1, 2, or 3 substituents independently selected from OH, CN, amino, NH(C₁₋₃ alkyl), N(C₁₋₃ alkyl)₂, halo, C₁₋₃ alkyl, C₁₋₃ alkoxy, C₁₋₃ haloalkyl, and C₁₋₃ haloalkoxy; or
R^{cCy22} and R^{dCy22} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₃alkyl, C₁₋₃ alkoxy, C₁₋₃ haloalkyl, and C₁₋₃ haloalkoxy;
R^{eCy21} and R^{eCy22} are each, independently, H, CN or NO₂;
R¹ is independently selected from halo, C₁₋₃ alkyl, C₂₋₃ alkenyl, C₂₋₃ alkynyl, C₃₋₁₀ cycloalkyl, 4-10 membered heterocycloalkyl, CN, OR^{1A}, NR^{1B}R^{1C}, C(O)NR^{1B}R^{1C}, and NR^{1B}C(O)R^{1C}; wherein the C₃₋₁₀ cycloalkyl and 4-10 membered heterocycloalkyl forming R¹ are each optionally substituted with 1, 2, or 3 substituents independently selected from R^{1D}; and wherein the C₁₋₃ alkyl, C₂₋₃ alkenyl, and C₂₋₃ alkynyl forming R¹ are each optionally substituted with 1, 2, or 3 substituents independently selected from R^{1E};
R^{1A} is selected from C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{1A} are each optionally substituted with 1, 2, or 3 substituents independently selected from R^{1D}; and wherein the C₁₋₃ alkyl, C₂₋₃ alkenyl, and C₂₋₃ alkynyl forming R^{1A} are each optionally substituted with 1, 2, or 3 substituents independently selected from R^{1E};
R^{1B} is selected from H, C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{1B} are each optionally substituted with 1, 2, or 3 substituents independently selected from R^{1D}; and wherein the C₁₋₃ alkyl, C₂₋₃ alkenyl, and C₂₋₃ alkynyl forming R^{1B} are each optionally substituted with 1, 2, or 3 substituents independently selected from R^{1E};
R^{1C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{1C} are each optionally substituted with 1, 2, or 3 substituents independently selected from R^{1E};
each R^{1D} is independently selected from C₁₋₃ alkyl, C₁₋₃ haloalkyl, C₂₋₃ alkenyl, C₂₋₃ alkynyl, and R^{1E}; wherein each of the C₁₋₃ alkyl, C₂₋₃ alkenyl, and C₂₋₃ alkynyl forming R^{1D} is optionally substituted with 1, 2, or 3 substituents independently selected from R^{1E};
each R^{1E} is independently selected from D, halo, CN, OH, C₁₋₃ alkoxy, C(O)OH, C(O)C₁₋₃ alkoxy, C(O)NH₂, C(O)NH(C₁₋₃ alkyl), C(O)N(C₁₋₃ alkyl)₂, and OC(O)C₁₋₃ alkyl;
R² is independently selected from halo, C₁₋₃ alkyl, C₂₋₃ alkenyl, C₂₋₃ alkynyl, C₃₋₁₀ cycloalkyl, 4-10 membered heterocycloalkyl, OR^{2A}, NR^{2B}R^{2C}, and NR^{2B}C(O)R^{2C}; wherein the C₃₋₁₀ cycloalkyl and 4-10 membered heterocycloalkyl forming R² are each optionally substituted with 1, 2, or 3 substituents independently selected from R^{2D}; and wherein the C₁₋₃ alkyl, C₂₋₃ alkenyl, and C₂₋₃ alkynyl forming R² are each optionally substituted with 1, 2, or 3 substituents independently selected from R^{2E};
R^{2A} is selected from C₁₋₃ alkyl, 2-3 alkenyl, 2-3 alkynyl, C₃₋₁₀ cycloalkyl, and 4-10 membered heterocycloalkyl; wherein the C₃₋₁₀ cycloalkyl and 4-10 membered heterocycloalkyl, forming R^{2A} are each optionally substituted with 1, 2, or 3 substituents independently selected from R^{2D}; and wherein the C₁₋₃ alkyl, C₂₋₃ alkenyl, and C₂₋₃ alkynyl forming R^{2A} are each optionally substituted with 1, 2, or 3 substituents independently selected from R^{2E};
R^{2B} is selected from H, C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{2B} are each optionally substituted with 1, 2, or 3 substituents independently selected from R^{2D}; and wherein the C₁₋₃ alkyl, C₂₋₃ alkenyl, and C₂₋₃ alkynyl forming R^{2B} are each optionally substituted with 1, 2, or 3 substituents independently selected from R^{2E};
R^{2C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{2C} are each optionally substituted with 1, 2, or 3 substituents independently selected from R^{2E};
each R^{2D} is independently selected from C₁₋₃ alkyl, C₁₋₃ haloalkyl, C₂₋₃ alkenyl, C₂₋₃ alkynyl, and R^{2E}; wherein each of the C₁-3 alkyl, C₂₋₃ alkenyl, and C₂₋₃ alkynyl forming R^{2D} is optionally substituted with 1, 2, or 3 substituents independently selected from R^{2E};
each R^{2E} is independently selected from O, halo, CN, OH, C₁₋₃ alkoxy, C(O)OH, C(O)C₁₋₃ alkoxy, C(O)NH₂, C(O)NH(C₁₋₃ alkyl), C(O)N(C₁₋₃ alkyl)₂, and OC(O)C₁₋₃ alkyl;
R³ is independently selected from H, halo, C₁₋₃ alkyl, C₂₋₃ alkenyl, C₂₋₄ alkynyl, C₃₋₁₀ cycloalkyl, 4-10 membered heterocycloalkyl, CN, OR^{3A}, NR^{3B}R^{3C}, and NR^{3B}C(O)R^{3C}; wherein the C₃₋₁₀ cycloalkyl and 4-10 membered heterocycloalkyl forming R³ are each optionally substituted with 1, 2, or 3 substituents independently selected from R^{3D}; and wherein the C₁₋₃ alkyl, C₂₋₃ alkenyl, and C₂₋₄ alkynyl forming R³ are each optionally substituted with 1, 2, or 3 substituents independently selected from R^{3E};
R^{3A} is selected from C₁₋₃ alkyl, C₂₋₃ alkenyl, C₂₋₃ alkynyl, C₃₋₁₀ cycloalkyl, 4-10 membered heterocycloalkyl, C₃₋₁₀ cycloalkyl-C₁₋₃ alkylene, and 4-10 membered heterocycloalkyl-C₁₋₃ alkylene; wherein the C₃₋₁₀ cycloalkyl 4-10 membered heterocycloalkyl, C₃₋₁₀ cycloalkyl-C₁₋₃ alkylene, and 4-10 membered heterocycloalkyl-C₁₋₃ alkylene forming R^{3A} are each optionally substituted with 1, 2, or 3 substituents independently selected from R^{3D}; and wherein the C₁₋₃ alkyl, C₂₋₃ alkenyl, and C₂₋₃ alkynyl forming R^{3A} are each optionally substituted with 1, 2, or 3 substituents independently selected from R^{3E};
R^{3B} is selected from H, C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{3B} are each optionally substituted with 1, 2, or 3 substituents independently selected from R^{3D}; and wherein the C₁₋₃ alkyl, C₂₋₃ alkenyl, and C₂₋₃ alkynyl forming R^{3B} are each optionally substituted with 1, 2, or 3 substituents independently selected from R^{3E};
R^{3C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{3C} are each optionally substituted with 1, 2, or 3 substituents independently selected from R^{3E};
each R^{3D} is independently selected from C₁₋₃ alkyl, C₁₋₃ haloalkyl, C₂₋₃ alkenyl, C₂₋₃ alkynyl, and R^{3E}; wherein each of the C₁₋₃ alkyl, C₂₋₃ alkenyl, and C₂₋₃ alkynyl forming R^{3D} is optionally substituted with 1, 2, or 3 substituents independently selected from R^{3E};
each R^{3E} is independently selected from D, C₁₋₃ alkyl, 4-6 membered heterocycloalkyl, halo, CN, OH, C₁₋₃ alkoxy, C(O)OH, C(O)C₁₋₃ alkoxy, C(O)NH₂, C(O)NH(C₁₋₃ alkyl), C(O)N(C₁₋₃ alkyl)₂, OC(O)C₁₋₃ alkyl, NH₂, NH(C₁₋₃ alkyl), and N(C₁₋₃ alkyl)₂, wherein the 4-6 membered heterocycloalkyl forming R^{3E} is optionally substituted with C₁₋₃ alkyl;
R⁴ is selected from C₁₋₃ alkyl, C₂₋₃ alkenyl, C₂₋₃ alkynyl, C₁₋₃ haloalkyl, C₃₋₅ cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃ alkylene, phenyl-C₁₋₃ alkylene, 5-6 membered heteroaryl-C₁₋₃ alkylene, halo, CN, OR^{a4}, C(O)R^{b4}, C(O)NR^{c4}R^{d4}, NR^{c4}R^{e4}, and NR^{c4}C(O)R^{b4}; wherein the C₃₋₅ cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃ alkylene, phenyl-C₁₋₃ alkylene, and 5-6 membered heteroaryl-C₁₋₃ alkylene forming R⁴ are each optionally substituted with 1, 2, or 3 substituents independently selected from R^{4A}; and wherein the C₁₋₃ alkyl, C₂₋₃ alkenyl, and C₂₋₃ alkynyl forming R⁴ are each optionally substituted with 1, 2, or 3 substituents independently selected from R^{4B};
each R^{a4} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a4} are each optionally substituted with 1, 2, or 3 substituents independently selected from R^{4A}; and wherein the C₁₋₆ alkyl, C₂₋₆ alkenyl, and C₂₋₆ alkynyl forming R^{a4} are each optionally substituted with 1, 2, or 3 substituents independently selected from R^{4B};
each R^{b4}, R^{c4}, and R^{d4} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b4}, R^{c4}, and R^{d4} are each optionally substituted with 1, 2, or 3 substituents independently selected from R^{4A}; and wherein the C₁₋₃ alkyl, C₂₋₃ alkenyl, and C₂₋₃ alkynyl forming R^{b4}, R^{c4}, and R^{d4} are each optionally substituted with 1, 2, or 3 substituents independently selected from R^{4B}; or
any R^{c4} and R^{d4} attached to the same N atom, together with the N atom to which they are attached, form a 4-, 5-, or 6-membered heterocycloalkyl group optionally substituted with 1, 2, or 3 substituents independently selected from R^{4B};
each R^{e4} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e4} are each optionally substituted with 1, 2, or 3 substituents independently selected from R^{4A}; and wherein the C₁₋₃ alkyl, C₂₋₃ alkenyl, and C₂₋₃ alkynyl, forming R^{e4} are each optionally substituted with 1, 2, or 3 substituents independently selected from R^{4B}; or
R^{c4} and R^{e4} attached to the same N atom, together with the N atom to which they are attached, form a 4-, 5-, or 6-membered heterocycloalkyl group optionally substituted with 1, 2, or 3 substituents independently selected from R^{4B};
each R^{4A} is independently selected from C₁₋₃ alkyl, C₂₋₃ alkenyl, C₂₋₃ alkynyl, C₁₋₃ haloalkyl, and R^{4B}, wherein the C₁₋₃ alkyl, C₂₋₃ alkenyl, and C₂₋₃ alkynyl, forming R^{4A} are each optionally substituted with 1, 2 or 3 substituents independently selected from R^{4B};
each R^{4B} is independently selected from C₃₋₆ cycloalkyl, 4-10 membered heterocycloalkyl, phenyl, 5-6 membered heteroaryl, halo, D, CN, OR^{a4B}, C(O)R^{b4B}, C(O)NR^{c4B}R^{d4B}, C(O)OR^{a4B}, NR^{c4B}R^{d4B}, and S(O)₂R^{b4B}; wherein the C₁₋₃ alkyl, C₃₋₆cycloalkyl, 4-6 membered heterocycloalkyl, phenyl, and 5-6 membered heteroaryl are each optionally substituted with 1, 2, or 3 substituents independently selected from R^{4C};
each R^{4C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a4C}, C(O)R^{b4C}, C(O)NR^{c4C}R^{d4C}, C(O)OR^{a4C}, NR^{c4C}R^{d4C}, and S(O)₂R^{b4C};
each R^{a4B}, R^{b4B}, R^{c4B} and R^{d4B} is independently selected from H, C₁₋₃ alkyl, and C₁₋₃ haloalkyl;
each R^{a4C}, R^{b4C}, R^{c4C} and R^{d4C} is independently selected from H, C₁₋₃ alkyl, and C₁₋₃ haloalkyl;
R⁵ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃alkylene, CN, OR^{a5}, C(O)R^{b5}, C(O)NR^{c5}R^{d5}, NR^{c5}R^{e5}, and NR^{c5}C(O)R^{b5}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁵ are each optionally substituted with 1, 2, or 3 substituents independently selected from R^{5A}; and wherein the C₁₋₃ alkyl, C₂₋₃ alkenyl, and C₂₋₃ alkynyl forming R⁵ are each optionally substituted with 1, 2, or 3 substituents independently selected from R^{5B};
each R^{a5} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a5} are each optionally substituted with 1, 2, or 3 substituents independently selected from R^{5A}; and wherein the C₁₋₆ alkyl, C₂₋₆ alkenyl, and C₂₋₆ alkynyl forming R^{a5} are each optionally substituted with 1, 2, or 3 substituents independently selected from R^{5B};
each R^{b5}, R^{c5}, and R^{d5} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b5}, R^{c5}, and R^{d5} are each optionally substituted with 1, 2, or 3 substituents independently selected from R^{5A}; and wherein the C₁₋₃ alkyl, C₂₋₃ alkenyl, and C₂₋₃ alkynyl forming R^{b5}, R^{c5}, and R^{d5} are each optionally substituted with 1, 2, or 3 substituents independently selected from R^{5B}; or
any R^{c5} and R^{d5} attached to the same N atom, together with the N atom to which they are attached, form a 4-, 5-, or 6-membered heterocycloalkyl group optionally substituted with 1, 2, or 3 substituents independently selected from R^{5B};
each R^{e5} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e5} are each optionally substituted with 1, 2, or 3 substituents independently selected from R^{5A}; and wherein the C₁₋₃ alkyl, C₂₋₃ alkenyl, and C₂₋₃ alkynyl, forming R^{e5} are each optionally substituted with 1, 2, or 3 substituents independently selected from R^{5B}; or
R^{c5} and R^{e5} attached to the same N atom, together with the N atom to which they are attached, form a 4-, 5-, or 6-membered heterocycloalkyl group optionally substituted with 1, 2, or 3 substituents independently selected from R^{5B};
each R^{5A} is independently selected from C₁₋₃ alkyl, C₂₋₃ alkenyl, C₂₋₃ alkynyl, C₁₋₃ haloalkyl, and R^{5B}, wherein the C₁₋₃ alkyl, C₂₋₃ alkenyl, and C₂₋₃ alkynyl forming R^{5A} are each optionally substituted with 1, 2 or 3 substituents independently selected from R^{5B};
each R^{5B} is independently selected from C₃₋₆ cycloalkyl, 4-10 membered heterocycloalkyl, phenyl, 5-6 membered heteroaryl, halo, D, CN, OR^{a5B}, C(O)R^{b5B}, C(O)NR^{c5B}R^{d5B}, C(O)OR^{a5B}, NR^{c5B}R^{d5B}, and S(O)₂R^{b5B}; wherein the C₁₋₃ alkyl, C₃₋₆cycloalkyl, 4-6 membered heterocycloalkyl, phenyl, and 5-6 membered heteroaryl are each optionally substituted with 1, 2, or 3 substituents independently selected from R^{5C};
each R^{5C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a5C}, C(O)R^{b5C}, C(O)NR^{c5C}R^{d5C}, C(O)OR^{a5C}, NR^{c5C}R^{d5C}, and S(O)₂R^{b5C};
each R^{a5B}, R^{b5B}, R^{c5B} and R^{d5B} is independently selected from H, C₁₋₃ alkyl, and C₁₋₃ haloalkyl; and
each R^{a5C}, R^{b5C}, R^{c5C} and R^{d5C} is independently selected from H, C₁₋₃ alkyl, and C₁₋₃ haloalkyl;
R⁶ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃alkylene, CN, OR^{a6}, C(O)R^{b6}, C(O)NR^{c6}R^{d6}, NR^{c6}R^{e6}, and NR^{c6}C(O)R^{b6}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁶ are each optionally substituted with 1, 2, or 3 substituents independently selected from R^{6A}; and wherein the C₁₋₃ alkyl, C₂₋₃ alkenyl, and C₂₋₃ alkynyl forming R⁶ are each optionally substituted with 1, 2, or 3 substituents independently selected from R^{6B};
each R^{a6} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a6} are each optionally substituted with 1, 2, or 3 substituents independently selected from R^{6A}; and wherein the C₁₋₆ alkyl, C₂₋₆ alkenyl, and C₂₋₆ alkynyl forming R^{a6} are each optionally substituted with 1, 2, or 3 substituents independently selected from R^{6B};
each R^{b6}, R^{c6}, and R^{d6} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b6}, R^{c6}, and R^{d6} are each optionally substituted with 1, 2, or 3 substituents independently selected from R^{6A}; and wherein the C₁₋₃ alkyl, C₂₋₃ alkenyl, and C₂₋₃ alkynyl forming R^{b6}, R^{c6}, and R^{d6} are each optionally substituted with 1, 2, or 3 substituents independently selected from R^{6B}; or
any R^{c6} and R^{d6} attached to the same N atom, together with the N atom to which they are attached, form a 4-, 5-, or 6-membered heterocycloalkyl group optionally substituted with 1, 2, or 3 substituents independently selected from R^{6B};
each R^{e6} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e6} are each optionally substituted with 1, 2, or 3 substituents independently selected from R^{6A}; and wherein the C₁₋₃ alkyl, C₂₋₃ alkenyl, and C₂₋₃ alkynyl, forming R^{e6} are each optionally substituted with 1, 2, or 3 substituents independently selected from R^{6B}; or
R^{c6} and R^{e6} attached to the same N atom, together with the N atom to which they are attached, form a 4-, 5-, or 6-membered heterocycloalkyl group optionally substituted with 1, 2, or 3 substituents independently selected from R^{6B};
each R^{6A} is independently selected from C₁₋₃ alkyl, C₂₋₃ alkenyl, C₂₋₃ alkynyl, C₁₋₃ haloalkyl, and R^{6B}, wherein the C₁₋₃ alkyl, C₂₋₃ alkenyl, and C₂₋₃ alkynyl forming R^{6A} are each optionally substituted with 1, 2 or 3 substituents independently selected from R^{6B};
each R^{6B} is independently selected from C₃₋₆ cycloalkyl, 4-10 membered heterocycloalkyl, phenyl, 5-6 membered heteroaryl, halo, D, CN, OR^{a6B}, C(O)R^{b6B}, C(O)NR^{c6B}R^{d6B}, C(O)OR^{a6B}, NR^{c6B}R^{d6B}, and S(O)₂R^{b6B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{6C};
each R^{6C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a6C}, C(O)R^{b6C}, C(O)NR^{c6C}R^{d6C}, C(O)OR^{a6C}, NR^{c6C}R^{d6C}, and S(O)₂R^{b6C};
each R^{a6B}, R^{b6B}, R^{c6B} and R^{d6B} is independently selected from H, C₁₋₃ alkyl, and C₁₋₃ haloalkyl; and
each R^{a6C}, R^{b6C}, R^{c6C} and R^{d6C} is independently selected from H, C₁₋₃ alkyl, and C₁₋₃ haloalkyl;
R⁷ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a7}, C(O)R^{b7}, C(O)NR^{c7}R^{d7}, NR^{c7}R^{e7}, and NR^{c7}C(O)R^{b7}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁷ are each optionally substituted with 1, 2, or 3 substituents independently selected from R^{7A}; and wherein the C₁₋₃ alkyl, C₂₋₃ alkenyl, and C₂₋₃ alkynyl forming R⁷ are each optionally substituted with 1, 2, or 3 substituents independently selected from R^{7B};
each R^{a7} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a7} are each optionally substituted with 1, 2, or 3 substituents independently selected from R^{7A}; and wherein the C₁₋₆ alkyl, C₂₋₆ alkenyl, and C₂₋₆ alkynyl forming R^{a7} are each optionally substituted with 1, 2, or 3 substituents independently selected from R^{7B};
each R^{b7}, R^{c7}, and R^{d7} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b7}, R^{c7}, and R^{d7} are each optionally substituted with 1, 2, or 3 substituents independently selected from R^{7A}; and wherein the C₁₋₃ alkyl, C₂₋₃ alkenyl, and C₂₋₃ alkynyl forming R^{b7}, R^{c7}, and R^{d7} are each optionally substituted with 1, 2, or 3 substituents independently selected from R^{7B}; or
any R^{c7} and R^{d7} attached to the same N atom, together with the N atom to which they are attached, form a 4-, 5-, or 6-membered heterocycloalkyl group optionally substituted with 1, 2, or 3 substituents independently selected from R^{7B};
each R^{e7} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e7} are each optionally substituted with 1, 2, or 3 substituents independently selected from R^{7A}; and wherein the C₁₋₃ alkyl, C₂₋₃ alkenyl, and C₂₋₃ alkynyl, forming R^{e7} are each optionally substituted with 1, 2, or 3 substituents independently selected from R^{7B}; or
R^{c7} and R^{e7} attached to the same N atom, together with the N atom to which they are attached, form a 4-, 5-, or 6-membered heterocycloalkyl group optionally substituted with 1, 2, or 3 substituents independently selected from R^{7B};
each R^{7A} is independently selected from C₁₋₃ alkyl, C₂₋₃ alkenyl, C₂₋₃ alkynyl, C₁₋₃ haloalkyl, and R^{7B}, wherein the C₁₋₃ alkyl, C₂₋₃ alkenyl, and C₂₋₃ alkynyl forming R^{7A} are each optionally substituted with 1, 2 or 3 substituents independently selected from R^{7B};
each R^{7B} is independently selected from C₃₋₆ cycloalkyl, 4-10 membered heterocycloalkyl, phenyl, 5-6 membered heteroaryl, halo, D, CN, OR^{a7B}, C(O)R^{b7B}, C(O)NR^{c7B}R^{d7B}, C(O)OR^{a7B}, NR^{c7B}R^{d7B}, and S(O)₂R^{b7B}; wherein the C₁₋₃ alkyl, C₃₋₆cycloalkyl, 4-6 membered heterocycloalkyl, phenyl, and 5-6 membered heteroaryl are each optionally substituted with 1, 2, or 3 substituents independently selected from R^{7C};
each R^{7C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a7C}, C(O)R^{b7C}, C(O)NR^{c7C}R^{d7C}, C(O)OR^{a7C}, NR^{c7c}R^{d7C}, and S(O)₂R^{b7c};
each R^{a7B}, R^{b7B}, R^{c7B} and R^{d7B} is independently selected from H, C₁₋₃ alkyl, and C₁₋₃ haloalkyl; and
each R^{a7C}, R^{b7C}, R^{c7C} and R^{d7C} is independently selected from H, C₁₋₃ alkyl, and C₁₋₃ haloalkyl;
R⁸ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a8}, C(O)R^{b8}, C(O)NR^{c8}R^{d8}, NR^{c8}R^{e8}, and NR^{c8}C(O)R^{b8}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁸ are each optionally substituted with 1, 2, or 3 substituents independently selected from R^{8A}; and wherein the C₁₋₃ alkyl, C₂₋₃ alkenyl, and C₂₋₃ alkynyl forming R⁸ are each optionally substituted with 1, 2, or 3 substituents independently selected from R^{8B};
each R^{a8} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a8} are each optionally substituted with 1, 2, or 3 substituents independently selected from R^{8A}; and wherein the C₁₋₆ alkyl, C₂₋₆ alkenyl, and C₂₋₆ alkynyl forming R^{a8} are each optionally substituted with 1, 2, or 3 substituents independently selected from R^{8B};
each R^{b8}, R^{c8}, and R^{d8} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b8}, R^{c8}, and R^{d8} are each optionally substituted with 1, 2, or 3 substituents independently selected from R^{8A}; and wherein the C₁₋₃ alkyl, C₂₋₃ alkenyl, and C₂₋₃ alkynyl forming R^{b8}, R^{c8}, and R^{d8} are each optionally substituted with 1, 2, or 3 substituents independently selected from R^{8B} or
any R^{c8} and R^{d8} attached to the same N atom, together with the N atom to which they are attached, form a 4-, 5-, or 6-membered heterocycloalkyl group optionally substituted with 1, 2, or 3 substituents independently selected from R^{8B};
each R^{e8} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e8} are each optionally substituted with 1, 2, or 3 substituents independently selected from R^{8A}; and wherein the C₁₋₃ alkyl, C₂₋₃ alkenyl, and C₂₋₃ alkynyl, forming R^{e8} are each optionally substituted with 1, 2, or 3 substituents independently selected from R^{8B}; or
R^{c8} and R^{e8} attached to the same N atom, together with the N atom to which they are attached, form a 4-, 5-, or 6-membered heterocycloalkyl group optionally substituted with 1, 2, or 3 substituents independently selected from R^{8B};
each R^{8A} is independently selected from C₁₋₃ alkyl, C₂₋₃ alkenyl, C₂₋₃ alkynyl, C₁₋₃ haloalkyl, and R^{8B}, wherein the C₁₋₃ alkyl, C₂₋₃ alkenyl, and C₂₋₃ alkynyl forming R^{8A} are each optionally substituted with 1, 2 or 3 substituents independently selected from R^{8B};
each R^{8B} is independently selected from C₃₋₆ cycloalkyl, 4-10 membered heterocycloalkyl, phenyl, 5-6 membered heteroaryl, halo, D, CN, OR^{a8B}, C(O)R^{b8B}, C(O)NR^{c8B}R^{d8B}, C(O)OR^{a8B}, NR^{c8B}R^{d8B}, and S(O)₂R^{b8B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{8C};
each R^{8C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a8C}, C(O)R^{b8C}, C(O)NR^{c8C}R^{d8C}, C(O)OR^{a8C}, NR^{c8C}R^{d8C}, and S(O)₂R^{b8C};
each R^{a8B}, R^{b8B}, R^{c8B} and R^{d8B} is independently selected from H, C₁₋₃ alkyl, and C₁₋₃ haloalkyl; and
each R^{a8c}, R^{b8C}, R^{c8C} and R^{d8C} is independently selected from H, C₁₋₃ alkyl, and C₁₋₃ haloalkyl;
wherein the ring-forming atoms of heterocycloalkyl and heteroaryl consist of at least one carbon atom and 1, 2, 3, or 4 heteroatoms independently selected from N, O, and S; and
wherein a ring-forming carbon atom of heterocycloalkyl and heteroaryl is optionally substituted by oxo to form a carbonyl group.

In an embodiment of Formula I,
R¹ is independently selected from halo, C₁₋₃ alkyl, C₂₋₃ alkenyl, C₂₋₃ alkynyl, C₃₋₁₀ cycloalkyl, 4-10 membered heterocycloalkyl, OR^{1A}, NR^{1B}R^{1C}, and NR^{1B}C(O)R^{1C}; wherein the C₃₋₁₀ cycloalkyl and 4-10 membered heterocycloalkyl forming R¹ are each optionally substituted with 1, 2, or 3 substituents independently selected from R^{1D}; and wherein the C₁₋₃ alkyl, C₂₋₃ alkenyl, and C₂₋₃ alkynyl forming R¹ are each optionally substituted with 1, 2, or 3 substituents independently selected from R^{1E};
R³ is independently selected from H, halo, C₁₋₃ alkyl, C₂₋₃ alkenyl, C₂₋₃ alkynyl, C₃₋₁₀ cycloalkyl, 4-10 membered heterocycloalkyl, CN, OR^{3A}, NR^{3B}R^{3C}, and NR^{3B}C(O)R^{3C}; wherein the C₃₋₁₀ cycloalkyl and 4-10 membered heterocycloalkyl forming R³ are each optionally substituted with 1, 2, or 3 substituents independently selected from R^{3D}; and wherein the C₁₋₃ alkyl, C₂₋₃ alkenyl, and C₂₋₃ alkynyl forming R³ are each optionally substituted with 1, 2, or 3 substituents independently selected from R^{3E}; and
R⁴ is selected from C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃alkylene, halo, CN, OR^{a4}, C(O)R^{b4}, C(O)NR^{c4}R^{d4}, NR^{c4}R^{e4}, and NR^{c4}C(O)R^{b4}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁴ are each optionally substituted with 1, 2, or 3 substituents independently selected from R^{4A}; and wherein the C₁₋₃ alkyl, C₂₋₃ alkenyl, and C₂₋₃ alkynyl forming R⁴ are each optionally substituted with 1, 2, or 3 substituents independently selected from R^{4B}.

In embodiments,
Cy is selected from C₃₋₇ cycloalkyl, 4-10 membered heterocycloalkyl, C₆₋₁₀ aryl, and 5-10 membered heteroaryl; wherein the C₃₋₇ cycloalkyl, 4-10 membered heterocycloalkyl, C₆₋₁₀ aryl, and 5-10 membered heteroaryl forming Cy is substituted with 1, 2, 3, or 4 substituents independently selected from R^{Cy};
each R^{Cy} is independently selected from D, C₁₋₃ alkyl, C₁₋₃ haloalkyl, C₂₋₃ alkenyl, C₂₋₃ alkynyl, C₃₋₆ cycloalkyl, 4-10 membered heterocycloalkyl, C₆₋₁₀ aryl, 5-10 membered heteroaryl, halo, CN, OR^{aCy21}, and NR^{cCy21}R^{dCy21}; wherein the C₃₋₆ cycloalkyl, 4-10 membered heterocycloalkyl, C₆₋₁₀ aryl, and 5-10 membered heteroaryl forming R^{Cy} are each optionally substituted by 1, 2, 3, or 4 substituents independently selected from R^{Cy2A}; and wherein the C₁₋₃ alkyl, C₂₋₃ alkenyl, and C₂₋₃ alkynyl forming R^{Cy} are each optionally substituted by 1, 2, or 3 substituents independently selected from R^{Cy2B};
each R^{Cy2A} is independently selected from C₁₋₃ alkyl, C₁₋₃ haloalkyl, O₂-3 alkenyl, and C₂₋₃ alkynyl; wherein the C₁₋₃ alkyl, C₂₋₃ alkenyl, and C₂₋₃ alkynyl forming R^{Cy2A} are each optionally substituted by 1, 2, or 3 substituents independently selected from R^{Cy2B},
each R^{Cy2B} is independently selected from D, halo, CN, OR^{aCy21}, C(O)R^{bCy21}, C(O)NR^{Cy21}R^{dCy21}, C(O)OR^{aCy21}, OC(O)R^{bCy21}, OC(O)NR^{Cy21}R^{dcCy21}, NR^{Cy21}R^{dCy21}, NR^{cCy21}C(O)R^{bCy21}, NR^{cCy21}C(O)NR^{cCy21}R^{dCy21}, and NR^{cCy21}C(O)OR^{aCy21};
R^{aCy21}, R^{bCy21}, R^{cCy21}, and R^{dCy21} are each independently selected from H, C₁₋₃ alkyl, C₁₋₃ haloalkyl, C₂₋₃ alkenyl, and C₂₋₃ alkynyl; R¹ is selected from halo, C₁₋₃ alkyl, C₂₋₃ alkenyl, C₂₋₃ alkynyl, CN, OR^{1A}, NR^{1B}R^{1C}, C(O)NR^{1B}R^{1C}, and NR^{1B}C(O)R^{1C};
R^{1A} is C₁₋₃ alkyl;
R^{1B} is selected from H, C₁₋₃ alkyl, C₂₋₃ alkenyl, and C₂₋₃ alkynyl;
R^{1C} is selected from H, C₁₋₃ alkyl, C₂₋₃ alkenyl, and C₂₋₃ alkynyl;
R² is selected from halo, C₁₋₃ alkyl, C₂₋₃ alkenyl, C₂₋₃ alkynyl, OR^{2A}, NR^{2B}R^{2C}, and NR^{2B}C(O)R^{2C};
R^{2A} is C₁₋₃ alkyl;
R^{2B} is selected from H, C₁₋₃ alkyl, C₂₋₃ alkenyl, and C₂₋₃ alkynyl;
R^{2C} is selected from H, C₁₋₃ alkyl, C₂₋₃ alkenyl, and C₂₋₃ alkynyl;
R³ is selected from H, halo, C₁₋₃ alkyl, C₂₋₃ alkenyl, C₂₋₄ alkynyl, 5-6 membered heteroaryl, OR^{3A}, NR^{3B}R^{3C}, and NR^{3B}C(O)R^{3C}, wherein the C₂₋₄ alkynyl forming R³ is optionally substituted with 1 or 2 substituents independently selected from R^{3E};
R^{3A} is C₁₋₃ alkyl or 4-10 membered heterocycloalkyl-C₁₋₃ alkylene optionally substituted with 1, 2, or 3 substituents independently selected from R^{3D};
R^{3B} is selected from H, C₁₋₃ alkyl, C₂₋₃ alkenyl, and C₂₋₃ alkynyl; R^{3C} is selected from H, C₁₋₃ alkyl, C₂₋₃ alkenyl, and C₂₋₃ alkynyl;
each R^{3D} is independently C₁₋₃ alkyl;
R⁴ is selected from C₁₋₃ alkyl, C₂₋₃ alkenyl, C₂₋₃ alkynyl, C₁₋₃ haloalkyl, C₃₋₅ cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃ alkylene, phenyl-C₁₋₃ alkylene, 5-6 membered heteroaryl-C₁₋₃ alkylene; wherein the C₃₋₅ cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃ alkylene, phenyl-C₁₋₃ alkylene, and 5-6 membered heteroaryl-C₁₋₃ alkylene forming R⁴ are each optionally substituted with 1, 2, or 3 substituents independently selected from R^{4A}; and wherein the C₁₋₃ alkyl, C₂₋₃ alkenyl, and C₂₋₃ alkynyl forming R⁴ are each optionally substituted with 1, 2, or 3 substituents independently selected from R^{4B}
each R^{4A} is independently selected from C₁₋₃ alkyl, C₂₋₃ alkenyl, C₂₋₃ alkynyl, C₁₋₃ haloalkyl, and R^{4B};
each R^{4B} is independently selected from C₃₋₆ cycloalkyl, 4-10 membered heterocycloalkyl, phenyl, 5-6 membered heteroaryl, halo, D, CN, OR^{a4B}, C(O)R^{b4B}, C(O)NR^{c4B}R^{d4B}, C(O)OR^{a4B}, and NR^{c4B}R^{d4B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{4C};
each R^{4C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a4C}, C(O)R^{b4C}, C(O)NR^{c4C}R^{d4C}, C(O)OR^{a4C}, and NR^{c4C}R^{d4C};
each R^{a4B}, R^{b4B}, R^{c4B} and R^{d4B} is independently selected from H and C₁₋₃ alkyl;
each R^{a4C}, R^{b4C}, R^{c4C} and R^{d4C} is independently selected from H and C₁₋₃ alkyl;
R⁵ is selected from H, D, and C₁₋₃ alkyl;
R⁶ is selected from H, D, and C₁₋₃ alkyl;
R⁷ is selected from H, D, and C₁₋₃ alkyl; and
R⁸ is selected from H, D, and C₁₋₃ alkyl.

In embodiments,
R¹ is selected from halo, C₁₋₃ alkyl, C₂₋₃ alkenyl, C₂₋₃ alkynyl, OR^{1A}, NR^{1B}R^{1C}, and NR^{1B}C(O)R^{1C};
R³ is selected from H, halo, C₁₋₃ alkyl, C₂₋₃ alkenyl, C₂₋₃ alkynyl, 5-6 membered heteroaryl, OR^{3A}, NR^{3B}R^{3C}, and NR^{3B}C(O)R^{3C}; and
R⁴ is selected from C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁴ are each optionally substituted with 1, 2, or 3 substituents independently selected from R^{4A}; and wherein the C₁₋₃ alkyl, C₂₋₃ alkenyl, and C₂₋₃ alkynyl forming R⁴ are each optionally substituted with 1, 2, or 3 substituents independently selected from R^{4B}.

In embodiments,
Cy is selected from C₆₋₁₀ aryl and 5-10 membered heteroaryl; wherein C₆₋₁₀ aryl and 5-10 membered heteroaryl forming Cy is substituted with 1, 2, 3, or 4 substituents independently selected from R^{Cy};
each R^{Cy} is independently selected from C₁₋₃ alkyl, C₁₋₃ haloalkyl, halo, CN, and NR^{cCy21}R^{dCy21};
R^{cCy21} and R^{dCy21} are each independently selected from H and C₁₋₃ alkyl;
R¹ is selected from halo, C₁₋₃ alkyl, CN, NR^{1B}R^{1C}, C(O)NR^{1B}R^{1C}, and NR^{1B}C(O)R^{1C};
R^{1B} is selected from H, C₁₋₃ alkyl, C₂₋₃ alkenyl, and C₂₋₃ alkynyl;
R^{1C} is selected from H, C₁₋₃ alkyl, C₂₋₃ alkenyl, and C₂₋₃ alkynyl;
R² is selected from halo, C₁₋₃ alkyl, NR^{2B}R^{2C}, and NR^{2B}C(O)R^{2C};
R^{2B} is selected from H, C₁₋₃ alkyl, C₂₋₃ alkenyl, and C₂₋₃ alkynyl;
R^{2C} is selected from H, C₁₋₃ alkyl, C₂₋₃ alkenyl, and C₂₋₃ alkynyl;
R³ is selected from halo, C₁₋₃ alkyl, C₂₋₄ alkynyl, 5-6 membered heteroaryl, NR^{3B}R^{3C}, and
NR^{3B}C(O)R^{3C}, wherein the C₂₋₄ alkynyl forming R³ is optionally substituted with 1 or 2 substituents independently selected from R^{3E};
R^{3B} is selected from H, C₁₋₃ alkyl, C₂₋₃ alkenyl, and C₂₋₃ alkynyl;
R^{3C} is selected from H, C₁₋₃ alkyl, C₂₋₃ alkenyl, and C₂₋₃ alkynyl;
R⁴ is selected from C₁₋₃ alkyl, 4-10 membered heterocycloalkyl, and 5-6 membered heteroaryl-C₁₋₃ alkylene; wherein the 4-10 membered heterocycloalkyl and 5-6 membered heteroaryl-C₁₋₃ alkylene forming R⁴ are each optionally substituted with 1, 2, or 3 substituents independently selected from R^{4A}; and wherein the C₁₋₃ alkyl forming R⁴ is optionally substituted with 1, 2, or 3 substituents independently selected from R^{4B};
each R^{4A} is independently selected from C₁₋₃ alkyl, C₁₋₃ haloalkyl, and R^{4B};
each R^{4B} is independently selected from C₃₋₆ cycloalkyl, 4-10 membered heterocycloalkyl, phenyl, 5-6 membered heteroaryl, and NR^{c4B}R^{d4B}; wherein the C₃-6 cycloalkyl, 4-6 membered heterocycloalkyl, phenyl, and 5-6 membered heteroaryl are each optionally substituted with 1, 2, or 3 substituents independently selected from R^{4C};
each R^{4C} is independently selected from C₁₋₃ alkyl, C₁₋₃ haloalkyl, halo, D, CN, OR^{a4C}, C(O)R^{b4C}, C(O)NR^{c4C}R^{d4C}, C(O)OR^{a4C}, and NR^{c4C}R^{d4C};
each Rc^{4B} and R^{d4B} is independently selected from H and C₁₋₃ alkyl;
each R^{a4C}, R^{b4C}, R^{c4C} and R^{d4C} is independently selected from H and C₁₋₃ alkyl;
R⁵ is selected from H and C₁₋₃ alkyl;
R⁶ is selected from H and C₁₋₃ alkyl
R⁷ is selected from H and C₁₋₃ alkyl; and

R⁸ is selected from H and C₁₋₃ alkyl.

In embodiments,

R¹ is selected from halo, C₁₋₃ alkyl, NR^{1B}R^{1C}, and NR^{1B}C(O)R^{1C};
R³ is selected from halo, C₁₋₃ alkyl, 5-6 membered heteroaryl, NR^{3B}R^{3C}, and NR^{3B}C(O)R^{3C};
R⁴ is selected from C₁₋₃ alkyl and 5-6 membered heteroaryl-C₁₋₃ alkylene; wherein the 5-6 membered heteroaryl-C₁₋₃ alkylene forming R⁴ is optionally substituted with 1, 2, or 3 substituents independently selected from R^{4A}; and wherein the C₁₋₃ alkyl forming R⁴ is optionally substituted with 1, 2, or 3 substituents independently selected from R^{4B}.

In embodiments of the compound of Formula (I), or pharmaceutically acceptable salt thereof, R⁴ is C₁₋₃ alkylene-R^{4B}, wherein C₁₋₃ alkylene is optionally substituted with Ra, wherein Ra is C₁₋₄ alkyl, e.g., C₁₋₃ alkyl, e.g., C₁₋₂ alkyl. In other embodiments of the compound of Formula (I), or pharmaceutically acceptable salt thereof, R⁴ is C(H)(R^{a})(R^{4B}), wherein R^{a} is C₁₋₂ alkyl.

In embodiments, the compound of Formula (I) is a compound of Formula (Ia)

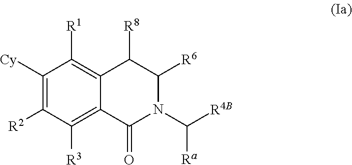

or a pharmaceutically acceptable salt thereof;
wherein R^{a} is C₁₋₂ alkyl.

In embodiments, the compound of Formula (I) is a compound of Formula (Ia′)

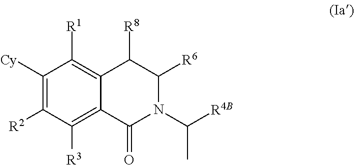

or a pharmaceutically acceptable salt thereof.

In embodiments, the compound of Formula (I) is a compound of Formula (Ib):

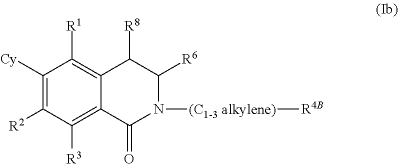

or a pharmaceutically acceptable salt thereof;
wherein
R^{4B} is 5-6 membered heteroaryl optionally substituted with 1, 2, or 3 substituents independently selected from R^{4C}.

In embodiments, the compound of Formula (I) is a compound of Formula (Ic) or Formula (Id):

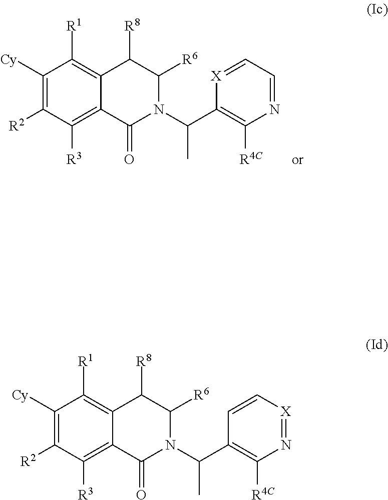

or a pharmaceutically acceptable salt thereof;
   wherein
X is CH or N.

In an embodiment, the compound of Formula (I) is a compound of Formula (Ic). In an embodiment, the compound of Formula (I) is a compound of Formula (Id).

In embodiments, the compound of Formula (I) is a compound of Formula (Ie) or Formula (If):

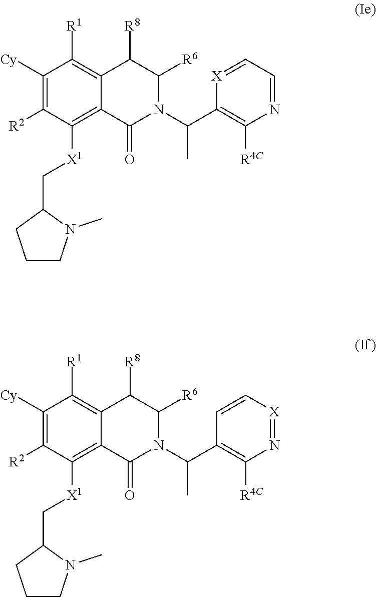

or a pharmaceutically acceptable salt thereof;
   wherein
X is CH or N; and
X¹ is O or NH.

In an embodiment, the compound of Formula (I) is a compound of Formula (Ie). In an embodiment, the compound of Formula (I) is a compound of Formula (If).

In embodiments, Cy is selected from phenyl and 5-10 membered heteroaryl; wherein the phenyl and 5-10 membered heteroaryl forming Cy is substituted with 1, 2, 3, or 4 substituents independently selected from R^{Cy}. In embodiments, Cy is selected from phenyl, benzothiophenyl, and benzothiazolyl, all of which are optionally substituted with 1, 2, 3, or 4 substituents independently selected from R^{Cy}. In other embodiments, Cy is selected from 1-cyano-2-amino-4-chloro-5-iodophen-3-yl, 1-cyano-4-methyl-3-(trifluoromethyl)phen-2-yl, 2-amino-5-chloro-3-cyano-benzo[b]thiophen-4-yl, 2-amino-5-fluoro-3-cyano-benzo[b]thiophen-4-yl, 2-amino-5-fluoro-benzo[b]thiophen-4-yl, 2-amino-7-fluoro-3-cyano-benzo[b]thiophen-4-yl, and 2-amino-7-fluorobenzo[d]thiazol-4-yl.

In other embodiments, each R^{Cy} is independently selected from D, C₁₋₃ alkyl, C₁₋₃ haloalkyl, C₂₋₃ alkenyl, C₂₋₃ alkynyl, halo, CN, OR^{aCy21}, and NR^{cCy21}R^{dCy21}. In other embodiments, each R^{Cy} is independently selected from halo, C₁₋₃ haloalkyl, CN, and NR^{cCy21}R^{dCy21}.

In other embodiments, R^{cCy21} and R^{dCy21} are each independently selected from H and C₁₋₃ alkyl.

In other embodiments, R⁴ is selected from C₁₋₃ alkyl, 4-10 membered heterocycloalkyl, and 5-6 membered heteroaryl-C₁₋₃ alkylene; wherein the 4-10 membered heterocycloalkyl and 5-6 membered heteroaryl-C₁₋₃ alkylene forming R⁴ are each optionally substituted with 1 or 2 substituents independently selected from R^{4A}; and wherein the C₁₋₃ alkyl forming R⁴ is optionally substituted with 1 or 2 substituents independently selected from R^{4B}.

In other embodiments, R⁴ is selected from C₁₋₃ alkyl and 5-6 membered heteroaryl-C₁₋₃ alkylene; wherein the 5-6 membered heteroaryl-C₁₋₃ alkylene forming R⁴ is optionally substituted with 1 or 2 substituents independently selected from R^{4A}; and wherein the C₁₋₃ alkyl forming R⁴ is optionally substituted with 1 or 2 substituents independently selected from R^{4B}.

In other embodiments, each R^{4B} is independently selected from 5-6 membered heteroaryl and NR^{c4B}R^{d4B}; wherein the 5-6 membered heteroaryl is optionally substituted with 1 or 2 substituents independently selected from R^{4C}. In other embodiments, R^{4B} is selected from 2-aminopyridin-3-yl, 2-aminopyrimidin-3-yl, 3-aminopyridazin-4-yl, and 1-methyl-3-aminopyrazol-4-yl.

In other embodiments, each R^{4C} is independently selected from NR^{c4c}R^{d4C}.

In other embodiments, R^{c4B} and R^{d4B} are each H.

In other embodiments, R^{c4C} and R^{d4C} are each H.

In other embodiments, R¹ is selected from halo, C₁₋₃ alkyl, CN, and C(O)NR^{1B}R^{1C}. In other embodiments, R¹ is selected from halo and C₁₋₃ alkyl. In other embodiments, R¹ is selected from chloro, fluoro, and methyl.

In other embodiments, R² is selected from halo and C₁₋₃ alkyl. In other embodiments, R² is selected from chloro, fluoro, and methyl.

In other embodiments, R³ is independently selected from H, halo, methyl, C₂₋₄ alkynyl, 5-6 membered heteroaryl, NR^{3B}R^{3C}, and NR^{3B}C(O)R^{3C}, wherein the C₂₋₄ alkynyl forming R³ is optionally substituted with 1 or 2 substituents independently selected from R^{3E}. In other embodiments, R³ is independently selected from H, halo, methyl, 5-6 membered heteroaryl, NR^{3B}R^{3C}, and NR^{3B}C(O)R^{3C}. In other embodiments, R³ is selected from H, methyl, 5 membered heteroaryl, NH₂, and NHC(O)CH₃. In other embodiments, R³ is selected from H, methyl, pyrazolyl, NH₂, and NHC(O)CH₃.

In other embodiments, R^{3B} is H; and R^{3C} is selected from H and C₁₋₃ alkyl. In other embodiments, R^{3B} is H. In other embodiments, R^{3C} is selected from H and C₁₋₃ alkyl.

In other embodiments, R⁵ is H. In other embodiments, R⁶ is H. In other embodiments, R⁷ is H. In other embodiments, R⁸ is H. In other embodiments, R⁷ is halo. In other embodiments, R⁸ is halo.

In other embodiments, R⁵ is H; R⁶ is H; R⁷ is H; and R⁸ is H. In other embodiments, R⁵ is H; R⁶ is H; R⁷ is halo; and Ra is halo.

In other embodiments, the compound of Formula (I) is selected from a compound in Table 1, and pharmaceutically acceptable salts thereof.

TABLE 1

| Entry | Compound Name |
|---|---|
| 1 | 2-amino-4-(2-(1-(2-aminopyridin-3-yl)ethyl)-5-chloro-7-fluoro-1-oxo-1,2,3,4- |
| | tetrahydroisoquinolin-6-yl)-7-fluorobenzo[b]thiophene-3-carbonitrile; |
| 2 | 2-amino-4-(2-(1-(2-aminopyridin-3-yl)ethyl)-7-fluoro-5-methyl-1-oxo-1,2,3,4- |
| | tetrahydroisoquinolin-6-yl)-7-fluorobenzo[b]thiophene-3-carbonitrile; |
| 3 | 2-amino-4-(2-(1-(2-aminopyridin-3-yl)ethyl)-5-chloro-7-fluoro-1-oxo-1,2,3,4- |
| | tetrahydroisoquinolin-6-yl)-5-fluorobenzo[b]thiophene-3-carbonitrile; |
| 4 | 6-amino-2-(2-(1-(2-aminopyridin-3-yl)ethyl)-5-chloro-7-fluoro-1-oxo-1,2,3,4- |
| | tetrahydroisoquinolin-6-yl)-4-methyl-3-(trifluoromethyl)benzonitrile; |
| 5 | 2-amino-4-(2-(1-(2-aminopyridin-3-yl)ethyl)-5-chloro-7-fluoro-1-oxo-1,2,3,4- |
| | tetrahydroisoquinolin-6-yl)-5-chlorobenzo[b]thiophene-3-carbonitrile; |
| 6 | 2-amino-4-(2-(1-(3-aminopyrazin-2-yl)ethyl)-5-chloro-7-fluoro-1-oxo-1,2,3,4- |
| | tetrahydroisoquinolin-6-yl)-7-fluorobenzo[b]thiophene-3-carbonitrile; |
| 7 | 2-amino-4-(2-((3-amino-1-methyl-1 H-pyrazol-4-yl)methyl)-5-chloro-7-fluoro-1-oxo- |
| | 1,2,3,4-tetrahydroisoquinolin-6-yl)-7-fluorobenzo[b]thiophene-3-carbonitrile; |
| 8 | 2-amino-4-(2-(1-(3-aminopyridazin-4-yl)ethyl)-5-chloro-7-fluoro-1-oxo-1,2,3,4- |
| | tetrahydroisoquinolin-6-yl)-7-fluorobenzo[b]thiophene-3-carbonitrile; |
| 9 | 4-(2-(1-(1 H-1,2,4-triazol-1-yl)propan-2-yl)-5-chloro-7-fluoro-1-oxo-1,2,3,4- |
| | tetrahydroisoquinolin-6-yl)-2-amino-7-fluorobenzo[b]thiophene-3-carbonitrile; |
| 10 | 2-amino-4-(8-amino-2-(1-(2-aminopyridin-3-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 11 | N-(6-(2-amino-3-cyano-7-fluorobenzo[b]thiophen-4-yl)-2-(1-(2-aminopyridin-3- |
| | yl)ethyl)-5-chloro-7-fluoro-1-oxo-1,2,3,4-tetrahydroisoquinolin-8-yl)acetamide; |
| 12 | 6-(2-amino-7-fluorobenzo[d]thiazol-4-yl)-2-(1-(2-aminopyridin-3-yl)ethyl)- |
| | 5-chloro-7-fluoro-8-(1 H-pyrazol-4-yl)-3,4-dihydroisoquinolin-1(2H)-one; |
| 13 | 8-amino-6-(2-amino-7-fluorobenzo[d]thiazol-4-yl)-2-(1-(2-aminopyridin-3-yl)ethyl)- |
| | 5-chloro-7-fluoro-3,4-dihydroisoquinolin-1(2H)-one; |
| 14 | 2-amino-4-(2-(1-(2-aminopyridin-3-yl)ethyl)-7-chloro-5-fluoro-1-oxo-1,2,3,4- |
| | tetrahydroisoquinolin-6-yl)-7-fluorobenzo[b]thiophene-3-carbonitrile |
| 15 | 2-amino-4-(2-(1-amino-6,7-dihydro-5H-cyclopenta[c]pyridin-7-yl)-5-chloro-7-fluoro- |
| | 1-oxo-1,2,3,4-tetrahydroisoquinolin-6-yl)-7-fluorobenzo[b]thiophene-3-carbonitrile; |
| 16 | 2-amino-4-(2-(1-(4-aminopyrimidin-5-yl)ethyl)-5-chloro-7-fluoro-1-oxo-1,2,3,4- |
| | tetrahydroisoquinolin-6-yl)-7-fluorobenzo[b]thiophene-3-carbonitrile; |
| 17 | 2-amino-4-(5-chloro-7-fluoro-2-(1-methyl-2-oxopyrrolidin-3-yl)-1-oxo-1,2,3,4- |
| | tetrahydroisoquinolin-6-yl)-7-fluorobenzo[b]thiophene-3-carbonitrile; |
| 18 | 2-amino-4-(2-((3-amino-1 H-pyrazol-4-yl)methyl)-5-chloro-7-fluoro-1-oxo-1,2,3,4- |
| | tetrahydroisoquinolin-6-yl)-7-fluorobenzo[b]thiophene-3-carbonitrile; |
| 19 | 6-(2-amino-3-cyano-7-fluorobenzo[b]thiophen-4-yl)-2-(1-(2-aminopyridin-3- |
| | yl)ethyl)-7-fluoro-1-oxo-1,2,3,4-tetrahydroisoquinoline-5-carbonitrile; |
| 20 | 2-amino-4-(2-(1-(2-aminopyridin-3-yl)ethyl)-5-ethynyl-7-fluoro-1-oxo-1,2,3,4- |
| | tetrahydroisoquinolin-6-yl)-7-fluorobenzo[b]thiophene-3-carbonitrile; |
| 21 | 2-amino-4-(2-(1-(2-aminopyridin-3-yl)ethyl)-7-fluoro-5-(hydroxymethyl)-1-oxo- |
| | 1,2,3,4-tetrahydroisoquinolin-6-yl)-7-fluorobenzo[b]thiophene-3-carbonitrile; |
| 22 | 2-amino-4-(2-(1-(2-aminopyridin-3-yl)ethyl)-5-(difluoromethyl)-7-fluoro-1-oxo- |
| | 1,2,3,4-tetrahydroisoquinolin-6-yl)-7-fluorobenzo[b]thiophene-3-carbonitrile; |
| 23 | 6-(2-amino-3-cyano-7-fluorobenzo[b]thiophen-4-yl)-2-(1-(2-aminopyridin-3- |
| | yl)ethyl)-7-fluoro-1-oxo-1,2,3,4-tetrahydroisoquinoline-5-carboxamide; |
| 24 | 2-amino-4-(2-(1-(2-aminopyridin-3-yl)ethyl)-5-chloro-4,4,7-trifluoro-1-oxo-1,2,3,4- |
| | tetrahydroisoquinolin-6-yl)-7-fluorobenzo[b]thiophene-3-carbonitrile; |
| 25 | 2-amino-4-(2-(1-(2-aminopyridin-3-yl)ethyl)-5-chloro-8-(3-(dimethylamino)prop-1- |
| | yn-1-yl)-7-fluoro-1-oxo-1,2,3,4-tetrahydroisoquinolin-6-yl)-7- |
| | fluorobenzo[b]thiophene-3-carbonitrile; |
| 26 | 2-amino-4-(2-(1-(2-aminopyridin-3-yl)ethyl)-5-chloro-8-(4-(dimethylamino)but-1-yn- |
| | 1-yl)-7-fluoro-1-oxo-1,2,3,4-tetrahydroisoquinolin-6-yl)-7-fluorobenzo[b] |
| | thiophene-3-carbonitrile; |
| 27 | 2-amino-4-(2-(1-(2-aminopyridin-3-yl)ethyl)-5-chloro-7-fluoro-8-((1-methylpyrrolidin- |
| | 2-yl)ethynyl)-1-oxo-1,2,3,4-tetrahydroisoquinolin-6-yl)-7-fluorobenzo[b] |
| | thiophene-3-carbonitrile; |
| 28 | 2-amino-4-(2-(1-(2-aminopyridin-3-yl)ethyl)-5-chloro-7-fluoro-8-methoxy-1-oxo- |
| | 1,2,3,4-tetrahydroisoquinolin-6-yl)-7-fluorobenzo[b]thiophene-3-carbonitrile; |
| 29 | 2-amino-4-(2-(1-(2-aminopyridin-3-yl)ethyl)-5-chloro-8-(2-(dimethylamino)ethoxy)- |
| | 7-fluoro-1-oxo-1,2,3,4-tetrahydroisoquinolin-6-yl)-7-fluorobenzo[b]thiophene-3- |
| | carbonitrile; |
| 30 | 6-(2-amino-3-cyano-7-fluorobenzo[b]thiophen-4-yl)-2-(1-(2-aminopyridin-3- |
| | yl)ethyl)-5-chloro-7-fluoro-1-oxo-1,2,3,4-tetrahydroisoquinoline-8-carbonitrile. |

In other embodiments, the compound of Formula (I) is selected from a compound in Table 2, and pharmaceutically acceptable salts thereof.

TABLE 2

| Entry | Compound Name |
|---|---|
| 1 | (Ra)-2-amino-4-(2-((R)-1-(2-aminopyridin-3-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 2 | (Sa)-2-amino-4-(2-((S)-1-(2-aminopyridin-3-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 3 | (Ra)-2-amino-4-(2-((S)-1-(2-aminopyridin-3-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 4 | (Sa)-2-amino-4-(2-((R)-1-(2-aminopyridin-3-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 5 | (Ra)-2-amino-4-(2-((R)-1-(2-aminopyridin-3-yl)ethyl)-7-fluoro-5-methyl-1-oxo- |
| | 1,2,3,4-tetrahydroisoquinolin-6-yl)-7-fluorobenzo[b]thiophene-3-carbonitrile; |
| 6 | (Sa)-2-amino-4-(2-((S)-1-(2-aminopyridin-3-yl)ethyl)-7-fluoro-5-methyl-1-oxo- |
| | 1,2,3,4-tetrahydroisoquinolin-6-yl)-7-fluorobenzo[b]thiophene-3-carbonitrile; |
| 7 | (Ra)-2-amino-4-(2-((S)-1-(2-aminopyridin-3-yl)ethyl)-7-fluoro-5-methyl-1-oxo- |
| | 1,2,3,4-tetrahydroisoquinolin-6-yl)-7-fluorobenzo[b]thiophene-3-carbonitrile; |
| 8 | (Sa)-2-amino-4-(2-((R)-1-(2-aminopyridin-3-yl)ethyl)-7-fluoro-5-methyl-1-oxo- |
| | 1,2,3,4-tetrahydroisoquinolin-6-yl)-7-fluorobenzo[b]thiophene-3-carbonitrile; |
| 9 | (Ra)-2-amino-4-(2-((R)-1-(2-aminopyridin-3-yl)ethyl)-5-chloro-7-fluoro-1-oxo- |
| | 1,2,3,4-tetrahydroisoquinolin-6-yl)-5-fluorobenzo[b]thiophene-3-carbonitrile; |
| 10 | (Sa)-2-amino-4-(2-((S)-1-(2-aminopyridin-3-yl)ethyl)-5-chloro-7-fluoro-1-oxo- |
| | 1,2,3,4-tetrahydroisoquinolin-6-yl)-5-fluorobenzo[b]thiophene-3-carbonitrile; |
| 11 | (Ra)-2-amino-4-(2-((S)-1-(2-aminopyridin-3-yl)ethyl)-5-chloro-7-fluoro-1-oxo- |
| | 1,2,3,4-tetrahydroisoquinolin-6-yl)-5-fluorobenzo[b]thiophene-3-carbonitrile; |
| 12 | (Sa)-2-amino-4-(2-((R)-1-(2-aminopyridin-3-yl)ethyl)-5-chloro-7-fluoro-1-oxo- |
| | 1,2,3,4-tetrahydroisoquinolin-6-yl)-5-fluorobenzo[b]thiophene-3-carbonitrile; |
| 13 | (Ra)-6-amino-2-(2-((R)-1-(2-aminopyridin-3-yl)ethyl)-5-chloro-7-fluoro-1-oxo- |
| | 1,2,3,4-tetrahydroisoquinolin-6-yl)-4-methyl-3-(trifluoromethyl)benzonitrile; |
| 14 | (Sa)-6-amino-2-(2-((S)-1-(2-aminopyridin-3-yl)ethyl)-5-chloro-7-fluoro-1-oxo- |
| | 1,2,3,4-tetrahydroisoquinolin-6-yl)-4-methyl-3-(trifluoromethyl)benzonitrile; |
| 15 | (Ra)-6-amino-2-(2-((S)-1-(2-aminopyridin-3-yl)ethyl)-5-chloro-7-fluoro-1-oxo- |
| | 1,2,3,4-tetrahydroisoquinolin-6-yl)-4-methyl-3-(trifluoromethyl)benzonitrile; |
| 16 | (Sa)-6-amino-2-(2-((R)-1-(2-aminopyridin-3-yl)ethyl)-5-chloro-7-fluoro-1-oxo- |
| | 1,2,3,4-tetrahydroisoquinolin-6-yl)-4-methyl-3-(trifluoromethyl)benzonitrile; |
| 17 | (Ra)-2-amino-4-(2-((R)-1-(2-aminopyridin-3-yl)ethyl)-5-chloro-7-fluoro-1-oxo- |
| | 1,2,3,4-tetrahydroisoquinolin-6-yl)-5-chlorobenzo[b]thiophene-3-carbonitrile; |
| 18 | (Sa)-2-amino-4-(2-((S)-1-(2-aminopyridin-3-yl)ethyl)-5-chloro-7-fluoro-1-oxo- |
| | 1,2,3,4-tetrahydroisoquinolin-6-yl)-5-chlorobenzo[b]thiophene-3-carbonitrile; |
| 19 | (Ra)-2-amino-4-(2-((S)-1-(2-aminopyridin-3-yl)ethyl)-5-chloro-7-fluoro-1-oxo- |
| | 1,2,3,4-tetrahydroisoquinolin-6-yl)-5-chlorobenzo[b]thiophene-3-carbonitrile; |
| 20 | (Sa)-2-amino-4-(2-((R)-1-(2-aminopyridin-3-yl)ethyl)-5-chloro-7-fluoro-1-oxo- |
| | 1,2,3,4-tetrahydroisoquinolin-6-yl)-5-chlorobenzo[b]thiophene-3-carbonitrile; |
| 21 | (Ra)-2-amino-4-(2-((R)-1-(3-aminopyrazin-2-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 22 | (Sa)-2-amino-4-(2-((S)-1-(3-aminopyrazin-2-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 23 | (Ra)-2-amino-4-(2-((S)-1-(3-aminopyrazin-2-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 24 | (Sa)-2-amino-4-(2-((R)-1-(3-aminopyrazin-2-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 25 | (Ra)-2-amino-4-(2-((3-amino-1-methyl-1 H-pyrazol-4-yl)methyl)-5-chloro-7-fluoro-1- |
| | oxo-1,2,3,4-tetrahydroisoquinolin-6-yl)-7-fluorobenzo[b]thiophene-3-carbonitrile; |
| 26 | (Sa)-2-amino-4-(2-((3-amino-1-methyl-1 H-pyrazol-4-yl)methyl)-5-chloro-7-fluoro-1- |
| | oxo-1,2,3,4-tetrahydroisoquinolin-6-yl)-7-fluorobenzo[b]thiophene-3-carbonitrile; |
| 27 | (Ra)-2-amino-4-(2-((R)-1-(3-aminopyridazin-4-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 28 | (Sa)-2-amino-4-(2-((S)-1-(3-aminopyridazin-4-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 29 | (Ra)-2-amino-4-(2-((S)-1-(3-aminopyridazin-4-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 30 | (Sa)-2-amino-4-(2-((R)-1-(3-aminopyridazin-4-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 31 | (Ra)-4-(2-((R)-1-(1H-1,2,4-triazol-1-yl)propan-2-yl)-5-chloro-7-fluoro-1-oxo-1,2,3,4- |
| | tetrahydroisoquinolin-6-yl)-2-amino-7-fluorobenzo[b]thiophene-3-carbonitrile; |
| 32 | (Sa)-4-(2-((S)-1-(1 H-1,2,4-triazol-1-yl)propan-2-yl)-5-chloro-7-fluoro-1-oxo-1,2,3,4- |
| | tetrahydroisoquinolin-6-yl)-2-amino-7-fluorobenzo[b]thiophene-3-carbonitrile; |
| 33 | (Ra)-4-(2-((S)-1-(1H-1,2,4-triazol-1-yl)propan-2-yl)-5-chloro-7-fluoro-1-oxo-1,2,3,4- |
| | tetrahydroisoquinolin-6-yl)-2-amino-7-fluorobenzo[b]thiophene-3-carbonitrile; |
| 34 | (Sa)-4-(2-((R)-1-(1H-1,2,4-triazol-1-yl)propan-2-yl)-5-chloro-7-fluoro-1-oxo-1,2,3,4- |
| | tetrahydroisoquinolin-6-yl)-2-amino-7-fluorobenzo[b]thiophene-3-carbonitrile; |
| 35 | (Ra)-2-amino-4-(8-amino-2-((R)-1-(2-aminopyridin-3-yl)ethyl)-5-chloro-7-fluoro-1- |
| | oxo-1,2,3,4-tetrahydroisoquinolin-6-yl)-7-fluorobenzo[b]thiophene-3-carbonitrile; |
| 36 | (Sa)-2-amino-4-(8-amino-2-((S)-1-(2-aminopyridin-3-yl)ethyl)-5-chloro-7-fluoro-1- |
| | oxo-1,2,3,4-tetrahydroisoquinolin-6-yl)-7-fluorobenzo[b]thiophene-3-carbonitrile; |
| 37 | (Ra)-2-amino-4-(8-amino-2-((S)-1-(2-aminopyridin-3-yl)ethyl)-5-chloro-7-fluoro-1- |
| | oxo-1,2,3,4-tetrahydroisoquinolin-6-yl)-7-fluorobenzo[b]thiophene-3-carbonitrile; |
| 38 | (Sa)-2-amino-4-(8-amino-2-((R)-1-(2-aminopyridin-3-yl)ethyl)-5-chloro-7-fluoro-1- |
| | oxo-1,2,3,4-tetrahydroisoquinolin-6-yl)-7-fluorobenzo[b]thiophene-3-carbonitrile; |
| 39 | N-(Ra)-(6-(2-amino-3-cyano-7-fluorobenzo[b]thiophen-4-yl)-2-((R)-1-(2- |
| | aminopyridin-3-yl)ethyl)-5-chloro-7-fluoro-1-oxo-1,2,3,4-tetrahydroisoquinolin-8- |
| | yl)acetamide; |
| 40 | N-(Sa)-(6-(2-amino-3-cyano-7-fluorobenzo[b]thiophen-4-yl)-2-((S)-1-(2- |
| | aminopyridin-3-yl)ethyl)-5-chloro-7-fluoro-1-oxo-1,2,3,4-tetrahydroisoquinolin-8- |
| | yl)acetamide; |
| 41 | N-(Ra)-(6-(2-amino-3-cyano-7-fluorobenzo[b]thiophen-4-yl)-2-((S)-1-(2- |
| | aminopyridin-3-yl)ethyl)-5-chloro-7-fluoro-1-oxo-1,2,3,4-tetrahydroisoquinolin-8- |
| | yl)acetamide; |
| 42 | N-(Sa)-(6-(2-amino-3-cyano-7-fluorobenzo[b]thiophen-4-yl)-2-((R)-1-(2- |
| | aminopyridin-3-yl)ethyl)-5-chloro-7-fluoro-1-oxo-1,2,3,4-tetrahydroisoquinolin-8- |
| | yl)acetamide; |
| 43 | (Ra)-6-(2-amino-7-fluorobenzo[d]thiazol-4-yl)-2-((R)-1-(2-aminopyridin-3-yl)ethyl)-5- |
| | chloro-7-fluoro-8-(1 H-pyrazol-4-yl)-3,4-dihydroisoquinolin-1(2H)-one; |
| 44 | (Sa)-6-(2-amino-7-fluorobenzo[d]thiazol-4-yl)-2-((S)-1-(2-aminopyridin-3-yl)ethyl)-5- |
| | chloro-7-fluoro-8-(1 H-pyrazol-4-yl)-3,4-dihydroisoquinolin-1(2H)-one; |
| 45 | (Ra)-6-(2-amino-7-fluorobenzo[d]thiazol-4-yl)-2-((S)-1-(2-aminopyridin-3-yl)ethyl)-5- |
| | chloro-7-fluoro-8-(1 H-pyrazol-4-yl)-3,4-dihydroisoquinolin-1(2H)-one; |
| 46 | (Sa)-6-(2-amino-7-fluorobenzo[d]thiazol-4-yl)-2-((R)-1-(2-aminopyridin-3-yl)ethyl)-5- |
| | chloro-7-fluoro-8-(1 H-pyrazol-4-yl)-3,4-dihydroisoquinolin-1(2H)-one; |
| 47 | (Ra)-8-amino-6-(2-amino-7-fluorobenzo[d]thiazol-4-yl)-2-((R)-1-(2-aminopyridin-3- |
| | yl)ethyl)-5-chloro-7-fluoro-3,4-dihydroisoquinolin-1(2H)-one; |
| 48 | (Sa)-8-amino-6-(2-amino-7-fluorobenzo[d]thiazol-4-yl)-2-((S)-1 -(2-aminopyridin-3- |
| | yl)ethyl)-5-chloro-7-fluoro-3,4-dihydroisoquinolin-1(2H)-one; |
| 49 | (Ra)-8-amino-6-(2-amino-7-fluorobenzo[d]thiazol-4-yl)-2-((S)-1-(2-aminopyridin-3- |
| | yl)ethyl)-5-chloro-7-fluoro-3,4-dihydroisoquinolin-1(2H)-one; |
| 50 | (Sa)-8-amino-6-(2-amino-7-fluorobenzo[d]thiazol-4-yl)-2-((R)-1-(2-aminopyridin-3- |
| | yl)ethyl)-5-chloro-7-fluoro-3,4-dihydroisoquinolin-1(2H)-one; |
| 51 | (Ra)-2-amino-4-(2-((R)-1-(2-aminopyridin-3-yl)ethyl)-7-chloro-5-fluoro-1-oxo- |
| | 1,2,3,4-tetrahydroisoquinolin-6-yl)-7-fluorobenzo[b]thiophene-3-carbonitrile; |
| 52 | (Sa)-2-amino-4-(2-((S)-1-(2-aminopyridin-3-yl)ethyl)-7-chloro-5-fluoro-1-oxo- |
| | 1,2,3,4-tetrahydroisoquinolin-6-yl)-7-fluorobenzo[b]thiophene-3-carbonitrile; |
| 53 | (Ra)-2-amino-4-(2-((S)-1-(2-aminopyridin-3-yl)ethyl)-7-chloro-5-fluoro-1-oxo- |
| | 1,2,3,4-tetrahydroisoquinolin-6-yl)-7-fluorobenzo[b]thiophene-3-carbonitrile; |
| 54 | (Sa)-2-amino-4-(2-((R)-1-(2-aminopyridin-3-yl)ethyl)-7-chloro-5-fluoro-1-oxo- |
| | 1,2,3,4-tetrahydroisoquinolin-6-yl)-7-fluorobenzo[b]thiophene-3-carbonitrile |
| 55 | 2-amino-4-((Ra)-2-(1-amino-6,7-dihydro-5H-cyclopenta[c]pyridin-7-yl)-5-chloro-7- |
| | fluoro-1-oxo-1,2,3,4-tetrahydroisoquinolin-6-yl)-7-fluorobenzo[b]thiophene-3- |
| | carbonitrile; |
| 56 | 2-amino-4-((Sa)-2-(1-amino-6,7-dihydro-5H-cyclopenta[c]pyridin-7-yl)-5-chloro-7- |
| | fluoro-1-oxo-1,2,3,4-tetrahydroisoquinolin-6-yl)-7-fluorobenzo[b]thiophene-3- |
| | carbonitrile; |
| 57 | 2-amino-4-((Ra)-2-((R)-1-(4-aminopyrimidin-5-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 58 | 2-amino-4-((Ra)-2-((S)-1-(4-aminopyrimidin-5-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 59 | 2-amino-4-((Sa)-2-((R)-1-(4-aminopyrimidin-5-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 60 | 2-amino-4-((Sa)-2-((S)-1-(4-aminopyrimidin-5-yl)ethyl)-5-chloro-7-fluoro-1-oxo- |
| | 1,2,3,4-tetrahydroisoquinolin-6-yl)-7-fluorobenzo[b]thiophene-3-carbonitrile; |
| 61 | 2-amino-4-((Ra)-5-chloro-7-fluoro-2-(1-methyl-2-oxopyrrolidin-3-yl)-1-oxo-1,2,3,4- |
| | tetrahydroisoquinolin-6-yl)-7-fluorobenzo[b]thiophene-3-carbonitrile; |
| 62 | 2-amino-4-((Sa)-5-chloro-7-fluoro-2-(1-methyl-2-oxopyrrolidin-3-yl)-1-oxo-1,2,3,4- |
| | tetrahydroisoquinolin-6-yl)-7-fluorobenzo[b]thiophene-3-carbonitrile; |
| 63 | (Ra)-2-amino-4-(2-((3-amino-1 H-pyrazol-4-yl)methyl)-5-chloro-7-fluoro-1-oxo- |
| | 1,2,3,4-tetrahydroisoquinolin-6-yl)-7-fluorobenzo[b]thiophene-3-carbonitrile; |
| 64 | (Sa)-2-amino-4-(2-((3-amino-1 H-pyrazol-4-yl)methyl)-5-chloro-7-fluoro-1-oxo- |
| | 1,2,3,4-tetrahydroisoquinolin-6-yl)-7-fluorobenzo[b]thiophene-3-carbonitrile; |
| 65 | (Ra)-6-(2-amino-3-cyano-7-fluorobenzo[b]thiophen-4-yl)-2-((R)-1-(2-aminopyridin- |
| | 3-yl)ethyl)-7-fluoro-1-oxo-1,2,3,4-tetrahydroisoquinoline-5-carbonitrile; |
| 66 | (Ra)-6-(2-amino-3-cyano-7-fluorobenzo[b]thiophen-4-yl)-2-((S)-1-(2-aminopyridin- |
| | 3-yl)ethyl)-7-fluoro-1-oxo-1,2,3,4-tetrahydroisoquinoline-5-carbonitrile; |
| 67 | (Sa)-6-(2-amino-3-cyano-7-fluorobenzo[b]thiophen-4-yl)-2-((R)-1-(2-aminopyridin- |
| | 3-yl)ethyl)-7-fluoro-1-oxo-1,2,3,4-tetrahydroisoquinoline-5-carbonitrile; |
| 68 | (Sa)-6-(2-amino-3-cyano-7-fluorobenzo[b]thiophen-4-yl)-2-((S)-1-(2-aminopyridin-3- |
| | yl)ethyl)-7-fluoro-1-oxo-1,2,3,4-tetrahydroisoquinoline-5-carbonitrile; |
| 69 | 2-amino-4-((Ra)-2-((R)-1-(2-aminopyridin-3-yl)ethyl)-5-ethynyl-7-fluoro-1-oxo- |
| | 1,2,3,4-tetrahydroisoquinolin-6-yl)-7-fluorobenzo[b]thiophene-3-carbonitrile; |
| 70 | 2-amino-4-((Ra)-2-((S)-1-(2-aminopyridin-3-yl)ethyl)-5-ethynyl-7-fluoro-1-oxo- |
| | 1,2,3,4-tetrahydroisoquinolin-6-yl)-7-fluorobenzo[b]thiophene-3-carbonitrile; |
| 71 | 2-amino-4-((Sa)-2-((R)-1-(2-aminopyridin-3-yl)ethyl)-5-ethynyl-7-fluoro-1-oxo- |
| | 1,2,3,4-tetrahydroisoquinolin-6-yl)-7-fluorobenzo[b]thiophene-3-carbonitrile; |
| 72 | 2-amino-4-((Sa)-2-((S)-1-(2-aminopyridin-3-yl)ethyl)-5-ethynyl-7-fluoro-1-oxo- |
| | 1,2,3,4-tetrahydroisoquinolin-6-yl)-7-fluorobenzo[b]thiophene-3-carbonitrile; |
| 73 | 2-amino-4-((Ra)-2-((R)-1-(2-aminopyridin-3-yl)ethyl)-7-fluoro-5-(hydroxymethyl)-1- |
| | oxo-1,2,3,4-tetrahydroisoquinolin-6-yl)-7-fluorobenzo[b]thiophene-3-carbonitrile; |
| 74 | 2-amino-4-((Ra)-2-((S)-1-(2-aminopyridin-3-yl)ethyl)-7-fluoro-5-(hydroxymethyl)-1- |
| | oxo-1,2,3,4-tetrahydroisoquinolin-6-yl)-7-fluorobenzo[b]thiophene-3-carbonitrile; |
| 75 | 2-amino-4-((Sa)-2-((R)-1-(2-aminopyridin-3-yl)ethyl)-7-fluoro-5-(hydroxymethyl)-1- |
| | oxo-1,2,3,4-tetrahydroisoquinolin-6-yl)-7-fluorobenzo[b]thiophene-3-carbonitrile; |
| 76 | 2-amino-4-((Sa)-2-((S)-1-(2-aminopyridin-3-yl)ethyl)-7-fluoro-5-(hydroxymethyl)-1- |
| | oxo-1,2,3,4-tetrahydroisoquinolin-6-yl)-7-fluorobenzo[b]thiophene-3-carbonitrile; |
| 77 | 2-amino-4-((Ra)-2-((R)-1-(2-aminopyridin-3-yl)ethyl)-5-(difluoromethyl)-7-fluoro-1- |
| | oxo-1,2,3,4-tetrahydroisoquinolin-6-yl)-7-fluorobenzo[b]thiophene-3-carbonitrile; |
| 78 | 2-amino-4-((Ra)-2-((S)-1-(2-aminopyridin-3-yl)ethyl)-5-(difluoromethyl)-7-fluoro-1- |
| | oxo-1,2,3,4-tetrahydroisoquinolin-6-yl)-7-fluorobenzo[b]thiophene-3-carbonitrile; |
| 79 | 2-amino-4-((Sa)-2-((R)-1-(2-aminopyridin-3-yl)ethyl)-5-(difluoromethyl)-7-fluoro-1- |
| | oxo-1,2,3,4-tetrahydroisoquinolin-6-yl)-7-fluorobenzo[b]thiophene-3-carbonitrile; |
| 80 | 2-amino-4-((Sa)-2-((S)-1-(2-aminopyridin-3-yl)ethyl)-5-(difluoromethyl)-7-fluoro-1- |
| | oxo-1,2,3,4-tetrahydroisoquinolin-6-yl)-7-fluorobenzo[b]thiophene-3-carbonitrile; |
| 81 | (Ra)-6-(2-amino-3-cyano-7-fluorobenzo[b]thiophen-4-yl)-2-((R)-1-(2-aminopyridin- |
| | 3-yl)ethyl)-7-fluoro-1-oxo-1,2,3,4-tetrahydroisoquinoline-5-carboxamide; |
| 82 | (Ra)-6-(2-amino-3-cyano-7-fluorobenzo[b]thiophen-4-yl)-2-((S)-1-(2-aminopyridin- |
| | 3-yl)ethyl)-7-fluoro-1-oxo-1,2,3,4-tetrahydroisoquinoline-5-carboxamide; |
| 83 | (Sa)-6-(2-amino-3-cyano-7-fluorobenzo[b]thiophen-4-yl)-2-((R)-1-(2-aminopyridin- |
| | 3-yl)ethyl)-7-fluoro-1-oxo-1,2,3,4-tetrahydroisoquinoline-5-carboxamide; |
| 84 | (Sa)-6-(2-amino-3-cyano-7-fluorobenzo[b]thiophen-4-yl)-2-((S)-1-(2-aminopyridin-3- |
| | yl)ethyl)-7-fluoro-1-oxo-1,2,3,4-tetrahydroisoquinoline-5-carboxamide; |
| 85 | 2-amino-4-((Ra)-2-((R)-1-(2-aminopyridin-3-yl)ethyl)-5-chloro-4,4,7-trifluoro-1-oxo- |
| | 1,2,3,4-tetrahydroisoquinolin-6-yl)-7-fluorobenzo[b]thiophene-3-carbonitrile; |
| 86 | 2-amino-4-((Ra)-2-((S)-1-(2-aminopyridin-3-yl)ethyl)-5-chloro-4,4,7-trifluoro-1-oxo- |
| | 1,2,3,4-tetrahydroisoquinolin-6-yl)-7-fluorobenzo[b]thiophene-3-carbonitrile; |
| 87 | 2-amino-4-((Sa)-2-((R)-1-(2-aminopyridin-3-yl)ethyl)-5-chloro-4,4,7-trifluoro-1-oxo- |
| | 1,2,3,4-tetrahydroisoquinolin-6-yl)-7-fluorobenzo[b]thiophene-3-carbonitrile; |
| 88 | 2-amino-4-((Sa)-2-((S)-1-(2-aminopyridin-3-yl)ethyl)-5-chloro-4,4,7-trifluoro-1-oxo- |
| | 1,2,3,4-tetrahydroisoquinolin-6-yl)-7-fluorobenzo[b]thiophene-3-carbonitrile; |
| 89 | 2-amino-4-((Ra)-2-((R)-1-(2-aminopyridin-3-yl)ethyl)-5-chloro-8-(3- |
| | (dimethylamino)prop-1-yn-1-yl)-7-fluoro-1-oxo-1,2,3,4-tetrahydroisoquinolin-6-yl)-7- |
| | fluorobenzo[b]thiophene-3-carbonitrile; |
| 90 | 2-amino-4-((Ra)-2-((S)-1-(2-aminopyridin-3-yl)ethyl)-5-chloro-8-(3- |
| | (dimethylamino)prop-1-yn-1-yl)-7-fluoro-1-oxo-1,2,3,4-tetrahydroisoquinolin-6-yl)-7- |
| | fluorobenzo[b]thiophene-3-carbonitrile; |
| 91 | 2-amino-4-((Sa)-2-((R)-1-(2-aminopyridin-3-yl)ethyl)-5-chloro-8-(3- |
| | (dimethylamino)prop-1-yn-1-yl)-7-fluoro-1-oxo-1,2,3,4-tetrahydroisoquinolin-6-yl)-7- |
| | fluorobenzo[b]thiophene-3-carbonitrile; |
| 92 | 2-amino-4-((Sa)-2-((S)-1-(2-aminopyridin-3-yl)ethyl)-5-chloro-8-(3- |
| | (dimethylamino)prop-1-yn-1-yl)-7-fluoro-1-oxo-1,2,3,4-tetrahydroisoquinolin-6-yl)-7- |
| | fluorobenzo[b]thiophene-3-carbonitrile; |
| 93 | 2-amino-4-((Ra)-2-((R)-1-(2-aminopyridin-3-yl)ethyl)-5-chloro-8-(4- |
| | (dimethylamino)but-1-yn-1-yl)-7-fluoro-1-oxo-1,2,3,4-tetrahydroisoquinolin-6-yl)-7- |
| | fluorobenzo[b]thiophene-3-carbonitrile; |
| 94 | 2-amino-4-((Ra)-2-((S)-1-(2-aminopyridin-3-yl)ethyl)-5-chloro-8-(4- |
| | (dimethylamino)but-1-yn-1-yl)-7-fluoro-1-oxo-1,2,3,4-tetrahydroisoquinolin-6-yl)-7- |
| | fluorobenzo[b]thiophene-3-carbonitrile; |
| 95 | 2-amino-4-((Sa)-2-((R)-1-(2-aminopyridin-3-yl)ethyl)-5-chloro-8-(4- |
| | (dimethylamino)but-1-yn-1-yl)-7-fluoro-1-oxo-1,2,3,4-tetrahydroisoquinolin-6-yl)-7- |
| | fluorobenzo[b]thiophene-3-carbonitrile; |
| 96 | 2-amino-4-((Sa)-2-((S)-1-(2-aminopyridin-3-yl)ethyl)-5-chloro-8-(4- |
| | (dimethylamino)but-1-yn-1-yl)-7-fluoro-1-oxo-1,2,3,4-tetrahydroisoquinolin-6-yl)-7- |
| | fluorobenzo[b]thiophene-3-carbonitrile; |
| 97 | 2-amino-4-((Ra)-2-((R)-1-(2-aminopyridin-3-yl)ethyl)-5-chloro-7-fluoro-8-((1- |
| | methylpyrrolidin-2-yl)ethynyl)-1-oxo-1,2,3,4-tetrahydroisoquinolin-6-yl)-7- |
| | fluorobenzo[b]thiophene-3-carbonitrile; |
| 98 | 2-amino-4-((Ra)-2-((S)-1-(2-aminopyridin-3-yl)ethyl)-5-chloro-7-fluoro-8-((1- |
| | methylpyrrolidin-2-yl)ethynyl)-1-oxo-1,2,3,4-tetrahydroisoquinolin-6-yl)-7- |
| | fluorobenzo[b]thiophene-3-carbonitrile; |
| 99 | 2-amino-4-((Sa)-2-((R)-1-(2-aminopyridin-3-yl)ethyl)-5-chloro-7-fluoro-8-((1- |
| | methylpyrrolidin-2-yl)ethynyl)-1-oxo-1,2,3,4-tetrahydroisoquinolin-6-yl)-7- |
| | fluorobenzo[b]thiophene-3-carbonitrile; |
| 100 | 2-amino-4-((Sa)-2-((S)-1-(2-aminopyridin-3-yl)ethyl)-5-chloro-7-fluoro-8-((1- |
| | methylpyrrolidin-2-yl)ethynyl)-1-oxo-1,2,3,4-tetrahydroisoquinolin-6-yl)-7- |
| | fluorobenzo[b]thiophene-3-carbonitrile; |
| 101 | 2-amino-4-((Ra)-2-((R)-1-(2-aminopyridin-3-yl)ethyl)-5-chloro-7-fluoro-8-methoxy-1- |
| | oxo-1,2,3,4-tetrahydroisoquinolin-6-yl)-7-fluorobenzo[b]thiophene-3-carbonitrile; |
| 102 | 2-amino-4-((Ra)-2-((S)-1-(2-aminopyridin-3-yl)ethyl)-5-chloro-7-fluoro-8-methoxy-1- |
| | oxo-1,2,3,4-tetrahydroisoquinolin-6-yl)-7-fluorobenzo[b]thiophene-3-carbonitrile; |
| 103 | 2-amino-4-((Sa)-2-((R)-1-(2-aminopyridin-3-yl)ethyl)-5-chloro-7-fluoro-8-methoxy-1- |
| | oxo-1,2,3,4-tetrahydroisoquinolin-6-yl)-7-fluorobenzo[b]thiophene-3-carbonitrile; |
| 104 | 2-amino-4-((Sa)-2-((S)-1-(2-aminopyridin-3-yl)ethyl)-5-chloro-7-fluoro-8-methoxy-1- |
| | oxo-1,2,3,4-tetrahydroisoquinolin-6-yl)-7-fluorobenzo[b]thiophene-3-carbonitrile; |
| 105 | 2-amino-4-((Ra)-2-((R)-1-(2-aminopyridin-3-yl)ethyl)-5-chloro-8-(2- |
| | (dimethylamino)ethoxy)-7-fluoro-1-oxo-1,2,3,4-tetrahydroisoquinolin-6-yl)-7- |
| | fluorobenzo[b]thiophene-3-carbonitrile; |
| 106 | 2-amino-4-((Ra)-2-((S)-1-(2-aminopyridin-3-yl)ethyl)-5-chloro-8-(2- |
| | (dimethylamino)ethoxy)-7-fluoro-1-oxo-1,2,3,4-tetrahydroisoquinolin-6-yl)-7- |
| | fluorobenzo[b]thiophene-3-carbonitrile; |
| 107 | 2-amino-4-((Sa)-2-((R)-1-(2-aminopyridin-3-yl)ethyl)-5-chloro-8-(2- |
| | (dimethylamino)ethoxy)-7-fluoro-1-oxo-1,2,3,4-tetrahydroisoquinolin-6-yl)-7- |
| | fluorobenzo[b]thiophene-3-carbonitrile; |
| 108 | 2-amino-4-((Sa)-2-((S)-1-(2-aminopyridin-3-yl)ethyl)-5-chloro-8-(2- |
| | (dimethylamino)ethoxy)-7-fluoro-1-oxo-1,2,3,4-tetrahydroisoquinolin-6-yl)-7- |
| | fluorobenzo[b]thiophene-3-carbonitrile; |
| 109 | (Ra)-6-(2-amino-3-cyano-7-fluorobenzo[b]thiophen-4-yl)-2-((R)-1-(2-aminopyridin- |
| | 3-yl)ethyl)-5-chloro-7-fluoro-1-oxo-1,2,3,4-tetrahydroisoquinoline-8-carbonitrile; |
| 110 | (Ra)-6-(2-amino-3-cyano-7-fluorobenzo[b]thiophen-4-yl)-2-((S)-1-(2-aminopyridin- |
| | 3-yl)ethyl)-5-chloro-7-fluoro-1-oxo-1,2,3,4-tetrahydroisoquinoline-8-carbonitrile; |
| 111 | (Sa)-6-(2-amino-3-cyano-7-fluorobenzo[b]thiophen-4-yl)-2-((R)-1-(2-aminopyridin- |
| | 3-yl)ethyl)-5-chloro-7-fluoro-1-oxo-1,2,3,4-tetrahydroisoquinoline-8-carbonitrile; |
| 112 | (Sa)-6-(2-amino-3-cyano-7-fluorobenzo[b]thiophen-4-yl)-2-((S)-1-(2-aminopyridin-3- |
| | yl)ethyl)-5-chloro-7-fluoro-1-oxo-1,2,3,4-tetrahydroisoquinoline-8-carbonitrile. |

In other embodiments, the compound of Formula (I) is in the form of a pharmaceutically acceptable salt. In other embodiments, the compound of Formula (I) is in the form of a free base or free acid, or other than in the form of a salt.

In another aspect, provided herein is a pharmaceutical composition comprising a compound of Formula (I), or any of the embodiments thereof, or a pharmaceutically acceptable salt thereof, and a pharmaceutically acceptable carrier.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The hydrogen atom is formally removed and replaced by a substituent. A single divalent substituent, e.g., oxo, can replace two hydrogen atoms. The term “optionally substituted” means unsubstituted or substituted.

The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule.

The term “Cₙ₋ₘ” indicates a range which includes the endpoints, wherein n and m are integers and indicate the number of carbons present in a chemical moiety. The term is intended to include each and every member in the indicated range. Thus, Cₙ₋ₘ includes each member in the series Cₙ, Cₙ₊₁, . . . Cₘ₋₁, and Cₘ. Examples include C₁₋₄(which includes C₁, C₂, C₃, and C₄), C₁₋₆ (which includes C₁, C₂, C₃, C₄, C₅, and C₆) and the like.

The term “alkyl” employed alone or in combination with other terms, refers to a saturated hydrocarbon group that may be straight-chained or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkenyl,” employed alone or in combination with other terms, refers to a straight-chain or branched hydrocarbon group corresponding to an alkyl group having one or more carbon-carbon double bonds. The term “Cₙ₋ₘ alkylenyl” refers to an alkenyl group having n to m carbon atoms. An alkenyl group formally corresponds to an alkene with one C—H bond replaced by the point of attachment of the alkenyl group to the remainder of the compound. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

The term “alkynyl,” employed alone or in combination with other terms, refers to a straight-chain or branched hydrocarbon group corresponding to an alkyl group having one or more carbon-carbon triple bonds. The term “Cₙ₋ₘ alkynyl” refers to an alkynyl group having n to m carbon atoms. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. The term “Cₙ₋ₘ dialkoxy refers to a linking group of formula —O—(Cₙ₋ₘ alkyl)-O—, the alkyl group of which has n to m carbons. Example dialkoxy groups include —OCH₂CH₂O— and OCH₂CH₂CH₂O—. In some embodiments, the two O atoms of a Cₙ₋ₘ dialkoxy group may be attached to the same B atom to form a 5- or 6-membered heterocycloalkyl group.

The term “amino,” employed alone or in combination with other terms, refers to a group of formula —NH₂, wherein the hydrogen atoms may be substituted with a substituent described herein. For example, “alkylamino” can refer to —NH(alkyl) and —N(alkyl)₂.

The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “perfluoroalkyl” refers to a haloalkyl group in which all hydrogen atoms have been replaced by fluorine atoms. Example perfluoroalkyl groups include CF₃, C₂F₅, and C₃F₇.

The term “haloalkoxy,” employed alone or in combination with other terms, refers to a group of formula —O-haloalkyl, wherein the haloalkyl group is as defined above. The term “Cₙ₋ₘ haloalkoxy refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” or “oxy refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “carbonyl,” employed alone or in combination with other terms, refers to a —C(═O)— group, which also may be written as C(O).

The term “sulfonyl” refers to a —SO₂— group wherein a sulfur atom is doubly bound to two oxygen atoms.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, isoindolyl, benzothiophenyl, benzofuranyl, benzisoxazolyl, imidazo[1,2-b]thiazolyl, purinyl, and the like. In some embodiments, the heteroaryl group is pyridone (e.g., 2-pyridone).

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl” or “cycloalkane” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” or “Cₙ₋ₘ cycloalkane” refers to a cycloalkyl or cycloalkane that has n to m ring member carbon atoms. Cycloalkyl groups can include mono- or polycyclic (e.g., having 2, 3 or 4 fused rings) groups and spirocycles. In the following, references to cycloalkyl groups apply equally to cycloalkane groups. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl. In some embodiments, the cycloalkyl group is tetrahydronaphthalenyl (e.g., 1,2,3,4-tetrahydronaphthalenyl).

The term “heterocycloalkyl” or “heterocycloalkane” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members.

The term “n-m-membered heterocycloalkyl” or “n-m-membered heterocycloalkane” where n and m are integers refer to a heterocycloalkyl or heterocyclalkane ring or ring system containing from n to m ring-forming atoms. An n-m-membered heterocycloalkyl or heterocycloalkane includes from 1 to m−1 carbon atoms and from 1 to m−1 heteroatoms. The term “n-m-membered heterocycloalkyl” or “n-m-membered heterocycloalkane” where n is an integer refers to a heterocycloalkyl ring or ring system containing from n to m ring-forming atoms. In the following, references to heterocycloalkyl groups, rings or ring systems apply equally to heterocycloalkane groups, rings or ring systems. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2,5-diazobicyclo[2.2.1]heptanyl; pyrrolidinyl; hexahydropyrrolo[3,4-b]pyrrol-1(2H)-yl; 1,6-dihydropyridinyl; morpholinyl; azetidinyl; piperazinyl; 4,7-diazaspiro[2.5]octan-7-yl, and 6,7-dihydro-5H-cyclopenta[c]pyridinyl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

When the compounds described herein contain a chiral center, unless otherwise indicated, the compounds can be any of the possible stereoisomers. In some embodiments, the compounds provided herein have the (R)-configuration. In other embodiments, the compounds have the (S)-configuration. In compounds with more than one chiral centers, each of the chiral centers in the compound may be independently (R) or (S), unless otherwise indicated. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution. Compounds herein identified by name or structure as one particular tautomeric form are intended to include other tautomeric forms unless otherwise specified (e.g., in the case of purine rings, unless otherwise indicated, if a compound name or structure described the 9H tautomer, it would be understood that the 7H tautomer is also encompassed).

Compounds provided herein can exist in the form of atropisomers (i.e., conformational diastereoisomers) that can be stable at ambient temperature and separable, e.g., by chromatography. For example, the compounds of Formula (I) can exist in the form of atropisomers that are interchangeable by rotation around the bond connecting Cy¹ (or any of the embodiments thereof) to the remainder of the molecule. Reference to the compounds described herein or any of the embodiments is understood to include all such atropisomeric forms of the compounds. Without being limited by any theory, it is understood that, for a given compound, one atropisomer may be more potent as an inhibitor of KRAS (including G12D mutated form of KRAS) than another atropisomer. For example, compounds of Formula (I) as described herein in which Cy¹ is 2,3-dichlorophenyl can exist in the form of atropisomers in which the conformation of the dichlorophenyl relative to the remainder of the molecule is as shown by the partial formulae Formula A or Formula B below. Atropisomeric forms may be isolable by methods such as chromatography. The stereochemistry of atropisomeric forms can be designated, e.g., as either Rₐ or Sₐ by applying IUPAC rules of nomenclature for stereochemistry. G. P. Moss, Pure & Appl. Chem., 1996, 68(12), 2193-2222. Without being limited by any theory, it is understood that, for a given compound, the atropisomer represented by Formula (A) is generally more potent as an inhibitor of KRAS (including G12C, G12D or G12V mutated forms of KRAS) than the atropisomer represented by Formula (B). In some embodiments, an atropisomer can be least partially or substantially separated from the alternative atropisomer of the compound, for example containing about 40% or less, about 30% or less, about 20% or less, about 10% or less, about 5% or less, about 2% or less, or about 1% or less of the alternative atropisomer.

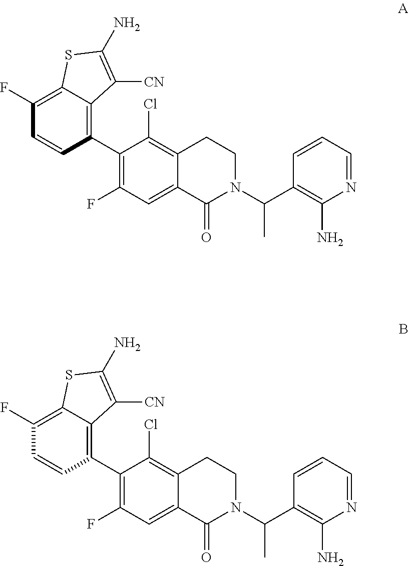

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. In the compounds provided herein, any atom not specifically designated as a particular isotope is meant to represent any stable isotope of that atom.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et.al. J. Med. Chem. 2011, 54, 201-210; R. Xu et.al. J. Label Compd. Radiopharm. 2015, 58, 308-312). Unless otherwise stated, when a position is designated specifically as “D” or “deuterium,” the position is understood to have deuterium at an abundance that is at least 3000 times greater than the natural abundance of deuterium, which is 0.015% (i.e., at least 45% incorporation of deuterium). In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “compound” is intended, unless otherwise specified, to include all stereoisomers, including without limitation, geometric isomers, configurational isomers, conformational isomers, rotational isomers, and atropisomers, of the structures depicted, including each of the embodiments thereof. The term is also intended to refer to compounds described herein regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds provided herein, or salts thereof, are substantially isolated. “Substantially isolated” means that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions “ambient temperature” and “room temperature” are understood in the art, and refer generally to a temperature, e.g., a reaction temperature, that is about the temperature of the room in which the reaction is carried out, e.g., a temperature from about 20° C. to about 30° C.

The present disclosure also includes pharmaceutically acceptable salts of the compounds described herein, including any of the embodiments thereof. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011). In some embodiments, the compounds described herein include the N-oxide forms.

### II. Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in P. Kocienski, Protecting Groups, 3^{rd} Ed. (Thieme, 2005); J. Robertson, Protecting Group Chemistry, (Oxford University Press, 2000); M. B. Smith et al., March's Advanced Organic Chemistry: Reactions, Mechanisms, and Structure, 8^{th} Ed. (Wiley, 2020); S. Pétursson, J. Chem. Educ., 1997, 74(11), 1297-303; and P. G. M. Wuts et al., Greene's Protective Groups in Organic Synthesis, 5^{th} Ed., (Wiley, 2014).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high-performance liquid chromatography (HPLC) or thin layer chromatography (TLC).

The Schemes below provide general guidance in connection with preparing the compounds of the present disclosure. One skilled in the art would understand that the preparations shown in the Schemes can be modified or optimized using general knowledge of organic chemistry to prepare various compounds provided herein.

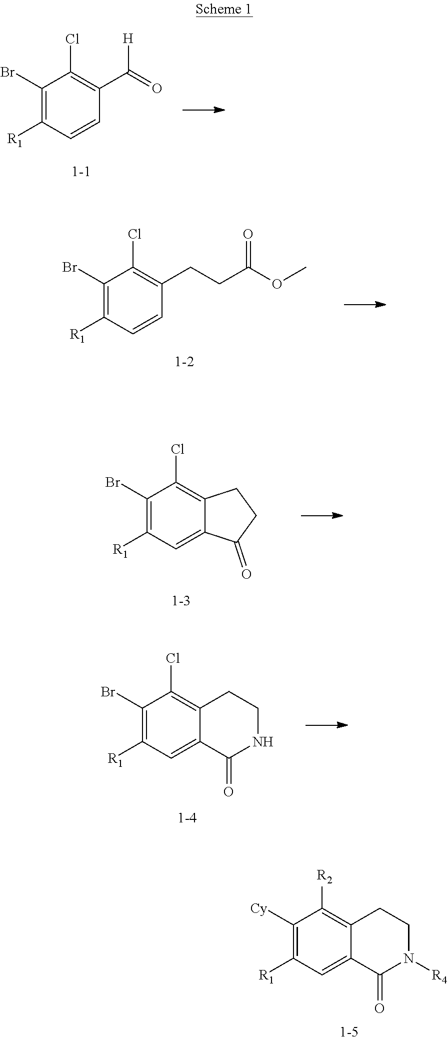

Compounds of this invention can be prepared via a number of key intermediates, depending on the modifications desired. In one example, compounds can be prepared from intermediate 1-3. An example synthesis is shown in Scheme 1. The aldehyde of functionalized starting material 1-1 is converted in a two-step sequence to a substituted aryl propionate derivative 1-2, through a sequence of Wittig olefination and appropriate conjugate reduction. Cyclization of the propionate 1-2 to provide 1-3 can proceed under a number of different conditions, including, for instance, Bronsted or Lewis acid catalyzed or mediated conditions. A rearrangement of 1-3 provides intermediate 1-4. The intermediate can be functionalized through a number of interchangeable steps. For instance, the amide nitrogen can be functionalized through a variety of conditions, including alkylation, SnAr displacement, Mitsunobu displacement or a cross coupling reaction involving an appropriate aryl halide or triflate. Alternatively, the aryl bromide of 1-4 can be reacted with an appropriate metal reagent under suitable catalytic palladium or nickel conditions, such as a Suzuki cross coupling reaction with an appropriate boronic acid or ester; a Stille coupling with the appropriate tin reagent, or a Negishi coupling with an appropriate zincate. Optionally, the aryl chloro substituent can be further functionalized using the same conditions.

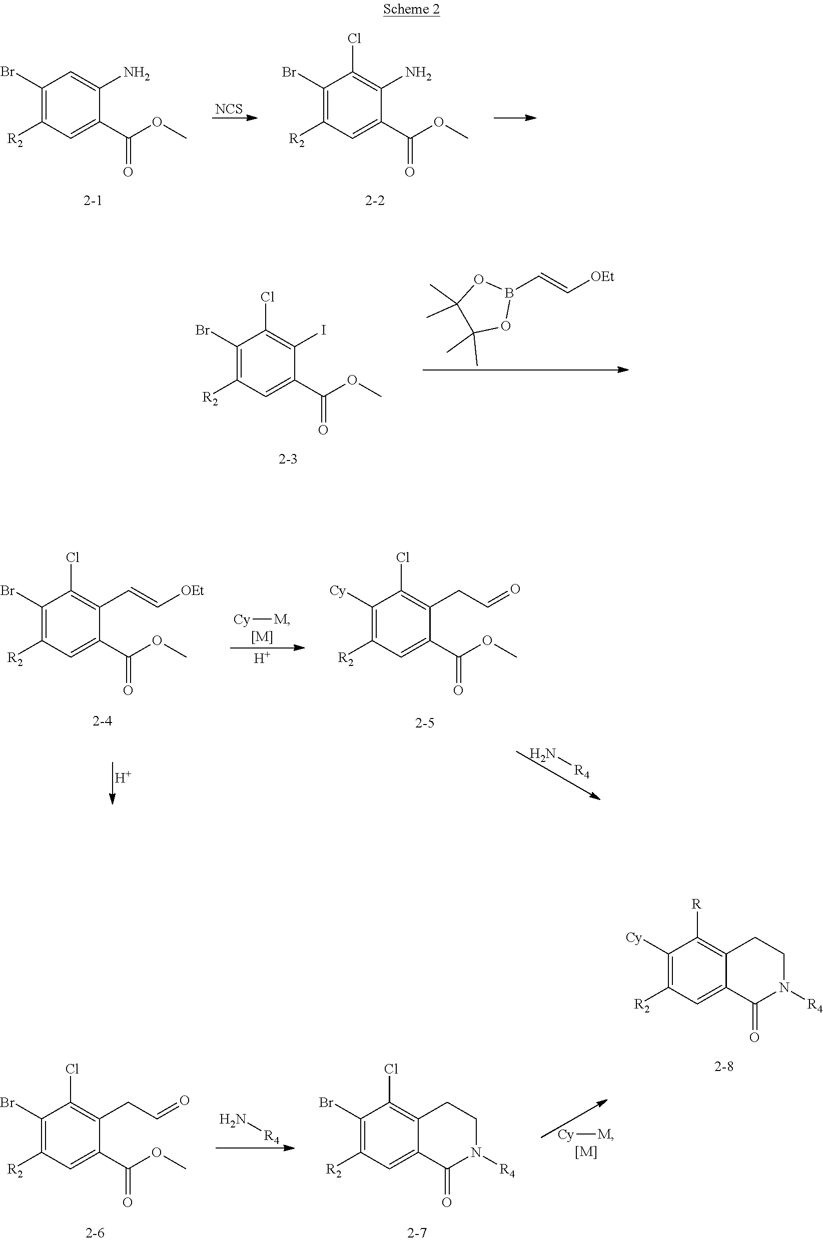

Alternatively, compounds can be synthesized from key intermediate 2-4. The route in Scheme 2 provides an example synthesis of this intermediate. A functionalized aniline of general formula 2-1 is chlorinated with NCS under suitable conditions to provide 2-2, which in turn undergoes a Sandmeyer halogenation to convert the aniline to the corresponding aryl iodide 2-3. Cross coupling with ethoxy vinyl boronic acid pinacol ester provides compounds of general structure 2-4. This intermediate can be functionalized interchangeably. In one option, the aryl bromide in 2-4 is converted to the desired cycle by an appropriate cross coupling reagent catalyzed by an appropriate palladium or nickel catalyst, such as a Suzuki cross coupling reaction with an appropriate boronic acid or ester; a Stille coupling with the appropriate tin reagent, or a Negishi coupling with an appropriate zincate. This is followed by mild acid hydrolysis of the vinyl ether to arrive at general aldehyde intermediate 2-5. Reductive amination with the appropriate amine is followed by cyclization, either over the course of the reaction or in a separate step with an appropriate base. In an alternative approach, the steps are reversed, with acid catalyzed vinyl ether hydrolysis and reductive amination/cyclization performed prior to installation of the appropriate Cy fragment. In either option, the final compound can be functionalized at the aryl chloride to provide compounds of general formula 2-8.

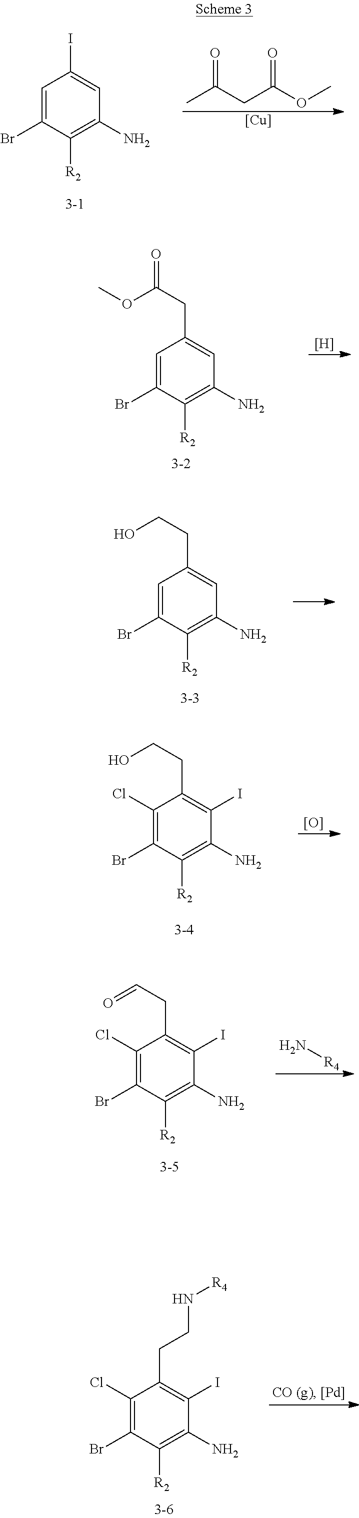

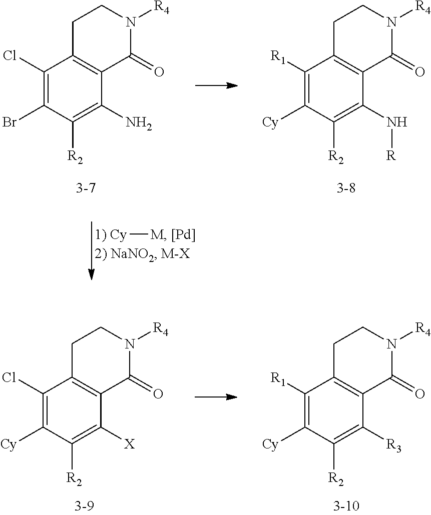

If substitution at R₃ is desired, key intermediate 3-7 may be used, which can be synthesized by, for instance, the route in Scheme 3. Starting from functionalized starting material 3-1, coupling with methyl 3-oxobutanoate under the influence of an appropriate copper catalyst provides 3-2. Reduction and iodination with an appropriate iodide source, such as molecular iodine, provides compound 3-4. Oxidation followed by reductive amination with an appropriate amine using a variety of conditions, i.e., NaBH(OAc)₃ with the optional use of TFA, provides 3-6. Carbonylative cyclization of 3-5 may be affected with an appropriate palladium catalyst, such as dppf-PdCl₂ under an atmosphere of carbon monoxide to provide cyclized product 3-7. This versatile intermediate can be transformed to the desired compounds in variety of approaches. For instance, the aniline may be reacted with a variety of reagents to affect nitrogen functionalization, for instance an acid and an appropriate amide bond forming reagent (e.g., HATU), an acid chloride and an appropriate base, or an appropriate aryl/heteroaryl bromide and an appropriate palladium catalyst/base combination. The Cy group may then be installed as described above to provide compounds of general formula 3-9. Alternatively, the Cy group may be installed first, followed by Sandmeyer reaction with an appropriate metal halide salt and source of nitrite. The resulting halogen can be functionalized through a variety of cross-coupling reactions, such as those described above. Final optional functionalization of the chloride can provide compounds of general formula 3-10.

Separation of atropisomers can be carried out on the compounds of Formula (I) or any of the intermediates. The separating can be performed using chromatography such as by HPLC or supercritical fluid chromatography.

Starting materials, reagents and intermediates whose synthesis is not described herein are either commercially available, known in the literature, or may be prepared by methods known to one skilled in the art.

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St. C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 6^{th} Ed. (Wiley, 2007); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022).

### III. Uses of the Compounds

Compounds of the present disclosure, including the compounds of Formula (I), or any of the embodiments thereof, are useful for therapy as described in further detail below.

The present disclosure provides compounds of Formula (I), for use as a medicament, or for use in medicine. The present disclosure provides compounds of Formula (I), for use as a medicament, or for use in treating disease, as described in further detail below. The present disclosure also provides the use of compounds of Formula (I), or any of the embodiments thereof, as a medicament, or for treating disease, as described in further detail below. The present disclosure also provides the use of compounds of Formula (I), or any of the embodiments thereof, in the manufacture of medicament for treating disease, as described in further detail below.

Compounds of the present disclosure are KRAS inhibitors and, thus, are useful in treating diseases and disorders associated with activity of KRAS. For the uses described herein, any of the compounds of Formula (I), including any of the embodiments thereof, may be used.

In particular, compounds of the invention are KRAS inhibitors having activity against one or more mutant forms of KRAS, and, thus, are useful in treating diseases and disorders associated with the presence or activity of mutant forms of KRAS, such as G12C, G12D, and/or the G12V mutant forms of KRAS.

The Ras family is comprised of three members: KRAS, NRAS and HRAS. RAS mutant cancers account for about 25% of human cancers. KRAS is the most frequently mutated isoform in human cancers: 85% of all RAS mutations are in KRAS, 12% in NRAS, and 3% in HRAS (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al., Nat. Rev. Drug. Discov., 2014, 13(11), 828-51). The majority of RAS mutations occur at amino acid residues/codons 12, 13, and 61; Codon 12 mutations are most frequent in KRAS. The frequency of specific mutations varied between RAS genes and G12D mutations are most predominant in KRAS whereas Q61R and G12R mutations are most frequent in NRAS and HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) (A. D. Cox, et al., Nat. Rev. Drug. Discov., 2014, 13(11), 828-51). In contrast, KRAS G12C mutations predominate in non-small cell lung cancer (NSCLC) comprising 11-16% of lung adenocarcinomas (nearly half of mutant KRAS is G12C), as well as 2-5% of pancreatic and colorectal adenocarcinomas, respectively (A. D. Cox, et al., Nat. Rev. Drug. Discov., 2014, 13(11), 828-51). Using shRNA knockdown thousands of genes across hundreds of cancer cell lines, genomic studies have demonstrated that cancer cells exhibiting KRAS mutations are highly dependent on KRAS function for cell growth (R. McDonald, et al., Cell, 2017, 170(3), 577-92).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

Diseases that can be treated with the compounds of Formula (I) include cancers. The cancers can include adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor. In some embodiments, the cancer can be adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, or Wilms' tumor.

The cancer types in which KRAS harboring G12C, G12V and G12D mutations are implicated and that can be treated using compounds of Formula (I), or any of the embodiments thereof, include, but are not limited to: carcinomas (e.g., pancreatic, colorectal, lung, bladder, gastric, esophageal, breast, head and neck, cervical skin, thyroid); hematopoietic malignancies (e.g., myeloproliferative neoplasms (MPN), myelodysplastic syndrome (MDS), chronic and juvenile myelomonocytic leukemia (CMML and JMML), acute myeloid leukemia (AML), acute lymphocytic leukemia (ALL) and multiple myeloma (MM)); and other neoplasms (e.g., glioblastoma and sarcomas). In addition, KRAS mutations were found in acquired resistance to anti-EGFR therapy (K. Knickelbein, et al., Genes Dis., 2015, 2(1), 4-12). KRAS mutations were found in immunological and inflammatory disorders (A. Femandez-Medarde, et al., Genes Cancer, 2011, 2(3), 344-358) such as Ras-associated lymphoproliferative disorder (RALD) or juvenile myelomonocytic leukemia (JMML) caused by somatic mutations of KRAS or NRAS.

Compounds of the present disclosure, including any of the embodiments thereof, can inhibit the activity of the KRAS protein. For example, compounds of the present disclosure can be used to inhibit activity of KRAS in a cell or in an individual or patient in need of inhibition of the enzyme by administering an inhibiting amount of one or more compounds of the present disclosure to the cell, individual, or patient.

As KRAS inhibitors, the compounds of the present disclosure, or any of the embodiments thereof, are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compounds of the present disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In an aspect, provided herein is a method of inhibiting KRAS activity, the method comprising contacting a compound of the instant disclosure with KRAS. In some embodiments, the contacting comprises administering the compound to a patient. In some embodiments, KRAS is characterized as having a somatic mutation of G12C. In other embodiments, KRAS is characterized as having a somatic mutation of G12D. In other embodiments, KRAS is characterized as having a somatic mutation of G12V.

In an aspect, provided herein is a method of inhibiting a KRAS protein harboring a G12C mutation, the method comprising contacting a compound of Formula (I), or any of the embodiments thereof, with KRAS.

In an aspect, provided herein is a method of inhibiting a KRAS protein harboring a G12D mutation, the method comprising contacting a compound of Formula (I), or any of the embodiments thereof, with KRAS harboring a G12D mutation.

In an aspect, provided herein is a method of inhibiting a KRAS protein harboring a G12V mutation, the method comprising contacting a compound of Formula (I), or any of the embodiments thereof, with KRAS harboring a G12V mutation.

In another aspect, provided herein is a method of treating a disease or disorder associated with inhibition of KRAS interaction, the method comprising administering to a patient in need thereof a therapeutically effective amount of a compound of Formula (I), or any of the embodiments thereof.

In some embodiments, the disease or disorder is an immunological or inflammatory disorder. In other embodiments, the immunological or inflammatory disorder is Ras-associated lymphoproliferative disorder or juvenile myelomonocytic leukemia caused by somatic mutations of KRAS. In some embodiments, the immunological or inflammatory disorder is caused by a somatic mutation of KRAS.

In other embodiments, the somatic mutation of KRAS is G12C. In other embodiments, the somatic mutation of KRAS is G12D. In other embodiments, the somatic mutation of KRAS is G12V.

In other embodiments, the immunological or inflammatory disorder is associated with activity of KRAS having a G12C mutation. In other embodiments, the immunological or inflammatory disorder is associated with activity of KRAS having a G12D mutation. In other embodiments, the immunological or inflammatory disorder is associated with activity of KRAS having a G12V mutation.

In yet another aspect, provided herein is a method of treating a disease or disorder associated with inhibiting a KRAS protein harboring a G12C mutation, the method comprising administering to a patient in need thereof a therapeutically effective amount of a compound of a compound of Formula (I), or any of the embodiments thereof,

In yet another aspect, provided herein is a method of treating a disease or disorder associated with inhibiting a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with inhibiting a KRAS protein harboring a G12V mutation, the method comprising administering to a patient in need thereof a therapeutically effective amount of a compound of Formula (I), or any of the embodiments thereof.

In yet another aspect, provided herein is a method of treating a disease or disorder associated with activity of a KRAS protein harboring a G12C mutation, the method comprising administering to a patient in need thereof a therapeutically effective amount of a compound of a compound of Formula (I), or any of the embodiments thereof,

In yet another aspect, provided herein is a method of treating a disease or disorder associated with activity of a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with activity of a KRAS protein harboring a G12V mutation, the method comprising administering to a patient in need thereof a therapeutically effective amount of a compound of Formula (I), or any of the embodiments thereof.

In another aspect, provided herein is also a method of treating cancer in a patient in need thereof comprising administering to the patient a therapeutically effective amount of a compound of Formula (I), or any of the embodiments thereof.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12C mutation.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12D mutation.

In another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12V mutation.

In yet another aspect, provided herein is a method for treating a cancer in a patient, the method comprising administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12C mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12D mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12V mutation, and administering to the patient a therapeutically effective amount of any one of the compounds disclosed herein, or pharmaceutically acceptable salt thereof.

In some embodiments, the cancer is selected from carcinomas, hematological cancers, sarcomas, and glioblastoma. In other embodiments, the hematological cancer is selected from myeloproliferative neoplasms, myelodysplastic syndrome, chronic and juvenile myelomonocytic leukemia, acute myeloid leukemia, acute lymphocytic leukemia, and multiple myeloma. In yet other embodiments, the carcinoma is selected from pancreatic, colorectal, lung, bladder, gastric, esophageal, breast, head and neck, cervical, skin, and thyroid. In other embodiments, the cancer is colorectal cancer, pancreatic cancer, or lung cancer. In yet other embodiments, the pancreatic cancer is pancreatic ductal adenocarcinoma (PDAC). In still other embodiments, the cancer is non-small cell lung cancer (NSCLC).

In some embodiments, the cancer is metastatic.

In an aspect, provided herein is a method for treating a disease or disorder associated with inhibition of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In an aspect, provided herein is a method for treating a disease or disorder associated with activity of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In some embodiments, the cancer is selected from hematological cancers, sarcomas, lung cancers, gastrointestinal cancers, genitourinary tract cancers, liver cancers, bone cancers, nervous system cancers, gynecological cancers, and skin cancers.

In other embodiments, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet other embodiments, the lung cancer is non-small cell lung cancer (NSCLC). In still other embodiments, the lung cancer is adenocarcinoma.

In some embodiments, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In some embodiments, the gastrointestinal cancer is colorectal cancer.

In other embodiments, the cancer is a carcinoma. In yet other embodiments, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still other embodiments, the cancer is a hematopoietic malignancy. In some embodiments, the hematopoietic malignancy is selected from multiple myeloma, acute myelogenous leukemia, and myeloproliferative neoplasms.

In other embodiments, the cancer is a neoplasm. In yet other embodiments, the neoplasm is glioblastoma or sarcomas.

In certain embodiments, the disclosure provides a method for treating a KRAS-mediated disorder in a patient in need thereof, comprising the step of administering to the patient a compound according to the invention, or a pharmaceutically acceptable composition thereof.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome, myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compounds of the present disclosure can also be useful in the inhibition of tumor metastasis.

In addition to oncogenic neoplasms, the compounds of the invention are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a patient suffering from a skeletal and chondrocyte disorder.

In some embodiments, compounds described herein can be used to treat Alzheimer's disease, HIV, or tuberculosis.

The term “8p11 myeloproliferative syndrome” refers to myeloid/lymphoid neoplasms associated with eosinophilia and abnormalities of FGFR1.

The term “cell” refers to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “contacting” refers to the bringing together of indicated moieties in an in vitro system or an in vivo system. For example, “contacting” KRAS with a compound described herein includes the administration of a compound described herein to an individual or patient, such as a human, having KRAS, as well as, for example, introducing a compound described herein into a sample containing a cellular or purified preparation containing KRAS.

The terms “individual,” “subject,” or “patient,” are used interchangeably, and refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, nontoxic and neither biologically nor otherwise undesirable and include excipients or carriers that are acceptable for veterinary use as well as human pharmaceutical use. In one embodiment, each component is “pharmaceutically acceptable” as defined herein. See, e.g., P. Beringer, et al., (Eds.), Remington: The Science and Practice of Pharmacy, 21^{st} Ed.; (Lippincott Williams & Wilkins: Philadelphia, Pa., 2005); A. Adejare (Ed.), Remington, The Science and Practice of Pharmacy, 23^{rd} Ed., (Elsevier, 2020); R. C. Rowe et al., Eds., Handbook of Pharmaceutical Excipients, 6^{th} Ed.; (Pharmaceutical Press, 2009); P. J. Shesky et al., Eds., Handbook of Pharmaceutical Excipients, 9^{th} Ed.; (The Pharmaceutical Press, 2020); M. Ash, et al., (Eds.), Handbook of Pharmaceutical Additives, 3^{rd} Ed.; (Gower Publishing Company: 2007); and M. Gibson (Ed.), Pharmaceutical Preformulation and Formulation, 2^{nd} Ed. (CRC Press LLC, 2009).

The term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” comprises the prevention of at least one symptom associated with or caused by the state, disease or disorder being prevented.

### III. Combination Therapies

Compounds of the present disclosure, including the compounds of Formula (I), or any of the embodiments thereof, may be useful in therapy when used in combination with one or more additional pharmaceutical agents, as described in further detail below.

a. Cancer Therapies

Compounds of the invention, including the compounds of Formula (I), or any of the embodiments thereof, may be useful in treatment of cancer when used in combination with one or more additional pharmaceutical agents, as described in further detail below.

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of KRAS-associated diseases, disorders or conditions. Other agents such as therapeutic antibodies can be used in combination with the compounds of the present disclosure for treatment of KRAS-associated diseases, disorders or conditions. The one or more additional pharmaceutical agents can be administered to a patient simultaneously or sequentially.

In some embodiments, the KRAS inhibitor is administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR—R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2; e.g., ruxolitinib or baricitinib; or JAK1; e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., zilurgisertib); or combinations thereof.

In some embodiments, the compound or salt described herein is administered with a PI3Kδ inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-1 agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Pat. No. 7,745,437.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK2 inhibitor of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the embodiments thereof as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the embodiments thereof as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, comovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

b. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IB1308), AB122 (GLS-010), AMP-224, AMP-514/MEDI-0680, BMS936559, JTX-4014, BGB-108, SHR-1210, MED14736, FAZ053, BCD-100, KN035, CS1001, BAT1306, LZM009, AK105, HLX10, SHR-1316, CBT-502 (TQB2450), A167 (KLA167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217,149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550.

In some embodiments, the PD-L1 inhibitor is selected from the compounds in Table 3, or a pharmaceutically acceptable salt thereof.

TABLE 3

| | US | |
|---|---|---|
| Cmpd | Publication | |
| No. | Appl. No. | Name and Structure |
| 1 | US 2018- | (R)-1-((7-cyano-2-(3′-(3-(((R)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example #24 | yl)methyl)pyrrolidine-3-carboxylic acid |
| | | 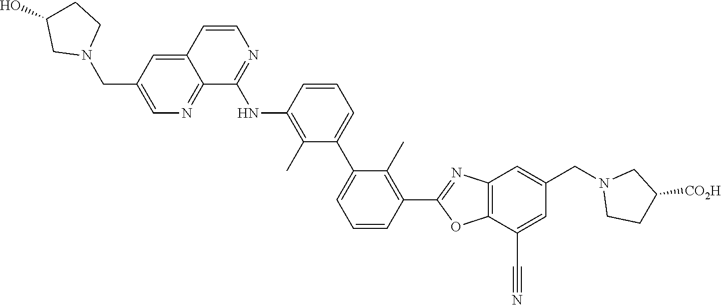 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C] |
| 2 | US 2018- | N-(2-chloro-3′-(8-chloro-6-((2-hydroxyethylamino)methyl)-[1,2,4]triazolo[1,5- |
| | 0179201, | a]pyridin-2-yl)-2′-methylbiphenyl-3-yl)-5-((2- |
| | Example #2 | hydroxyethylamino)methyl)picolinamide |
| | | 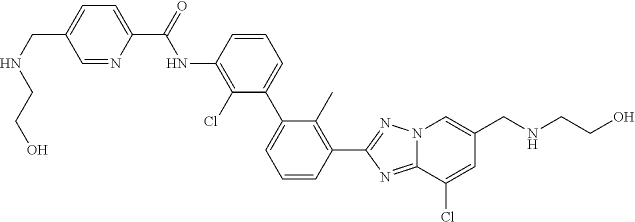 [SMILES: Cc1c(-c2nc3c(Cl)cc(CNCCO)cn3n2)cccc1-c1cccc(NC(=O)c2ccc(CNCCO)cn2)c1Cl] |
| 3 | US 2018- | (S)-1-((7-cyano-2-(3′-(3-(((S)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example #25 | yl)methyl)pyrrolidine-3-carboxylic acid |
| | | 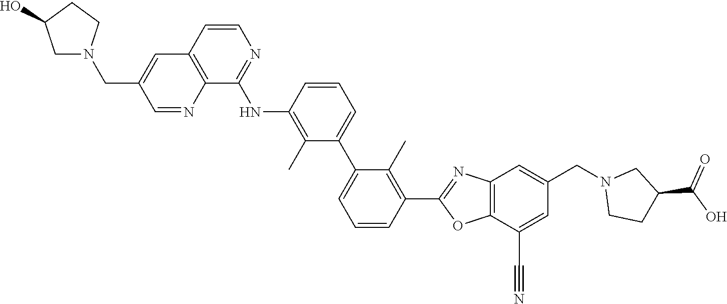 [SMILES: Cc1c(Nc2nccc3cc(CN4CC[C@H](O)C4)cnc23)cccc1-c1cccc(-c2nc3cc(CN4CC[C@H](C(=O)O)C4)cc(C#N)c3o2)c1C] |
| 4 | US 2018- | (R)-1-((7-cyano-2-(3′-(3-(((S)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example #26 | yl)methyl)pyrrolidine-3-carboxylic acid |
| | | 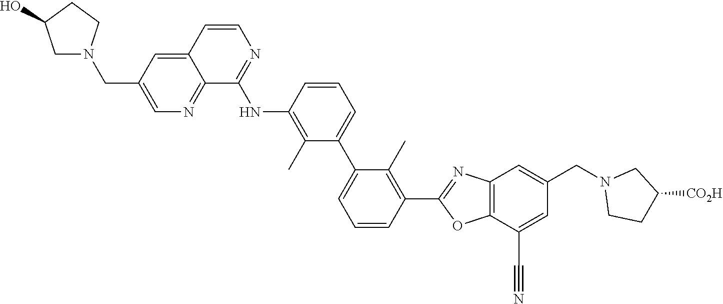 [SMILES: Cc1c(Nc2nccc3cc(CN4CC[C@H](O)C4)cnc23)cccc1-c1cccc(-c2nc3cc(CN4CC[C@@H](C(=O)O)C4)cc(C#N)c3o2)c1C] |
| 5 | US 2018- | (S)-1-((7-cyano-2-(3′-(3-(((R)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example #28 | yl)methyl)pyrrolidine-3-carboxylic acid |
| | | 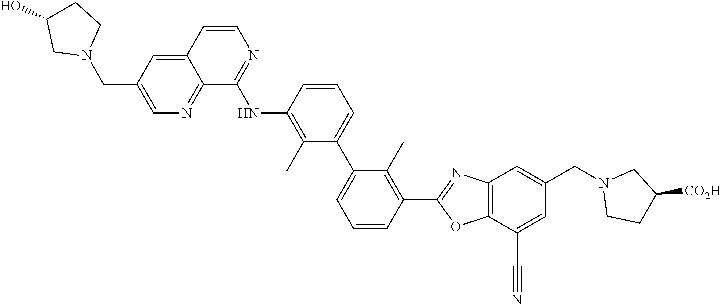 [SMILES: Cc1c(Nc2nccc3cc(CN4CC[C@@H](O)C4)cnc23)cccc1-c1cccc(-c2nc3cc(CN4CC[C@H](C(=O)O)C4)cc(C#N)c3o2)c1C] |
| 6 | US 2018- | 1-((7-cyano-2-(3′-(5-(2-(dimethylamino)acetyl)-5,6-dihydro-4H-pyrrolo[3,4- |
| | 0179197, | d]thiazol-2-yl)-2,2′-dimethylbiphenyl-3-yl)benzo[d]oxazol-5- |
| | Example | yl)methyl)piperidine-4-carboxylic acid |
| | #236 | 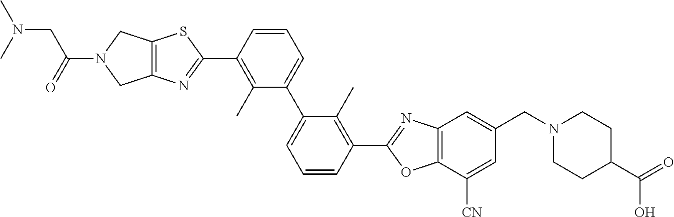 [SMILES: Cc1c(-c2nc3cc(CN4CCC(C(=O)O)CC4)cc(C#N)c3o2)cccc1-c1cccc(-c2nc3c(s2)CN(C(=O)CN(C)C)C3)c1C] |
| 7 | US 2018- | N,N′-(2-chloro-2′-methylbiphenyl-3,3′-diyl)bis(5-((2-hydroxyethylamino) |
| | 0179179, | methyl)picolinamide) |
| | Example #1 | 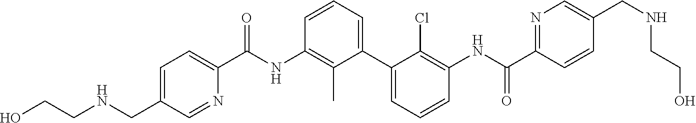 [SMILES: Cc1c(NC(=O)c2ccc(CNCCO)cn2)cccc1-c1cccc(NC(=O)c2ccc(CNCCO)cn2)c1Cl] |
| 8 | US 2018- | (R)-1-((6-(2′-chloro-3′-(5-((3-hydroxypyrrolidin-1-yl)methyl)picolinamido)-2- |
| | 0179179, | methylbiphenyl-3-ylcarbamoyl)pyridin-3-yl)methyl)piperidine-4-carboxylic acid |
| | Example #9 | 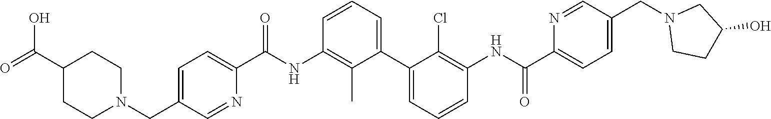 [SMILES: Cc1c(NC(=O)c2ccc(CN3CCC(C(=O)O)CC3)cn2)cccc1-c1cccc(NC(=O)c2ccc(CN3CC[C@@H](O)C3)cn2)c1Cl] |
| 9 | US 2018- | (S)-1-((6-((2′-chloro-2-methyl-3′-(5-(pyrrolidin-1-ylmethyl)picolinamido)-[1,1′- |
| | 0179179, | biphenyl]-3-yl)carbamoyl)-4-methylpyridin-3-yl)methyl)piperidine-2-carboxylic |
| | Example #12 | acid |
| | | 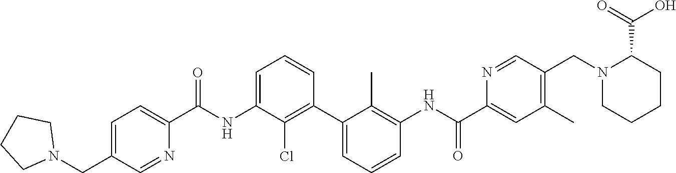 [SMILES: Cc1cc(C(=O)Nc2cccc(-c3cccc(NC(=O)c4ccc(CN5CCCC5)cn4)c3Cl)c2C)ncc1CN1CCCC[C@H]1C(=O)O] |
| 10 | US 2018- | trans 4-(2-(2-(2-chloro-3′-(3-(((R)-3-hydroxypyrrolidin-1-yl)methyl)-1,7- |
| | 0179202, | naphthyridin-8-ylamino)-2′-methylbiphenyl-3-ylcarbamoyl)-1-methyl-6,7- |
| | Example #52 | dihydro-1H-imidazo[4,5-c]pyridin-5(4H)-yl)ethyl)cyclohexanecarboxylic acid |
| | | 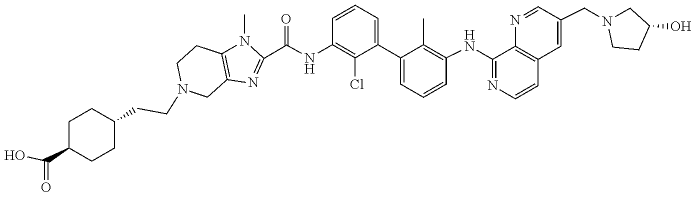 [SMILES: Cc1c(Nc2nccc3cc(CN4CC[C@@H](O)C4)cnc23)cccc1-c1cccc(NC(=O)c2nc3c(n2C)CCN(CC[C@H]2CC[C@H](C(=O)O)CC2)C3)c1Cl] |
| 11 | US 2018- | cis-4-((2-(2-chloro-3′-(3-(((R)-3-hydroxy-3-methylpyrrolidin-1-yl)methyl)-1,7- |
| | 0179202, | naphthyridin-8-ylamino)-2′-methylbiphenyl-3-ylcarbamoyl)-1-methyl-6,7- |
| | Example #56 | dihydro-1H-imidazo[4,5-c]pyridin-5(4H)-yl)methyl)cyclohexanecarboxylic acid |
| | | 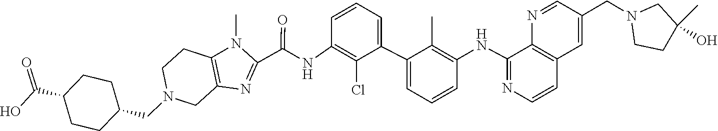 [SMILES: Cc1c(Nc2nccc3cc(CN4CC[C@@](C)(O)C4)cnc23)cccc1-c1cccc(NC(=O)c2nc3c(n2C)CCN(C[C@H]2CC[C@@H](C(=O)O)CC2)C3)c1Cl] |
| 12 | US 2018- | (R)-4-(2-(2-chloro-3′-(7-((3-hydroxypyrrolidin-1-yl)methyl)pyrido[3,2- |
| | 0179202, | d]pyrimidin-4-ylamino)-2′-methylbiphenyl-3-ylcarbamoyl)-1-methyl-6,7- |
| | Example #68 | dihydro-1H-imidazo[4,5-c]pyridin-5(4H)-yl)-1-methylcyclohexanecarboxylic |
| | | 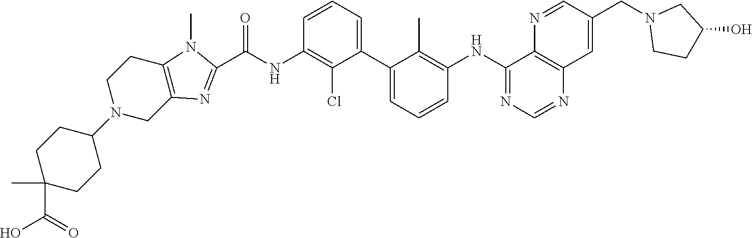 [SMILES: Cc1c(Nc2ncnc3cc(CN4CC[C@@H](O)C4)cnc23)cccc1-c1cccc(NC(=O)c2nc3c(n2C)CCN(C2CCC(C)(C(=O)O)CC2)C3)c1Cl] |
| 13 | US 2018- | (R)-1-((8-((2-chloro-3′-(5-(N-ethyl-N-methylglycyl)-5,6-dihydro-4H-pyrrolo[3,4- |
| | 0179202, | d]thiazol-2-yl)-2′-methyl-[1,1′-biphenyl]-3-yl)amino)-1,7-naphthyridin-3- |
| | Example #90 | yl)methyl)pyrrolidine-3-carboxylic acid |
| | | 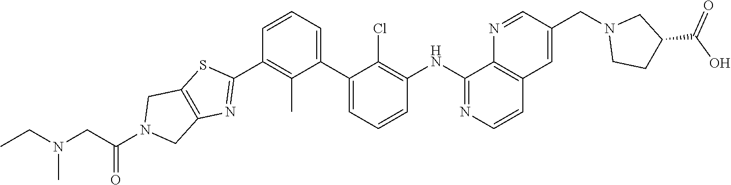 [SMILES: CCN(C)CC(=O)N1Cc2nc(-c3cccc(-c4cccc(Nc5nccc6cc(CN7CC[C@@H](C(=O)O)C7)cnc56)c4Cl)c3C)sc2C1] |
| 14 | US 2018- | (R)-2-(dimethylamino)-1-(2-(3′-(5-(2-(3-hydroxypyrrolidin-1-yl)acetyl)-5,6- |
| | 0177784, | dihydro-4H-pyrrolo[3,4-d]thiazol-2-yl)-2,2′-dimethylbiphenyl-3-yl)-4H- |
| | Example #35 | pyrrolo[3,4-d]thiazol-5(6H)-yl)ethanone |
| | | 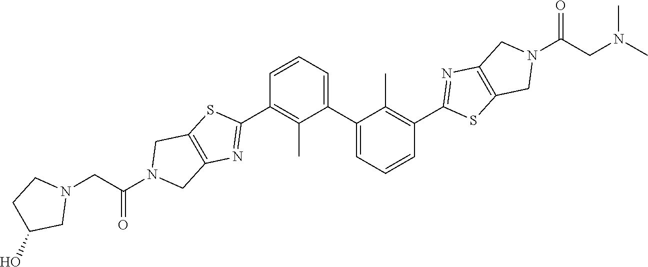 [SMILES: Cc1c(-c2nc3c(s2)CN(C(=O)CN(C)C)C3)cccc1-c1cccc(-c2nc3c(s2)CN(C(=O)CN2CC[C@@H](O)C2)C3)c1C] |
| 15 | US 2018- | trans-4-((2-(2′-chloro-3′-(1,5-dimethyl-4,5,6,7-tetrahydro-1H-imidazo[4,5- |
| | 0177870, | c]pyridine-2-carboxamido)-2-methylbiphenyl-3-ylcarbamoyl)-1-methyl-6,7- |
| | Example #37 | dihydro-1H-imidazo[4,5-c]pyridin-5(4H)-yl)methyl)cyclohexane-1-carboxylic |
| | | acid |
| | | 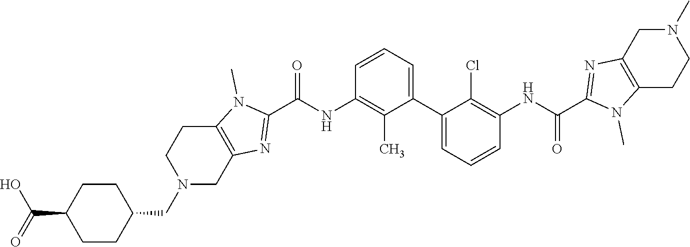 [SMILES: Cc1c(NC(=O)c2nc3c(n2C)CCN(C[C@H]2CC[C@H](C(=O)O)CC2)C3)cccc1-c1cccc(NC(=O)c2nc3c(n2C)CCN(C)C3)c1Cl] |
| 16 | US 2018- | trans-4-(2-(2-((2′-chloro-3′-(1,5-dimethyl-4,5,6,7-tetrahydro-1H-imidazo[4,5- |
| | 0177870, | c]pyridine-2-carboxamido)-2-methyl-[1,1′-biphenyl]-3-yl)carbamoyl)-1-methyl- |
| | Example | 1,4,6,7-tetrahydro-5H-imidazo[4,5-c]pyridin-5-yl)ethyl)cyclohexane-1- |
| | #100 | carboxylic acid |
| | | 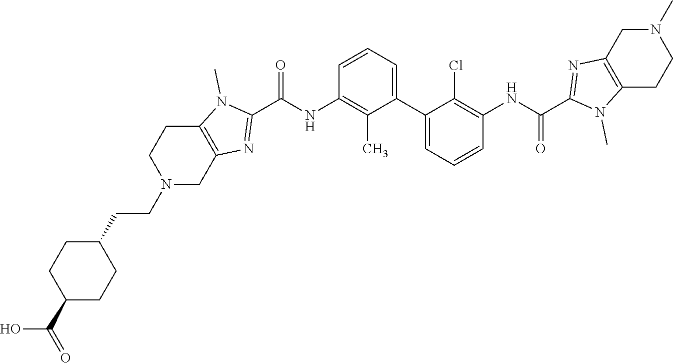 [SMILES: Cc1c(NC(=O)c2nc3c(n2C)CCN(CC[C@H]2CC[C@H](C(=O)O)CC2)C3)cccc1-c1cccc(NC(=O)c2nc3c(n2C)CCN(C)C3)c1Cl] |
| 17 | US 2018- | cis-4-((2-((2′-chloro-3′-(1,5-dimethyl-4,5,6,7-tetrahydro-1H-imidazo[4,5- |
| | 0177870, | c]pyridine-2-carboxamido)-2-methyl-[1,1′-biphenyl]-3-yl)carbamoyl)-1-methyl- |
| | Example | 1,4,6,7-tetrahydro-5H-imidazo[4,5-c]pyridin-5-yl)methyl)cyclohexane-1- |
| | #114 | carboxylic acid |
| | | 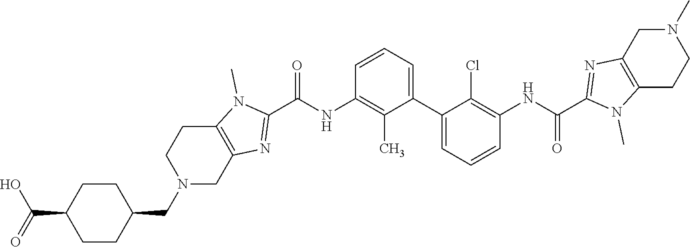 [SMILES: Cc1c(NC(=O)c2nc3c(n2C)CCN(C[C@H]2CC[C@@H](C(=O)O)CC2)C3)cccc1-c1cccc(NC(=O)c2nc3c(n2C)CCN(C)C3)c1Cl] |
| 18 | US 2018- | cis-4-((2-((2-chloro-3′-(1,5-dimethyl-4,5,6,7-tetrahydro-1 H-imidazo[4,5- |
| | 0177870, | c]pyridine-2-carboxamido)-2′-methyl-[1,1′-biphenyl]-3-yl)carbamoyl)-1-methyl- |
| | Example | 1,4,6,7-tetrahydro-5H-imidazo[4,5-c]pyridin-5-yl)methyl)cyclohexane-1- |
| | #135 | carboxylic acid |
| | | 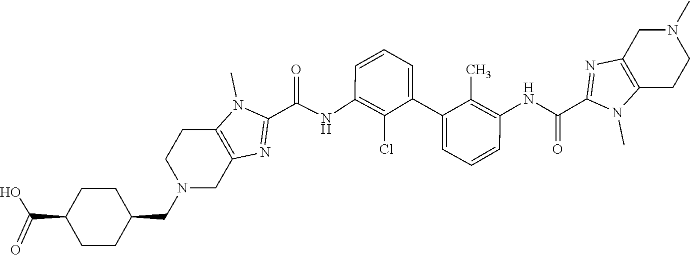 [SMILES: Cc1c(NC(=O)c2nc3c(n2C)CCN(C)C3)cccc1-c1cccc(NC(=O)c2nc3c(n2C)CCN(C[C@H]2CC[C@@H](C(=O)O)CC2)C3)c1Cl] |
| 19 | US 2018- | trans-4-(2-(2-((2′-chloro-2-cyano-3′-(1,5-dimethyl-4,5,6,7-tetrahydro-1H- |
| | 0177870, | imidazo[4,5-c]pyridine-2-carboxamido)-[1,1′-biphenyl]-3-yl)carbamoyl)-1- |
| | Example | methyl-1,4,6,7-tetrahydro-5H-imidazo[4,5-c]pyridin-5-yl)ethyl)cyclohexane-1- |
| | #148 | carboxylic acid |
| | | 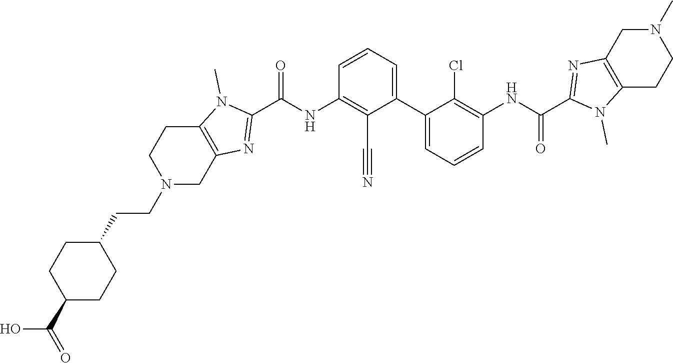 [SMILES: CN1CCc2c(nc(C(=O)Nc3cccc(-c4cccc(NC(=O)c5nc6c(n5C)CCN(CC[C@H]5CC[C@H](C(=O)O)CC5)C6)c4C#N)c3Cl)n2C)C1] |
| 20 | US 2018- | trans-4-((2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)methyl)cyclohexane-1-carboxylic |
| | #159 | acid |
| | | 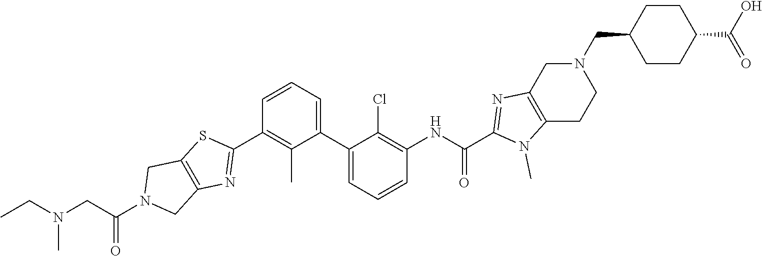 [SMILES: CCN(C)CC(=O)N1Cc2nc(-c3cccc(-c4cccc(NC(=O)c5nc6c(n5C)CCN(C[C@H]5CC[C@H](C(=O)O)CC5)C6)c4Cl)c3C)sc2C1] |
| 21 | US 2018- | cis-4-((2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1 H-imidazo[4,5-c]pyridin-5(4H)-yl)methyl)cyclohexane-1-carboxylic |
| | #160 | acid |
| | | 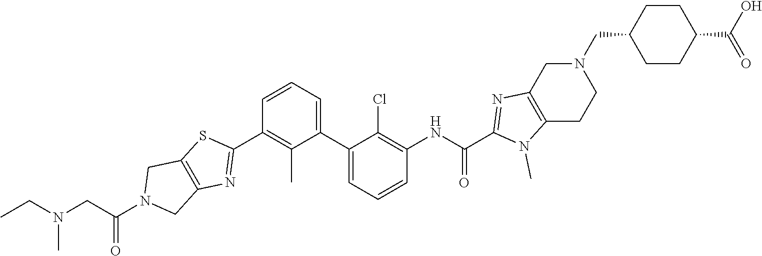 [SMILES: CCN(C)CC(=O)N1Cc2nc(-c3cccc(-c4cccc(NC(=O)c5nc6c(n5C)CCN(C[C@H]5CC[C@@H](C(=O)O)CC5)C6)c4Cl)c3C)sc2C1] |
| 22 | US 2018- | 4-(2-(2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)ethyl)cyclohexane-1-carboxylic acid |
| | #161 | 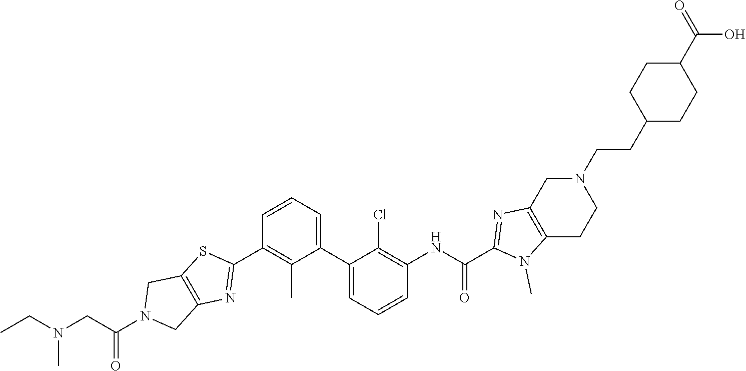 [SMILES: CCN(C)CC(=O)N1Cc2nc(-c3cccc(-c4cccc(NC(=O)c5nc6c(n5C)CCN(CCC5CCC(C(=O)O)CC5)C6)c4Cl)c3C)sc2C1] |
| 23 | US 2018- | 4-(2-(2-(2-chloro-3′-(5-(2-(isopropyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)ethyl)cyclohexane-1-carboxylic acid |
| | #162 | 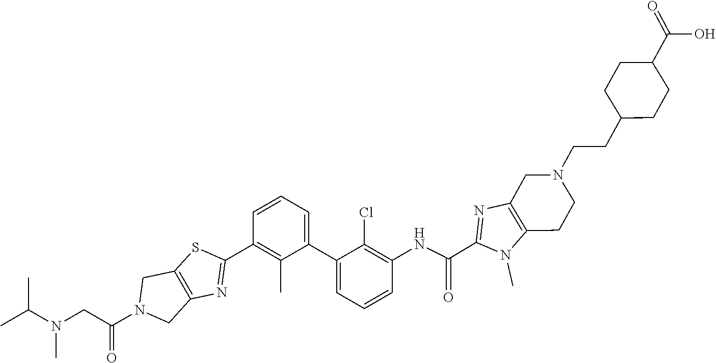 [SMILES: Cc1c(-c2nc3c(s2)CN(C(=O)CN(C)C(C)C)C3)cccc1-c1cccc(NC(=O)c2nc3c(n2C)CCN(CCC2CCC(C(=O)O)CC2)C3)c1Cl] |
| 24 | US 2019- | (R)-1-((7-cyano-2-(3′-(2-(difluoromethyl)-7-((3-hydroxypyrrolidin-1- |
| | 0300524, | yl)methyl)pyrido[3,2-d]pyrimidin-4-ylamino)-2,2′-dimethylbiphenyl-3- |
| | Example #16 | yl)benzo[d]oxazol-5-yl)methyl)piperidine-4-carboxylic acid |
| | | 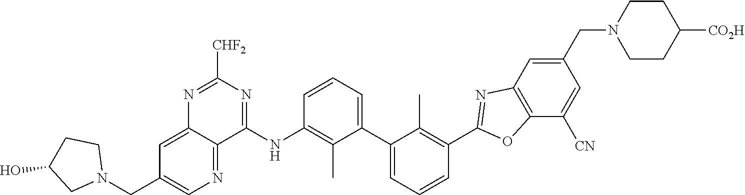 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C] |
| 25 | US 2019- | (R)-1-((7-cyano-2-(3′-(2-(difluoromethyl)-7-(((R)-3-hydroxypyrrolidin-1- |
| | 0300524, | yl)methyl)pyrido[3,2-d]pyrimidin-4-ylamino)-2,2′-dimethylbiphenyl-3- |
| | Example #17 | yl)benzo[d]oxazol-5-yl)methyl)pyrrolidine-3-carboxylic acid |
| | | 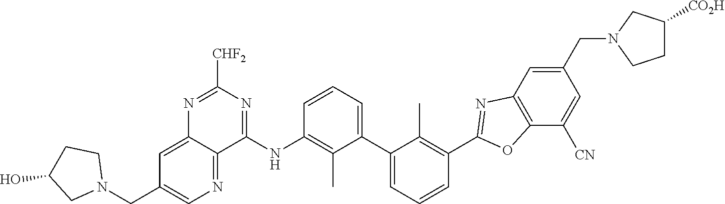 [SMILES: Cc1c(Nc2nc(C(F)F)nc3cc(CN4CC[C@@H](O)C4)cnc23)cccc1-c1cccc(-c2nc3cc(CN4CC[C@@H](C(=O)O)C4)cc(C#N)c3o2)c1C] |
| 26 | US 2019- | (R)-1-((7-cyano-2-(3′-(2-(difluoromethyl)-7-(((R)-3-hydroxypyrrolidin-1- |
| | 0300524, | yl)methyl)pyrido[3,2-d]pyrimidin-4-ylamino)-2,2′-dimethylbiphenyl-3- |
| | Example #18 | yl)benzo[d]oxazol-5-yl)methyl)-3-methylpyrrolidine-3-carboxylic acid |
| | | 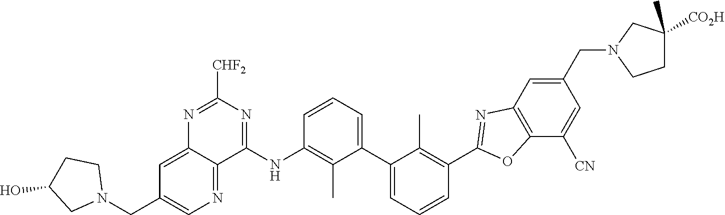 [SMILES: Cc1c(Nc2nc(C(F)F)nc3cc(CN4CC[C@@H](O)C4)cnc23)cccc1-c1cccc(-c2nc3cc(CN4CC[C@@](C)(C(=O)O)C4)cc(C#N)c3o2)c1C] |
| 27 | US 2019- | (R)-1-((7-cyano-2-(3′-(2-(difluoromethyl)-7-((3-hydroxy-3-methylpyrrolidin-1- |
| | 0300524, | yl)methyl)pyrido[3,2-d]pyrimidin-4-ylamino)-2,2′-dimethylbiphenyl-3- |
| | Example #30 | yl)benzo[d]oxazol-5-yl)methyl)piperidine-4-carboxylic acid |
| | | 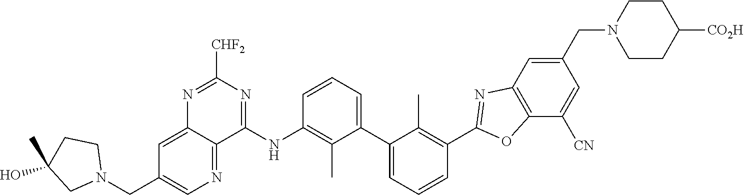 [SMILES: Cc1c(Nc2nc(C(F)F)nc3cc(CN4CC[C@@](C)(O)C4)cnc23)cccc1-c1cccc(-c2nc3cc(CN4CCC(C(=O)O)CC4)cc(C#N)c3o2)c1C] |
| 28 | US 2019- | (S)-1-((7-cyano-2-(3′-(2-(difluoromethyl)-7-((3-hydroxy-3-methylpyrrolidin-1- |
| | 0300524, | yl)methyl)pyrido[3,2-d]pyrimidin-4-ylamino)-2,2′-dimethylbiphenyl-3- |
| | Example #31 | yl)benzo[d]oxazol-5-yl)methyl)piperidine-4-carboxylic acid |
| | | 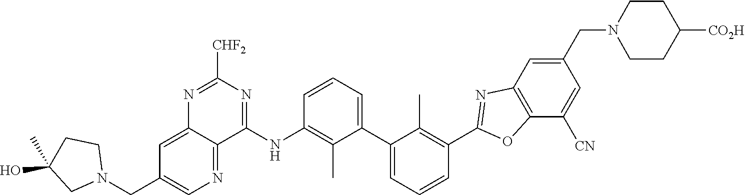 [SMILES: Cc1c(Nc2nc(C(F)F)nc3cc(CN4CC[C@](C)(O)C4)cnc23)cccc1-c1cccc(-c2nc3cc(CN4CCC(C(=O)O)CC4)cc(C#N)c3o2)c1C] |
| 29 | US 2019- | (R)-4-(2-(2-((2,2′-dichloro-3′-(5-(2-hydroxypropyl)-1-methyl-4,5,6,7-tetrahydro- |
| | 0345170, | 1H-imidazo[4,5-c]pyridine-2-carboxamido)-[1,1′-biphenyl]-3-yl)carbamoyl)-1- |
| | Example #13 | methyl-1,4,6,7-tetrahydro-5H-imidazo[4,5-c]pyridin-5- |
| | | yl)ethyl)bicyclo[2.2.1]heptane-1-carboxylic acid |
| | | 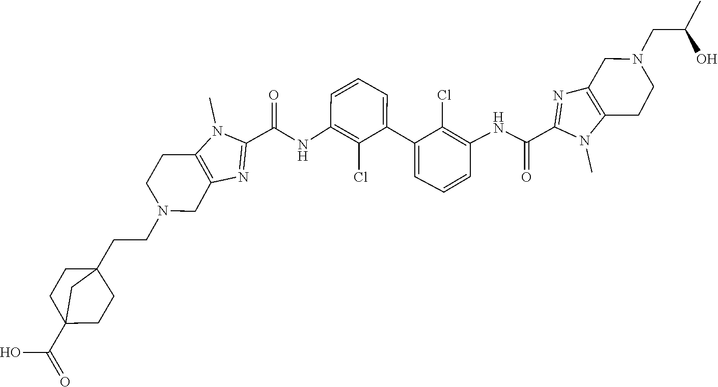 [SMILES: C[C@@H](O)CN1CCc2c(nc(C(=O)Nc3cccc(-c4cccc(NC(=O)c5nc6c(n5C)CCN(CCC57CCC(C(=O)O)(CC5)C7)C6)c4Cl)c3Cl)n2C)C1] |
| 30 | US 2019- | 4,4′-(((((2,2′-dichloro-[1,1′-biphenyl]-3,3′- |
| | 0345170, | diyl)bis(azanediyl))bis(carbonyl))bis(1-methyl-1,4,6,7-tetrahydro-5H- |
| | Example #17 | imidazo[4,5-c]pyridine-2,5-diyl))bis(ethane-2,1-diyl))bis(bicyclo[2.2.1]heptane- |
| | | 1-carboxylic acid) |
| | | 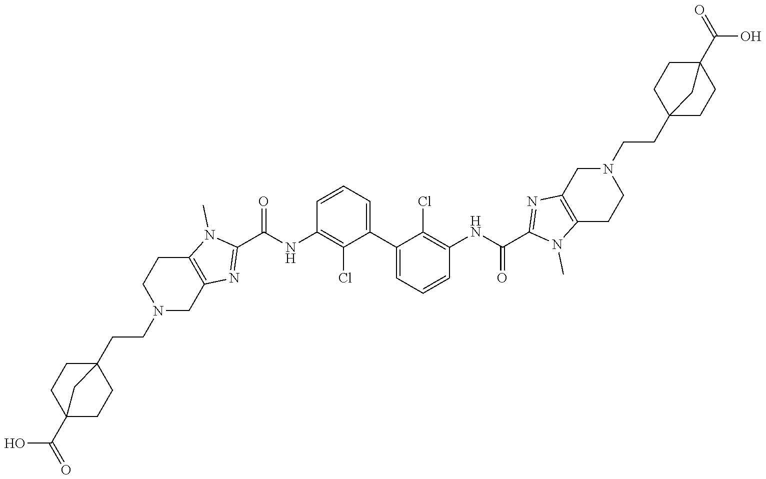 [SMILES: Cn1c(C(=O)Nc2cccc(-c3cccc(NC(=O)c4nc5c(n4C)CCN(CCC46CCC(C(=O)O)(CC4)C6)C5)c3Cl)c2Cl)nc2c1CCN(CCC13CCC(C(=O)O)(CC1)C3)C2] |
| 31 | US 2019- | 4-((2-((3′-(5-(2-(4-carboxybicyclo[2.2.1]heptan-1-yl)ethyl)-1-methyl-4,5,6,7- |
| | 0345170, | tetrahydro-1H-imidazo[4,5-c]pyridine-2-carboxamido)-2,2′-dichloro-[1,1′- |
| | Example #18 | biphenyl]-3-yl)carbamoyl)-1-methyl-1,4,6,7-tetrahydro-5H-imidazo[4,5- |
| | | c]pyridin-5-yl)methyl)bicyclo[2.2.1]heptane-1-carboxylic acid |
| | | 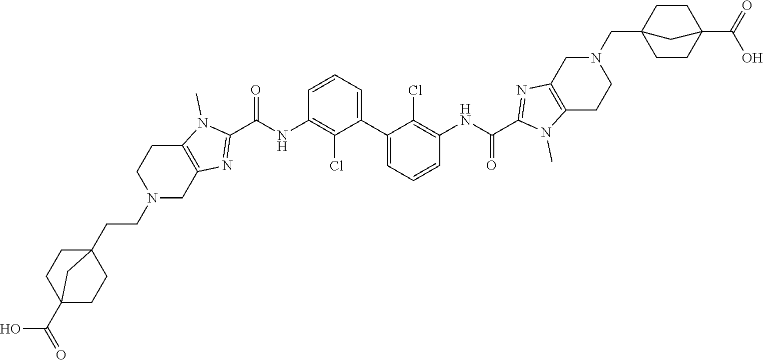 [SMILES: Cn1c(C(=O)Nc2cccc(-c3cccc(NC(=O)c4nc5c(n4C)CCN(CC46CCC(C(=O)O)(CC4)C6)C5)c3Cl)c2Cl)nc2c1CCN(CCC13CCC(C(=O)O)(CC1)C3)C2] |
| 32 | US 2019- | 4,4′-(((((2-chloro-2'-methyl-[1,1′-biphenyl]-3,3′- |
| | 0345170, | diyl)bis(azanediyl))bis(carbonyl))bis(1-methyl-1,4,6,7-tetrahydro-5H- |
| | Example #34 | imidazo[4,5-c]pyridine-2,5-diyl))bis(ethane-2,1-diyl))bis(bicyclo[2.2.1 ]heptane- |
| | | 1-carboxylic acid) |
| | | 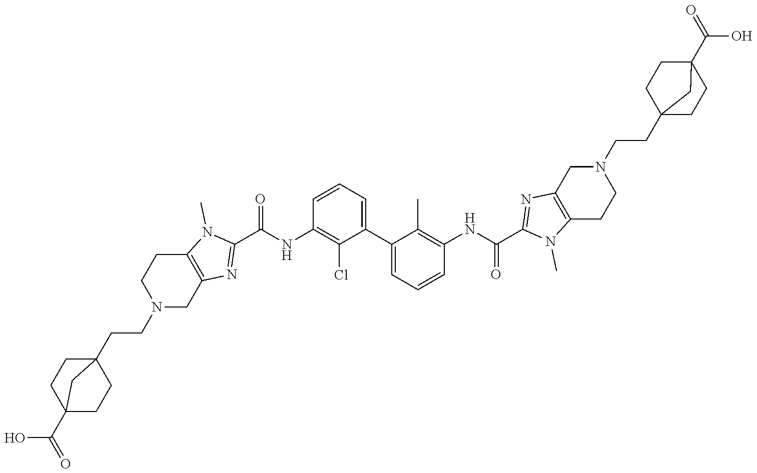 [SMILES: Cc1c(NC(=O)c2nc3c(n2C)CCN(CCC24CCC(C(=O)O)(CC2)C4)C3)cccc1-c1cccc(NC(=O)c2nc3c(n2C)CCN(CCC24CCC(C(=O)O)(CC2)C4)C3)c1Cl] |
| 33 | US 2019- | 4,4′-(((((2-chloro-2′-cyano-[1,1′-biphenyl]-3,3′- |
| | 0345170, | diyl)bis(azanediyl))bis(carbonyl))bis(1-methyl-1,4,6,7-tetrahydro-5H- |
| | Example #51 | imidazo[4,5-c]pyridine-2,5-diyl))bis(ethane-2,1-diyl))bis(bicyclo[2.2.1]heptane- |
| | | 1-carboxylic acid) |
| | | 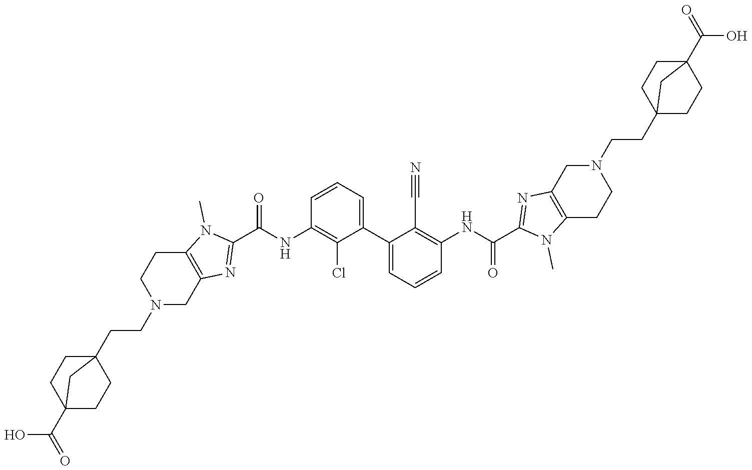 [SMILES: Cn1c(C(=O)Nc2cccc(-c3cccc(NC(=O)c4nc5c(n4C)CCN(CCC46CCC(C(=O)O)(CC4)C6)C5)c3C#N)c2Cl)nc2c1CCN(CCC13CCC(C(=O)O)(CC1)C3)C2] |
| 34 | US 2021- | (R)-4-(2-(2-((2-chloro-3′-((2-(difluoromethyl)-7-((3-hydroxypyrrolidin-1- |
| | 0094976, | yl)methyl)pyrido[3,2-d]pyrimidin-4-yl) amino)-2′-methyl-[1,1′-biphenyl]-3- |
| | Example #1 | yl)carbamoyl)-1-methyl-1,4,6,7-tetrahydro-5H-imidazo[4,5-c]pyridin-5- |
| | | yl)ethyl)bicyclo[2.2.1]heptane-1-carboxylic acid |
| | | 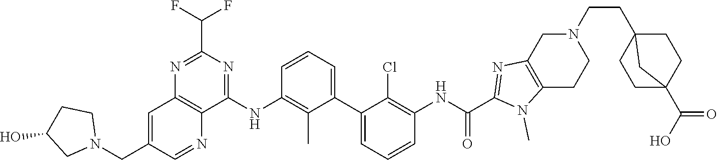 [SMILES: Cc1c(Nc2nc(C(F)F)nc3cc(CN4CC[C@@H](O)C4)cnc23)cccc1-c1cccc(NC(=O)c2nc3c(n2C)CCN(CCC24CCC(C(=O)O)(CC2)C4)C3)c1Cl] |

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AM 05, HLX10, or TSR-042. In some embodiments, the anti-PD-1 antibody is nivolumab, pembrolizumab, cemiplimab, spartalizumab, camrelizumab, cetrelimab, tornpalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is tornpalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012 (INCMGA0012; retifanlimab). In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MED14736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MED14736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PDL1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, US 2019/0300524, and US 2019/0345170, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MED19447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MED11873, or MED16469. In some embodiments, the agonist of an immune checkpoint molecule is an agonist of OX40, e.g., OX40 agonist antibody or OX40L fusion protein. In some embodiments, the anti-OX40 antibody is INCAGN01949, MED10562 (tavolimab), MOXR-0916, PF-04518600, GSK3174998, BMS-986178, or 9B12. In some embodiments, the OX40L fusion protein is MED16383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MED19197.

The compounds of the present disclosure can be used in combination with bispecific antibodies. In some embodiments, one of the domains of the bispecific antibody targets PD1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AKi04.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### IV. Formulation, Dosage Forms and Administration

When employed as pharmaceuticals, the compounds of the present disclosure can be administered in the form of pharmaceutical compositions. Thus, the present disclosure provides a composition comprising a compound of Formula (I), a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105M).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetyistearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. Labeled Compounds and Assay Methods

Another aspect of the present invention relates to labeled compounds of the disclosure (radio-labeled, fluorescent-labeled, etc.) that would be useful not only in imaging techniques but also in assays, both in vitro and in vivo, for localizing and quantitating KRAS protein in tissue samples, including human, and for identifying KRAS ligands by inhibition binding of a labeled compound. Substitution of one or more of the atoms of the compounds of the present disclosure can also be useful in generating differentiated ADME (Adsorption, Distribution, Metabolism and Excretion). Accordingly, the present invention includes KRAS binding assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). A “radio-labelled” compound can refer to an isotopically-labelled compound in which one or more atoms are replaced or substituted by an atom of an isotope that is radioactive.

Suitable isotopes that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁶F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated. The symbol D included in a chemical formula or as a substituent indicates that deuterium is incorporated in the position labelled at greater than natural abundance, and typically indicates an abundance of equal to or greater than 50%, preferably equal to or greater than 90% or equal to or greater than 95%, 96%, 97%, 98%, 99%, 99.5%, 99.9%, 99.95%, or 99.99% relative to other forms of hydrogen.

Synthetic methods for including isotopes into organic compounds are known in the art (A. F. Thomas, Deuterium Labeling in Organic Chemistry, (Appleton-Century-Crofts, New York, N.Y., 1971); J. Atzrodt, et al., Angew. Chem. Int. Ed., 2007, 7744-65; J. R. Hanson, The Organic Chemistry of Isotopic Labelling, (Royal Society of Chemistry, 2011)). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes, et al., J. Med. Chem. 2011, 54(1), 201-10; R. Xu et al., J. Label. Compd. Radiopharm. 2015, 58, 308-12). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷Br can be useful.

It is understood that a “radio-labeled” or “labeled compound” is a compound that has incorporated at least one radionuclide. In some embodiments, the radionuclide is selected from ³H, ¹⁴C, ¹²⁵I, ³⁵S and ⁸²Br.

The present disclosure can further include synthetic methods for incorporating radioisotopes into compounds of the disclosure. Synthetic methods for incorporating radioisotopes into organic compounds are well known in the art, and an ordinary skill in the art will readily recognize the methods applicable for the compounds of disclosure.

A labeled compound of the invention can be used in a screening assay to identify and/or evaluate compounds. For example, a newly synthesized or identified compound (i.e., test compound) which is labeled can be evaluated for its ability to bind a KRAS protein by monitoring its concentration variation when contacting with the KRAS, through tracking of the labeling. For example, a test compound (labeled) can be evaluated for its ability to reduce binding of another compound which is known to bind to a KRAS protein (i.e., standard compound). Accordingly, the ability of a test compound to compete with the standard compound for binding to the KRAS protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### VI. Kits

The present disclosure also includes pharmaceutical kits useful, e.g., in the treatment or prevention of diseases or disorders associated with the activity of KRAS, such as cancer or infections, which include one or more containers containing a pharmaceutical composition comprising a therapeutically effective amount of a compound of Formula (I), or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compounds of the Examples have been found to inhibit the activity of KRAS according to at least one assay described herein.

### EXAMPLES

Experimental procedures for compounds of the invention are provided below. Preparatory LCMS purifications of some of the compounds prepared were performed on Waters mass directed fractionation systems. The basic equipment setup, protocols, and control software for the operation of these systems have been described in detail in the literature. See e.g., K. F. Blom, J. Combi. Chem., 2002, 4(4), 295-301; K. F. Blom, et al., J. Combi. Chem., 2003, 5(5), 670-683; and K. F. Blom, et al., J. Combi. Chem. 2004, 6(6), 874-83. The compounds separated were typically subjected to analytical liquid chromatography mass spectrometry (LCMS) for purity check.

The compounds separated were typically subjected to analytical LCMS for purity check under the following conditions: Instrument; Agilent 1100 series, LC/MSD, Column: Waters SUNFIRE® C₁₈ 5 μm particle size, 2.1×5.0 mm, Buffers: mobile phase A: 0.025% TFA in water and mobile phase B: MeCN; gradient 2% to 80% of B in 3 min. with flow rate 2.0 mL/min.

Some of the compounds prepared were also separated on a preparative scale by RP-HPLC with MS detector or FCC (silica gel) as indicated in the Examples. Typical prep. RP-HPLC column conditions are as follows:
pH=2 purifications: Waters SUNFIRE® C₁₈ 5 μm particle size, 19×100 mm column, eluting with mobile phase A: 0.1% TFA in water and mobile phase B: MeCN; the flow rate was 30 mL/min., the separating gradient was optimized for each compound using the Compound Specific Method Optimization protocol as described in the literature [see K. F. Blom, et al., J. Combi. Chem. 2004, 6(6), 874-83]. Typically, the flow rate used with the 30×100 mm column was 60 mL/min.

pH=10 purifications: Waters XBRIDGE® C₁₈ 5 μm particle size, 19×100 mm column, eluting with mobile phase A: 0.15% NH₄OH in water and mobile phase B: MeCN; the flow rate was 30 mL/min., the separating gradient was optimized for each compound using the Compound Specific Method Optimization protocol as described in the literature [See K. F. Blom, et al., J. Combi. Chem. 2004, 6(6), 874-83]. Typically, the flow rate used with 30×100 mm column was 60 mL/min.

The following abbreviations may be used herein: AcOH (acetic acid); Ac₂O (acetic anhydride); aq. (aqueous); atm. (atmosphere(s)); BH₃-DMS (borane dimethyl sulfide complex); Boc (t-butoxycarbonyl); Boc₂O (di-t-butyl dicarbonate); BOP ((benzotriazol-1-yloxy)tris(dimethylamino)phosphonium hexafluorophosphate); B₂pin₂ (bis(pinacolato)diboron); br (broad); cataCXium® A Pd G3 ([(di(1-adamantyl)butylphosphine)-2-(2′-amino-1,1′-biphenyl)]palladium(II) methanesulfonate); Cbz (carboxybenzyl); calc. (calculated); CHCl₃ (chloroform); CO (carbon monoxide); CO₂ (carbon dioxide); COD (cycloocta-1,5-diene); Cs₂CO₃ (cesium carbonate); CsF (cesium fluoride); DAST (diethylaminosulfur trifluoride); d (doublet); dd (doublet of doublets); DBU (1,8-diazabicyclo[5.4.0]undec-7-ene); DCE (1,2-dichloroethane); DCM (dichloromethane); DIAD (N,N′-diisopropyl azidodicarboxylate); DIPEA (N,N-diisopropylethylamine); DIBAL (diisobutylaluminium hydride); DMF (N,N-dimethytformamide); DMAP (4-dimethylaminopyridine); DMSO (dimethylsulfoxide); eq. (equivalent(s)); Et (ethyl); EtOH (ethanol); EtOAc (ethyl acetate); Ex. (Example); FCC (flash column chromatography); g (gram(s)); h (hour(s)); H₂ (hydrogen); H₃PO₄ (phosphoric acid); HATU (N,N, N′, N′-tetramethyl-O-(7-azabenzotriazol-1-yl)uronium hexafluorophosphate); HCl (hydrochloric acid or hydrogen chloride); HFIP (hexafluoro-2-propanol); HPLC (high performance liquid chromatography); Hz (hertz); [Ir(OMe)(COD)]₂ ((1,5-cyclooctadiene)(methoxy)iridium(I) dimer); J (coupling constant); K₂CO₃ (potassium carbonate); KI (potassium iodide); KOAc (potassium acetate); KOtBu (potassium tert-butoxide); K₃PO₄ (tribasic potassium phosphate); LCMS (liquid chromatography-mass spectrometry); LiBH₄ (lithium borohydride); LDA (lithium diisopropylamide); LHMDS (lithium bis(trimethylsilyl)amide); m (multiplet); M (molar); mCPBA (3-chloroperoxybenzoic acid); MS (Mass spectrometry); Me (methyl); MeCN (acetonitrile); MeOH (methanol); mg (milligram(s)); MgSO₄ (magnesium sulfate); min. (minutes(s)); mL (milliliter(s)); mmol (millimole(s)); N (normal); N₂ (nitrogen); NaBH₄ (sodium borohydride); NaBH(OAc)₃ (sodium triacetoxyborohydride); Na₂CO₃ (sodium carbonate); NaBH₃CN (sodium cyanoborohydride); NaCl (sodium chloride); NADPH (nicotinamide adenine dinucleotide phosphate); NaH (sodium hydride); NaHCO₃ (sodium bicarbonate); NaHMDS (sodium bis(trimethylsilyl)amide); NaIO₄ (sodium metaperiodate); NaOH (sodium hydroxide); Na₂SO₄ (sodium sulfate); Na₂SO₃ (sodium sulfite); Na₂S₂O₃ (sodium thiosulfate); NCS (N-chlorosuccinimide); NEt₃ (triethylamine); NH₄Cl (ammonium chloride); NH₄OH (ammonium hydroxide); nM (nanomolar); NMP (N-methylpyrrolidinone); NMR (nuclear magnetic resonance spectroscopy); OTf (trifluoromethanesulfonate); Pd(amphos)Cl₂ (bis(ditert-butyl(4-dimethylaminophenyl)phosphine)dichloropalladium(II)); Ph (phenyl); Pd(PPh₃)₄ (tetrakis(triphenylphosphine) palladium(0)); pM (picomolar); PPT (precipitate); PPTS (pyridinium p-toluenesulfonate); prep. (preparative); RP-HPLC (reverse phase high performance liquid chromatography); r.t. (room temperature), s (singlet); sat. (saturated); Selectfluor (1-chloromethyl-4-fluoro-1,4-diazoniabicyclo[2.2.2]octane bis(tetrafluoroborate)); t (triplet or tertiary); TBAF (tetra-n-butylammonium fluoride); TBDPS (tert-butyldiphenylsilyl); TBS (tert-butyldimethylsilyl); TEMPO (2,2,6,6-tetramethylpiperidine-N-oxyl); tert (tertiary); tt (triplet of triplets); TFA (trifluoroacetic acid); THF (tetrahydrofuran); TMS-OTf (trimethylsilyl trifluoromethanesulfonate); p-TsOH (p-toluenesulfonic acid); μg (microgram(s)); μL (microliter(s)); μM (micromolar); wt % (weight percent); xantphos (4,5-bis(diphenylphosphino)-9,9-dimethylxanthene); XPhos Pd G4 ((SP-4-3)-[dicyclohexyl[2′,4′,6′-tris(1-methylethyl)[1,1′-biphenyl]-2-ylphosphine](methanesulfonato-κO)[2′-(methylamino-κN)[1,1′-biphenyl]-2-yl-KC]palladium). t-BuXPhos Pd G4 (methanesulfonato(2-di-tbutylphosphino-2′,4′,6′-tri-1-propyl-1,1′-biphenyl)(2′-methylamino-1,1′-biphenyl-2-yl)palladium(II) dichloromethane adduct); SPhos Pd G4 ((methanesulfonato-κO)[2′-(methylamino)-2-biphenyly]balladium-dicyclohexyl(2′,6′-dimethoxy-2-biphenylyl)phosphine). Brine is sat. aq. NaCl. In vacuo is under vacuum.

### Intermediate 1. Methyl 4-bromo-3-chloro-5-fluoro-2-iodobenzoate

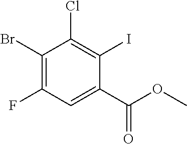

### Step 1. Methyl 2-amino-4-bromo-3-chloro-5-fluorobenzoate

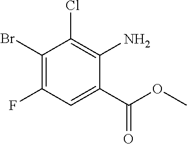

To a solution of methyl 2-amino-4-bromo-5-fluorobenzoate (25 g, 101 mmol) in DMF (160 mL) was added NCS (15.1 g, 111 mmol) and the mixture was heated at 65° C. for 1 h, then allowed to cool to r.t. The mixture was then slowly added to ice water with stirring. After stirring for 20 min., the resulting solid was collected by filtration, washed with water and allowed to air dry. The crude product was used in the next step without further purification (27.8 g, 98% yield). LCMS calc. for C₈H₇BrClFNO₂ (M+H)⁺: m/z=281.9, 283.9; found 281.9, 283.9.

### Step 2. Methyl 4-bromo-3-chloro-5-fluoro-2-iodobenzoate

A solution of methyl 2-amino-4-bromo-3-chloro-5-fluorobenzoate (60.5 g, 214 mmol) in 2-MeTHF (221 mL) was cooled to 0° C. and tetrafluoroboric acid (63.7 mL, 428 mmol) was added slowly, maintaining the internal temperature <10° C. The mixture was stirred at 0° C. for 5 min., then tert-butyl nitrite (42.5 mL, 321 mmol) was added slowly to maintain temperature below 5° C. Additional 2-MeTHF can be added at this stage to maintain stirring as necessary. After addition was complete, the mixture was stirred for an additional 5 min., then MTBE (600 mL) was added to complete precipitation of the intermediate tetrafluoroborate diazonium salt. This solid was collected by filtration, washed with MTBE (500 mL) and diethyl ether (100 mL), then air dried.

In a separate vessel, KI (70.7 g, 426 mmol) was dissolved in water (900 mL) and cooled to 0° C. with rapid overhead stirring. The solid diazonium tetrafluoroborate salt was then added portion-wise to this mixture while maintaining internal temperature <10° C. After addition was complete, the mixture was removed from the bath and allowed to warm to r.t. The resulting solid was collected by filtration and washed with water. The solid was taken up in water/EtOAc and the organic layer separated, washed with brine, dried over MgSO₄ and concentrated to provide the title compound (63.6 g, 96% yield). LCMS calc. for C₈H₅BrClFIO₂ (M+H)⁺: m/z=392.8, 394.8; found 392.8, 394.8.

### Intermediate 2. Methyl 4-(2-((tert-butoxycarbonyl)amino)-3-cyano-7-fluorobenzo[b]thiophen-4-yl)-3-chloro-5-fluoro-2-(2-oxoethyl)benzoate

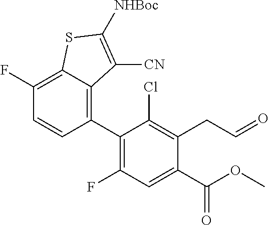 [SMILES: COC(=O)c1cc(F)c(-c2ccc(F)c3sc(NC(=O)OC(C)(C)C)c(C#N)c23)c(Cl)c1CC=O]

### Step 1. methyl (E)-4-bromo-3-chloro-2-(2-ethoxyvinyl)-5-fluorobenzoate

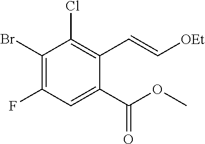 [SMILES: CCO/C=C/c1c(C(=O)OC)cc(F)c(Br)c1Cl]

To a mixture of Intermediate 1 (20 g, 50.8 mmol), dppf-PdCl₂ (2.68 g, 3.7 mmol) and Na₂CO₃ (13.5 g, 127 mmol) were added 1,4-dioxane (200 mL), water (50 mL) and (E)-2-(2-ethoxyvinyl)-4,4,5,5-tetramethyl-1,3,2-dioxaborolane (11.9 g, 60 mmol) and the mixture was placed under N₂, then heated at 90° C. for 90 min. then 60° C. overnight. The mixture was diluted with 1:1 water/brine (60 mL), then extracted with EtOAc. The organic layer was dried over MgSO₄, filtered and concentrated. The crude product was purified by FCC (0-20% heptane/MTBE) to provide the title compound (15.3 g, 89% yield). LCMS calc. for C₁₂H₁₂BrClFO₃ (M+H)⁺: m/z=337.0, 339.0; found 337.0, 339.0.

### Step 2. Methyl (E)-4-(2-((tert-butoxycarbonyl)amino)-3-cyano-7-fluorobenzo[b]thiophen-4-yl)-3-chloro-2-(2-ethoxyvinyl)-5-fluorobenzoate

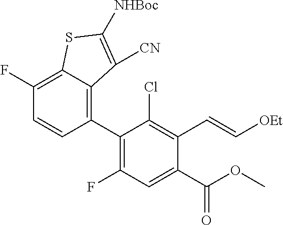 [SMILES: CCO/C=C/c1c(C(=O)OC)cc(F)c(-c2ccc(F)c3sc(NC(=O)OC(C)(C)C)c(C#N)c23)c1Cl]

To a mixture of methyl (E)-4-bromo-3-chloro-2-(2-ethoxyvinyl)-5-fluorobenzoate (500 mg, 1.48 mmoL), Cs₂CO₃ (1.2 g, 3.7 mmol), tert-butyl (3-cyano-4-(5,5-dimethyl-1,3,2-dioxaborinan-2-yl)-7-fluorobenzo[b]thiophen-2-yl)carbamate (990 mg, 2.4 mmol) and dichloro[bis(diphenylphosphinophenyl)etherballadium (11) (Pd-117, 212 mg, 0.3 mmol) was added 1,4-dioxane (15 mL) and the mixture was placed under N₂, then stirred at 85° C. for 12 h. The mixture was then diluted with EtOAc and filtered through a plug of diatomaceous earth. The filtrate was concentrated and the crude product was purified by FCC (10-100% EtOAc in hexanes) to provide the title compound (520 mg, 64% yield). LCMS calc. for C₂₆H₂₄ClF2N₂O₅S (M+H)⁺: m/z=549.1; found 549.1.

### Step 3. Methyl 4-(2-((tert-butoxycarbonyl)amino)-3-cyano-7-fluorobenzo[b]thiophen-4-yl)-3-chloro-5-fluoro-2-(2-oxoethyl)benzoate

To a solution of methyl (E)-4-(2-((tert-butoxycarbonyl)amino)-3-cyano-7-fluorobenzo[b]thiophen-4-yl)-3-chloro-2-(2-ethoxyvinyl)-5-fluorobenzoate (570 mg, 1.04 mmol) in DCM (25 mL) was added TFA (0.4 mL, 5.18 mmol) and the mixture was stirred at r.t. for 1 h, then concentrated. The crude product was purified by FCC (5-100% EtOAc in hexanes) to provide the title compound (510 mg, 94% yield). LCMS calc. for C₂₄H₂₃ClF₂N₃O₅S (M+NH₄)⁺: m/z=538.1; found 538.2

### Intermediate 3. 3-(1-Aminoethyl)-N,N-bls(4-methoxybenzyl)pyridin-2-amine

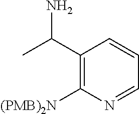

### Step 1. 1-(2-(Bis(4-methoxybenzyl)amino)pyridin-3-yl)ethan-1-one

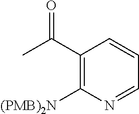

To a solution of 1-(2-chloropyridin-3-yl)ethan-1-one (15.3 g, 98 mmol) in 1,4-dioxane (50 mL) was added bis(4-methoxybenzyl)amine (50.5 g, 196 mmol) and the mixture was stirred under N₂ at 110° C. overnight. The mixture was then allowed to cool to r.t., diluted with ether and filtered. The filtrate was concentrated and the crude product was purified by FCC (0-70% EtOAc in hexanes) to provide the title compound (29.3 g, 79% yield). LCMS calc. for C₂₃H₂₅N₂O₃(M+H)⁺: m/z=377.2; found 377.2.

### Step 2. 1-(2-(Bis(4-methoxybenzyl)amino)pyridin-3-yl)ethan-1-ol

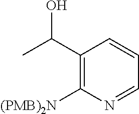

To a solution of 1-(2-bis(4-methoxybenzylamino)pyridin-3-yl)ethan-1-one (29.3 g, 78 mmol) in methanol (130 mL) at 0° C. was added NaBH₄ (4.4 g, 117 mmol) portionwise. The mixture was removed from the ice bath and stirred at r.t. for 30 min., then concentrated. The residue was partitioned between brine and EtOAc. The organic layer was separated, washed with brine, dried over MgSO₄ and concentrated. The crude product was purified by FCC (50-70% EtOAc in hexanes) to provide the title compound (27 g, 92% yield). LCMS calc. for C₂₃H₂₇N₂O₃(M+H)⁺: m/z=379.2; found 379.2.

### Step 3. 3-(1-Azidoethyl)-N,N-bis(4-methoxybenzyl)pyridin-2-amine

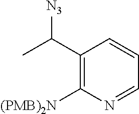

To a solution of 1-(2-(bis(4-methoxybenzyl)amino)pyridin-3-yl)ethan-1-ol (10.8 g, 28.6 mmol) and diphenylphosphoryl azide (12.3 mL, 57.2 mmol) in toluene (100 mL) at 0° C. was added DBU (8.6 mL, 57.2 mmol). The mixture was allowed to warm to r.t. overnight, then quenched with water and extracted with EtOAc. The organic layer was washed with brine, dried over MgSO₄ and concentrated. The crude product was purified by FCC (0-75% MTBE in hexanes) to provide the title compound (10.9 g, 94% yield). LCMS calc. for C₂₃H₂₆N₅O₂(M+H)⁺: m/z=404.2; found 404.2.

### Step 4. 3-(1-Aminoethyl)-N,N-bis(4-methoxybenzyl)pyridin-2-amine

To a solution of 3-(1-azidoethyl)-N,N-bis(4-methoxybenzyl)pyridin-2-amine (7.4 g, 18.4 mmol) in THF (80 mL) under N₂ was added trimethylphosphine (1 M in THF, 27.7 mL, 27.7 mmol). The mixture was stirred at r.t. for 1 h, then quenched with water (13 mL) and stirred at r.t. for an additional 30 min. The mixture was diluted with EtOAc/brine and the organic layer was separated, then 2N HCl was added. The layers were once again separated and the organic layer was discarded. The aqueous layer was basified to pH 10 with 2 M NaOH, then extracted with EtOAc. The organic layer was washed with water and brine, dried over MgSO₄ and concentrated. The crude product was used in the next step without further purification (7.1 g, ˜100%). LCMS calc. for C₂₃H₂₈N₃O₂(M+H)⁺: m/z=378.2; found 378.2.

### Intermediate 4. 3-(1-Bromoethyl)-2-nitropyridine

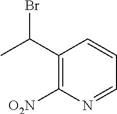

### Step 1. 2-Nitro-3-(prop-1-en-2-yl)pyridine

To a mixture of 3-bromo-2-nitropyridine (7.66 g, 37.7 mmol), isopropenyl boronic acid pinacol ester (9.2 mL, 49.1 mmol), XPhos Pd G4 (0.98 g, 1.1 mmol) and K₃PO₄ (24 g, 113 mmol) was added 5:1 1,4-dioxane/water (75 mL total volume) and the mixture was heated at 80° C. under N₂ for 30 min. The reaction was quenched with water and the mixture was extracted with EtOAc. The organic layer was washed with brine, dried over MgSO₄ and concentrated. The crude product was purified by FCC (0-100% EtOAc in heptane) to provide the title compound (5.5 g, 90% yield). LCMS calc. for C₈H₉N₂O₂ (M+H)⁺: m/z=165.1; found 165.1.

### Step 2. 1-(2-Nitropyridin-3-yl)ethan-1-one

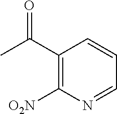

To a solution of 2-nitro-3-(prop-1-en-2-yl)pyridine (5.5 g, 34 mmol) in THF (135 mL)/water (34 mL) was added osmium tetroxide solution (0.05 w/v in THF, 20 mL, 3.39 mmol) and NaIO₄ (36 g, 169 mmol). The mixture was heated at 55° C. for 4 h, then overnight at 45° C. The mixture was diluted with EtOAc/water and the layers were separated. The organic layer was washed with sat. aq. Na₂SO₃ and brine, dried over MgSO₄ and concentrated. The crude product was purified by FCC (0-100% EtOAc in heptane) to provide the title compound (4.8 g, 85% yield). LCMS calc. for C₇H7N₂O₃(M+H)⁺: m/z=167.1; found 167.1.

### Step 3. 1-(2-Nitropyridin-3-yl)ethan-1-ol

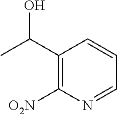

To a solution of 1-(2-nitropyridin-3-yl)ethan-1-one (1.03 g, 6.2 mmol) in ethanol (20 mL) at 0° C. was added NaBH₄ (470 mg, 12.4 mmol) and the mixture was warmed to r.t. and stirred for 30 min., then quenched with water and extracted with EtOAc. The organic layer was washed with water and brine, dried over MgSO₄ and concentrated. The crude product was used in the next step without further purification. LCMS calc. for C₇H9N₂03 (M+H)⁺: m/z=169.1; found 169.1.

### Step 4. 3-(1-Bromoethyl)-2-nitropyridine

To a solution of 1-(2-nitropyridin-3-yl)ethan-1-ol (319 mg, 1.9 mmol) and triphenylphosphine (905 mg, 3.45 mmol) in THF (12 mL) at 0° C. was added carbon tetrabromide (956 mg, 2.88 mmol) and the mixture was allowed to warm to r.t. and stir for 30 min. The mixture was filtered through diatomaceous earth and the solid washed with cold THF. The filtrate was concentrated and the crude product was purified by FCC (0-30% EtOAc in heptane) to provide the title compound (387 mg, 88% yield). LCMS calc. for C₇H₈BrN₂O₂(M+H)⁺: m/z=231.0, 233.0; found 231.0, 233.0.

### Intermediate 5. 2-(1-(2-Aminopyridin-3-yl)ethyl)-6-bromo-5-chloro-7-fluoro-3,4-dihydroisoquinolin-1(2H)-one

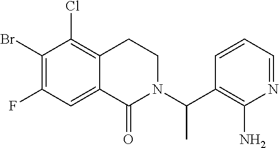 [SMILES: CC(c1cccnc1N)N1CCc2c(cc(F)c(Br)c2Cl)C1=O]

### Step 1. Methyl (E)-3-(3-bromo-2-chloro-4-fluorophenyl)acrylate

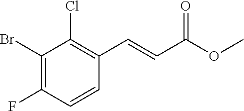

A solution of 3-bromo-2-chloro-4-fluorobenzaldehyde (1.2 g, 5.1 mmol) and methyl (triphenylphosphoranylidene)acetate (2.03 g, 6.1 mmol) were dissolved in DCM (13 mL) and stirred at r.t. for 1 h, then quenched by pouring into sat. aq. NaHCO₃. The mixture was extracted with DCM. The organic layer was washed with brine, dried over MgSO₄ and concentrated. The crude product was purified by FCC (0-40% EtOAc in heptane) to provide the title compound. LCMS calc. for C₁₀H₈BrClFO₂ (M+H)⁺: m/z=292.9, 294.9; found 293.0, 295.0.

### Step 2. 3-(3-Bromo-2-chloro-4-fluorophenyl)propanoic acid

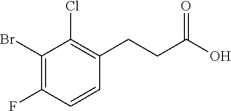 [SMILES: O=C(O)CCc1ccc(F)c(Br)c1Cl]

BDPCuH was prepared according to literature procedure (B. A. Barker et al., Org. Lett., 2008, 10, 289-92) and kept as a 0.001 M stock solution in toluene. An aliquot of this solution (53.1 mL, 0.053 mmol) was added to methyl (E)-3-(3-bromo-2-chloro-4-fluorophenyl)acrylate (6.5 g, 22.1 mmol), followed by PMHS (14.76 mL, 44.3 mmol) and tertbutanol (12.7 mL, 133 mmol). The resulting mixture was stirred at r.t. overnight. The mixture was concentrated, then dissolved in a mixture of 2 M NaOH/ethanol/THF (2:1:0.5, 100 mL total). The resulting solution was stirred at 45° C. for 1 h, then added to a mixture of 2 M HCl and ice with stirring. The resulting solid was collected by filtration, washed with water and air dried to provide the title compound, which was used in the next step without further purification (4.6 g, 74% yield). LCMS calc. for C₉H₈BrClFO₂ (M+H)⁺: m/z=280.9, 282.9; found 281.0, 283.0.

### Step 3. 5-Bromo-4-chloro-6-fluoro-2,3-dihydro-1H-inden-1-one

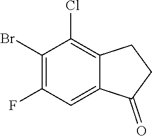 [SMILES: O=C1CCc2c1cc(F)c(Br)c2Cl]

3-(3-Bromo-2-chloro-4-fluorophenyl)propanoic acid (4.6 g, 16.5 mmol) was placed in a 250 mL roundbottom flask and cooled to 0° C. with stirring. To this solid was added chlorosulfonic acid (33.1 mL, 494 mmol) slowly. The mixture was allowed to warm to r.t. overnight, then slowly added into ice water. The mixture was extracted with DCM. The organic layer was washed with sat. aq. NaHCO₃ and brine, dried over MgSO₄ and concentrated. The crude product was used in the next step without further purification (3.8 g, 87% yield). LCMS calc. for C₉H₆BrClFO (M+H)⁺: m/z=262.9, 264.9; found 263.0, 265.0.

### Step 4. 6-Bromo-5-chloro-7-fluoro-3,4-dihydroisoquinolin-1(2H)-one

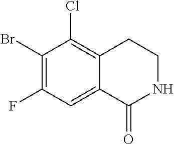 [SMILES: O=C1NCCc2c1cc(F)c(Br)c2Cl]

To a solution of 5-bromo-4-chloro-6-fluoro-2,3-dihydro-1H-inden-1-one (3.8 g, 14.4 mmol) in DCM (22 mL) at −5° C. was added methanesulfonic acid (9.3 mL, 144 mmol) and the mixture was stirred for 5 min. at this temperature. Sodium azide (2.4 g, 37.1 mmol) was added portionwise and the mixture was stirred at −5° C. for 2 h. The reaction was then quenched by the addition of 2 M NaOH (100 mL) at −5° C. [CAUTION: hydrazoic acid is formed during this reaction; care should be taken to ensure quenched mixture reaches pH 10 before the ice bath is removed. Low temperature must be obtained during quenching to avoid formation of diazidoethane, which is potentially explosive.] The mixture was then diluted with EtOAc. The layers were separated and the organic layer was washed with brine, dried over MgSO₄ and concentrated. The crude product was recrystallized from boiling toluene to provide the title compound (3.5 g, 87% yield). LCMS calc. for C₉H₇BrClFNO (M+H)⁺: m/z=277.9, 279.9; found 278.0, 280.0.

### Step 5. 6-Bromo-5-chloro-7-fluoro-2-(1-(2-nitropyridin-3-yl)ethyl)-3,4-dihydroisoquinolin-1(2H)-one

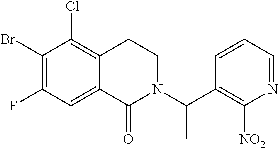 [SMILES: CC(c1cccnc1[N+](=O)[O-])N1CCc2c(cc(F)c(Br)c2Cl)C1=O]

To solid KH (35 mg, 0.43 mmol, 50 wt %) at 0° C. under N₂ was added a solution of 6-bromo-5-chloro-7-fluoro-3,4-dihydroisoquinolin-1(2H)-one (100 mg, 0.36 mmol) in DMF (3 mL). The mixture was stirred at this temperature for 10 min., then a solution of 3-(1-bromoethyl)-2-nitropyridine (Intermediate 4,124 mg, 0.54 mmol) in DMF (1 mL) was added and the mixture was stirred at r.t. for 1 h. The reaction was quenched with sat. aq. NH₄Cl, and the mixture was diluted with water and extracted with DCM. The organic layer was washed with water and brine, dried over Na₂SO₄ and concentrated. The crude product was used in the next step without further purification. LCMS calc. for C₁₆H₁₃BrClFN₃O₃ (M+H)⁺: m/z=428.0, 430.0; found 428.0, 430.0.

### Step 6. 2-(1-(2-Aminopyridin-3-yl)ethyl)-6-bromo-5-chloro-7-fluoro-3,4-dihydroisoquinolin-1(2H)-one

To a solution of 6-bromo-5-chloro-7-fluoro-2-(1-(2-nitropyridin-3-yl)ethyl)-3,4-dihydroisoquinolin-1(2H)-one (40 mg, 0.096 mmol) dissolved in a 1:1:1 THF/water/MeOH (1.5 mL total volume) iron (53 mg, 0.95 mmol) and NH₄Cl (51 mg, 0.95 mmol) were added. The mixture was heated at 60° C. for 2 h, then diluted with DCM and filtered through a plug of diatomaceous earth. The filtrate was washed with brine, dried over MgSO₄ and concentrated. The crude product was used in the next step without further purification (29 mg, 79% yield). LCMS calc. for C₁₆H₁₅BrClFN₃O (M+H)⁺: m/z=398.0, 400.0; found 398.0, 400.0.

### Example 1. 2-Amino-4-(2-(1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile

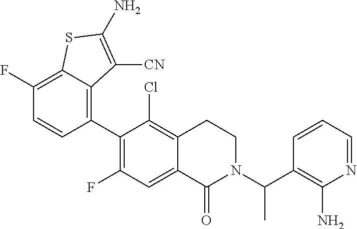 [SMILES: CC(c1cccnc1N)N1CCc2c(cc(F)c(-c3ccc(F)c4sc(N)c(C#N)c34)c2Cl)C1=O]

### Step 1. Methyl 4-bromo-3-chloro-5-fluoro-2-(2-oxoethyl)benzoate

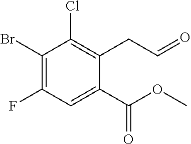 [SMILES: COC(=O)c1cc(F)c(Br)c(Cl)c1CC=O]

To a solution of methyl (E)-4-bromo-3-chloro-2-(2-ethoxyvinyl)-5-fluorobenzoate (Intermediate 2, Step 1, 12.6 g, 37.4 mmol) in DCM (180 mL) at 0° C. was added difluoroacetic acid (8 mL, 127 mmol) and the mixture was stirred at this temperature for 15 min. The reaction was quenched with sat. aq. NaHCO₃ and the mixture was extracted with DCM. The organic layer was dried over MgSO₄ and concentrated. The crude product was purified by FCC (0-40% EtOAc in heptane) to provide the title compound (7.2 g, 62% yield). LCMS calc. for C₁₀H₈BrClFO₃ (M+H)⁺: m/z=309.0, 311.0; found 309.0, 311.0.

### Step 2. 2-(1-(2-(Bis(4-methoxybenzyl)amino)pyridin-3-yl)ethyl)-6-bromo-5-chloro-7-fluoro-3,4-dihydroisoquinolin-1(2H)-one

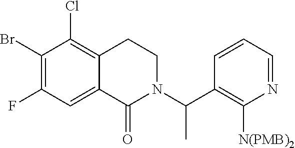

To a solution of 3-(1-aminoethyl)-N,N-bis(4-methoxybenzyl)pyridin-2-amine (Intermediate 3, 5.4 g, 14.3 mmol) in DCE (76 mL) at 0° C. were added methyl 4-bromo-3-chloro-5-fluoro-2-(2-oxoethyl)benzoate (5.1 g, 16.3 mmol), NaBH(OAc)₃ (9.1 g, 42.9 mmol) and HFIP (19 mL). TFA (2.2 mL, 28.6 mmol) was then added and the mixture was stirred at 0° C. for 1 h, then poured into sat. aq. NaHCO₃. The mixture was extracted with DCM. The organic layer was washed with water and brine, dried over MgSO₄ and concentrated.

The crude product was taken up in toluene (100 mL) and DIPEA (25 mL, 143 mmol) was added. The mixture was heated at 100° C. and stirred for 1 h, then cooled to 60° C. and stirred overnight. The crude mixture was concentrated and purified by FCC (0-80% EtOAc in heptane) to provide the title compound (5.9 g, 65% yield). LCMS calc. for C₃₂H₃₁BrClFN₃O₃ (M+H)⁺: m/z=638.1, 640.1; found 638.1, 640.1.

LCMS calc. for C₂₅H₁₉ClF₂N₅OS (M+H)⁺: m/z=510.1; found 510.1.

### Step 3. tert-Butyl (3-(1-(6-bromo-5-chloro-7-fluoro-1-oxo-3,4-dihydroisoquinolin-2(1H)-yl)ethyl)pyridin-2-yl)carbamate

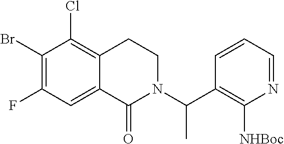 [SMILES: CC(c1cccnc1NC(=O)OC(C)(C)C)N1CCc2c(cc(F)c(Br)c2Cl)C1=O]

In a 40 mL vial 2-(1-(2-(bis(4-methoxybenzyl)amino)pyridin-3-yl)ethyl)-6-bromo-5-chloro-7-fluoro-3,4-dihydroisoquinolin-1(2H)-one (2.2 g, 3.44 mmol) was dissolved in TFA (17 mL) under N₂. The mixture was heated at 45° C. for 3 h. Volatiles were removed in vacuo and the residue was dissolved in DCM and washed with sat. aq. NaHCO₃. The organic phase was dried over MgSO₄ and concentrated. The crude product was dissolved in DCM (13.6 mL) and Boc-anhydride (1.198 mL, 5.16 mmol), NEt₃ (4.79 mL, 34.4 mmol), and DMAP (105 mg, 0.860 mmol) were added under N₂. The mixture was stirred at r.t. for 2 h and then quenched with sat. aq. NaHCO₃, extracted with DCM, dried over MgSO₄, and concentrated. The crude residue was purified by FCC (0-70% acetone in heptane) to provide the title compound (1.33 g, 2.7 mmol, 77% yield). LCMS calc. for C₂₁H₂₃BrClFN₃O₃ (M+H)⁺: m/z=498.1, 500.1; found 498.1, 500.1.

### Step 4. tert-Butyl (3-(1-(6-(2-((tert-butoxycarbonyl)amino)-3-cyano-7-fluorobenzo[b]thiophen-4-yl)-5-chloro-7-fluoro-1-oxo-3,4-dihydroisoquinolin-2(1H)-yl)ethyl)pyridin-2-yl)carbamate

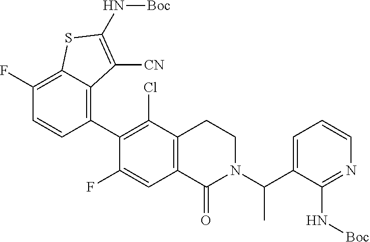 [SMILES: CC(c1cccnc1NC(=O)OC(C)(C)C)N1CCc2c(cc(F)c(-c3ccc(F)c4sc(NC(=O)OC(C)(C)C)c(C#N)c34)c2Cl)C1=O]

In a 40 mL vial tert-butyl (3-(1-(6-bromo-5-chloro-7-fluoro-1-oxo-3,4-dihydroisoquinolin-2(1H)-yl)ethyl)pyridin-2-yl)carbamate (360 mg, 0.72 mmol), tert-butyl (3-cyano-4-(5,5-dimethyl-1,3,2-dioxaborinan-2-yl)-7-fluorobenzo[b]thiophen-2-yl)carbamate (443 mg, 1.1 mmol), Dichloro[bis(diphenylphosphinophenyl)etherballadium(II) (59 mg, 0.08 mmol), and Cs₂CO₃ (588 mg, 1.80 mmol) were dissolved in toluene (7.2 mL) under N₂. The mixture was stirred rapidly and heated at 105° C. for 2 h. The mixture was cooled, diluted with EtOAc and water, extracted, dried over MgSO₄ and concentrated. The crude residue was purified via FCC (0-70% acetone in heptane) to provide the title compound (345 mg, 0.72 mmol, 78%). LCMS calc. for C₃₅H₃₅ClF₂N₅O₅S (M+H)⁺: m/z=709.2; found 709.2.

### Step 5. 2-Amino-4-(2-(1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile

tert-Butyl (4-(2-(1-(2-((tert-butoxycarbonyl)amino)pyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate (53 mg, 0.075 mmol) was dissolved in TFA (0.4 mL) under N₂ and stirred for 2 h at r.t. The mixture was diluted with MeCN (4 mL) and purified by prep. LCMS (XBRIDGE® C₁₈ column, eluting with a gradient of MeCN/water containing 0.1% TFA, at flow rate of 60 mL/min.) to afford the title compound. LCMS calc. for C₂₅H₁₉ClF₂N₅OS (M+H)⁺: m/z=510.1; found 510.1.

The title compound comprises a mixture of diastereoisomers, i.e.,
(Rₐ)-2-amino-4-(2-((R)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile;
(Sₐ)-2-amino-4-(2-((S)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile;
(Rₐ)-2-amino-4-(2-((S)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile; and
(Sₐ)-2-amino-4-(2-((R)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile.

### Example 2. 2-Amino-4-(2-(1-(2-aminopyridin-3-yl)ethyl)-7-fluoro-5-methyl-1-oxo-1,2,3,4-tetrahydroisoquinolin-6-yl)-7-fluorobenzo[b]thiophene-3-carbonitrile

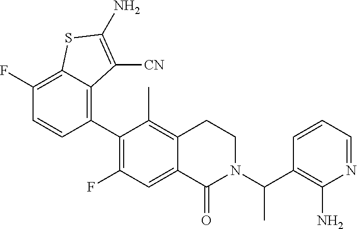 [SMILES: Cc1c2c(cc(F)c1-c1ccc(F)c3sc(N)c(C#N)c13)C(=O)N(C(C)c1cccnc1N)CC2]

### Step 1. tert-Butyl(4-(2-(1-(2-aminopyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate

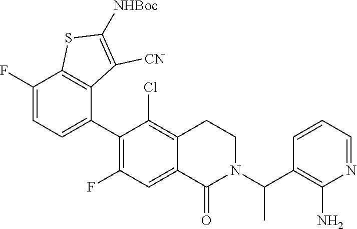 [SMILES: CC(c1cccnc1N)N1CCc2c(cc(F)c(-c3ccc(F)c4sc(NC(=O)OC(C)(C)C)c(C#N)c34)c2Cl)C1=O]

To a mixture of Intermediate 5 (159 mg, 0.4 mmol), tert-butyl (3-cyano-4-(5,5-dimethyl-1,3,2-dioxaborinan-2-yl)-7-fluorobenzo[b]thiophen-2-yl)carbamate (322 mg, 0.9 mmol), K₂CO₃ (276 mg, 2.0 mmol) and dppf-PdCl₂ (44 mg, 0.06 mmol) was added 1,4-dioxane (4 mL) and the mixture was placed under a N₂ and stirred at 115° C. for 2 h. The reaction was quenched with water and the mixture was extracted with EtOAc. The organic layer was washed with brine, dried over MgSO₄ and concentrated. The crude product was purified by prep.-SFC (Waters DEA column, eluting with a gradient of MeOH/CO₂, 4-24% at 140 mL/min.) to provide the title compound (65 mg, 27% yield). LCMS calc. for C₃₀H₂₇ClF₂N₅O₃S (M+H)⁺: m/z=610.1; found 610.1.

### Step 2. 2-Amino-4-(2-(1-(2-aminopyridin-3-yl)ethyl)-7-fluoro-5-methyl-1-oxo-1,2,3,4-tetrahydroisoquinolin-6-yl)-7-fluorobenzo[b]thiophene-3-carbonitrile

To a mixture of tert-butyl (4-(2-(1-(2-aminopyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate (35 mg, 0.06 mmol), methylboronic acid (34 mg, 0.57 mmol) and SPhos Pd G4 (7 mg, 8.6 μmol) were added 1,4-dioxane (0.7 mL) and 0.5 M aq. K₃PO₄ (50 μL) and the mixture was placed under N₂ and heated at 85° C. overnight. The mixture was concentrated, then treated with 4 M HCl in 1,4-dioxane (1 mL) for 30 min. at r.t. The mixture was diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₆H₂₂F₂N₅OS (M+H)⁺: m/z=490.1; found 490.1.

The title compound comprises a mixture of diastereoisomers, i.e.,
(Rₐ)-2-amino-4-(2-((R)-1-(2-aminopyridin-3-yl)ethyl)-7-fluoro-5-methyl-1-oxo-1,2,3,4-tetrahydroisoquinolin-6-yl)-7-fluorobenzo[b]thiophene-3-carbonitrile;
(Sₐ)-2-amino-4-(2-((S)-1-(2-aminopyridin-3-yl)ethyl)-7-fluoro-5-methyl-1-oxo-1,2,3,4-tetrahydroisoquinolin-6-yl)-7-fluorobenzo[b]thiophene-3-carbonitrile;
(Rₐ)-2-amino-4-(2-((S)-1-(2-aminopyridin-3-yl)ethyl)-7-fluoro-5-methyl-1-oxo-1,2,3,4-tetrahydroisoquinolin-6-yl)-7-fluorobenzo[b]thiophene-3-carbonitrile; and
(Sₐ)-2-amino-4-(2-((R)-1-(2-aminopyridin-3-yl)ethyl)-7-fluoro-5-methyl-1-oxo-1,2,3,4-tetrahydroisoquinolin-6-yl)-7-fluorobenzo[b]thiophene-3-carbonitrile.

### Example 3. 2-Amino-4-(2-(1-(2-aminopyridin-3-yl)ethyl)-5-chloro-7-fluoro-1-oxo-1,2,3,4-tetrahydroisoquinolin-6-yl)-5-fluorobenzo[b]thiophene-3-carbonitrile

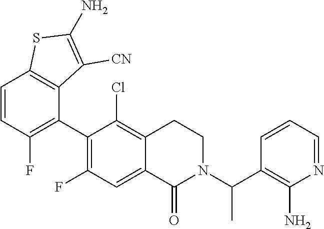 [SMILES: CC(c1cccnc1N)N1CCc2c(cc(F)c(-c3c(F)ccc4sc(N)c(C#N)c34)c2Cl)C1=O]

### Step 1. tert-Butyl (3-(1-(6-bromo-5-chloro-7-fluoro-1-oxo-3,4-dihydroisoquinolin-2(1H)-yl)ethyl)pyridin-2-yl)carbamate

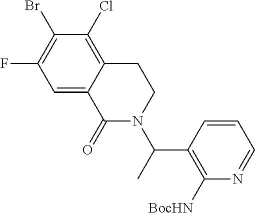 [SMILES: CC(c1cccnc1NC(=O)OC(C)(C)C)N1CCc2c(cc(F)c(Br)c2Cl)C1=O]

To a solution of Intermediate 5 (1.0 g, 2.5 mmol) in THF (12 mL) were added boc-anhydride (1.1 g, 5 mmol), NEt₃ (1.05 mL, 7.5 mmol) and DMAP (153 mg, 1.25 mmol) and the mixture was stirred at 40° C. for 5 h. The mixture was concentrated and the crude product was purified by FCC (0-80% EtOAc in hexanes) to provide the title compound. LCMS calc. for C₂₁H₂₃BrClFN₃O₃(M+H)⁺: m/z=498.1, 500.1; found 498.1, 500.1.

### Step 2. tert-Butyl (4-(2-(1-(2-((tert-butoxycarbonyl)amino)pyridin-3-yl)ethyl)-5-chloro-7-fluoro-1-oxo-1,2,3,4-tetrahydroisoquinolin-6-yl)-5-fluorobenzo[b]thiophen-2-yl)carbamate

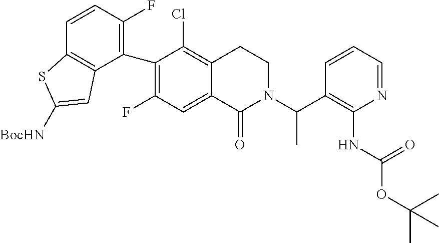 [SMILES: CC(c1cccnc1NC(=O)OC(C)(C)C)N1CCc2c(cc(F)c(-c3c(F)ccc4sc(NC(=O)OC(C)(C)C)cc34)c2Cl)C1=O]

To a mixture of the product from the previous step (130 mg, 0.26 mmol), tert-butyl (4-(5,5-dimethyl-1,3,2-dioxaborinan-2-yl)-5-fluorobenzo[b]thiophen-2-yl)carbamate (148 mg, 0.39 mmol), XPhos Pd G4 (45 mg, 0.052 mmol) and K₃PO₄ (111 mg, 0.52 mmol) was added 5:1 1,4-dioxane/water (2.5 mL total volume) and the mixture was placed under N₂, then heated at 50° C. for 30 min. The reaction was quenched with water and the mixture was extracted with EtOAc. The organic layer was washed with water and brine, dried over Na₂SO₄ and concentrated. The crude product was purified by FCC (0-80% EtOAc in hexanes) to provide the title compound. LCMS calc. for C₃₄H₃₆ClF₂N₄O₅S (M+H)⁺: m/z=685.2; found 685.2.

### Step 3. 2-Amino-4-(2-(1-(2-aminopyridin-3-yl)ethyl)-5-chloro-7-fluoro-1-oxo-1,2,3,4-tetrahydroisoquinolin-6-yl)-5-fluorobenzo[b]thiophene-3-carbonitrile

To a solution of the product from the previous step (80 mg, 0.117 mmol) in MeCN (1 mL) at −20° C. was added chlorosulfonyl isocyanate (51 μL, 0.58 mmol) dropwise. The mixture was stirred at this temperature for 30 min., then DMF (200 μL) was added. The reaction was quenched with sat. aq. NH₄Cl and the mixture was extracted with EtOAc. The organic layer was washed with water and brine, dried over MgSO₄ and concentrated. The crude product was diluted with DCM/TFA (1:1, 1 mL) and stirred at r.t. for 3 hr. The mixture was diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₅H₁₅ClF₂N₅OS (M+H)⁺: m/z=510.1; found 510.1.

The title compound comprises a mixture of diastereoisomers, i.e.,
(Rₐ)-2-amino-4-(2-((R)-1-(2-aminopyridin-3-yl)ethyl)-5-chloro-7-fluoro-1-oxo-1,2,3,4-tetrahydroisoquinolin-6-yl)-5-fluorobenzo[b]thiophene-3-carbonitrile;
(Sₐ)-2-amino-4-(2-((S)-1-(2-aminopyridin-3-yl)ethyl)-5-chloro-7-fluoro-1-oxo-1,2,3,4-tetrahydroisoquinolin-6-yl)-5-fluorobenzo[b]thiophene-3-carbonitrile;
(Rₐ)-2-amino-4-(2-((S)-1-(2-aminopyridin-3-yl)ethyl)-5-chloro-7-fluoro-1-oxo-1,2,3,4-tetrahydroisoquinolin-6-yl)-5-fluorobenzo[b]thiophene-3-carbonitrile; and
(Sₐ)-2-amino-4-(2-((R)-1-(2-aminopyridin-3-yl)ethyl)-5-chloro-7-fluoro-1-oxo-1,2,3,4-tetrahydroisoquinolin-6-yl)-5-fluorobenzo[b]thiophene-3-carbonitrile.

### Example 4. 6-Amino-2-(2-(1-(2-aminopyridin-3-yl)ethyl)-5-chloro-7-fluoro-1-oxo-1,2,3,4-tetrahydroisoquinolin-6-yl)-4-methyl-3-(trifluoromethyl)benzonitrile

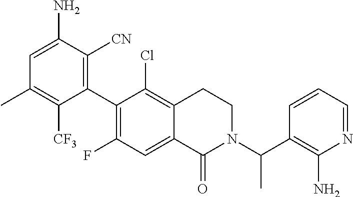 [SMILES: Cc1cc(N)c(C#N)c(-c2c(F)cc3c(c2Cl)CCN(C(C)c2cccnc2N)C3=O)c1C(F)(F)F]

### Step 1. tert-Butyl(3-(1-(6-(3-(bis(4-methoxybenzyl)amino)-2-cyano-5-methylphenyl)-5-chloro-7-fluoro-1-oxo-3,4-dihydroisoquinolin-2(1H)-yl)ethyl)pyridin-2-yl)carbamate

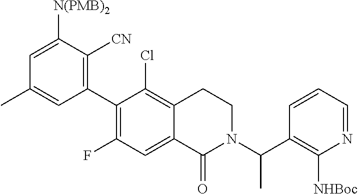

The product of Example 3, Step 1 (170 mg, 0.34 mmol) was placed in a vial with 2-(bis(4-methoxybenzyl)amino)-4-methyl-6-(4,4,5,5-tetramethyl-1,3,2-dioxaborolan-2-yl)benzonitrile (255 mg, 0.51 mmol), K₃PO₄ (217 mg, 1.02 mmol) and Pd(PPh₃)₄ (79 mg, 0.07 mmol). 1,4-dioxane (3.5 mL) was added and the mixture was placed under N₂ and stirred at 100° C. for 1 h. At this time, the mixture was concentrated and purified by FCC (0-100% EtOAc in hexanes) to provide the title compound (95 mg, 35% yield). LCMS calc. for C₄₅H₄₆ClFN₅O₅(M+H)⁺: m/z=790.3; found 790.3.

### Step 2. tert-Butyl (3-(1-(5-chloro-6-(2-cyano-6-iodo-3-((4-methoxybenzyl)amino)-5-methylphenyl)-7-fluoro-1-oxo-3,4-dihydroisoquinolin-2(1H)-yl)ethyl)pyridin-2-yl)carbamate

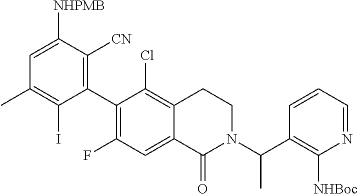

To a solution of the product from the previous step (150 mg, 0.19 mmol) in MeCN (2 mL) were added NIS (12 mg, 0.57 mmol) and TFA (29 μL, 0.38 mmol) and the mixture was stirred at r.t. for 3 h. The reaction was quenched with sat. aq. NaHCO₃ and the mixture was extracted with EtOAc. The organic layer was dried over MgSO₄ and concentrated, then purified by FCC (0-100% EtOAc in hexanes) to provide the title compound (85 mg, 56% yield). LCMS calc. for C₃₇H₃₇ClFIN₅O₄(M+H)⁺: m/z=796.2; found 796.2.

### Step 3. 6-Amino-2-(2-(1-(2-aminopyridin-3-yl)ethyl)-5-chloro-7-fluoro-1-oxo-1,2,3,4-tetrahydroisoquinolin-6-yl)-4-methyl-3-(trifluoromethyl)benzonitrile

To a vial containing the product from the last step (5 mg, 6.3 μmol) in DMF (0.5 mL) was added copper (4 mg, 63 μmol) at r.t. After stirring for 5 min., bis(2,2-difluoro-2-(fluorosulfonyl)acetoxy) copper (26 mg, 63 μmol) was added and the mixture was placed under N₂, then heated at 80° C. for 10 min. The mixture was diluted with EtOAc and filtered through a plug of diatomaceous earth. The filtrate was washed with brine, dried over Na₂SO₄and concentrated. The crude product was dissolved in TFA (1 mL) and heated at 50° C. for 10 min., then diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₅H₂₁ClF₄N₅O (M+H)⁺: m/z=518.1; found 518.1.

The title compound comprises a mixture of diastereoisomers, i.e.,
(Rₐ)-6-amino-2-(2-((R)-1-(2-aminopyridin-3-yl)ethyl)-5-chloro-7-fluoro-1-oxo-1,2,3,4-tetrahydroisoquinolin-6-yl)-4-methyl-3-(trifluoromethyl)benzonitrile;
(Sₐ)-6-amino-2-(2-((S)-1-(2-aminopyridin-3-yl)ethyl)-5-chloro-7-fluoro-1-oxo-1,2,3,4-tetrahydroisoquinolin-6-yl)-4-methyl-3-(trifluoromethyl)benzonitrile;
(Rₐ)-6-amino-2-(2-((S)-1-(2-aminopyridin-3-yl)ethyl)-5-chloro-7-fluoro-1-oxo-1,2,3,4-tetrahydroisoquinolin-6-yl)-4-methyl-3-(trifluoromethyl)benzonitrile; and
(Sₐ)-6-amino-2-(2-((R)-1-(2-aminopyridin-3-yl)ethyl)-5-chloro-7-fluoro-1-oxo-1,2,3,4-tetrahydroisoquinolin-6-yl)-4-methyl-3-(trifluoromethyl)benzonitrile.

### Example 5. 2-Amino-4-(2-(1-(2-aminopyridin-3-yl)ethyl)-5-chloro-7-fluoro-1-oxo-1,2,3,4-tetrahydroisoquinolin-6-yl)-5-chlorobenzo[b]thiophene-3-carbonitrile

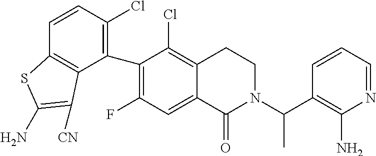 [SMILES: CC(c1cccnc1N)N1CCc2c(cc(F)c(-c3c(Cl)ccc4sc(N)c(C#N)c34)c2Cl)C1=O]

The title compound was prepared in an analogous fashion to Example 3, using tert-butyl (5-chloro-4-(5,5-dimethyl-1,3,2-dioxaborinan-2-yl)benzo[b]thiophen-2-yl)carbamate as the coupling partner in step 2. LCMS calc. for C₂₅H₁₉Cl₂FN₅OS (M+H)⁺: m/z=526.1; found 526.1.

The title compound comprises a mixture of diastereoisomers, i.e.,
(Rₐ)-2-amino-4-(2-((R)-1-(2-aminopyridin-3-yl)ethyl)-5-chloro-7-fluoro-1-oxo-1,2,3,4-tetrahydroisoquinolin-6-yl)-5-chlorobenzo[b]thiophene-3-carbonitrile;
(Sₐ)-2-amino-4-(2-((S)-1-(2-aminopyridin-3-yl)ethyl)-5-chloro-7-fluoro-1-oxo-1,2,3,4-tetrahydroisoquinolin-6-yl)-5-chlorobenzo[b]thiophene-3-carbonitrile;
(Rₐ)-2-amino-4-(2-((S)-1-(2-aminopyridin-3-yl)ethyl)-5-chloro-7-fluoro-1-oxo-1,2,3,4-tetrahydroisoquinolin-6-yl)-5-chlorobenzo[b]thiophene-3-carbonitrile; and
(Sₐ)-2-amino-4-(2-((R)-1-(2-aminopyridin-3-yl)ethyl)-5-chloro-7-fluoro-1-oxo-1,2,3,4-tetrahydroisoquinolin-6-yl)-5-chlorobenzo[b]thiophene-3-carbonitrile.

### Example 6. 2-Amino-4-(2-(1-(3-aminopyrazin-2-yl)ethyl)-5-chloro-7-fluoro-1-oxo-1,2,3,4-tetrahydroisoquinolin-6-yl)-7-fluorobenzo[b]thiophene-3-carbonitrile

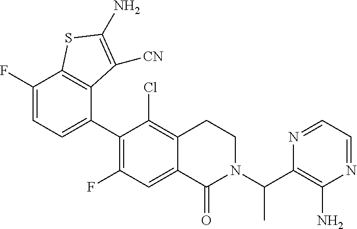 [SMILES: CC(c1nccnc1N)N1CCc2c(cc(F)c(-c3ccc(F)c4sc(N)c(C#N)c34)c2Cl)C1=O]

### Step 1. 1-(3-(Bis(4-methoxybenzyl)amino)pyrazin-2-yl)ethan-1-on

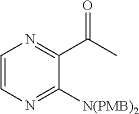

To a solution of 1-(3-chloropyrazin-2-yl)ethan-1-one (0.4 g, 2.5 mmol) in 1,4-dioxane (1.3 mL) was added bis(4-methoxybenzyl)amine (1.3 g, 5.1 mmol) and the mixture was heated at 110° C. for 90 min. The mixture was then allowed to cool to r.t., diluted with diethyl ether and filtered through a plug of diatomaceous earth. The filtrate was concentrated. The crude product was purified by FCC (0-70% EtOAc in hexanes) to provide the title compound (0.9 g, ˜100% yield). LCMS calc. for C₂₂H₂₄N₃O₃ (M+H)⁺: m/z=378.2; found 378.1.

### Step 2. 3-(1-Aminoethyl)-N,N-bis(4-methoxybenzyl)pyrazin-2-amine

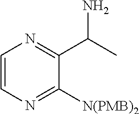

To a solution of 1-(3-(bis(4-methoxybenzyl)amino)pyrazin-2-yl)ethan-1-one (0.45 mg, 1.2 mmol) in methanol (12 mL)/MeCN (12 mL) was added ammonium acetate (0.92 g, 12 mmol) and the mixture was stirred at 65° C. for 45 min., then allowed to cool to r.t. Sodium cyanoborohydride (150 mg, 2.4 mmol) was then added and heating was continued at 65° C. overnight. The reaction was quenched with sat. aq. NaHCO₃ and the mixture was extracted with DCM. The organic layer was dried over Na₂SO₄ and concentrated. The crude product was purified by FCC (0-10% methanol in DCM) to provide the title compound (92 mg, 20% yield). LCMS calc. for C₂₂H₂₇N₄O₂(M+H)⁺: m/z=379.2; found 379.2.

### Step 3. 2-(1-(3-(Bis(4-methoxybenzyl)amino)pyrazin-2-yl)ethyl)-6-bromo-5-chloro-7-fluoro-3,4-dihydroisoquinolin-1(2H)-one

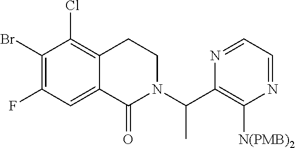

To a solution of methyl 4-bromo-3-chloro-5-fluoro-2-(2-oxoethyl)benzoate (Example 1, Step 1, 31 mg, 0.1 mmol) and 3-(1-aminoethyl)-N,N-bis(4-methoxybenzyl)pyrazin-2-amine (42 mg, 0.11 mmol) in DCE (4 mL) was added acetic acid (17 μL, 0.3 mmol) and the mixture was stirred at 80° C. for 10 min. NaBH(OAc)₃ (42 mg, 0.2 mmol) was then added and stirring was continued at 80° C. for 15 min. The mixture was then concentrated and redissolved in toluene (3 mL). DIPEA (70 μL, 0.4 mmol) was then added and the mixture was stirred at 100° C. for 15 min. At this point, the mixture was concentrated and purified by FCC (0-30% EtOAc in hexanes) to provide the title compound (29 mg, 46% yield). LCMS calc. for C₃₁H₃₀BrClFN₄O₃(M+H)⁺: m/z=639.1, 641.1; found 639.2, 641.2.

### Step 4. 2-Amino-4-(2-(1-(3-aminopyrazin-2-yl)ethyl)-5-chloro-7-fluoro-1-oxo-1,2,3,4-tetrahydroisoquinolin-6-yl)-7-fluorobenzo[b]thiophene-3-carbonitrile

A solution of the material from the previous step (29 mg, 0.045 mmol) in TFA (1.5 mL) was stirred at 70° C. for 1 h, then concentrated and azeotroped with toluene to remove residual TFA. To the resulting solid were added tert-butyl (3-cyano-4-(5,5-dimethyl-1,3,2-dioxaborinan-2-yl)-7-fluorobenzo[b]thiophen-2-yl)carbamate (18 mg, 0.045 mmol), K₂CO₃ (16 mg, 0.11 mmol), dppf-PdCl₂ (2.5 mg, 3.4 μmol) and 1,4-dioxane (1 mL). The mixture was placed under N₂, then heated at 115° C. for 50 min. The mixture was then diluted with EtOAc and filtered through a plug of diatomaceous earth. The filtrate was concentrated, then redissolved in MeCN (1 mL). 4 M HCl in 1,4-dioxane (0.5 mL) was then added and the mixture was stirred at r.t. for 1 h, then diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₄H₁₈ClF₂N₆OS (M+H)⁺: m/z=511.1; found 511.1.

The title compound comprises a mixture of diastereoisomers, i.e.,
(Rₐ)-2-amino-4-(2-((R)-1-(3-aminopyrazin-2-yl)ethyl)-5-chloro-7-fluoro-1-oxo-1,2,3,4-tetrahydroisoquinolin-6-yl)-7-fluorobenzo[b]thiophene-3-carbonitrile;
(Sₐ)-2-amino-4-(2-((S)-1-(3-aminopyrazin-2-yl)ethyl)-5-chloro-7-fluoro-1-oxo-1,2,3,4-tetrahydroisoquinolin-6-yl)-7-fluorobenzo[b]thiophene-3-carbonitrile;
(Rₐ)-2-amino-4-(2-((S)-1-(3-aminopyrazin-2-yl)ethyl)-5-chloro-7-fluoro-1-oxo-1,2,3,4-tetrahydroisoquinolin-6-yl)-7-fluorobenzo[b]thiophene-3-carbonitrile; and
(Sₐ)-2-amino-4-(2-((R)-1-(3-aminopyrazin-2-yl)ethyl)-5-chloro-7-fluoro-1-oxo-1,2,3,4-tetrahydroisoquinolin-6-yl)-7-fluorobenzo[b]thiophene-3-carbonitrile.

### Example 7. 2-Amino-4-(2-((3-amino-1-methyl-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile

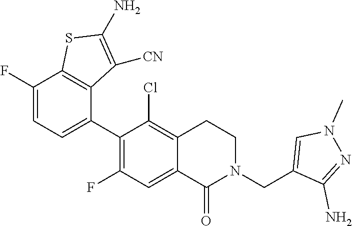 [SMILES: Cn1cc(CN2CCc3c(cc(F)c(-c4ccc(F)c5sc(N)c(C#N)c45)c3Cl)C2=O)c(N)n1]

To a solution of Intermediate 2 (50 mg, 0.1 mmol), 4-(aminomethyl)-1-methyl-1H-pyrazol-3-amine (12 mg, 0.1 mmol) and NaBH(OAc)₃ (81 mg, 0.38 mmol) in DCE (1 mL)/HFIP (0.25 mL) was added TFA (22 μL, 0.29 mmol) and the mixture was stirred at r.t. for 30 min. Toluene (1 mL) and DIPEA (0.33 mL, 1.92 mmol) were added and the mixture was heated at 100° C. for 30 min. The mixture was then concentrated and the residue redissolved in MeCN (3 mL)/TFA (2 mL). After stirring for 1 h, the mixture was filtered and purified by prep. LCMS (XBRIDGE® C₁₈ column, eluting with a gradient of MeCN/water containing 0.1% TFA, at flow rate of 60 mL/min.) to afford the title compound. LCMS calc. for C₂₃H₁₈ClF₂N₆OS (M+H)⁺: m/z=499.1; found 499.1.

The title compound comprises a mixture of diastereoisomers, i.e.,
(Rₐ)-2-amino-4-(2-((3-amino-1-methyl-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile; and
(Sₐ)-2-amino-4-(2-((3-amino-1-methyl-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile.

### Example 8. 2-Amino-4-(2-(1-(3-aminopyridazin-4-yl)ethyl)-5-chloro-7-fluoro-1-oxo-1,2,3,4-tetrahydroisoquinolin-6-yl)-7-fluorobenzo[b]thiophene-3-carbonitrile

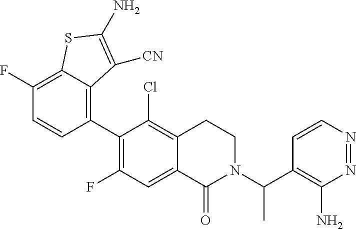 [SMILES: CC(c1ccnnc1N)N1CCc2c(cc(F)c(-c3ccc(F)c4sc(N)c(C#N)c34)c2Cl)C1=O]

### Step 1. 3-((4-Methoxybenzyl)amino)pyridazine-4-carbonitrile

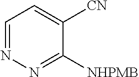

A solution of (4-methoxyphenyl)methanamine (1.1 mL, 8.4 mmol), 3-chloropyridazine-4-carbonitrile (1.0 g, 7 mmol) and DIPEA (2.4 mL, 14 mmol) in MeCN (10 mL) was stirred at r.t. for 4 h, then concentrated and purified by FCC (0-100% EtOAc in hexanes) to provide the title compound (804 mg, 48% yield). LCMS calc. for C₁₃H₁₃N₄O (M+H)⁺: m/z=241.1; found 241.1.

### Step 2. 4-(1-Aminoethyl)-N-(4-methoxybenzyl)pyridazin-3-amine

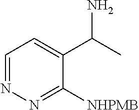

To a solution of the product from the previous step (240 mg, 1 mmol) in THF (10 mL) was added methylmagnesium bromide (3.0 M in diethyl ether, 680 μL, 2.05 mmol) at 0° C. The mixture was warmed to r.t. and stirred overnight. MeOH (1 mL) was then added to the reaction, followed by NaBH₄ (38 mg, 1.0 mmol). The mixture was stirred at r.t. for 4 h, then quenched with sat. aq. NaHCO₃ and extracted with EtOAc. The organic layer was washed with water and brine, dried over Na₂SO₄ and concentrated. The crude product was purified by FCC (0-100% EtOAc in hexanes, then 15-20% MeOH in DCM) to provide the title compound (28 mg, 11% yield). LCMS calc. for C₁₄H₁₉N₄O (M+H)⁺: m/z=259.2; found 259.2,

### Step 3. 2-amino-4-(2-(1-(3-aminopyridazin-4-yl)ethyl)-5-chloro-7-fluoro-1-oxo-1,2,3,4-tetrahydroisoquinolin-6-yl)-7-fluorobenzo[b]thiophene-3-carbonitrile

The title compound was prepared in a similar fashion to Example 7, with the appropriate starting material. For deprotection, the TFA solution of the intermediate was heated at 90° C. for 20 min. to affect deprotection. Purification was performed by prep. LCMS (XBRIDGE® C₁₈ column, eluting with a gradient of MeCN/water containing 0.1% NH₄OH, at flow rate of 60 mL/min.) to afford the title compound. LCMS calc. for C₂₄H₁₈ClF₂N₆OS (M+H)⁺: m/z=511.1; found 511.1.

The title compound comprises a mixture of diastereoisomers, i.e.,
(Rₐ)-2-amino-4-(2-((R)-1-(3-aminopyridazin-4-yl)ethyl)-5-chloro-7-fluoro-1-oxo-1,2,3,4-tetrahydroisoquinolin-6-yl)-7-fluorobenzo[b]thiophene-3-carbonitrile;
(Sₐ)-2-amino-4-(2-((S)-1-(3-aminopyridazin-4-yl)ethyl)-5-chloro-7-fluoro-1-oxo-1,2,3,4-tetrahydroisoquinolin-6-yl)-7-fluorobenzo[b]thiophene-3-carbonitrile;
(Rₐ)-2-amino-4-(2-((S)-1-(3-aminopyridazin-4-yl)ethyl)-5-chloro-7-fluoro-1-oxo-1,2,3,4-tetrahydroisoquinolin-6-yl)-7-fluorobenzo[b]thiophene-3-carbonitrile; and
(Sₐ)-2-amino-4-(2-((R)-1-(3-aminopyridazin-4-yl)ethyl)-5-chloro-7-fluoro-1-oxo-1,2,3,4-tetrahydroisoquinolin-6-yl)-7-fluorobenzo[b]thiophene-3-carbonitrile.

### Example 9. 4-(2-(1-(1H*1,2,4-Triazol-1-yl)propan-2-yl)-5-chloro-7-fluoro-1-oxo-1,2,3,4-tetrahydroisoquinolin-6-yl)-2-amino-7-fluorobenzo[b]thiophene-3-carbonitrile

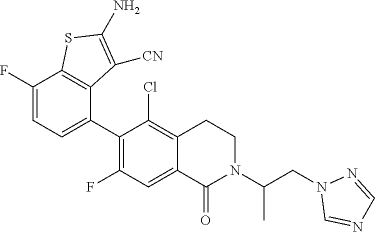 [SMILES: CC(Cn1cncn1)N1CCc2c(cc(F)c(-c3ccc(F)c4sc(N)c(C#N)c34)c2Cl)C1=O]

The title compound was prepared according to the procedure of Example 7, using 1-(1H-1,2,4-triazol-1-yl)propan-2-amine as the coupling partner. LCMS calc. for C₂₃H₁₈ClF₂N₆OS (M+H)⁺: m/z=499.1; found 499.1.

The title compound comprises a mixture of diastereoisomers, i.e.,
(Rₐ)-4-(2-((R)-1-(1H-1,2,4-triazol-1-yl)propan-2-yl)-5-chloro-7-fluoro-1-oxo-1,2,3,4-tetrahydroisoquinolin-6-yl)-2-amino-7-fluorobenzo[b]thiophene-3-carbonitrile;
(Sₐ)-4-(2-((S)-1-(1H-1,2,4-triazol-1-yl)propan-2-yl)-5-chloro-7-fluoro-1-oxo-1,2,3,4-tetrahydroisoquinolin-6-yl)-2-amino-7-fluorobenzo[b]thiophene-3-carbonitrile;
(Rₐ)-4-(2-((S)-1-(1H-1,2,4-triazol-1-yl)propan-2-yl)-5-chloro-7-fluoro-1-oxo-1,2,3,4-tetrahydroisoquinolin-6-yl)-2-amino-7-fluorobenzo[b]thiophene-3-carbonitrile; and
(Sₐ)-4-(2-((R)-1-(1H-1,2,4-triazol-1-yl)propan-2-yl)-5-chloro-7-fluoro-1-oxo-1,2,3,4-tetrahydroisoquinolin-6-yl)-2-amino-7-fluorobenzo[b]thiophene-3-carbonitrile.

### Example 10. 2-Amino-4-(8-amino-2-(1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile

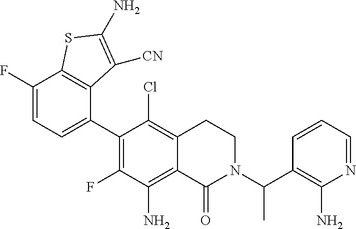 [SMILES: CC(c1cccnc1N)N1CCc2c(Cl)c(-c3ccc(F)c4sc(N)c(C#N)c34)c(F)c(N)c2C1=O]

### Step 1. (3-Amino-5-bromo-4-fluorophenyl)methanol

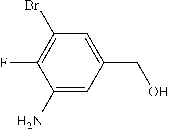 [SMILES: Nc1cc(CO)cc(Br)c1F]

LiBH₄ (2.0 M in THF, 168 mL, 335 mmol) was added to a solution of methyl 3-amino-5-bromo-4-fluorobenzoate (20.8 g, 84 mmol) in THF (200 mL) at r.t., followed by methanol (13.57 mL, 335 mmol). The mixture was stirred at r.t. for 2 h. Sat. aq. NH₄Cl was added and the resulting mixture was extracted with EtOAc. The combined organics were washed with brine, dried over Na₂SO₄ and concentrated. The crude product was used in the next step without further purification (18.4 g, ˜100% yield). LCMS calc. for C₇H₈BrFNO (M+H)⁺: m/z=220.0, 222.0; found 220.0, 222.0.

### Step 2. (3-Amino-4-fluoro-5-(4,4,5,5-tetramethyl-1,3,2-dioxaborolan-2-yl)phenyl)methanol

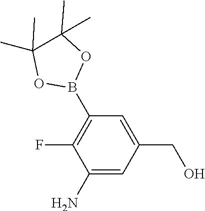 [SMILES: CC1(C)OB(c2cc(CO)cc(N)c2F)OC1(C)C]

A mixture of (3-amino-5-bromo-4-fluorophenyl)methanol (18.4 g, 84 mmol), bis(pinacolato) diboron (25.5 g, 100 mmol), dppf-PdCl₂ (6.83 g, 8.36 mmol) and potassium acetate (18.05 g, 184 mmol) in 1,4-dioxane (418 mL) was stirred at 100° C. under N₂ for 5 h. The crude mixture was filtered through diatomaceous earth and washed with EtOAc. The filtrate was concentrated. The residue was purified with FCC (0-70% EtOAc in hexanes) to give the title compound (16 g, 72% yield). LCMS calc. for C₁₃H₂₀BFNO₃ (M+H)⁺: m/z=268.1; found 268.1.

### Step 3. tert-Butyl (4-(3-amino-2-fluoro-5-(hydroxymethyl)phenyl)-3-cyano-7-fluorobenzo[b]thiophen-2-yl)carbamate

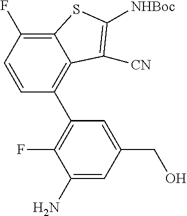 [SMILES: CC(C)(C)OC(=O)Nc1sc2c(F)ccc(-c3cc(CO)cc(N)c3F)c2c1C#N]

To a mixture of tert-butyl (4-bromo-3-cyano-7-fluorobenzo[b]thiophen-2-yl)carbamate (16.5 g, 44.4 mmol), (3-amino-4-fluoro-5-(4,4,5,5-tetramethyl,1-3,2-dioxaborolan-2-yl)phenyl)methanol (15.4 g, 57.8 mmol), XPhos Pd G2 (2.9 g, 3.56 mmol) and K₃PO₄ (10.4 g, 98 mmol) were added 1,4-dioxane (200 mL) and water (40 mL) and the mixture was placed under N₂, then heated at 70° C. for 3 h. The mixture was diluted with EtOAc, washed with water, dried over Na₂SO₄ and concentrated. The crude product was purified by FCC (0-70% EtOAc in hexanes) to provide the title compound (18.8 g, 98% yield). LCMS calc. for C₂₁H₂₀F₂N₃O₃S (M+H)⁺: m/z=432.1; found 432.1.

### Step 4. tert-Butyl (4-(3-amino-6-chloro-2-fluoro-5-(hydroxymethyl)phenyl)-3-cyano-7-fluorobenzo[b]thiophen-2-yl)carbamate

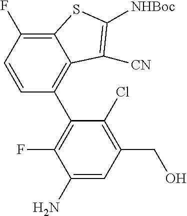 [SMILES: CC(C)(C)OC(=O)Nc1sc2c(F)ccc(-c3c(F)c(N)cc(CO)c3Cl)c2c1C#N]

To a solution of tert-butyl (4-(3-amino-2-fluoro-5-(hydroxymethyl)phenyl)-3-cyano-7-fluorobenzo[b]thiophen-2-yl)carbamate (19.2 g, 44.5 mmol) in DMF (111 mL) was added NCS (6.24 g, 46.7 mmol). The resulting mixture was stirred at 40° C. for 3 h. The mixture was diluted with EtOAc and water. The layers were separated and the organic layer was washed with water and brine, dried over Na₂SO₄ and concentrated. The crude product was purified by FCC (0-30% EtOAc in hexanes) to give the title compound as the later eluting peak (15.3 g, 74% yield). LCMS calc. for C₂₁H₁₉ClF₂N₃O₃S (M+H)⁺: m/z=466.1; found 466.1.

### Step 5. tert-Butyl (4-(3-amino-6-chloro-2-fluoro-5-(hydroxymethyl)-4-iodophenyl)-3-cyano-7-fluorobenzo[b]thiophen-2-yl)carbamate

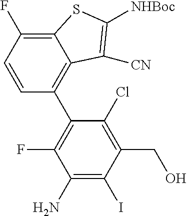 [SMILES: CC(C)(C)OC(=O)Nc1sc2c(F)ccc(-c3c(F)c(N)c(I)c(CO)c3Cl)c2c1C#N]

To a solution of tert-butyl (4-(3-amino-6-chloro-2-fluoro-5-(hydroxymethyl)phenyl)-3-cyano-7-fluorobenzo[b]thiophen-2-yl)carbamate (15.5 g, 33.3 mmol) in ethanol (222 mL) were added silver sulfate (11.41 g, 36.6 mmol) and iodine (9.29 g, 36.6 mmol) portionwise, and the resultant mixture stirred at r.t. overnight. The mixture was poured onto ice-water (100 mL) and extracted with EtOAc. The organic layer was washed with water (2×), brine, dried over Na₂SO₄ and concentrated. The crude was purified with FCC (0-30% EtOAc in hexanes) to give the title compound (19.7 g, ˜100% yield). LCMS calc. for C₂₁H₁₈ClF₂₁N₃O₃S (M+H)⁺: m/z=592.0; found 592.0.

### Step 6. tert-Butyl (4-(3-amino-5-(((tert-butyldimethylsilyl)oxy)methyl)-6-chloro-2-fluoro-4-iodophenyl)-3-cyano-7-fluorobenzo[b]thiophen-2-yl)carbamate

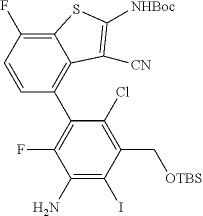 [SMILES: CC(C)(C)OC(=O)Nc1sc2c(F)ccc(-c3c(F)c(N)c(I)c(CO[Si](C)(C)C(C)(C)C)c3Cl)c2c1C#N]

To a solution of the product from the previous step (19.7 g, 33.3 mmol) in DMF (67 mL) were added imidazole (2.95 g, 43.3 mmol) and TBS-CI (6.0 g, 40 mmol) and the mixture was stirred at r.t. for 2 h, then quenched with water and extracted with EtOAc. The organic layer was washed with water and brine, dried over Na₂SO₄ and concentrated. The crude product was purified by FCC (20% EtOAc in hexanes) to provide the title compound (21.6 g, 92% yield). LCMS calc. for C₂₇H₃₂ClF₂IN₃O₃SSi (M+H)⁺: m/z=706.1; found 706.1.

### Step 7. tert-Butyl(4-(3-(((tert-butyldimethylsilyl)oxy)methyl)-2-chloro-6-fluoro-4-iodo-5-(2,2,2-trifluoroacetamido)phenyl)-3-cyano-7-fluorobenzo[b]thiophen-2-yl)carbamate

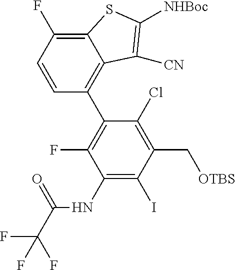 [SMILES: CC(C)(C)OC(=O)Nc1sc2c(F)ccc(-c3c(F)c(NC(=O)C(F)(F)F)c(I)c(CO[Si](C)(C)C(C)(C)C)c3Cl)c2c1C#N]

To a solution of tert-butyl (4-(3-amino-5-(((tert-butyldimethylsilyl)oxy)methyl)-6-chloro-2-fluoro-4-iodophenyl)-3-cyano-7-fluorobenzo[b]thiophen-2-yl)carbamate (3.04 g, 4.31 mmol) in DCM (2.0 mL) were added DIPEA (1.88 mL, 10.76 mmol) and 2,2,2-trifluoroacetic anhydride (1.2 mL, 8.61 mmol). After stirring at r.t. overnight, another equivalent of reagents were added and stirring was continued for a further 16 h. Upon completion, the mixture was diluted with water. The organic layer was separated and dried over Na₂SO₄ and concentrated. The crude product was purified by FCC (0-20% EtOAc in hexanes) to provide the title compound (2.9 g, 84% yield). LCMS calc. for C₂₉H₃₁ClF₅IN₃O₄SSi (M+H)⁺: m/z=802.1; found 802.1.

### Step 8. tert-Butyl (4-(2-chloro-6-fluoro-3-(hydroxymethyl)-4-iodo-5-(2,2,2-trifluoroacetamido)phenyl)-3-cyano-7-fluorobenzo[b]thiophen-2-yl)carbamate

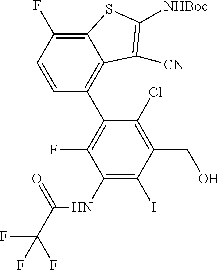 [SMILES: CC(C)(C)OC(=O)Nc1sc2c(F)ccc(-c3c(F)c(NC(=O)C(F)(F)F)c(I)c(CO)c3Cl)c2c1C#N]

To a solution of the product from the previous step (2.9 g, 3.62 mmol) in THF (20 mL) was added TBAF (1 M in THF, 6.5 mL, 6.5 mmol) and the mixture was stirred at r.t. for 3 days. The mixture was diluted with EtOAc and washed with water and brine, then dried over Na₂SO₄ and concentrated. The crude product was purified by FCC (0-40% EtOAc in hexanes) to provide the title compound (1.8 g, 72% yield). LCMS calc. for C₂₃H₁₇ClF₅IN₃O₄S (M+H)⁺: m/z=688.0; found 688.0.

### Step 9. tert-Butyl (4-(2-chloro-6-fluoro-3-formyl-4-iodo-5-(2,2,2-trifluoroacetamido)phenyl)-3-cyano-7-fluorobenzo[b]thiophen-2-yl)carbamate

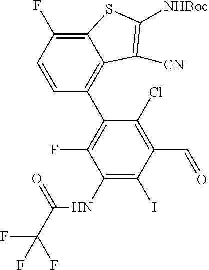 [SMILES: CC(C)(C)OC(=O)Nc1sc2c(F)ccc(-c3c(F)c(NC(=O)C(F)(F)F)c(I)c(C=O)c3Cl)c2c1C#N]

To a solution of the product from the previous step (1.47 g, 2.14 mmol) in DCM (16 mL)/MeCN (5.3 mL) were added acetic acid (0.37 mL, 6.41 mmol) and IBX (1.8 g, 6.41 mmol) and the mixture was heated at 38° C. overnight. The mixture was then filtered through a pad of diatomaceous earth and the filtrate concentrated. The crude product was purified by FCC (0-50% DCM in hexanes) to provide the title compound (1.45 g, 99% yield). LCMS calc. for C₂₃H₁₅ClF₅IN₃O₄S (M+H)⁺: m/z=686.0; found 686.0.

### Step 10. tert-Butyl (E)-(4-(2-chloro-6-fluoro-4-iodo-3-(2-methoxyvinyl)-5-(2,2,2-trifluoroacetamido)phenyl)-3-cyano-7-fluorobenzo[b]thiophen-2-yl)carbamate

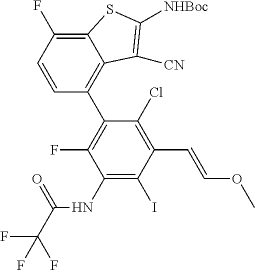 [SMILES: CO/C=C/c1c(Cl)c(-c2ccc(F)c3sc(NC(=O)OC(C)(C)C)c(C#N)c23)c(F)c(NC(=O)C(F)(F)F)c1I]

To a solution of (methoxymethyl)triphenylphosphonium chloride (2.54 g, 7.40 mmol) in toluene (20 mL) was added KOtBu (1.0 M in THF, 7.6 mL, 7.6 mmol). After stirring at r.t. for 30 min., a solution of tert-butyl (4-(2-chloro-6-fluoro-3-formyl-4-iodo-5-(2,2,2-trifluoroacetamido)phenyl)-3-cyano-7-fluorobenzo[b]thiophen-2-yl)carbamate (1.45 g, 2.114 mmol) in THF (15 mL) was cannulated into the reaction flask. The resulting solution was stirred at r.t. for 1 h. The reaction was quenched with 1 M HCl and the mixture was diluted with EtOAc. The aqueous layer was extracted with EtOAc and the combined organics were washed with brine, dried over Na₂SO₄ and concentrated. The residue was purified with FCC (0-50% EtOAc in hexanes with 5% DCM as additive) to give the title compound (900 mg, 60% yield). LCMS calc. for C₂₅H₁₉ClF₅IN₃O₄S (M+H)⁺: m/z=714.0; found 714.0.

### Step 11. tert-Butyl (4-(2-chloro-6-fluoro-4-iodo-3-(2-oxoethyl)-5-(2,2,2-trifluoroacetamido)phenyl)-3-cyano-7-fluorobenzo[b]thiophen-2-yl)carbamate

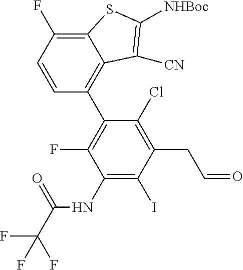 [SMILES: CC(C)(C)OC(=O)Nc1sc2c(F)ccc(-c3c(F)c(NC(=O)C(F)(F)F)c(I)c(CC=O)c3Cl)c2c1C#N]

To a solution of tert-butyl (4-(2-chloro-6-fluoro-4-iodo-3-(2-methoxyvinyl)-5-(2,2,2-trifluoroacetamido)phenyl)-3-cyano-7-fluorobenzo[b]thiophen-2-yl)carbamate (900 mg, 1.261 mmol) was added 1,4-dioxane (12 mL). To this solution was added 4 M HCl in 1,4-dioxane (6.3 mL, 25.2 mmol) and the mixture was stirred at r.t. for 1.5 h. The mixture was diluted with EtOAc and water, the phases were separated and the aqueous phase was extracted with EtOAc. The combined organics were washed with brine, dried over Na₂SO₄, filtered and concentrated to a white solid. The crude product was used in the next step without further purification. LCMS calc. for C₂₄H₁₇ClF₅IN₃O₄S (M+H)⁺: m/z=700.0; found 700.0.

### Step 12. tert-Butyl(4-(3-(2-((1-(2-(bis(4-methoxybenzyl)amino)pyridin-3-yl)ethyl)amino)ethyl)-2-chloro-6-fluoro-4-iodo-5-(2,2,2-trifluoroacetamido)phenyl)-3-cyano-7-fluorobenzo[b]thiophen-2-yl)carbamate

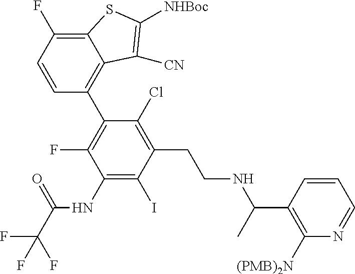

3-(1-Aminoethyl)-N,N-bis(4-methoxybenzyl)pyridin-2-amine (283 mg, 0.750 mmol) was added to a solution of tert-butyl (4-(2-chloro-6-fluoro-4-iodo-3-(2-oxoethyl)-5-(2,2,2-trifluoroacetamido) phenyl)-3-cyano-7-fluorobenzo[b]thiophen-2-yl)carbamate (500 mg, 0.714 mmol) in THF (3.6 mL). The mixture was stirred at r.t. for 30 min. In a separate reaction vial, NaBH(OAc)₃ (606 mg, 2.86 mmol), DCM (3.6 mL) and TFA (1.1 mL, 14.29 mmol) were stirred for 30 min., then added to the mixture of aldehyde and amine. The resulting mixture was stirred at r.t. overnight. Additional reagents can be added as necessary to push the reaction to full conversion. Upon completion, the reaction was quenched with water and the mixture was extracted with DCM. The organic layer was dried over Na₂SO₄ and concentrated. The crude product was purified by FCC (0-100% EtOAc in hexanes) to provide the title compound (609 mg, 80% yield). LCMS calc. for C₄₇H44ClF₅IN₆O₅S (M+H)⁺: m/z=1061.2; found 1061.2.

### Step 13. tert-Butyl(4-(2-(1-(2-(bis(4-methoxybenzyl)amino)pyridin-3-yl)ethyl)-5-chloro-7-fluoro-1-oxo-8-(2,2,2-trifluoroacetamido)-1,2,3,4-tetrahydroisoquinolin-6-yl)-3-cyano-7-fluorobenzo[b]thiophen-2-yl)carbamate

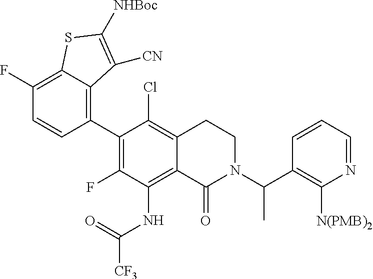

A solution of tert-butyl (4-(3-(2-((1-(2-(bis(4-methoxybenzyl)amino)pyridin-3-yl)ethyl)amino)ethyl)-2-chloro-6-fluoro-4-iodo-5-(2,2,2-trifluoroacetamido)phenyl)-3-cyano-7-fluorobenzo[b]thiophen-2-yl)carbamate (609 mg, 0.574 mmol), dppf-PdCl₂ (46.9 mg, 0.057 mmol) and K₂CO₃ (174 mg, 1.262 mmol) in 1,4-Dioxane (6 mL) was degassed for 10 min. with CO gas. The solution was stirred at 100° C. under a CO atmosphere for 3 h. The mixture was filtered through a pad of diatomaceous earth and washed with EtOAc. The filtrate was concentrated in vacuo. The crude product was purified by FCC (80% EtOAc in hexanes) to afford the title compound (380 mg, 69% yield). LCMS calc. for C₄₈H₄₃ClF₅N₆O₆S (M+H)⁺: m/z=961.2; found 961.2.

### Step 14. 2-Amino-4-(8-amino-2-(1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile

To a solution of the product from the previous step (36 mg, 0.04 mmol) in 2:1 water/methanol (350 μL total volume) was added K₂CO₃ (10 mg, 0.074 mmol) and the mixture was heated at 60° C. overnight. The mixture was diluted with water and extracted with DCM. The organic layer was dried over Na₂SO₄ and concentrated. The crude product was dissolved in TFA (1 mL), then heated at 90° C. for 30 min. The crude mixture was diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₅H₂₀ClF₂N₆OS (M+H)⁺: m/z=525.1; found 525.1.

The title compound comprises a mixture of diastereoisomers, i.e.,
(Rₐ)-2-amino-4-(8-amino-2-((R)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile;
(Sₐ)-2-amino-4-(8-amino-2-((S)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile;
(Rₐ)-2-amino-4-(8-amino-2-((S)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile; and
(Sₐ)-2-amino-4-(8-amino-2-((R)-1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile.

### Example 11. N-(6-(2-Amino-3-cyano-7-fluorobenzo[b]thiophen-4-yl)-2-(1-(2-aminopyridin-3-yl)ethyl)-5-chloro-7-fluoro-1-oxo-1,2,3,4-tetrahydroisoquinolin-8-yl)acetamide

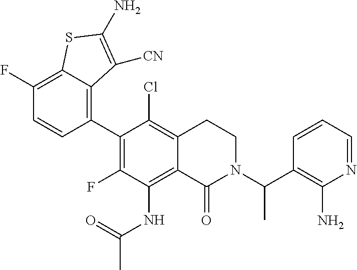 [SMILES: CC(=O)Nc1c(F)c(-c2ccc(F)c3sc(N)c(C#N)c23)c(Cl)c2c1C(=O)N(C(C)c1cccnc1N)CC2]

### Step 1. 1-Bromo-2-fluoro-5-iodo-3-nitrobenzene

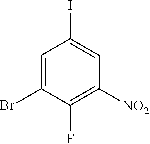 [SMILES: O=[N+]([O-])c1cc(I)cc(Br)c1F]

1-bromo-2-fluoro-3-nitrobenzene (60.0 g, 273 mmol) was dissolved in trifluoromethanesulfonic acid (89 mL, 1004 mmol) and cooled to 0° C. NIS (73.6 g, 327 mmol) was added in portions over 6 h and the mixture was allowed to warm to r.t. gradually over 72 h. The mixture was then poured into 200 mL of water and the resulting mixture was neutralized using 40% aq. NaOH and extracted with heptane. The organic extract was washed with 10% aq. Na₂SO₃ until colorless, then with brine, and finally dried over MgSO₄. After concentration, the crude product was used in the next step without further purification (93 g, 99% yield). LCMS calc. for C₆H₃BrFINO₂ (M+H)⁺: m/z=347.8, 349.8; found 347.8, 349.8.

### Step 2. 3-Bromo-2-fluoro-5-iodoaniline

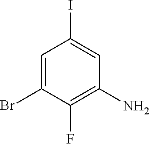

To a solution of 1-bromo-2-fluoro-5-iodo-3-nitrobenzene (93 g, 271 mmol) in ethanol/water (5:1, 700 mL total volume) were added iron (44 g, 786 mmol) and NH₄Cl (72.4 g, 1.3 mol) and the mixture was stirred at 80° C. for 2 h, then diluted with DCM and filtered through a plug of diatomaceous earth. The filtrate was concentrated and the purified by flash column chromography (0-20% EtOAc in hexanes) to provide the title compound (71.3 g, 83% yield). LCMS calc. for C₆H₅BrFIN (M+H)⁺: m/z=315.8, 317.9; found 315.9, 317.9.

### Step 3. Methyl 2-(3-amino-5-bromo-4-fluorophenyl)acetate

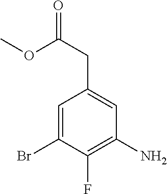

To a solution of 3-bromo-2-fluoro-5-iodoaniline (9.2 g, 29.3 mmol) in DCM (100 mL) were added methyl 3-oxobutanoate (10.2 g, 88 mmol), K₃PO₄ (18.6 g, 88 mmol), CuI (1.11 g, 5.86 mmol) and ethanol (5.1 mL, 88 mmol) and the mixture was heated at 80° C. for 24 h under N₂. The reaction was quenched with 1 M HCl and the mixture was extracted with diethyl ether. The organic layer was washed with brine, dried over Na₂SO₄ and concentrated. The crude product was purified by FCC (0-30% EtOAc in hexanes) to provide the title compound (3.2 g, 42% yield). LCMS calc. for C₉H₁₀BrFNO₂ (M+H)⁺: m/z=263.0, 265.0; found 263.0, 265.0.

### Step 4. 2-(3-Amino-5-bromo-4-fluorophenyl)ethan-1-ol

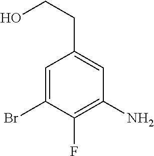 [SMILES: Nc1cc(CCO)cc(Br)c1F]

LiBH₄ (2.0 M in THF, 160 mL, 321 mmol) was added to a mixture of methyl 2-(3-amino-5-bromo-4-fluorophenyl)acetate (21.0 g, 80 mmol) in THF (300 mL) at r.t., followed by methanol (13 mL, 321 mmol). The mixture was stirred at r.t. for 2 h. Sat. aq. NH₄Cl was added and the resulting mixture was extracted with EtOAc. The combined organics were washed with brine, dried over Na₂SO₄ and concentrated. The crude was purified by FCC (0-25% EtOAc in hexanes) to give the title compound (14.5 g, 77% yield). LCMS calc. for C₆H₁₀BrFNO (M+H)⁺: m/z=234.0, 236.0; found 234.0, 236.0.

### Step 5. 2-(5-Amino-3-bromo-2-chloro-4-fluorophenyl)ethan-1-ol

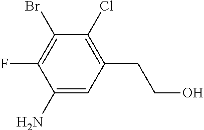 [SMILES: Nc1cc(CCO)c(Cl)c(Br)c1F]

To a solution of 2-(3-amino-5-bromo-4-fluorophenyl)ethan-1-ol (3.95 g, 16.88 mmol) in DMF (56 mL) was added NCS (2.37 g, 17.72 mmol). The resulting mixture was stirred at 50° C. 1.5 h. The mixture was diluted with EtOAc and water. The organic layer was washed with water, brine, dried over Na₂SO₄ and concentrated. The residue was purified with FCC (0-30% EtOAc in hexanes) to provide the title compound (2.7 g, 60% yield). LCMS calc. for C₈H₉BrClFNO (M+H)⁺: m/z=268.0, 270.0; found 268.0, 270.0.

### Step 6. 2-(3-Amino-5-bromo-6-chloro-4-fluoro-2-iodophenyl)ethan-1-ol

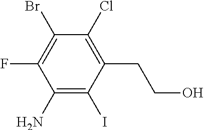 [SMILES: Nc1c(F)c(Br)c(Cl)c(CCO)c1I]

To a solution of 2-(5-amino-3-bromo-2-chloro-4-fluorophenyl)ethan-1-ol (4.55 g, 16.95 mmol) in ethanol (113 mL) was added silver sulfate (5.81 g, 18.64 mmol) and iodine (4.73 g, 18.64 mmol) portionwise, and the resulting mixture stirred at 45° C. for 2 h. The mixture was poured onto ice-water (100 mL) and extracted with EtOAc. The organic layer was washed with water (2×), brine, dried over Na₂SO₄ and concentrated. The crude was purified by FCC (0-30% EtOAc in hexanes) to provide the title compound (5.7 g, 85% yield). LCMS calc. for C₈H₈BrClFINO (M+H)⁺: m/z=393.8, 395.8; found 393.8, 395.8.

### Step 7. 2-(3-Amino-5-bromo-6-chloro-4-fluoro-2-iodophenyl)acetaldehyde

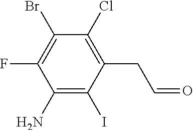 [SMILES: Nc1c(F)c(Br)c(Cl)c(CC=O)c1I]

To a solution of 2-(3-amino-5-bromo-6-chloro-4-fluoro-2-iodophenyl)ethan-1-ol (420 mg, 1.065 mmol) in DCM (10 mL) was added Dess-Martin periodinane (497 mg, 1.171 mmol). The resulting mixture was stirred for 2 h at r.t. The reaction was quenched with sat. aq. NaHCO₃ and stirring was continued for 10 min. The organic layer was separated and dried over Na₂SO₄ and concentrated. The residue was triturated with EtOAc/Hexanes (1:4) to give the title compound as white solid (1.48 g, 90%). LCMS calc. for C₈H₆BrClFINO (M+H)⁺: m/z=391.8, 393.8; found 391.8, 393.8.

### Step 8. 3-(1-((3-Amino-5-bromo-6-chloro-4-fluoro-2-iodophenethyl)amino)ethyl)-N,N-bis(4-methoxybenzyl)pyridin-2-amine

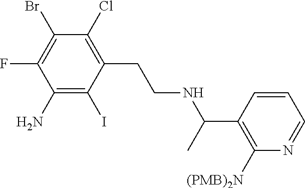

3-(1-Aminoethyl)-N,N-bis(4-methoxybenzyl)pyridin-2-amine (1.905 g, 5.05 mmol) was added to a solution of 2-(3-amino-5-bromo-6-chloro-4-fluoro-2-iodophenyl)acetaldehyde (1.65 g, 4.20 mmol) in THF (21 mL). The mixture was stirred at r.t. for 30 min. To another vial was added NaBH(OAc)₃ (1.782 g, 8.41 mmol), DCM (21 mL) and TFA (3.24 mL, 42.0 mmol), this was also stirred for 30 min. at r.t., then added to the amine/aldehyde solution. The resulting mixture was stirred at r.t. overnight. Additional reagents were added as necessary to push reaction to full conversion. Upon completion, the reaction was quenched with water and the mixture was extracted with DCM. The organic layer was dried over Na₂SO₄ and concentrated. The crude product was purified by FCC (0-100% EtOAc in hexanes) to provide the title compound (2.9 g, 91% yield). LCMS calc. for C₃₁H₃₃BrClFIN₄O₂(M+H)⁺: m/z=753.0, 755.0; found 753.0, 755.0.

### Step 9. 8-Amino-2-(1-(2-(bis(4-methoxybenzyl)amino)pyridin-3-yl)ethyl)-6-bromo-5-chloro-7-fluoro-3,4-dihydroisoquinolin-1(2H)-one

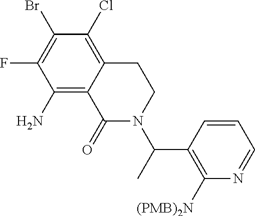

A solution of 3-(1-((3-amino-5-bromo-6-chloro-4-fluoro-2-iodophenethyl)amino)ethyl)N,N-bis(4-methoxybenzyl)pyridin-2-amine (2.9 g, 3.85 mmol) and dppf-PdCl₂ (0.314 g, 0.385 mmol) in Toluene (38.5 mL) was degassed for 10 min. with CO gas. Then NEt₃ (1.61 mL, 11.54 mmol) was added and the solution was stirred at 70° C. under a CO atmosphere for 72 h. The mixture was diluted with sat. aq. NH₄Cl and extracted with EtOAc. The combined organic layers were washed with 1 M HCl and brine, then dried over MgSO₄ and concentrated. The crude product was purified by FCC (20% EtOAc in hexanes) to provide the title compound (0.51 g, 20% yield). LCMS calc. for C₃₂H₃₂BrClFN₄O₃(M+H)⁺: m/z=653.1, 655.1; found 653.1, 655.1.

### Step 10. N-(2-(1-(2-(Bis(4-methoxybenzyl)amino)pyridin-3-yl)ethyl)-6-bromo-5-chloro-7-fluoro-1-oxo-1,2,3,4-tetrahydroisoquinolin-8-yl)acetamide

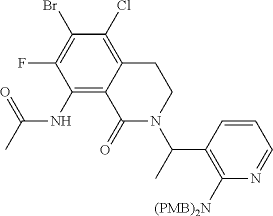

To a solution of 8-amino-2-(1-(2-(bis(4-methoxybenzyl)amino)pyridin-3-yl)ethyl)-6-bromo-5-chloro-7-fluoro-3,4-dihydroisoquinolin-1(2H)-one (121 mg, 0.185 mmol) in DCE (1.8 mL) was added acetyl chloride (17 μL, 0.241 mmol) at r.t. The mixture was heated at 80° C. for 1.5 h. The mixture was cooled to r.t. and diluted with water. The resulting precipitate was collected by filtration and washed with water and EtOAc/hexane (1-2) to provide the title compound, which was used in the next step without further purification. LCMS calc. for C₃₄H₃₄BrClFN₄O₄(M+H)⁺: m/z=695.1, 697.1; found 655.1, 697.1.

### Step 11. N-(6-(2-Amino-3-cyano-7-fluorobenzo[b]thiophen-4-yl)-2-(1-(2-aminopyridin-3-yl)ethyl)-5-chloro-7-fluoro-1-oxo-1,2,3,4- tetrahydroisoquinolin-8-yl)acetamide

A mixture of 8-amino-2-(1-(2-(bis(4-methoxybenzyl)amino)pyridin-3-yl)ethyl)-6-bromo-5-chloro-7-fluoro-3,4-dihydroisoquinolin-1(2H)-one (12 mg, 0.029 mmol), tert-butyl (3-cyano-4-(5,5-dimethyl-1,3,2-dioxaborinan-2-yl)-7-fluorobenzo[b]thiophen-2-yl)carbamate (23.45 mg, 0.058 mmol), dppf-PdCl₂ (4.74 mg, 5.80 μmol) and K₂CO₃ (12.03 mg, 0.087 mmol) in 1,4-dioxane (200 μL) was stirred under N₂ at 85° C. for 16 h. The reaction was quenched with brine and the mixture was extracted with EtOAc. The organic layer was dried over Na₂SO₄ and concentrated. The crude product was dissolved in TFA (1 mL) and stirred at 90° C. for 30 min., then diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₇H₂₂ClF₂N₆O₂S (M+H)⁺: m/z=567.1; found 567.1.

The title compound comprises a mixture of diastereoisomers, i.e.,
N—(Rₐ)-(6-(2-amino-3-cyano-7-fluorobenzo[b]thiophen-4-yl)-2-((R)-1-(2-aminopyridin-3-yl)ethyl)-5-chloro-7-fluoro-1-oxo-1,2,3,4-tetrahydroisoquinolin-8-yl)acetamide;
N—(Sₐ)-(6-(2-amino-3-cyano-7-fluorobenzo[b]thiophen-4-yl)-2-((S)-1-(2-aminopyridin-3-yl)ethyl)-5-chloro-7-fluoro-1-oxo-1,2,3,4-tetrahydroisoquinolin-8-yl)acetamide;
N—(Rₐ)-(6-(2-amino-3-cyano-7-fluorobenzo[b]thiophen-4-yl)-2-((S)-1-(2-aminopyridin-3-yl)ethyl)-5-chloro-7-fluoro-1-oxo-1,2,3,4-tetrahydroisoquinolin-8-yl)acetamide; and
N—(Sₐ)-(6-(2-amino-3-cyano-7-fluorobenzo[b]thiophen-4-yl)-2-((R)-1-(2-aminopyridin-3-yl)ethyl)-5-chloro-7-fluoro-1-oxo-1,2,3,4-tetrahydroisoquinolin-8-yl)acetamide.

### Example 12. 6-(2-Amino-7-fluorobenzo[d]thiazol-4-yl)-2-(1-(2-aminopyridin-3-yl)ethyl)-5-chloro-7-fluoro-8-(1H-pyrazol-4-yl)-3,4-dihydroisoquinolin-1(2H)-one

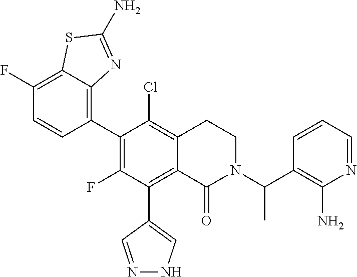 [SMILES: CC(c1cccnc1N)N1CCc2c(Cl)c(-c3ccc(F)c4sc(N)nc34)c(F)c(-c3cn[nH]c3)c2C1=O]

### Step 1. tert-Butyl(4-(8-amino-2-(1-(2-(bis(4-methoxybenzyl)amino)pyridin-3-yl)ethyl)-5-chloro-7-fluoro-1-oxo-1,2,3,4-tetrahydroisoquinolin-6-yl)-7-fluorobenzo[d]thiazol-2-yl)carbamate

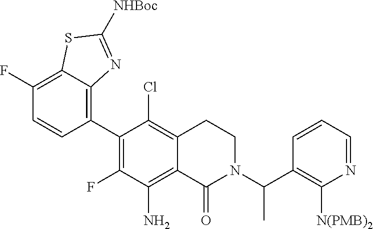

To a mixture of 8-amino-2-(1-(2-bis(4-methoxybenzyl)amino)pyridin-3-yl)ethyl)-6-bromo-5-chloro-7-fluoro-3,4-dihydroisoquinolin-1(2H)-one (Example 11, Step 9, 760 mg, 1.16 mmol), (2-((tert-butoxycarbonyl)amino-7-fluorobenzo[d]thiazol-4-yl)boronic acid (653 mg, 2.09 mmol), K₃PO₄ (740 mg, 3.5 mmol) and XPhos Pd G2 (91 mg, 0.12 mmol) were added 1,4-dioxane (10 mL) and water (2 mL) and the mixture was placed under N₂, then stirred at 80° C. for 3 h. The reaction was quenched with water and the mixture was extracted with EtOAc. The organic layer was washed with brine, dried over Na₂SO₄ and concentrated. The crude product was purified by FCC (0-20% DCM in MeOH) to provide the title compound. LCMS calc. for C_{4H}4ClF₂N₆O₅S (M+H)⁺: m/z=841.3; found 841.3.

### Step 2. tert-Butyl (4-(2-(1-(2-(bis(4-methoxybenzyl)amino)pyridin-3-yl)ethyl)-5-chloro-7-fluoro-8-iodo-1-oxo-1,2,3,4-tetrahydroisoquinolin-6-yl)-7-fluorobenzo[d]thiazol-2-yl)carbamate

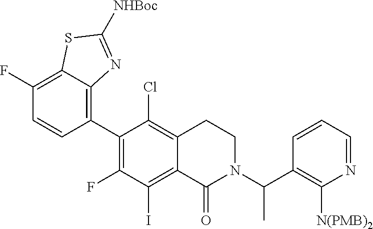

To a solution of the product of the previous step (238 mg, 0.28 mmol) in MeCN (1.5 mL) was added sulfuric acid (4.0 M in water, 177 μL, 0.71 mmol) at −20° C. Then a solution of sodium nitrite (39 mg, 0.57 mmol) in water (400 μL) was added slowly, maintaining internal temperature below −10° C. After stirring for 5 min., a solution of KI (188 mg, 1.13 mmol) in water (400 μL) was added slowly, continuing to maintain internal temperature below −10° C. The mixture was stirred at r.t. for 10 min., then quenched with water and extracted with EtOAc. The organic layer was washed with sat. aq, Na₂S₂O₃, sat. aq. NaHCO₃ and brine, then dried over Na₂SO₄ and concentrated. The crude product was used in the next step without further purification. LCMS calc. for C₄₄H₄₂ClF₂IN₅O₅S (M+H)⁺: m/z=952.1; found 952.1.

### Step 3. 6-(2-Amino-7-fluorobenzo[d]thiazol-4-yl)-2-(1-(2-aminopyridin-3-yl)ethyl)-5-chloro-7-fluoro-8-(1H-pyrazol-4-yl)-3,4-dihydroisoquinolin-1(2H)-one

To a mixture of the product from the previous step (20 mg, 0.021 mmol), tert-butyl 4-(4,4,5,5-tetramethyl-1,3,2-dioxaborolan-2-yl)-1H-pyrazole-1-carboxylate (11 mg, 0.038 mmol), K₃PO₄ (13 mg, 0.063 mmol) and dppf-PdCl₂ (1.7 mg, 2.1 μmol) were added 1,4-dioxane (1 mL)/water (200 μL) and the mixture was placed under N₂, then stirred at 90° C. for 3 h. The reaction was quenched with water and the mixture was extracted with EtOAc. The organic layer was dried over Na₂SO₄ and concentrated. The crude product was dissolved in TFA (1 mL) and heated at 90° C. for 1 h, then diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₆H₂₁ClF₂N₇OS (M+H)⁺: m/z=552.1; found 552.1.

The title compound comprises a mixture of diastereoisomers, i.e.,
(Rₐ)-6-(2-amino-7-fluorobenzo[d]thiazol-4-yl)-2-((R)-1-(2-aminopyridin-3-yl)ethyl)-5-chloro-7-fluoro-8-(1H-pyrazol-4-yl)-3,4-dihydroisoquinolin-1(2H)-one;
(Sₐ)-6-(2-amino-7-fluorobenzo[thiazol-4-yl)-2-((S)-1-(2-aminopyridin-3-yl)ethyl)-5-chloro-7-fluoro-8-(1H-pyrazol-4-yl)-3,4-dihydroisoquinolin-1(2H)-one;
(Rₐ)-6-(2-amino-7-fluorobenzo[d]thiazol-4-yl)-2-((S)-1-(2-aminopyridin-3-yl)ethyl)-5-chloro-7-fluoro-8-(1H-pyrazol-4-yl)-3,4-dihydroisoquinolin-1(2H)-one; and
(Sₐ)-6-(2-amino-7-fluorobenzo[d]thiazol-4-yl)-2-((R)-1-(2-aminopyridin-3-yl)ethyl)-5-chloro-7-fluoro-8-(1H-pyrazol-4-yl)-3,4-dihydroisoquinolin-1(2H)-one.

### Example 13. 8-Amino-6-(2-amino-7-fluorobenzo[d]thiazol-4-yl)-2-(1-(2-aminopyridin-3-yl)ethyl)-5-chloro-7-fluoro-3,4-dihydroisoquinolin-1(2H)-one

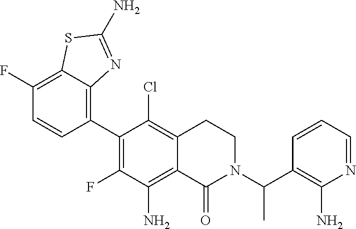 [SMILES: CC(c1cccnc1N)N1CCc2c(Cl)c(-c3ccc(F)c4sc(N)nc34)c(F)c(N)c2C1=O]

After the procedure described in Example 13, Step 1, the resulting product was stirred in TFA (1 mL) at 90° C. for 30 min., then diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₃H₂₀ClF₂N₆OS (M+H)⁺: m/z=501.1; found 501.1.

The title compound comprises a mixture of diastereoisomers, i.e.,
(Rₐ)-8-amino-6-(2-amino-7-fluorobenzo[d]thiazol-4-yl)-2-((R)-1-(2-aminopyridin-3-yl)ethyl)-5-chloro-7-fluoro-3,4-dihydroisoquinolin-1(2H)-one;
(Sₐ)-8-amino-6-(2-amino-7-fluorobenzo[thiazol-4-yl)-2-((S)-1-(2-aminopyridin-3-yl)ethyl)-5-chloro-7-fluoro-3,4-dihydroisoquinolin-1(2H)-one;
(Rₐ)-8-amino-6-(2-amino-7-fluorobenzo[thiazol-4-yl)-2-((S)-1-(2-aminopyridin-3-yl)ethyl)-5-chloro-7-fluoro-3,4-dihydroisoquinolin-1(2H)-one; and
(Sₐ)-8-amino-6-(2-amino-7-fluorobenzo[thiazol-4-yl)-2-((R)-1-(2-aminopyridin-3-yl)ethyl)-5-chloro-7-fluoro-3,4-dihydroisoquinolin-1(2H)-one.

### Example 14. 2-Amino-4-(2-(1-(2-aminopyridin-3-yl)ethyl)-7-chloro-5-fluoro-1-oxo-1,2,3,4-tetrahydroisoquinolin-6-yl)-7-fluorobenzo[b]thiophene-3-carbonitrile

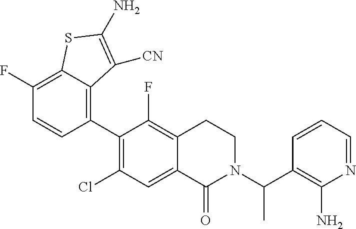 [SMILES: CC(c1cccnc1N)N1CCc2c(cc(Cl)c(-c3ccc(F)c4sc(N)c(C#N)c34)c2F)C1=O]

### Step 1. Methyl 4-bromo-5-chloro-3-fluoro-2-methylbenzoate

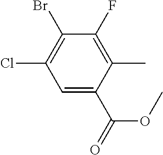

4-Bromo-3-fluoro-2-methylbenzoate (16.3 g, 66 mmol) and NCS (10.3 g, 76 mmol) were dissolved in DCM (50 mL) and sulfuric acid (50 mL) was added slowly. The mixture was then heated at 40° C. for 2 h. The mixture was allowed to cool to r.t. and poured into ice water and the mixture was then extracted with DCM. The organic layer was washed with sat. aq. Na₂S₂O₃ and sat. aq. NaHCO₃, then dried over MgSO₄ and concentrated. The crude product was purified by FCC (0-60% toluene in heptane) to provide the title compound. LCMS calc. for C₉H₈BrClFO₂ (M+H)⁺: m/z=281.0, 283.0; found 281.0, 283.0.

### Step 2. Methyl 4-bromo-2-(bromomethyl)-5-chloro-3-fluorobenzoate

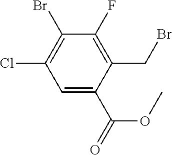

To a solution of methyl 4-bromo-5-choro-3-fluoro-2-methylbenzoate (5.1 g, 19.6 mmol) in trifluorotoluene (82 mL) were added NBS (4.05 g, 22.5 mmol) and AIBN (685 mg, 4.2 mmol) and the mixture was heated at 110° C. overnight. After cooling, the mixture was washed with water. The aqueous layer was extracted with EtOAc and the combined organics were dried over MgSO₄ and concentrated. The crude product was purified by FCC (0-70% toluene in heptane) to provide the title compound. LCMS calc. for C₉H₇Br₂ClFO₂ (M+H)⁺: m/z=358.8, 360.8, 362.8; found 358.8, 360.8, 362.8.

### Step 3. Methyl 4-bromo-5-chloro-2-(cyanomethyl)-3-fluorobenzoate

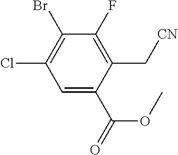 [SMILES: COC(=O)c1cc(Cl)c(Br)c(F)c1CC#N]

To a solution of methyl 4-bromo-2-(bromomethyl)-5-chloro-3-fluorobenzoate (7.51 g, 20.8 mmol) in 1,4-dioxane/water (2:1, 70 mL total volume) at 0° C. was added sodium cyanide (2.04 g, 41.7 mmol) and the mixture was stirred at r.t. overnight. Additional sodium cyanide (1.0 g, 20.4 mmol) was added and stirring was continued for an additional 4 h. The reaction was quenched with sat. aq. NH₄Cl. The mixture was extracted with EtOAc and the organic layer was washed with brine, dried over MgSO₄ and concentrated. The crude product was purified by FCC (0-40% EtOAc in heptane) to provide the title compound (3.73 g, 53% yield). LCMS calc. for C₁₀H₇BrClFNO₂ (M+H)⁺: m/z=305.9, 307.9; found 305.9, 307.9.

### Step 4. 6-Bromo-7-chloro-5-fluoro-3,4-dihydroisoquinolin-1(2H)-one

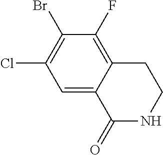 [SMILES: O=C1NCCc2c1cc(Cl)c(Br)c2F]

To a solution of methyl 4-bromo-5-chloro-2-(cyanomethyl)-3-fluorobenzoate (1.27 g, 4.14 mmol) in THF (20 mL) was added borane-DMS complex (1.0 M in THF, 0.6 mL, 6.32 mmol) dropwise and the mixture was stirred at 45° C. for 3 h. In a separate flask, HCl (1 M, 50 mL) was heated at 60° C. and the first solution was added slowly. The mixture was stirred at 60° C. for 5 min., then allowed to cool to r.t. and basified to pH 11 with solid NaOH pellets. The mixture was extracted with EtOAc and the organic layer washed with water and brine, dried over Na₂SO₄ and concentrated. The crude product was purified by FCC (0-75% EtOAc in heptane) to provide the title compound (210 mg, 18% yield). LCMS calc. for C₉H7BrClFNO (M+H)⁺: m/z=277.9, 279.9; found 277.9, 279.9.

### Step 5. 6-Bromo-7-chloro-5-fluoro-2-(1-(2-nitropyridin-3-yl)ethyl)-3,4-dihydroisoquinolin-1(2H)-one

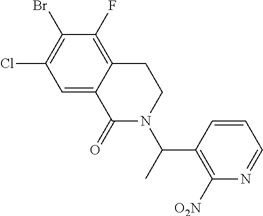 [SMILES: CC(c1cccnc1[N+](=O)[O-])N1CCc2c(cc(Cl)c(Br)c2F)C1=O]

To potassium hydride (86 mg, 1.08 mmol) at 0° C. was added a DMF (3 mL) solution of 6-bromo-7-chloro-5-fluoro-3,4-dihydroisoquinolin-1(2H)-one (200 mg, 0.72 mmol) and the mixture was stirred at this temp for 10 min., then a solution of 3-(1-bromoethyl)-2-nitropyridine (237 μL, 1.44 mmol) in DMF (1 mL) was added and the mixture was stirred at 0° C. for an additional 30 min., then quenched with sat. aq. NH₄Cl. The mixture was extracted with EtOAc and the organics were washed with water and brine, dried over MgSO₄ and concentrated. The crude product was used in the next step without further purification. LCMS calc. for C₁₆H₁₃BrClFN₃O₃(M+H)⁺: m/z=428.0, 430.0; found 428.0, 430.0.

### Step 6. 2-(1-(2-Aminopyridin-3-yl)ethyl)-6-bromo-7-chloro-5-fluoro-3,4-dihydroisoquinolin-1(2H)-one

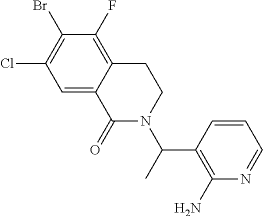 [SMILES: CC(c1cccnc1N)N1CCc2c(cc(Cl)c(Br)c2F)C1=O]

To a solution of the product from the previous step (300 mg, 0.7 mmol) in a mixture of THF/MeOH/water (1:1:1, 6 mL total volume) were added iron (586 mg, 10.5 mmol) and NH₄Cl (562 mg, 10.5 mmol) and the mixture was heated at 70° C. for 30 min., then diluted with DCM and filtered through a pad of diatomaceous earth. The filtrate was concentrated and the crude product was used in the next step without further purification (279 mg, ˜100% yield). LCMS calc. for C₁₆H₁₅BrClFN₃O (M+H)⁺: m/z=398.0, 400.0; found 398.0, 400.0.

### Step 7. 2-Amino-4-(2-(1-(2-aminopyridin-3-yl)ethyl)-7-chloro-5-fluoro-1-oxo-1,2,3,4-tetrahydroisoquinolin-6-yl)-7-fluorobenzo[b]thiophene-3-carbonitrile

To a mixture of the product from the previous step (300 mg, 0.75 mmol), tert-butyl (3-cyano-4-(5,5-dimethyl-1,3,2-dioxaborinan-2-yl)-7-fluorobenzo[b]thiophen-2-yl)carbamate (608 mg, 1.5 mmol), K₂CO₃ (520 mg, 3.7 mmol) and dppf-PdCl₂ (83 mg, 0.11 mmol) was added 1,4-dioxane (4 mL) and the mixture was placed under N₂, then heated at 115° C. for 2 h. The reaction was quenched with water and the mixture was extracted with DCM. The organic layer was dried over MgSO₄ and concentrated. The crude product was purified by SFC (Waters 2-Pic column, eluting with a gradient of MeOH/CO₂, 6-28% at 140 mL/min.). The intermediate was dissolved in 1:1 MeCN/4 M HCl in 1,4-dioxane (1 mL total volume), then stirred for 30 min., diluted with MeCN and purified by prep. LCMS (XBRIDGE® C₁₈ column, eluting with a gradient of MeCN/water containing 0.1% TFA, at flow rate of 60 mL/min.) to afford the title compound. LCMS calc. for C₂₅H₁₉ClF₂N₅OS (M+H)⁺: m/z=510.1; found 510.1.

The title compound comprises a mixture of diastereoisomers, i.e.,
(Rₐ)-2-amino-4-(2-((R)-1-(2-aminopyridin-3-yl)ethyl)-7-chloro-5-fluoro-1-oxo-1,2,3,4-tetrahydroisoquinolin-6-yl)-7-fluorobenzo[b]thiophene-3-carbonitrile;
(Sₐ)-2-amino-4-(2-((S)-1-(2-aminopyridin-3-yl)ethyl)-7-chloro-5-fluoro-1-oxo-1,2,3,4-tetrahydroisoquinolin-6-yl)-7-fluorobenzo[b]thiophene-3-carbonitrile;
(Rₐ)-2-amino-4-(2-((S)-1-(2-aminopyridin-3-yl)ethyl)-7-chloro-5-fluoro-1-oxo-1,2,3,4-tetrahydroisoquinolin-6-yl)-7-fluorobenzo[b]thiophene-3-carbonitrile; and
(Sₐ)-2-amino-4-(2-((R)-1-(2-aminopyridin-3-yl)ethyl)-7-chloro-5-fluoro-1-oxo-1,2,3,4-tetrahydroisoquinolin-6-yl)-7-fluorobenzo[b]thiophene-3-carbonitrile.

### Example 15. 2-Amino-4-(2-(1-amino-6,7-dihydro-5H-cyclopenta[c]pyridin-7-yl)-5-chloro-7-fluoro-1-oxo-1,2,3,4-tetrahydroisoquinolin-6-yl)-7-fluorobenzo[b]thiophene-3-carbonitrile

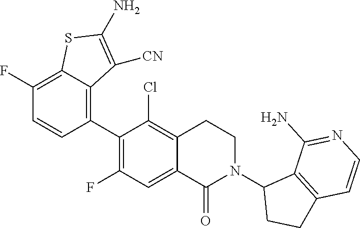 [SMILES: N#Cc1c(N)sc2c(F)ccc(-c3c(F)cc4c(c3Cl)CCN(C3CCc5ccnc(N)c53)C4=O)c12]

To a solution of Intermediate 2 (46 mg, 0.088 mmol) and AcOH (5.1 μL 0.088 mmol) in DCE (1 mL)/MeOH (0.33 mL) was added 6,7-dihydro-5H-cyclopenta[c]pyridine-1,7-diamine dihydrochloride (39 mg, 0.18 mmol) followed by NaBH(OAc)₃ (75 mg, 0.35 mmol). The mixture was stirred at r.t. for 15 min., then DIPEA (0.15 mL, 0.88 mmol) was added by syringe and stirring continued at r.t. for 30 min. The mixture was then concentrated and the residue was redissolved in MeCN (1 mL) and 4 M HCl in 1,4-dioxane (1 mL). After stirring for 45 min., the mixture was filtered, concentrated, and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₆H₁₉ClF₂N₅OS (M+H)⁺: m/z=522.1; found 522.1.

The title compound comprises a mixture of diastereoisomers, i.e.,
2-amino-4-((Rₐ)-2-(1-amino-6,7-dihydro-5H-cyclopenta[c]pyridin-7-yl)-5-chloro-7-fluoro-1-oxo-1,2,3,4-tetrahydroisoquinolin-6-yl)-7-fluorobenzo[b]thiophene-3-carbonitrile; and
2-amino-4-((Sₐ)-2-(1-amino-6,7-dihydro-5H-cyclopenta[c]pyridin-7-yl)-5-chloro-7-fluoro-1-oxo-1,2,3,4-tetrahydroisoquinolin-6-yl)-7-fluorobenzo[b]thiophene-3-carbonitrile.

### Example 16. 2-Amino-4-(2-(1-(4-aminopyrimidin-5-yl)ethyl)-5-chloro-7-fluoro-1-oxo-1,2,3,4-tetrahydroisoquinolin-6-yl)-7-fluorobenzo[b]thiophene-3-carbonitrile

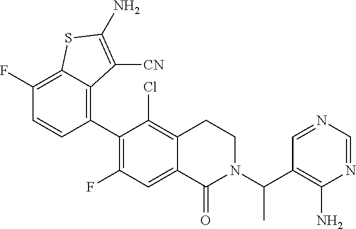 [SMILES: CC(c1cncnc1N)N1CCc2c(cc(F)c(-c3ccc(F)c4sc(N)c(C#N)c34)c2Cl)C1=O]

### Step 1:4-((4-Methoxybenzyl)amino)pyrimidine-5-carbonitrile

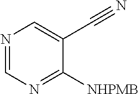

A solution of (4-methoxyphenyl)methanamine (1.1 mL, 8.7 mmol) 4-chloropyrimidine-5-carbonitrile (1.0 g, 7.2 mmol) and DIPEA (2.5 mL, 14 mmol) in MeCN (10 mL) was stirred at r.t. for 4 h, then concentrated and purified by FCC (0-100% EtOAc in hexanes) to provide the title compound (1.23 g, 71% yield). LCMS calc. for C₁₃H₁₃N₄O (M+H)⁺: m/z=241.1; found 241.1.

### Step 2: 5-(1-Aminoethyl)-N-(4-methoxybenzyl)pyrimidin-4-amine

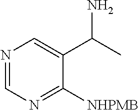

To a solution of 4-((4-methoxybenzyl)amino)pyrimidine-5-carbonitrile (481 mg, 2.0 mmol) in THF (18 mL) was added MeMgBr (3.0 M in Et₂O, 1.37 mL, 4.10 mmol) at 0° C. The mixture was allowed to warm to r.t. and stirred overnight. MeOH (1 mL) was then added to the mixture followed by the addition of NaBH₄ (151 mg, 4.0 mmol). The mixture was stirred at r.t. for 4 h, then quenched with sat. aq. NaHCO₃ and extracted with EtOAc. The organic layer was washed with water and brine, dried over Na₂SO₄ and concentrated. The crude product was purified by FCC (0-100% EtOAc in hexanes, then 15-20% MeOH in DCM) to provide the title compound (241 mg, 47% yield). LCMS calc. for C₁₄H₁₉N₄O (M+H)⁺: m/z=259.2; found 259.2.

### Step 3. 2-Amino-4-(2-(1-(4-aminopyrimidin-5-yl)ethyl)-5-chloro-7-fluoro-1-oxo-1,2,3,4-tetrahydroisoquinolin-6-yl)-7-fluorobenzo[b]thiophene-3-carbonitrile

The title compound was prepared in a similar fashion to Example 7, using 5-(1-aminoethyl)-N-(4-methoxybenzyl)pyrimidin-4-amine instead of 4-(aminomethyl)-1-methyl-1H-pyrazol-3-amine. To affect deprotection, the residue was dissolved in TFA (2 mL) and heated at 90° C. for 20 min. Purification was performed by prep. LCMS (XBRIDGE® C₁₈ column, eluting with a gradient of MeCN/water containing 0.1% NH₄OH, at flow rate of 60 mL/min.) to afford the title compound. LCMS calc. for C₂₄H₁₈ClF₂N₆OS (M+H)⁺: m/z=511.1; found 511.1.

The title compound comprises a mixture of diastereoisomers, i.e.,
2-amino-4-((Rₐ)-2-((R)-1-(4-aminopyrimidin-5-yl)ethyl)-5-chloro-7-fluoro-1-oxo-1,2,3,4-tetrahydroisoquinolin-6-yl)-7-fluorobenzo[b]thiophene-3-carbonitrile;
2-amino-4-((Rₐ)-2-((S)-1-(4-aminopyrimidin-5-yl)ethyl)-5-chloro-7-fluoro-1-oxo-1,2,3,4-tetrahydroisoquinolin-6-yl)-7-fluorobenzo[b]thiophene-3-carbonitrile;
2-amino-4-((Sₐ)-2-((R)-1-(4-aminopyrimidin-5-yl)ethyl)-5-chloro-7-fluoro-1-oxo-1,2,3,4-tetrahydroisoquinolin-6-yl)-7-fluorobenzo[b]thiophene-3-carbonitrile; and
2-amino-4-((Sₐ)-2-((S)-1-(4-aminopyrimidin-5-yl)ethyl)-5-chloro-7-fluoro-1-oxo-1,2,3,4-tetrahydroisoquinolin-6-yl)-7-fluorobenzo[b]thiophene-3-carbonitrile.

### Example 17. 2-Amino-4-(5-chloro-7-fluoro-2-(1-methyl-2-oxopyrrolidin-3-yl)-1-oxo-1,2,3,4-tetrahydroisoquinolin-6-yl)-7-fluorobenzo[b]thiophene-3-carbonitrile

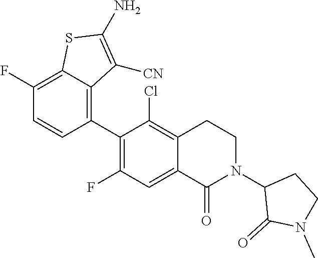 [SMILES: CN1CCC(N2CCc3c(cc(F)c(-c4ccc(F)c5sc(N)c(C#N)c45)c3Cl)C2=O)C1=O]

To a stirred mixture of Intermediate 2 (20 mg, 0.038 mmol), 3-amino-N-methyl-2-pyrrolidinone (8.76 mg, 0.077 mmol) and NaBH(OAc)₃ (32.5 mg, 0.154 mmol) in HFIP (0.250 mL) and DCE (1 mL) was added TFA (8.87 μL, 0.115 mmol) and the mixture was stirred at r.t. for 30 min. The mixture was diluted with toluene (2 mL) and DIPEA (1 mL) and the mixture was heated at 100° C. for 30 min. The mixture was subsequently concentrated and was resuspended in MeCN (3 mL) and TFA (2 mL) and was stirred at r.t. for 1 h. The mixture was subsequently filtered and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₃H₁₈ClF₂N₄O₂S (M+H)⁺: m/z=487.1; found 487.1.

The title compound comprises a mixture of diastereoisomers, i.e.,
2-amino-4-((Rₐ)-5-chloro-7-fluoro-2-(1-methyl-2-oxopyrrolidin-3-yl)-1-oxo-1,2,3,4-tetrahydroisoquinolin-6-yl)-7-fluorobenzo[b]thiophene-3-carbonitrile; and
2-amino-4-((Sₐ)-5-chloro-7-fluoro-2-(1-methyl-2-oxopyrrolidin-3-yl)-1-oxo-1,2,3,4-tetrahydroisoquinolin-6-yl)-7-fluorobenzo[b]thiophene-3-carbonitrile.

### Example 18. 2-Amino-4-(2-((3-amino-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile

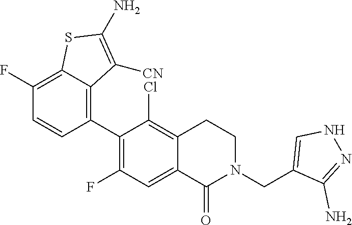 [SMILES: N#Cc1c(N)sc2c(F)ccc(-c3c(F)cc4c(c3Cl)CCN(Cc3c[nH]nc3N)C4=O)c12]

### Step 1: 4-(Aminomethyl)-1H-pyrazol-3-amine

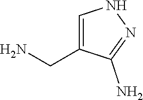

To a solution of 3-amino-1H-pyrazole-4-carbonitrile (250 mg, 2.31 mmol) and THF (1 mL) was added BH₃.DMS (5.78 mL, 11.56 mmol). The mixture was heated at 80° C. for 24 h. Afterwards the mixture was cooled down, concentrated, and was taken forward to the next step without further purification. LCMS calc. for C₄H₁₂N₅(M+NH₄)*: m/z=130.1; found 130.1.

### Step 2: 2-Amino-4-(2-((3-amino-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile

The title compound was prepared in a similar fashion to Example 17, using 4-(aminomethyl)-1H-pyrazol-3-amine instead of 3-amino-N-methyl-2-pyrrolidinone. Purification was performed by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₂H₁₆ClF₂N₆OS (M+H)⁺: m/z=485.1; found=485.1.

The title compound comprises a mixture of diastereoisomers, i.e.,
(Rₐ)-2-amino-4-(2-((3-amino-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile; and
(Sₐ)-2-amino-4-(2-((3-amino-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile.

### Example 19. 6-(2-Amino-3-cyano-7-fluorobenzo[b]thiophen-4-yl)-2-(1-(2-aminopyridin-3-yl)ethyl)-7-fluoro-1-oxo-1,2,3,4-tetrahydroisoquinoline-5-carbonitrile

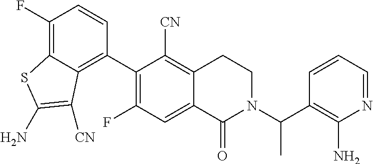 [SMILES: CC(c1cccnc1N)N1CCc2c(cc(F)c(-c3ccc(F)c4sc(N)c(C#N)c34)c2C#N)C1=O]

In a 2-dram vial tert-butyl (3-(1-(6-(2-((tert-butoxycarbonyl)amino)-3-cyano-7-fluorobenzo[b]thiophen-4-yl)-5-chloro-7-fluoro-1-oxo-3,4-dihydroisoquinolin-2(1H)-yl)ethyl)pyridin-2-yl)carbamate (from Example 1, Step 4; 50 mg, 0.070 mmol), t-BuXPhos Pd G4 (5.70 mg, 7.04 μmol), potassium ferrocyanide(II) trihydrate (20.82 mg, 0.049 mmol), and potassium acetate (2.7 mg, 0.028 mmol) were dissolved in 1,4-dioxane (1.05 mL) and water (0.35 mL) under nitrogen. The mixture was stirred for 3 h at 85° C. Additional water (0.5 mL) and KOAc (10 mg) were added and the mixture was heated for an additional 3 h at 85° C. to affect full Boc deprotection. The mixture was cooled to r.t., poured into a vial containing 4 mL of brine, and extracted with DCM (3×5 mL). The DCM layer was passed through a phase-separator, dried with MgSO₄, filtered, and concentrated in vacuo. The crude residue was dissolved in MeCN (4 mL) and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₆H₁₉F₂N₆OS (M+H)⁺: m/z=501.1; found=501.2.

The title compound comprises a mixture of diastereoisomers, i.e.,
(Rₐ)-6-(2-amino-3-cyano-7-fluorobenzo[b]thiophen-4-yl)-2-((R)-1-(2-aminopyridin-3-yl)ethyl)-7-fluoro-1-oxo-1,2,3,4-tetrahydroisoquinoline-5-carbonitrile;
(Rₐ)-6-(2-amino-3-cyano-7-fluorobenzo[b]thiophen-4-yl)-2-((S)-1-(2-aminopyridin-3-yl)ethyl)-7-fluoro-1-oxo-1,2,3,4-tetrahydroisoquinoline-5-carbonitrile;
(Sₐ)-6-(2-amino-3-cyano-7-fluorobenzo[b]thiophen-4-yl)-2-((R)-1-(2-aminopyridin-3-yl)ethyl)-7-fluoro-1-oxo-1,2,3,4-tetrahydroisoquinoline-5-carbonitrile; and
(Sₐ)-6-(2-amino-3-cyano-7-fluorobenzo[b]thiophen-4-yl)-2-((S)-1-(2-aminopyridin-3-yl)ethyl)-7-fluoro-1-oxo-1,2,3,4-tetrahydroisoquinoline-5-carbonitrile.

### Example 20. 2-Amino-4-(2-(1-(2-aminopyridin-3-yl)ethyl)-5-ethynyl-7-fluoro-1-oxo-1,2,3,4-tetrahydroisoquinolin-6-yl)-7-fluorobenzo[b]thiophene-3-carbonitrile

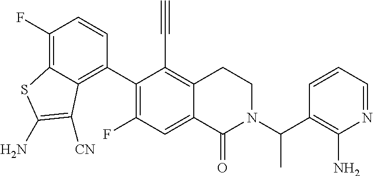 [SMILES: C#Cc1c2c(cc(F)c1-c1ccc(F)c3sc(N)c(C#N)c13)C(=O)N(C(C)c1cccnc1N)CC2]

### Step 1. tert-Butyl (4-(2-(1-(2-(bis(4-methoxybenzyl)amino)pyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate

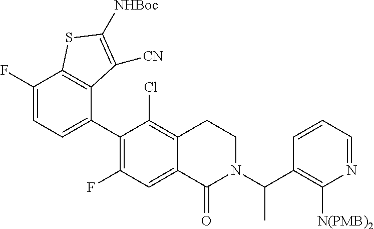

In a 40 mL vial 2-(1-(2-(bis(4-methoxybenzyl)amino)pyridin-3-yl)ethyl)-6-bromo-5-chloro-7-fluoro-3,4-dihydroisoquinolin-1(2H)-one (from Example 1, Step 2; 360 mg, 0.722 mmol), tert-butyl (3-cyano-4-(5,5-dimethyl-1,3,2-dioxaborinan-2-yl)-7-fluorobenzo[b]thiophen-2-yl)carbamate (443 mg, 1.096 mmol), dichloro[bis(diphenylphosphinophenyl)ether]palladium(II) (58.7 mg, 0.082 mmol), and Cs₂CO₃ (588 mg, 1.804 mmol) were dissolved in toluene (7.2 mL) under N₂. The mixture was stirred rapidly and heated at 105° C. for 2 h. The mixture was cooled, diluted with water (20 mL), and extracted with EtOAc (3×20 mL). Combined organic layers were dried over MgSO₄, filtered, and concentrated in vacuo. The crude residue was purified via FCC (0-70% acetone in heptane) to provide the title compound (345 mg, 0.72 mmol, 78%) as a light-orange solid. LCMS calc. for C₄₆H₄₃ClF₂N₅O₅S (M+H)⁺: m/z=850.3; found 850.2.

### Step 2. 2-Amino-4-(2-(1-(2-aminopyridin-3-yl)ethyl)-7-fluoro-1-oxo-5-((triisopropylsilyl)ethynyl)-1,2,3,4-tetrahydroisoquinolin-6-yl)-7-fluorobenzo[b]thiophene-3-carbonitrile

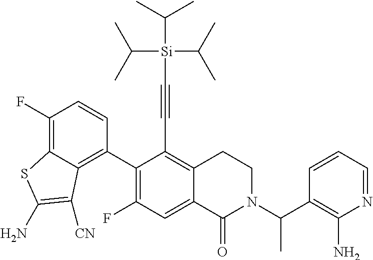 [SMILES: CC(c1cccnc1N)N1CCc2c(cc(F)c(-c3ccc(F)c4sc(N)c(C#N)c34)c2C#C[Si](C(C)C)(C(C)C)C(C)C)C1=O]

In a 1 dram vial tert-butyl (4-(2-(1-(2-(bis(4-methoxybenzyl)amino)pyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate (50 mg, 0.059 mmol), Cs₂CO₃ (96 mg, 0.294 mmol), SPhos Pd G4 (14.01 mg, 0.018 mmol), and (triisopropylsilyl)acetylene (132 μL, 0.588 mmol) were dissolved in DMF (294 μL) under N₂. The mixture was heated at 110° C. for 2 h. The mixture was poured into a 40 mL vial containing 5 mL of brine and then extracted with EtOAc (3×5 mL). The organic layers were combined and washed with 10% w/v aq. LiCl solution (2×4 mL). The organic layer was dried with MgSO₄, filtered, and concentrated in vacuo. The crude material was treated with TFA (0.45 mL) and HFIP (0.3 mL), heated at 60° C., and stirred at 500 RPM for 90 min. Volatiles were removed in vacuo and purified by prep. LCMS (XBRIDGE® C₁₈ column, eluting with a gradient of MeCN/water containing 0.1% TFA, at flow rate of 60 mL/min.) to afford the title compound (18 mg, 0.028 mmol, 47%). LCMS calc. for C₃₆H₄₀F₂N₅OSSi (M+H)⁺: m/z=656.3; found=656.3.

### Step 3. 2-Amino-4-(2-(1-(2-aminopyridin-3-yl)ethyl)-5-ethynyl-7-fluoro-1-oxo-1,2,3,4-tetrahydroisoquinolin-6-yl)-7-fluorobenzo[b]thiophene-3-carbonitrile

To a vial containing 2-amino-4-(2-(1-(2-aminopyridin-3-yl)ethyl)-7-fluoro-1-oxo-5-((triisopropylsilyl)ethynyl)-1,2,3,4-tetrahydroisoquinolin-6-yl)-7-fluorobenzo[b]thiophene-3-carbonitrile (18.1 mg, 0.028 mmol) dissolved in MeCN (0.3 mL) was added CsF (89 mg, 0.588 mmol). The mixture was heated at 60° C., stirred for 30 min., then cooled to 21° C. and diluted with MeCN to a total volume of 4 mL. The mixture was filtered (syringe filter) and purified by prep. LCMS (XBRIDGE® C₁₈ column, eluting with a gradient of MeCN/water containing 0.1% TFA, at flow rate of 60 mL/min.) to yield the title compound as a TFA salt. LCMS calc. for C₂₇H₂₀F₂N₅OS⁺ (M+H)⁺: m/z=500.1; found 500.2.

The title compound comprises a mixture of diastereoisomers, i.e.,
2-amino-4-((Rₐ)-2-((R)-1-(2-aminopyridin-3-yl)ethyl)-5-ethynyl-7-fluoro-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-5-ethynyl-7-fluoro-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-5-ethynyl-7-fluoro-1-oxo-1,2,3,4-tetrahydroisoquinolin-6-yl)-7-fluorobenzo[b]thiophene-3-carbonitrile; and
2-amino-4-((S)-2-((S)-1-(2-aminopyridin-3-yl)ethyl)-5-ethynyl-7-fluoro-1-oxo-1,2,3,4-tetrahydroisoquinolin-6-yl)-7-fluorobenzo[b]thiophene-3-carbonitrile.

### Example 21. 2-Amino-4-(2-(1-(2-aminopyridin-3-yl)ethyl)-7-fluoro-5-(hydroxymethyl)-1-oxo-1,2,3,4-tetrahydroisoquinolin-6-yl)-7-fluorobenzo[b]thiophene-3-carbonitrile

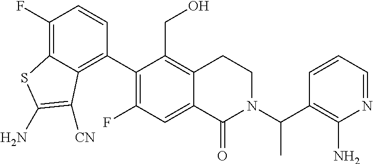 [SMILES: CC(c1cccnc1N)N1CCc2c(cc(F)c(-c3ccc(F)c4sc(N)c(C#N)c34)c2CO)C1=O]

### Step 1. tert-Butyl(4-(2-(1-(2-(bis(4-methoxybenzyl)amino)pyridin-3-yl)ethyl)-7-fluoro-1-oxo-5-vinyl-1,2,3,4-tetrahydroisoquinolin-6-yl)-3-cyano-7-fluorobenzo[b]thiophen-2-yl)carbamate

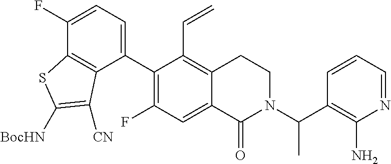 [SMILES: C=Cc1c2c(cc(F)c1-c1ccc(F)c3sc(NC(=O)OC(C)(C)C)c(C#N)c13)C(=O)N(C(C)c1cccnc1N)CC2]

In a 1 dram vial tert-butyl (4-(2-(1-(2-(bis(4-methoxybenzyl)amino)pyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate (from Example 20, Step 1; 209 mg, 0.246 mmol), 2-vinylboronic acid pinacol ester (189 μl, 1.229 mmol), and SPhos Pd G4 (48.8 mg, 0.061 mmol) were dissolved in 1,4-dioxane (2.1 mL)/water (0.3 mL) under N₂. The mixture was heated to 105° C. for 45 min. The mixture was poured into water (4 mL) and extracted with EtOAc (3×5 mL). Combined organic phases were washed with 4 mL of brine, dried over MgSO₄, and concentrated in vacuo. The crude residue was purified by FCC (0-100% acetone/heptane over 20 CVs) to obtain the title compound (136 mg, 0.162 mmol, 66% yield). LCMS calc. for C₄₈H₄₆F₂N₅O₅S⁺ (M+H)⁺: m/z=842.3; found 842.3.

Step 2. tert-Butyl (4-(2-(1-(2-(bis(4-methoxybenzyl)amino)pyridin-3-yl)ethyl)-7-fluoro-5-formyl-1-oxo-1,2,3,4-tetrahydroisoquinolin-6-yl)-3-cyano-7-fluorobenzo[b]thiophen-2-yl)carbamate

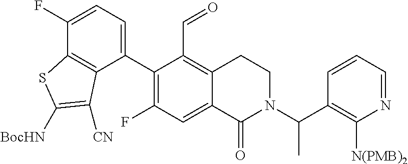

In a 40 mL vial tert-butyl (4-(2-(1-(2-(bis(4-methoxybenzyl)amino)pyridin-3-yl)ethyl)-7-fluoro-1-oxo-5-vinyl-1,2,3,4-tetrahydroisoquinolin-6-yl)-3-cyano-7-fluorobenzo[b]thiophen-2-yl)carbamate (920 mg, 1.093 mmol) was dissolved in THF (9.4 mL) and water (1.6 mL) open to air and then stirred rapidly. An OsO₄ solution (4 wt % in H₂O, 2.083 mL, 0.328 mmol) was added to the mixture at once. After 5 min. of stirring, NaIO₄ (1.08 g, 5.05 mmol) was added portionwise over 5 min. (5 portions). The mixture was stirred rapidly at 21° C. for 3 h and then quenched by pouring into 30 mL of 1:1 sat. aq. Na₂S₂O₃/sat. aq. NaHCO₃ solution. The quenched mixture was diluted with EtOAc (30 mL) and transferred to a separatory funnel. The organic layer was collected and the aqueous layer was further extracted with EtOAc (3×20 mL). Combined organic layers were washed with sat. aq. Na₂S₂O₃ solution (10 mL) followed by brine (10 mL). The organic layer was dried with Na₂SO₄, filtered, and concentrated in vacuo to afford the title compound. The crude material was of suitable purity to be used directly in the next reaction. LCMS calc. for C₄₇H₄₄F₂N₅O₆S⁺ (M+H)⁺: m/z=844.3; found 844.3.

### Step 3. 2-Amino-4-(2-(1-(2-aminopyridin-3-yl)ethyl)-7-fluoro-5-(hydroxymethyl)-1-oxo-1,2,3,4-tetrahydroisoquinolin-6-yl)-7-fluorobenzo[b]thiophene-3-carbonitrile

In a 2 dram vial tert-butyl (4-(2-(1-(2-(bis(4-methoxybenzyl)amino)pyridin-3-yl)ethyl)-7-fluoro-5-formyl-1-oxo-1,2,3,4-tetrahydroisoquinolin-6-yl)-3-cyano-7-fluorobenzo[b]thiophen-2-yl)carbamate (40 mg, 0.047 mmol) was dissolved in THF (0.4 mL)/MeOH (1.3 mL) under N₂. The mixture was stirred rapidly and a single pellet of NaBH₄ (10.6 mg, 0.280 mmol) was added at once. The mixture was capped under N₂ and stirred at 21° C. for 30 min. The mixture was quenched with saturated NH₄Cl solution (2 mL) and extracted with DCM (3×4 mL). Combined DCM extracts were washed with brine, dried with MgSO₄, filtered, and concentrated in vacuo. The crude product was treated with HFIP (0.4 mL)/TFA (0.3 mL) and heated to 90° C. with stirring for 5 min. The mixture was cooled to 21° C. and volatiles were removed in vacuo. The crude residue was dissolved in MeCN (4 mL) and purified by prep. LCMS (XBRIDGE® C₁₈ column, eluting with a gradient of MeOH/water containing 0.1% NH₄OH, at flow rate of 60 mL/min.) to afford the title compound. LCMS calc. for C₂₆H₂₂F₂N₅O₂S⁺ (M+H)⁺: m/z=506.2; found 506.1.

The title compound comprises a mixture of diastereoisomers, i.e.,
2-amino-4-((Rₐ)-2-((R)-1-(2-aminopyridin-3-yl)ethyl)-7-fluoro-5-(hydroxymethyl)-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-7-fluoro-5-(hydroxymethyl)-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-7-fluoro-5-(hydroxymethyl)-1-oxo-1,2,3,4-tetrahydroisoquinolin-6-yl)-7-fluorobenzo[b]thiophene-3-carbonitrile; and
2-amino-4-((Sₐ)-2-((S)-1-(2-aminopyridin-3-yl)ethyl)-7-fluoro-5-(hydroxymethyl)-1-oxo-1,2,3,4-tetrahydroisoquinolin-6-yl)-7-fluorobenzo[b]thiophene-3-carbonitrile.

### Example 22. 2-Amino-4-(2-(1-(2-aminopyridin-3-yl)ethyl)-5-(difluoromethyl)-7-fluoro-1-oxo-1,2,3,4-tetrahydroisoquinolin-6-yl)-7-fluorobenzo[b]thiophene-3-carbonitrile

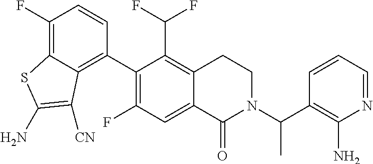 [SMILES: CC(c1cccnc1N)N1CCc2c(cc(F)c(-c3ccc(F)c4sc(N)c(C#N)c34)c2C(F)F)C1=O]

In a 2 dram vial tert-butyl (4-(2-(1-(2-(bis(4-methoxybenzyl)amino)pyridin-3-yl)ethyl)-7-fluoro-5-formyl-1-oxo-1,2,3,4-tetrahydroisoquinolin-6-yl)-3-cyano-7-fluorobenzo[b]thiophen-2-yl)carbamate (from Example 21, Step 2; 27.8 mg, 0.033 mmol) was dissolved in DCM (0.5 mL) under N₂. The mixture was cooled in an ice bath to 0° C. and DAST (0.013 mL, 0.099 mmol) was added at once by micropipette. The mixture was stirred for 10 min. at 0° C. and then removed from the ice bath and allowed to warm to r.t. After stirring for 1 h, additional DAST (0.044 mL, 0.329 mmol) was added and the mixture was stirred an additional 2 h. The mixture was cooled to 0° C. in an ice bath and diluted with DCM (2 mL). Sat. aq. NaHCO₃ (1.5 mL) was added very slowly (to control rate of gas evolution) to the stirring mixture. After stirring for 30 min., the mixture was poured into a 40 mL vial containing 5 mL water and 3 mL DCM. The organic layer was isolated and the aqueous layer was further extracted with DCM (3×5 mL). Combined organic layers were washed with brine (5 mL), dried over MgSO₄, and concentrated in vacuo. The residue was dissolved in TFA (0.25 mL) and HFIP (0.35 mL) and heated to 75° C. for 30 min. The mixture was cooled to r.t. and concentrated in vacuo. The residue was purified by prep. LCMS (XBRIDGE® C₁₈ column, eluting with a gradient of MeCN/water containing 0.1% NH₄OH, at flow rate of 60 mL/min.) to afford the title compound. LCMS calc. for C₂₆H₂₀F₄N₅OS⁺ (M+H)⁺: m/z=526.1; found 526.1.

The title compound comprises a mixture of diastereoisomers, i.e.,
2-amino-4-((Rₐ)-2-((R)-1-(2-aminopyridin-3-yl)ethyl)-5-(difluoromethyl)-7-fluoro-1-oxo-1,2,3,4-tetrahydroisoquinolin-6-yl)-7-fluorobenzo[b]thiophene-3-carbonitrile;
2-amino-4-((R)-2-((S)-1-(2-aminopyridin-3-yl)ethyl)-5-(difluoromethyl)-7-fluoro-1-oxo-1,2,3,4-tetrahydroisoquinolin-6-yl)-7-fluorobenzo[b]thiophene-3-carbonitrile;
2-amino-4-((S)-2-((R)-1-(2-aminopyridin-3-yl)ethyl)-5-(difluoromethyl)-7-fluoro-1-oxo-1,2,3,4-tetrahydroisoquinolin-6-yl)-7-fluorobenzo[b]thiophene-3-carbonitrile; and
2-amino-4-((S)-2-((S)-1-(2-aminopyridin-3-yl)ethyl)-5-(difluoromethyl)-7-fluoro-1-oxo-1,2,3,4-tetrahydroisoquinolin-6-yl)-7-fluorobenzo[b]thiophene-3-carbonitrile.

### Example 23. 6-(2-Amino-3-cyano-7-fluorobenzo[b]thiophen-4-yl)-2-(1-(2-aminopyridin-3-yl)ethyl)-7-fluoro-1-oxo-1,2,3,4-tetrahydroisoquinoline-5-carboxamide

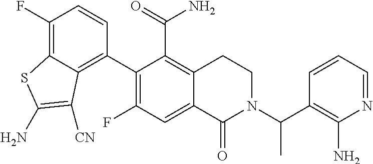 [SMILES: CC(c1cccnc1N)N1CCc2c(cc(F)c(-c3ccc(F)c4sc(N)c(C#N)c34)c2C(N)=O)C1=O]

### Step 1. 2-(1-(2-(bis(4-Methoxybenzyl)amino)pyridin-3-yl)ethyl)-6-(2-((tertbutoxycarbonyl)amino)-3-cyano-7-fluorobenzo[b]thiophen-4-yl)-7-fluoro-1-oxo-1,2,3,4-tetrahydroisoquinoline-5-carboxylic acid

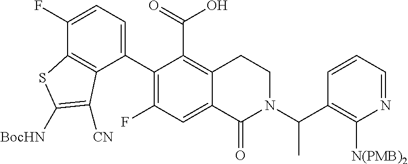

In a 40 mL vial tert-butyl (4-(2-(1-(2-(bis(4-methoxybenzyl)amino)pyridin-3-yl)ethyl)-7-fluoro-5-formyl-1-oxo-1,2,3,4-tetrahydroisoquinolin-6-yl)-3-cyano-7-fluorobenzo[b]thiophen-2-yl)carbamate (from Example 21, Step 2; 278 mg, 0.329 mmol), 2-methyl-2-butene (0.872 mL, 8.23 mmol), potassium phosphate (monobasic, 224 mg, 1.647 mmol) were dissolved in t-BuOH (3.0 mL), water (1.5 mL), and MeCN (1.0 mL) open to air. Sodium chlorite (74.5 mg, 0.824 mmol) was added to the mixture as a solid and the mixture was stirred at r.t. for 30 min. The mixture was diluted with water (5 mL) and acidified to pH 2.7 with 1 N aq. H₃PO₄. The mixture was extracted with DCM (3×8 mL) and combined organic extracts were washed with brine (5 mL), dried over MgSO₄, filtered, and concentrated in vacuo to obtain the title compound (283 mg, 0.329 mmol, 100%) which was used in the next step without further purification. LCMS calc. for C₄₇H₄₄F₂N₅O₇S⁺ (M+H)⁺: m/z=860.3; found 860.4.

### Step 2. 6-(2-Amino-3-cyano-7-fluorobenzo[b]thiophen-4-yl)-2-(1-(2-aminopyridin-3-yl)ethyl)-7-fluoro-1-oxo-1,2,3,4-tetrahydroisoquinoline-5-carboxamide

In a 1 dram vial 2-(1-(2-(bis(4-methoxybenzyl)amino)pyridin-3-yl)ethyl)-6-(2-((tertbutoxycarbonyl)amino)-3-cyano-7-fluorobenzo[b]thiophen-4-yl)-7-fluoro-1-oxo-1,2,3,4-tetrahydroisoquinoline-5-carboxylic acid (20 mg, 0.023 mmol), DIPEA (0.020 mL, 0.116 mmol), HATU (11.05 mg, 0.029 mmol), and NH₄Cl (1.244 mg, 0.023 mmol) were dissolved in DMF (0.1 mL) under N₂. The mixture was stirred at r.t. for 3 h and then diluted with MTBE (2 mL). The organic layer was retained and the aqueous layer was further extracted with MTBE (3×3 mL). Combined organic layers were washed with brine (4 mL) followed by 10% w/v aq. LiCl solution (4 mL), dried over MgSO₄, filtered, and concentrated in vacuo. The crude residue was dissolved in TFA (0.2 mL)/HFIP (0.2 mL) and heated to 75° C. for 30 min. Volatiles were removed in vacuo and the crude residue was purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₆H₂₁F₂N₆O₂S⁺ (M+H)⁺: m/z=519.1; found 519.2.

The title compound comprises a mixture of diastereoisomers, i.e.,
(Rₐ)-6-(2-amino-3-cyano-7-fluorobenzo[b]thiophen-4-yl)-2-((R)-1-(2-aminopyridin-3-yl)ethyl)-7-fluoro-1-oxo-1,2,3,4-tetrahydroisoquinoline-5-carboxamide;
(Rₐ)-6-(2-amino-3-cyano-7-fluorobenzo[b]thiophen-4-yl)-2-((S)-1-(2-aminopyridin-3-yl)ethyl)-7-fluoro-1-oxo-1,2,3,4-tetrahydroisoquinoline-5-carboxamide;
(Sₐ)-6-(2-amino-3-cyano-7-fluorobenzo[b]thiophen-4-yl)-2-((R)-1-(2-aminopyridin-3-yl)ethyl)-7-fluoro-1-oxo-1,2,3,4-tetrahydroisoquinoline-5-carboxamide; and
(Sₐ)-6-(2-amino-3-cyano-7-fluorobenzo[b]thiophen-4-yl)-2-((S)-1-(2-aminopyridin-3-yl)ethyl)-7-fluoro-1-oxo-1,2,3,4-tetrahydroisoquinoline-5-carboxamide.

### Example 24. 2-Amino-4-(2-(1-(2-aminopyridin-3-yl)ethyl)-5-chloro-4,4,7-trifluoro-1-oxo-1,2,3,4-tetrahydroisoquinolin-6-yl)-7-fluorobenzo[b]thiophene-3-carbonitrile

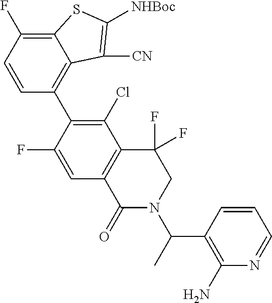 [SMILES: CC(c1cccnc1N)N1CC(F)(F)c2c(cc(F)c(-c3ccc(F)c4sc(NC(=O)OC(C)(C)C)c(C#N)c34)c2Cl)C1=O]

### Step 1. Methyl 4-bromo-3-chloro-2-(1,1-difluoro-2-oxoethyl)-5-fluorobenzoate

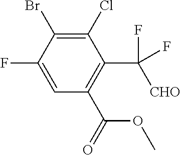 [SMILES: COC(=O)c1cc(F)c(Br)c(Cl)c1C(F)(F)C=O]

To a vial containing methyl 4-bromo-3-chloro-5-fluoro-2-(2-oxoethyl)benzoate (from Example 1, Step 1; 0.150 g, 0.49 mmol) and N-fluorobenzenesulfonimide (0.611 g, 1.94 mmol) was added anhydrous (R)-2-(diphenyl(trimethylsilyl)oxy)methyl)pyrrolidine (0.158 g, 0.49 mmol) and THF (1.0 mL). The mixture was stirred under N₂ for 16 h and concentrated under reduced pressure. The crude product was purified by FCC (0-40% EtOAc/hexanes) to provide the title compound. LCMS calc. for C₁₀H₆BrClF₃O₃(M+H)⁺: m/z=344.9; found 345.0.

### Step 2. Methyl 2-(2-((1-(2-(bis(4-methoxybenzyl)amino)pyridin-3-yl)ethyl)amino)-1,1-difluoroethyl)-4-bromo-3-chloro-5-fluorobenzoate

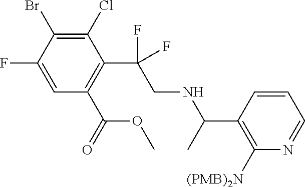

To a DCM (0.5 mL) solution containing methyl 4-bromo-3-chloro-2-(1,1-difluoro-2-oxoethyl)-5-fluorobenzoate (0.056 g, 0.098 mmol) was added PPTS (0.024 g, 0.098 mmol) and Intermediate 3 (0.076 g, 0.20 mmol). The mixture was stirred at r.t. for 0.5 h before NaBH₃CN (0.012 g, 0.20 mmol) was added. After stirring at r.t. for another 0.5 h, the mixture was quenched with water and extracted with DCM three times. The combined organic layers were dried over Na₂SO₄, filtered and concentrated. The residue was used for next step without purification. LCMS calc. for C₃₃H₃₃BrClF₃N₃O₄ (M+H)⁺: m/z=706.1; found 706.1

### Step 3. 2-(1-(2-(bis(4-Methoxybenzyl)amino)pyridin-3-yl)ethyl)-6-bromo-5-chloro-4,4,7-trifluoro-3,4-dihydroisoquinolin-1(2H)-one

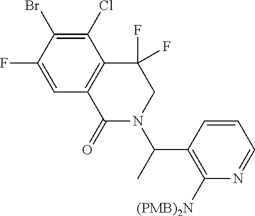

To methyl 2-(2-((1-(2-(bis(4-methoxybenzyl)amino)pyridin-3-yl)ethyl)amino)-1,1-difluoroethyl)-4-bromo-3-chloro-5-fluorobenzoate (0.068 g, 0.098 mmol) in THF (0.5 mL) was added DIPEA (0.05 mL, 0.29 mmol). The mixture was heated at 60° C. for 0.5 h. Upon completion, water was added. The mixture was extracted with EtOAc three times. The combined organic layers were dried over Na₂SO₄, filtered, and concentrated. The crude product was purified by FCC (0-40% EtOAc/hexanes) to provide the title compound. LCMS calc. for C₃₂H₂₉BrClF₃N₃O₃ (M+H)⁺: m/z=674.1; found 674.2.

### Step 4. 2-(1-(2-Aminopyridin-3-yl)ethyl)-6-bromo-5-chloro-4,4,7-trifluoro-3,4-dihydroisoquinolin-1(2H)-one

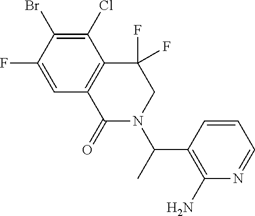 [SMILES: CC(c1cccnc1N)N1CC(F)(F)c2c(cc(F)c(Br)c2Cl)C1=O]

A TFA (0.5 mL) solution containing 2-(1-(2-(bis(4-methoxybenzyl)amino)pyridin-3-yl)ethyl)-6-bromo-5-chloro-4,4,7-trifluoro-3,4-dihydroisoquinolin-1(2H)-one (0.064 g, 0.095 mmol) was heated at 90° C. for 0.5 h. After cooling to r.t., the residue was dissolved in DCM and treated with sat. aq. NaHCO₃. The mixture was extracted with DCM (3×). The combined organic layers were dried over Na₂SO₄, filtered and concentrated to afford the title compound. LCMS calc. for C₁₆H₁₃BrClF₃N₃O (M+H)⁺: m/z=434.0; found 433.9.

### Step 5. 2-Amino-4-(2-(1-(2-aminopyridin-3-yl)ethyl)-5-chloro-4,4,7-trifluoro-1-oxo-1,2,3,4-tetrahydroisoquinolin-6-yl)-7-fluorobenzo[b]thiophene-3-carbonitrile

To a vial containing 2-(1-(2-aminopyridin-3-yl)ethyl)-6-bromo-5-chloro-4,4,7-trifluoro-3,4-dihydroisoquinolin-1(2H)-one (0.052 g, 0.12 mmol), tert-butyl (3-cyano-4-(5,5-dimethyl-1,3,2-dioxaborinan-2-yl)-7-fluorobenzo[b]thiophen-2-yl)carbamate (0.097 g, 0.24 mmol), Pd(dppf)Cl₂·CH₂Cl₂ (0.018 g, 0.024 mmol) and K₂CO₃ (0.083 g, 0.60 mmol) was added 1,4-dioxane (1.8 mL). The mixture was heated at 100° C. under N₂ for 1.5 h. After cooling to r.t., the mixture was diluted with EtOAc and washed with brine. The organic phase was dried over Na₂SO₄, filtered, and concentrated. To the residue dissolved in DCM (0.5 mL) was added TFA (0.5 mL). The mixture was stirred at r.t. for 0.5 h. Upon completion, volatiles were removed under reduced pressure and the residue was dissolved in MeCN (4 mL) and water (1 mL) and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₅H₁₇ClF₄N₅OS (M+H)⁺: m/z=546.1; found 546.1.

The title compound comprises a mixture of diastereoisomers, i.e.,
2-amino-4-((Rₐ)-2-((R)-1-(2-aminopyridin-3-yl)ethyl)-5-chloro-4,4,7-trifluoro-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-5-chloro-4,4,7-trifluoro-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-5-chloro-4,4,7-trifluoro-1-oxo-1,2,3,4-tetrahydroisoquinolin-6-yl)-7-fluorobenzo[b]thiophene-3-carbonitrile; and
2-amino-4-((Sₐ)-2-((S)-1-(2-aminopyridin-3-yl)ethyl)-5-chloro-4,4,7-trifluoro-1-oxo-1,2,3,4-tetrahydroisoquinolin-6-yl)-7-fluorobenzo[b]thiophene-3-carbonitrile.

### Example 25. 2-Amino-4-(2-(1-(2-aminopyridin-3-yl)ethyl)-5-chloro-8-(3-(dimethylamino)prop-1-yn-1-yl)-7-fluoro-1-oxo-1,2,3,4-tetrahydroisoquinolin-6-yl)-7-fluorobenzo[b]thiophene-3-carbonitrile

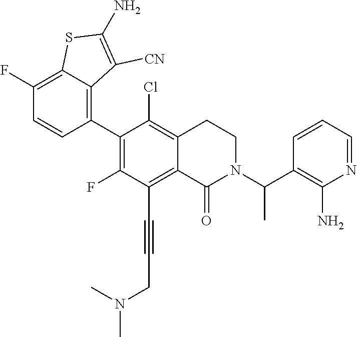 [SMILES: CC(c1cccnc1N)N1CCc2c(Cl)c(-c3ccc(F)c4sc(N)c(C#N)c34)c(F)c(C#CCN(C)C)c2C1=O]

### Step 1. tert-Butyl (4-(8-amino-2-(1-(2-(bis(4-methoxybenzyl)amino)pyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate

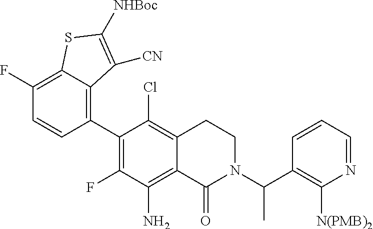

The title compound was prepared according to the procedure of Intermediate 2, Step 2, using 8-amino-2-(1-(2-(bis(4-methoxybenzyl)amino)pyridin-3-yl)ethyl)-6-bromo-5-chloro-7-fluoro-3,4-dihydroisoquinolin-1(2H)-one (from Example 11, Step 9) instead of (E)-4-bromo-3-chloro-2-(2-ethoxyvinyl)-5-fluorobenzoate. LCMS calc. for C₄₆H₄₄ClF₂N₆O₅S (M+H)⁺: m/z=865.3; found 865.3.

### Step 2. tert-Butyl (4-(2-(1-(2-(bis(4-methoxybenzyl)amino)pyridin-3-yl)ethyl)-5-chloro-7-fluoro-8-iodo-1-oxo-1,2,3,4-tetrahydroisoquinolin-6-yl)-3-cyano-7-fluorobenzo[b]thiophen-2-yl)carbamate

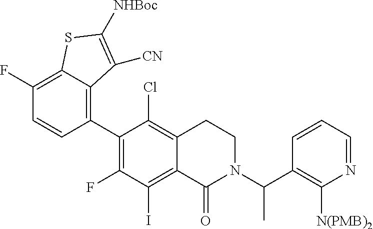

The title compound was prepared according to the procedure of Example 12, Step 2, using tert-butyl (4-(8-amino-2-(1-(2-(bis(4-methoxybenzyl)amino)pyridin-3-yl)ethyl)-5-chloro-7-fluoro-1-oxo-1,2,3,4-tetrahydroisoquinolin-6-yl)-3-cyano-7-fluorobenzo[b]thiophen-2-yl)carbamate instead of tert-butyl (4-(8-amino-2-(1-(2-(bis(4-methoxybenzyl)amino)pyridin-3-yl)ethyl)-5-chloro-7-fluoro-1-oxo-1,2,3,4-tetrahydroisoquinolin-6-yl)-7-fluorobenzo[d]thiazol-2-yl)carbamate. LCMS calc. for C₄₆H₄₂ClF₂IN₅O₅S (M+H)⁺: m/z=976.2; found 976.2.

### Step 3. 2-Amino-4-(2-(1-(2-aminopyridin-3-yl)ethyl)-5-chloro-8-(3-(dimethylamino)prop-1-yn-1-yl)-7-fluoro-1-oxo-1,2,3,4-tetrahydroisoquinolin-6-yl)-7-fluorobenzo[b]thiophene-3-carbonitrile

To a mixture of tert-butyl (4-(2-(1-(2-(bis(4-methoxybenzyl)amino)pyridin-3-yl)ethyl)-5-chloro-7-fluoro-8-iodo-1-oxo-1,2,3,4-tetrahydroisoquinolin-6-yl)-3-cyano-7-fluorobenzo[b]thiophen-2-yl)carbamate (20 mg, 0.020 mmol) and N,N-dimethylprop-2-yn-1-amine (5.11 mg, 0.061 mmol) were added DMF (0.5 mL) and NEt₃ (0.017 mL, 0.123 mmol), followed by Pd(PPh₃)₄ (4.73 mg, 4.10 μmol) and copper(I) iodide (1.17 mg, 6.15 μmol). The mixture was sparged with N₂ for 5 min. and then stirred at 80° C. for 2 h. After cooling to r.t., the mixture was quenched with water (0.4 mL) and sat. aq. NH₄OH (˜0.5 mL). The mixture was then extracted with EtOAc (3×2 mL). The combined organic phase was dried over Na₂SO₄ and concentrated. The crude product was dissolved in TFA (1 mL) and heated to 90° C. for 1 h, then diluted with MeCN and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₃₀H₂₆ClF₂N₆OS (M+H)⁺: m/z=591.2; found 591.2.

The title compound comprises a mixture of diastereoisomers, i.e.,
2-amino-4-((Rₐ)-2-((R)-1-(2-aminopyridin-3-yl)ethyl)-5-chloro-8-(3-(dimethylamino)prop-1-yn-1-yl)-7-fluoro-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-5-chloro-8-(3-(dimethylamino)prop-1-yn-1-yl)-7-fluoro-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-5-chloro-8-(3-(dimethylamino)prop-1-yn-1-yl)-7-fluoro-1-oxo-1,2,3,4-tetrahydroisoquinolin-6-yl)-7-fluorobenzo[b]thiophene-3-carbonitrile; and
2-amino-4-((Sₐ)-2-((S)-1-(2-aminopyridin-3-yl)ethyl)-5-chloro-8-(3-(dimethylamino)prop-1-yn-1-yl)-7-fluoro-1-oxo-1,2,3,4-tetrahydroisoquinolin-6-yl)-7-fluorobenzo[b]thiophene-3-carbonitrile.

### Example 26. 2-Amino-4-(2-(1-(2-aminopyridin-3-yl)ethyl)-5-chloro-8-(4-(dimethylamino)but-1-yn-1-yl)-7-fluoro-1-oxo-1,2,3,4-tetrahydroisoquinolin-6-yl)-7-fluorobenzo[b]thiophene-3-carbonitrile

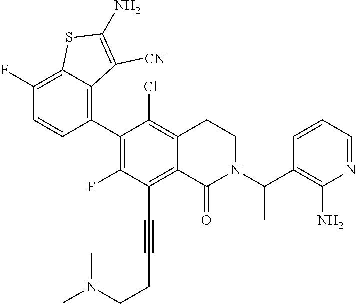 [SMILES: CC(c1cccnc1N)N1CCc2c(Cl)c(-c3ccc(F)c4sc(N)c(C#N)c34)c(F)c(C#CCCN(C)C)c2C1=O]

The title compound was prepared according to the procedure of Example 25, Step 3, using N,N-dimethylbut-3-yn-1-amine instead of N,N-dimethylprop-2-yn-1-amine. LCMS calc. for C₃₁H₂₈ClF₂N₆OS (M+H)⁺: m/z=605.2; found 605.2.

The title compound comprises a mixture of diastereoisomers, i.e.,
2-amino-4-((Rₐ)-2-((R)-1-(2-aminopyridin-3-yl)ethyl)-5-chloro-8-(4-(dimethylamino)but-1-yn-1-yl)-7-fluoro-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-5-chloro-8-(4-(dimethylamino)but-1-yn-1-yl)-7-fluoro-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-5-chloro-8-(4-(dimethylamino)but-1-yn-1-yl)-7-fluoro-1-oxo-1,2,3,4-tetrahydroisoquinolin-6-yl)-7-fluorobenzo[b]thiophene-3-carbonitrile; and
2-amino-4-((Sₐ)-2-((S)-1-(2-aminopyridin-3-yl)ethyl)-5-chloro-8-(4-(dimethylamino)but-1-yn-1-yl)-7-fluoro-1-oxo-1,2,3,4-tetrahydroisoquinolin-6-yl)-7-fluorobenzo[b]thiophene-3-carbonitrile.

### Example 27. 2-Amino-4-(2-(1-(2-aminopyridin-3-yl)ethyl)-5-chloro-7-fluoro-8-((1-methylpyrrolidin-2-yl)ethynyl)-1-oxo-1,2,3,4-tetrahydroisoquinolin-6-yl)-7-fluorobenzo[b]thiophene-3-carbonitrile

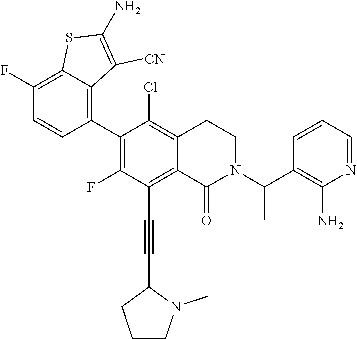 [SMILES: CC(c1cccnc1N)N1CCc2c(Cl)c(-c3ccc(F)c4sc(N)c(C#N)c34)c(F)c(C#CC3CCCN3C)c2C1=O]

The title compound was prepared according to the procedure of Example 25, Step 3, using 2-ethynyl-1-methylpyrrolidine instead of N,N-dimethylprop-2-yn-1-amine. LCMS calc. for C₃₂H₂₈ClF₂NeOS (M+H)⁺: m/z=617.2; found 617.2.

The title compound comprises a mixture of diastereoisomers, i.e.,
2-amino-4-((Rₐ)-2-((R)-1-(2-aminopyridin-3-yl)ethyl)-5-chloro-7-fluoro-8-((1-methylpyrrolidin-2-yl)ethynyl)-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-5-chloro-7-fluoro-8-((1-methylpyrrolidin-2-yl)ethynyl)-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-5-chloro-7-fluoro-8-((1-methylpyrrolidin-2-yl)ethynyl)-1-oxo-1,2,3,4-tetrahydroisoquinolin-6-yl)-7-fluorobenzo[b]thiophene-3-carbonitrile; and
2-amino-4-((Sₐ)-2-((S)-1-(2-aminopyridin-3-yl)ethyl)-5-chloro-7-fluoro-8-((1-methylpyrrolidin-2-yl)ethynyl)-1-oxo-1,2,3,4-tetrahydroisoquinolin-6-yl)-7-fluorobenzo[b]thiophene-3-carbonitrile.

### Example 28. 2-Amino-4-(2-(1-(2-aminopyridin-3-yl)ethyl)-5-chloro-7-fluoro-8-methoxy-1-oxo-1,2,3,4-tetrahydroisoquinolin-6-yl)-7-fluorobenzo[b]thiophene-3-carbonitrile

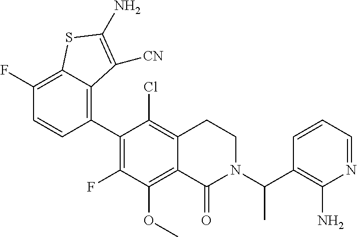 [SMILES: COc1c(F)c(-c2ccc(F)c3sc(N)c(C#N)c23)c(Cl)c2c1C(=O)N(C(C)c1cccnc1N)CC2]

To a mixture of tert-butyl (4-(2-(1-(2-(bis(4-methoxybenzyl)amino)pyridin-3-yl)ethyl)-5-chloro-7-fluoro-8-iodo-1-oxo-1,2,3,4-tetrahydroisoquinolin-6-yl)-3-cyano-7-fluorobenzo[b]thiophen-2-yl)carbamate (from Example 25, Step 2, 30 mg, 0.031 mmol) in MeOH (0.4 mL) was added copper(I) iodide (1.17 mg, 6.15 μmol) and 1,10-phenanthroline (2.22 mg, 0.012 mmol). The mixture was sparged with N₂ for 5 min. and then stirred at 100° C. for 24 h. After cooling to r.t., the mixture was quenched with water (0.4 mL) and sat. aq. NH₄OH (˜0.5 mL). The mixture was then extracted with EtOAc (3×2 mL). The combined organic phase was dried over Na₂SO₄ and concentrated. The crude product was dissolved in TFA (1 mL) and heated to 90° C. for 1 h, then diluted with MeCN and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₆H₂₁ClF₂N₅O₂S (M+H)⁺: m/z=540.2; found 540.2.

The title compound comprises a mixture of diastereoisomers, i.e.,
2-amino-4-((Rₐ)-2-((R)-1-(2-aminopyridin-3-yl)ethyl)-5-chloro-7-fluoro-8-methoxy-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-5-chloro-7-fluoro-8-methoxy-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-5-chloro-7-fluoro-8-methoxy-1-oxo-1,2,3,4-tetrahydroisoquinolin-6-yl)-7-fluorobenzo[b]thiophene-3-carbonitrile; and
2-amino-4-((S)-2-((S)-1-(2-aminopyridin-3-yl)ethyl)-5-chloro-7-fluoro-8-methoxy-1-oxo-1,2,3,4-tetrahydroisoquinolin-6-yl)-7-fluorobenzo[b]thiophene-3-carbonitrile.

### Example 29. 2-Amino-4-(2-(1-(2-aminopyridin-3-yl)ethyl)-5-chloro-8-(2-(dimethylamino)ethoxy)-7-fluoro-1-oxo-1,2,3,4-tetrahydroisoquinolin-6-yl)-7-fluorobenzo[b]thiophene-3-carbonitrile

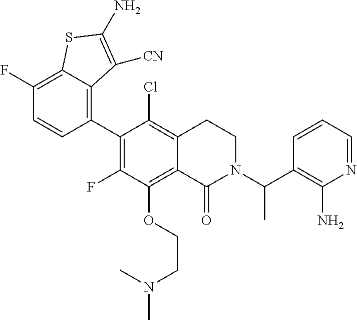 [SMILES: CC(c1cccnc1N)N1CCc2c(Cl)c(-c3ccc(F)c4sc(N)c(C#N)c34)c(F)c(OCCN(C)C)c2C1=O]

### Step 1. tert-Butyl(4-(2-(1-(2-(bis(4-methoxybenzyl)amino)pyridin-3-yl)ethyl)-5-chloro-7-fluoro-8-hydroxy-1-oxo-1,2,3,4-tetrahydroisoquinolin-6-yl)-3-cyano-7-fluorobenzo[b]thiophen-2-yl)carbamate

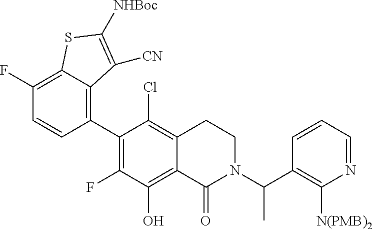

To a mixture of tert-butyl (4-(2-(1-(2-(bis(4-methoxybenzyl)amino)pyridin-3-yl)ethyl)-5-chloro-7-fluoro-8-iodo-1-oxo-1,2,3,4-tetrahydroisoquinolin-6-yl)-3-cyano-7-fluorobenzo[b]thiophen-2-yl)carbamate (from Example 25, Step 2; 250 mg, 0.256 mmol) in DMSO (2.6 mL) was added KOH (57.5 mg, 1.02 mmol), copper(I) iodide (9.75 mg, 0.051 mmol) and 1,10-phenanthroline (18.5 mg, 0.102 mmol). The mixture was sparged with N₂ for 5 min. and then stirred at 100° C. for 24 h. After cooling to r.t., the mixture was quenched with water (1 mL) and sat. aq. NH₄OH (˜2 mL). The mixture was then extracted with EtOAc (3×2 mL). The combined organic phase was dried over Na₂SO₄ and concentrated. The crude residue was purified by FCC (0-100% acetone/hexanes) to obtain the title compound (38 mg, 0.256 mmol, 17% yield). LCMS calc. for C₄₆H₄₃ClF₂N₅O₆S (M+H)⁺: m/z=866.3; found 866.3.

### Step 2. 2-Amino-4-(2-(1-(2-aminopyridin-3-yl)ethyl)-5-chloro-8-(2-(dimethylamino)ethoxy)-7-fluoro-1-oxo-1,2,3,4-tetrahydroisoquinolin-6-yl)-7-fluorobenzo[b]thiophene-3-carbonitrile

To a mixture of tert-butyl (4-(2-(1-(2-(bis(4-methoxybenzyl)amino)pyridin-3-yl)ethyl)-5-chloro-7-fluoro-8-hydroxy-1-oxo-1,2,3,4-tetrahydroisoquinolin-6-yl)-3-cyano-7-fluorobenzo[b]thiophen-2-yl)carbamate (10 mg, 0.031 mmol) in DMF (0.2 mL) was added K₂CO (7.98 mg, 0.058 mmol). The mixture was stirred at 90° C. for 2 h. After cooling to r.t., the mixture was added dimethylamine in THF (0.29 mL, 0.577 mmol, 2.0 M) and heated again at 90° C. for 2 h. The mixture was quenched with water (˜1 mL) and extracted with EtOAc (3×2 mL). The combined organic phase was dried over Na₂SO₄ and concentrated. The crude product was dissolved in TFA (1 mL) and heated to 90° C. for 1 h, then diluted with MeCN and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₉H₂₆ClF₂N₆O₂S (M+H)⁺: m/z=597.2; found 597.2.

The title compound comprises a mixture of diastereoisomers, i.e.,
2-amino-4-((Rₐ)-2-((R)-1-(2-aminopyridin-3-yl)ethyl)-5-chloro-8-(2-(dimethylamino)ethoxy)-7-fluoro-1-oxo-1,2,3,4-tetrahydroisoquinolin-6-yl)-7-fluorobenzo[b]thiophene-3-carbonitrile;
2-amino-4-((Rₐ)-2-((S)-1-(2-aminopyridin-3-yl)ethyl)-5-chloro-8-(2-(dimethylamino)ethoxy)-7-fluoro-1-oxo-1,2,3,4-tetrahydroisoquinolin-6-yl)-7-fluorobenzo[b]thiophene-3-carbonitrile;
2-amino-4-((Sₐ)-2-((R)-1-(2-aminopyridin-3-yl)ethyl)-5-chloro-8-(2-(dimethylamino)ethoxy)-7-fluoro-1-oxo-1,2,3,4-tetrahydroisoquinolin-6-yl)-7-fluorobenzo[b]thiophene-3-carbonitrile; and
2-amino-4-((Sₐ)-2-((S)-1-(2-aminopyridin-3-yl)ethyl)-5-chloro-8-(2-(dimethylamino)ethoxy)-7-fluoro-1-oxo-1,2,3,4-tetrahydroisoquinolin-6-yl)-7-fluorobenzo[b]thiophene-3-carbonitrile.

### Example 30. 6-(2-Amino-3-cyano-7-fluorobenzo[b]thiophen-4-yl)-2-(1-(2-aminopyridin-3-yl)ethyl)-5-chloro-7-fluoro-1-oxo-1,2,3,4-tetrahydroisoquinoline-8-carbonitrile

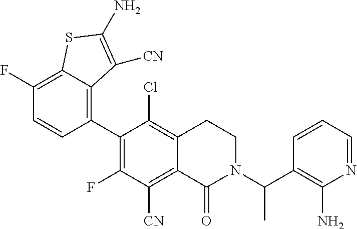 [SMILES: CC(c1cccnc1N)N1CCc2c(Cl)c(-c3ccc(F)c4sc(N)c(C#N)c34)c(F)c(C#N)c2C1=O]

To a 2-dram vial was added tert-butyl (4-(2-(1-(2-(bis(4-methoxybenzyl)amino)pyridin-3-yl)ethyl)-5-chloro-7-fluoro-8-iodo-1-oxo-1,2,3,4-tetrahydroisoquinolin-6-yl)-3-cyano-7-fluorobenzo[b]thiophen-2-yl)carbamate (from Example 25, Step 2, 20 mg, 0.020 mmol), Pd(PPh₃)₄ (4.73 mg, 4.10 μmol), and Zn(CN)₂ (4.81 mg, 0.041 mmol) followed by the addition of DMF (0.3 mL). The mixture was sparged with N₂ for 5 min. and then stirred at 100° C. for 1 h. After cooling to r.t., the mixture was quenched with water (0.4 mL) and extracted with EtOAc (3×1 mL). The combined organic phase was dried over Na₂SO₄ and concentrated. The crude product was dissolved in TFA (1 mL) and heated at 90° C. for 1 h, then diluted with MeCN and purified by prep. LCMS (XBRIDGE® C₁₈ column, eluting with a gradient of MeCN/water containing 0.1% TFA, at flow rate of 60 mL/min.) to afford the title compound as a TFA salt. LCMS calc. for C₂₆H₁₈ClF₂N₆OS (M+H)⁺: m/z=535.1; found 535.1.

The title compound comprises a mixture of diastereoisomers, i.e.,
(Rₐ)-6-(2-amino-3-cyano-7-fluorobenzo[b]thiophen-4-yl)-2-((R)-1-(2-aminopyridin-3-yl)ethyl)-5-chloro-7-fluoro-1-oxo-1,2,3,4-tetrahydroisoquinoline-8-carbonitrile;
(Rₐ)-6-(2-amino-3-cyano-7-fluorobenzo[b]thiophen-4-yl)-2-((S)-1-(2-aminopyridin-3-yl)ethyl)-5-chloro-7-fluoro-1-oxo-1,2,3,4-tetrahydroisoquinoline-8-carbonitrile;
(Sₐ)-6-(2-amino-3-cyano-7-fluorobenzo[b]thiophen-4-yl)-2-((R)-1-(2-aminopyridin-3-yl)ethyl)-5-chloro-7-fluoro-1-oxo-1,2,3,4-tetrahydroisoquinoline-8-carbonitrile; and
(Sₐ)-6-(2-amino-3-cyano-7-fluorobenzo[b]thiophen-4-yl)-2-((S)-1-(2-aminopyridin-3-yl)ethyl)-5-chloro-7-fluoro-1-oxo-1,2,3,4-tetrahydroisoquinoline-8-carbonitrile.

### Example A. GDP-GTP Exchange Assay

The inhibitor potency of the exemplified compounds was determined in a fluorescence based guanine nucleotide exchange assay, which measures the exchange of bodipy-GDP (fluorescently labeled GDP) for GTP to generate the active state of KRAS in the presence of SOS1 (guanine nucleotide exchange factor). Inhibitors were serially diluted in DMSO and a volume of 0.1 μL was transferred to the wells of a black low volume 384-well plate. 5 μL/well volume of bodipy-loaded KRAS G12V diluted to 2.5 nM in assay buffer (25 mM Hepes pH 7.5, 50 mM NaCl, 10 mM MgCl₂ and 0.01% Brij-35) was added to the plate and pre-incubated with inhibitor for 1 h at r.t. Appropriate controls (enzyme with no inhibitor or with a G12V inhibitor) were included on the plate. The exchange was initiated by the addition of a 5 μL/well volume containing 1 mM GTP and 300 nM SOS1 in assay buffer. The 10 μL/well reaction concentration of the bodipy-loaded KRAS G12V, GTP, and SOS1 were 2.5 nM, 500 μM, and 150 nM, respectively. The reaction plates were incubated at r.t. for 3 h, a time estimated for complete GDP-GTP exchange in the absence of inhibitor. For the KRAS WT, similar guanine nucleotide exchange assays were used with 2.5 nM as final concentration for the bodipy loaded KRAS proteins and 2 h incubation after adding GTP-SOS1 mixture. Internal compounds with confirmed binding were used as positive controls in the assay plates. Fluorescence intensities were measured on a PheraStar plate reader instrument (BMG Labtech) with excitation at 485 nm and emission at 520 nm.

Either GraphPad prism or Genedata Screener SmartFit was used to analyze the data. The IC₅₀ values were derived by fitting the data to a four parameter logistic equation producing a sigmoidal dose-response curve with a variable Hill coefficient.

The KRAS Wild Type (WT) and KRAS_G12V exchange assay IC₅₀ data are provided in Table A below. The symbol “†” indicates IC₅₀≤100 nM, “††” indicates IC₅₀>100 nM but ≤1 μM; and “†††” indicates IC₅₀ is >1 μM but ≤5 μM, “††††” indicates IC₅₀ is >5 μM but ≤5 μM. “NA” indicates IC₅₀ not available.

For certain compounds that were isolated as one or more atropisomers and/or diastereoisomers, the value for the most potent isomer is quoted.

TABLE A

| Ex. No. | G12V_exchange | WT_exchange |
|---|---|---|
| 1 | † | † |
| 2 | † | †† |
| 3 | † | †† |
| 4 | †† | †† |
| 5 | †† | ††† |
| 6 | † | †† |
| 7 | †††† | †††† |
| 8 | † | †† |
| 9 | †††† | †††† |
| 10 | † | †† |
| 11 | †† | ††† |
| 12 | ††† | NA |
| 13 | †† | †††† |
| 14 | † | †† |
| 15 | †† | †† |
| 16 | † | †† |
| 17 | †† | ††† |
| 18 | †† | ††† |
| 19 | † | †† |
| 20 | † | †† |
| 21 | † | †† |
| 22 | † | †† |
| 23 | †† | †††† |
| 24 | †† | †† |
| 25 | † | † |
| 26 | † | † |
| 27 | † | †† |
| 28 | † | †† |
| 29 | † | † |
| 30 | † | † |

### Example B: Caco2 Assay

Caco-2 cells are grown at 37° C. in an atmosphere of 5% CO₂ in DMEM growth medium supplemented with 10% (v/v) fetal bovine serum, 1% (v/v) nonessential amino acids, penicillin (100 U/mL), and streptomycin (100 μg/mL). Confluent cell monolayers are subcultured every 7 days or 4 days for Caco-2 by treatment with 0.05% trypsin containing 1 μM EDTA. Caco-2 cells are seeded in 96-well Transwell plates. The seeding density for Caco-2 cells is 14,000 cells/well. DMEM growth medium is replaced every other day after seeding. Cell monolayers are used for transport assays between 22 and 25 days for Caco-2 cells.

Cell culture medium is removed and replaced with HBSS. To measure the TEER, the HBSS is added into the donor compartment (apical side) and receiver compartment (basolateral side). The TEER is measured by using a REMS Autosampler to ensure the integrity of the cell monolayers. Caco-2 cell monolayers with TEER values a 300 Ω·cm² are used for transport experiments. To determine the Pₐₚₚ in the absorptive direction (A-B), solution of test compound (50 μM) in HBSS is added to the donor compartment (apical side), while HBSS solution with 4% BSA is added to the receiver compartment (basolateral side). The apical volume was 0.075 mL, and the basolateral volume is 0.25 mL. The incubation period is 120 min. at 37° C. in an atmosphere of 5% CO₂. At the end of the incubation period, samples from the donor and receiver sides are removed and an equal volume of MeCN is added for protein precipitation. The supernatants are collected after centrifugation (3000 rpm, Allegra X-14R Centrifuge from Beckman Coulter, Indianapolis, IN) for LCMS analysis. The permeability value is determined according to the equation:

$${P}_{app} ( cm / s ) = ( F * V D ) / ( S A * M D ) ,$$

where the flux rate (F, mass/time) is calculated from the slope of cumulative amounts of compound of interest on the receiver side, SA is the surface area of the cell membrane, VD is the donor volume, and MD is the initial amount of the solution in the donor chamber.

The Caco-2 data are provided in Table B below. The symbol “+” indicates a Caco-2 value of ≤0.5, “++” indicates a Caco-2 value of >0.5 but ≤1; and “+++” indicates a Caco-2 value of >1. “NA” indicates IC₅₀ not available.

TABLE B

| Ex. No. | Caco-2 |
|---|---|
| 1 | +++ |
| 2 | +++ |
| 3 | +++ |
| 14 | +++ |
| 15 | +++ |
| 16 | +++ |
| 30 | +++ |

### Example C: Luminescent Viability Assay

NCI-H838 (KRAS WT; ATCC CRL-5844), BEN (KRAS WT; DSMZ ACC254), and SW620 (KRAS G12V; DSMZ ACC382) cells are cultured in media supplemented with 10% FBS. Eight hundred cells per well in media supplemented with 2% FBS are seeded into white, flat 384-well tissue culture microtiter plates containing 50 nL dots of test compounds (final concentration is a 1:500 dilution, with a final concentration in 0.2% DMSO). Plates are incubated for 3 days at 37° C., 5% CO₂. At the end of the assay, 25 μL/well of CellTiter-Glo reagent (Promega) is added. Luminescence is read after 10 min. with a PHERAstar (BMG). Data are analyzed in Genedata Screener using SmartFit for IC₅₀ values.

### Example D: Cellular pERK HTRF Assay

NCI-H838 (KRAS WT; ATCC® CRL-5844), BEN (KRAS WT; DSMZ ACC254), and SW620 (KRAS G12V; DSMZ ACC382) cells were maintained in media supplemented with 10% FBS. The cells are plated at 5000 cells per well (8 μL) into 384-well low volume, flatbottom, and tissue culture treated white plates and incubated overnight at 37° C., 5% CO₂. The next morning, test compound stock solutions are diluted in media at 3× the final concentration and 4 μL are added to the cells, with a final concentration of 0.1% of DMSO. The cells are incubated with the test compounds for 4 h at 37° C., 5% CO₂. 4 μL of 4× lysis buffer with blocking reagent Revvity are added to each well and plates are rotated gently (300 rpm) for 30 min. at r.t. 4 μL per well of anti Phospho-ERK 1/2 d2 is mixed with anti Phospho-ERK 1/2 Cryptate (1:1), and added to each well, incubated overnight in the dark at r.t. Plates are read on the Pherastar plate reader at 665 nm and 620 nm wavelengths. Data are analyzed in Genedata Screener using SmartFit for IC₅₀ values.

### Example E: Whole Blood pERK1/2 HTRF Assay

BEN (KRAS WT; DSMZ ACC254), and YAPC (KRAS G12V; DSMZ ACC382) are maintained in media supplemented with 10% FBS. Cells are seeded in 96 well tissue culture plates at 50000 cells per well in 100 μL media and cultured for 1 day before the assay. Whole Blood is added to the 1 μL dots of compounds (prepared in DMSO) in 96 well plates and mixed gently by pipetting up and down so that the concentration of the compound in blood is 1× of desired concentration, in 0.6% DMSO. The media is aspirated from the cells and 50 μL per well of whole blood with test compound is added and incubated for 4 h at 37° C., 5% CO₂. After the 4 h incubation, the plates are washed once in PBS using the centrifugal Blue Washer (BlueCat Bio) with PBS. 50 μL/well of 1× lysis buffer (Revvity) with blocking reagent (Revvity) and Benzonase nuclease (1:10000 dilution Sigma) is then added and incubated at room temperature for 30 min. with shaking (250 rpm). Following lysis, 16 μL of lysate is transferred into 384-well small volume white plate (Greiner) using an Assist Plus (Integra Biosciences, NH). 4 μL of 1:1 mixture of anti Phospho-ERK 1/2 d2 and anti Phospho-ERK 1/2 Cryptate (Revvity) is added to the wells using the Assist Plus and incubated at room temperature overnight in the dark. Plates are read on the Pherastar plate reader at 665 nm and 620 nm wavelengths. Data are analyzed in Genedata Screener using SmartFit for IC₅₀ values.

### Example F: In Vivo Efficacy Studies

SW620(KRAS G12V) or YAPC(KRAS G12V) human cancer cells are obtained from the American Type Culture Collection and maintained in RPMI media supplemented with 10% FBS. For efficacy studies experiments, 5×10⁶ cells are inoculated subcutaneously into the right hind flank of 6- to 8-week-old NCr nude mice (Taconic Biosciences, Rensselear, NY, USA). When tumor volumes are approximately 150-300 mm³, mice are randomized by tumor volume and compounds are orally administered. Tumor volume is calculated using the formula (L×W²)/2, where L and W refer to the length and width dimensions, respectively. Tumor growth inhibition is calculated using the formula (1−(V_{T}/V_{C}))×100, where V_{T} is the average tumor volume of the treatment group on the last day of treatment, and V_{C} is the average tumor volume of the control group on the last day of treatment. Two-way analysis of variance with Dunnett's multiple comparisons test is used to determine statistical differences between treatment groups (GraphPad Prism). Mice are housed at 10-12 animals per cage and are provided enrichment and exposed to 12 h light/dark cycles. Mice whose tumor volumes exceeded limits (10% of body weight) for two consecutive measurements are humanely euthanized by CO₂ inhalation. Animals are maintained in a barrier facility fully accredited by the Association for Assessment and Accreditation of Laboratory Animal Care, International. All of the procedures are conducted in accordance with the US Public Service Policy on Human Care and Use of Laboratory Animals and with Incyte Animal Care and Use Committee Guidelines.

### Example G: Human Whole Blood Stability

The whole blood stability of the exemplified compounds is determined by LCMS/MS. The 96-Well Flexi-Tier™ Block (Analytical Sales & Services, Inc, Flanders, NJ) is used for the incubation plate containing 1.0 mL glass vials with 0.5 mL of blood per vial (pooled gender, human whole blood sourced from BIOIVT, Hicksville, NY or similar). Blood is prewarmed in water bath to 37° C. for 30 min. A 96-deep well analysis plate is prepared with the addition of 100 μL ultrapure water/well. 50 μL chilled ultrapure water/well is added to 96-deep well sample collection plate and covered with a sealing mat. 1 μL of 0.5 μM compound working solution (DMSO:water) is added to the blood in incubation plate to reach final concentrations of 1 μM, mixed by pipetting thoroughly and 50 μL is transferred 50 into the T=0 wells of the sample collection plate. Blood is allowed to sit in the water for 2 min. and then 400 μL stop solution/well is added (MeCN containing an internal standard). The incubation plate is placed in the Incu-Shaker CO₂ Mini incubator (Benchmark Scientific, Sayreville, NJ) at 37° C. with shaking at 150 rpm. At 1, 2 and 4 h, the blood samples are mixed thoroughly by pipetting and 50 μL is transferred into the corresponding wells of the sample collection plate. Blood is allowed to sit in the water for 2 min. and then 400 μL of stop solution/well is added. The collection plate is sealed and vortexed at 1700 rpm for 3 min. (VX-2500 Multi-Tube Vortexer, VWR International, Radnor, PA), and samples are then centrifuged in the collection plate at 3500 rpm for 10 min. (Allegra X-14R Centrifuge Beckman Coulter, Indianapolis, IN). 100 μL of supernatant/well is transferred from the sample collection plate into the corresponding wells of the analysis plate. The final plate is vortexed at 1700 rpm for 1 min. and analyze samples by LCMS/MS. The peak area ratio of the 1, 2, and 4 h samples relative to T=0 is used to determine the percent remaining. The natural log of the percent remaining versus time is used determine a slope to calculate the compounds half-life in blood (t_{1/2}=0.693/slope).

### Example H: In Vitro Intrinsic Clearance Protocol

For in vitro metabolic stability experiments, test compounds are incubated with human liver microsomes at 37° C. The incubation mixture contains test compounds (1 μM), NADPH (2 mM), and human liver microsomes (0.5 mg protein/mL) in 100 mM phosphate buffer (pH 7.4). The mixture is pre-incubated for 2 min. at 37° C. before the addition of NADPH. Reactions are commenced upon the addition of NADPH and quenched with ice-cold MeOH at 0, 10, 20, and 30 min. Terminated incubation mixtures are analyzed using LCMS/MS system. The analytical system consisted of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard is achieved using a Hypersil Gold C₁₈ column (50×2.1 mm, 5 μM, 175 Å) from ThermoFisher Scientific (Waltham, MA). Mobile phase A consists of 0.1% formic acid in water, and mobile phase B consists of 0.1% formic acid in MeCN. The total LCMS/MS runtime can be 2.75 min. with a flow rate of 0.75 mL/min. Peak area integrations and peak area ratio calculations are performed using Analyst software (version 1.6.3) from Applied Biosystems.

The in vitro intrinsic clearance, CL_{int, in vitro}, is calculated from the t_{1/2} of test compound disappearance as CL_{int, in vitro}=(0.693/t_{1/2})×(1/Cₚᵣₒₜₑᵢₙ), where Cₚᵣₒₜₑᵢₙ is the protein concentration during the incubation, and t_{1/2} is determined by the slope (k) of the log-linear regression analysis of the concentration versus time profiles; thus, t_{1/2}=ln2/k. The CL_{int, in vitro} values are scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(mg protein/g liver weight)×(g liver weight/kg body weight) is used. The in vivo hepatic clearance (CL_{H}) is then calculated by using CLᵢₙₜ and hepatic blood flow, Q (20 mL·min⁻¹·kg⁻¹ in humans) in the well-stirred liver model disregarding all binding from CL_{H}=(Q×CLᵢₙₜ)/(Q+CLᵢₙₜ). The hepatic extraction ratio was calculated as CL_{H} divided by Q.

### Example I: In vivo Pharmacokinetics Protocol

For in vivo pharmacokinetic experiments, test compounds are administered to male Sprague Dawley rats or male and female Cynomolgus monkeys intravenously or via oral gavage. For intravenous (IV) dosing, test compounds are dosed at 0.5 to 1 mg/kg using a formulation of 10% dimethylacetamide (DMAC) in acidified saline via IV bolus for rat and 5 min. or 10 min. IV infusion for monkey. For oral (PO) dosing, test compounds are dosed at 1.0 to 3.0 mg/kg using 5% DMAC in 0.5% methylcellulose in citrate buffer (pH 2.5). Blood samples are collected at predose and various time points up to 24 h postdose. All blood samples are collected using EDTA as the anticoagulant and centrifuged to obtain plasma samples. The plasma concentrations of test compounds are determined by LCMS methods. The measured plasma concentrations are used to calculate PK parameters by standard noncompartmental methods using Phoenix® WinNonlin software program (version 8.0, Pharsight Corporation).

In rats and monkeys, cassette dosing of test compounds are conducted to obtain preliminary PK parameters.

In vivo pharmacokinetic experiments with male beagle dogs may be performed under the conditions described above.

### Example J: Time Dependent Inhibition (TDI) of CYP Protocol

This assay is designed to characterize an increase in CYP inhibition as a test compounds is metabolized over time. Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CYP inhibitor could result in a positive result.

The results are from a cocktail of CYP specific probe substrates at 4 times their Km concentrations for CYP2C9, 2C19, 2D6 and 3A4 (midazolam) using human liver microsomes (HLM). The HLMs can be pre-incubated with test compounds at a concentration 10 μM for 30 min. in the presence (+N) or absence (−N) of a NADPH regenerating system, diluted 10-fold, and incubated for 8.min in the presence of the substrate cocktail with the addition of a fresh aliquot of NADPH regenerating system. A calibration curve of metabolite standards can be used to quantitatively measure the enzyme activity using LCMS/MS. In addition, incubations with known time dependent inhibitors, tienilic aicd (CYP2C9), ticlopidine (CYP2C19), paroxetine (CYP2D6), and troleandomycin (CYP3A4), used as positive controls are pre-incubated 30 min. with or without a NADPH regenerating system.

The analytical system consists of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard can be achieved using an ACQUITY UPLC BEH 130A, 2.1×50 mm, 1.7 μm HPLC column (Waters Corp, Milford, MA). Mobile phase A consists of 0.1% formic acid in water, and mobile phase B consists of 0.1% formic acid in MeCN. The total LCMS/MS runtime will be 2.50 min. with a flow rate of 0.9 mL/min. Peak area integrations and peak area ratio calculations are performed using Analyst software (version 1.6.3) from Applied Biosystems.

The percentage of control CYP2C9, CYP2C19, CYP2D6, and CYP3A4 activity remaining following preincubation of the compounds with NADPH is corrected for the corresponding control vehicle activity and then calculated based on 0 min. as 100%. A linear regression plot of the natural log of % activity remaining versus time for each isozyme is used to calculate the slope. The -slope is equal to the rate of enzyme loss, or the K_{obs}.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound having Formula (I): 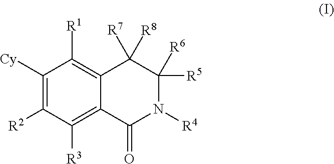
or a pharmaceutically acceptable salt thereof, wherein:
Cy is selected from C₃₋₇ cycloalkyl, 4-10 membered heterocycloalkyl, C₆₋₁₀ aryl, and 5-10 membered heteroaryl; wherein the C₃₋₇ cycloalkyl, 4-10 membered heterocycloalkyl, C₆₋₁₀ aryl, 5-10 membered heteroaryl, C₆₋₁₀ aryl, and 5-10 membered heteroaryl forming Cy is substituted with 1, 2, 3, or 4 substituents each independently selected from R^{Cy};
each R^{Cy} is independently selected from D, C₁₋₃ alkyl, C₁₋₃ haloalkyl, C₂₋₃ alkenyl, C₂₋₃ alkynyl, C₃₋₆ cycloalkyl, 4-10 membered heterocycloalkyl, C₆₋₁₀ aryl, 5-10 membered heteroaryl, halo, CN, OR^{aCy21}, SR^{aCy21}, NR^{cCy21}R^{dCy21}, S(O)R^{bCy21}, S(O)NR^{cCy21}R^{dCy21}, S(O)₂R^{bCy21}, NR^{cCy21}S(O)₂R^{bcCy21}, and S(O)₂NR^{cCy2l}R^{dCy21}; wherein the C₃₋₆ cycloalkyl, 4-10 membered heterocycloalkyl, C₆₋₁₀ aryl, and 5-10 membered heteroaryl forming R^{Cy} are each optionally substituted by 1, 2, 3, or 4 substituents independently selected from R^{Cy2A}; and wherein the C₁₋₃ alkyl, C₂₋₃ alkenyl, and C₂₋₃ alkynyl forming R^{Cy} are each optionally substituted by 1, 2, or 3 substituents independently selected from R^{Cy2B};
each RC^{y2A} is independently selected from C₁₋₃ alkyl, C₁₋₃ haloalkyl, C₂₋₃ alkenyl, C₂₋₃ alkynyl, and R^{Cy2B}; wherein the C₁₋₃ alkyl, C₂₋₃ alkenyl, and C₂₋₃ alkynyl forming R^{Cy2A} are each optionally substituted by 1, 2, or 3 substituents independently selected from R^{Cy2B};
each R^{Cy2B} is independently selected from D, halo, CN, OR^{aCy21}, SR^{aCy21}, C(O)R^{bCy21}, C(O)NR^{cCy21}R^{dCy21}, C(O)OR^{aCy21}, OC(O)R^{bCy21}, OC(O)NR^{cCy21}R^{dCy21}, NR^{cCy21}R^{dCy21}, NR^{cCy21}C(O)R^{bCy21}, NR^{cCy21}C(O)NR^{cCy21}R^{dCy21}, NR^{cCy21}C(O)OR^{aCy21}, C(═NR^{eCy21})NR^{cCy21}R^{dCy21}, NR^{cCy21}C(═NR^{eCy21})NR^{cCy21}R^{dCy21}, S(O)R^{bCy21}, S(O)NR^{cCy21}R^{dCy21}, S(O)₂R^{bCy21}, NR^{cCy21}S(O)₂R^{bCy21}, and S(O)₂NR^{cCy21}R^{dCy21};
R^{aCy21}, R^{bCy21}, R^{cCy21}, and R^{dCy21} are each independently selected from H, C₁₋₃ alkyl, C₁₋₃ haloalkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkylene, 5-10 membered heteroaryl-C₁₋₃ alkylene, C₃₋₇ cycloalkyl-C₁₋₃ alkylene, and 4-10 membered heterocycloalkyl-C₁₋₃ alkylene; wherein the C₆₋₁₀ aryl-C₁₋₃ alkylene, 5-10 membered heteroaryl-C₁₋₃ alkylene, C₃₋₇cycloalkyl-C₁₋₃ alkylene, and 4-10 membered heterocycloalkyl-C₁₋₃ alkylene forming R^{aCy21}, R^{bCy21}, R^{cCy21}, and R^{dCy21} are each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)R^{bCy22}, OC(O)NR^{cCy22}R^{dCy22}, NR^{cCy22}R^{dCy22}, NR^{cCy22}C(O)R^{bCy22}, NR^{cCy22}C(O)NR^{cCy22}R^{dCy22}, NR^{cCy22}C(O)OR^{aCy22}, C(═NR^{eCy22})NR^{cCy22}R^{dCy22}, NR^{cCy22}C(═NR^{eCy22})NR^{cCy22}R^{dCy22}, S(O)R^{bCy22}, S(O)NR^{cCy22}R^{dCy22}, S(O)₂R^{bCy22}, NR^{cCy22}S(O)₂R^{bCy22}, and S(O)₂NR^{cCy22}R^{dCy22};
or R^{cCy21} and R^{dCy21} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{aCy22}, SR^{aCy22}, C(O)R^{bCy22}, C(O)NR^{cCy22}R^{dCy22}, C(O)OR^{aCy22}, OC(O)R^{bCy22}, OC(O)NR^{cCy22}R^{dCy22}, NR^{cCy22}R^{dCy22}, NR^{cCy22}C(O)R^{bCy22}, NR^{cCy22}C(O)NR^{cCy22}R^{dCy22}, NR^{cCy22}C(O)OR^{aCy22}, C(═NR^{eCy22})NR^{cCy22}R^{dCy22}, NR^{cCy22}C(═NR^{eCy22})NR^{cCy22}R^{dCy22}, S(O)R^{bCy22}, S(O)NR^{cCy22}R^{dCy22}, S(O)₂R^{bCy22}, NR^{cCy22}S(O)₂R^{bCy22}, and S(O)₂NR^{cCy22}R^{dCy22};
R^{aCy22}, R^{bCy22}, R^{cCy22}, and R^{dCy22} are each independently selected from H, C₁₋₃ alkyl, C₁₋₃ haloalkyl, C₂₋₃ alkenyl, C₂₋₃ alkynyl, C₆₋₁₀ aryl, C₆₋₁₀ aryl-C₁₋₃ alkylene, 5-10 membered heteroaryl-C₁₋₃ alkylene, C₃₋₇ cycloalkyl-C₁₋₃ alkylene, and 4-10 membered heterocycloalkyl-C₁₋₃ alkylene; wherein the C₁₋₃ alkyl, C₂₋₃ alkenyl, C₂₋₃ alkynyl, C₆₋₁₀ aryl-C₁₋₃ alkylene, 5-10 membered heteroaryl-C₁₋₃ alkylene, C₃₋₇ cycloalkyl-C₁₋₃ alkylene, and 4-10 membered heterocycloalkyl-C₁₋₃ alkylene forming R^{aCy22}, R^{bCy22}, R^{cCy22}, and R^{dCy22} are each optionally substituted with 1, 2, or 3 substituents independently selected from OH, CN, amino, NH(C₁₋₃ alkyl), N(C₁₋₃ alkyl)₂, halo, C₁₋₃ alkyl, C₁₋₃ alkoxy, C₁₋₃ haloalkyl, and C₁₋₃ haloalkoxy; or
R^{cCy22} and R^{dCy22} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₃ alkyl, C₁₋₃ alkoxy, C₁₋₃ haloalkyl, and C₁₋₃ haloalkoxy;
R^{eCy21} and R^{eCy22} are each, independently, H, CN or NO₂;
R¹ is independently selected from halo, C₁₋₃ alkyl, C₂₋₃ alkenyl, C₂₋₃ alkynyl, C₃₋₁₀ cycloalkyl, 4-10 membered heterocycloalkyl, CN, OR^{1A}, NR^{1B}R^{1C}, C(O)NR^{1B}R^{1C}, and NR^{1B}C(O)R^{1C}; wherein the C₃₋₁₀ cycloalkyl and 4-10 membered heterocycloalkyl forming R¹ are each optionally substituted with 1, 2, or 3 substituents independently selected from R^{1D}; and wherein the C₁₋₃ alkyl, C₂₋₃ alkenyl, and C₂₋₃ alkynyl forming R¹ are each optionally substituted with 1, 2, or 3 substituents independently selected from R^{1E};
R^{1A} is selected from C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{1A} are each optionally substituted with 1, 2, or 3 substituents independently selected from R^{1D}; and wherein the C₁₋₃ alkyl, C₂₋₃ alkenyl, and C₂₋₃ alkynyl forming R^{1A} are each optionally substituted with 1, 2, or 3 substituents independently selected from R^{1E};
R^{1B} is selected from H, C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{1B} are each optionally substituted with 1, 2, or 3 substituents independently selected from R^{1D}; and wherein the C₁₋₃ alkyl, C₂₋₃ alkenyl, and C₂₋₃ alkynyl forming R^{1B} are each optionally substituted with 1, 2, or 3 substituents independently selected from R^{1E};
R^{1C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{1C} are each optionally substituted with 1, 2, or 3 substituents independently selected from R^{1E};
each R^{1D} is independently selected from C₁₋₃ alkyl, C₁₋₃ haloalkyl, C₂₋₃ alkenyl, C₂₋₃ alkynyl, and R^{1E}; wherein each of the C₁₋₃ alkyl, C₂₋₃ alkenyl, and C₂₋₃ alkynyl forming R^{1D} is optionally substituted with 1, 2, or 3 substituents independently selected from R^{1E};
each R^{1E} is independently selected from D, halo, CN, OH, C₁₋₃ alkoxy, C(O)OH, C(O)C₁₋₃ alkoxy, C(O)NH₂, C(O)NH(C₁₋₃ alkyl), C(O)N(C₁₋₃ alkyl)₂, and OC(O)C₁₋₃ alkyl;
R² is independently selected from halo, C₁₋₃ alkyl, C₂₋₃ alkenyl, C₂₋₃ alkynyl, C₃₋₁₀ cycloalkyl, 4-10 membered heterocycloalkyl, OR^{2A}, NR^{2B}R^{2C}, and NR^{2B}C(O)R^{2C}; wherein the C₃₋₁₀ cycloalkyl and 4-10 membered heterocycloalkyl forming R² are each optionally substituted with 1, 2, or 3 substituents independently selected from R^{2D}; and wherein the C₁₋₃ alkyl, C₂₋₃ alkenyl, and C₂₋₃ alkynyl forming R² are each optionally substituted with 1, 2, or 3 substituents independently selected from R^{2E};
R^{2A} is selected from C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{2A} are each optionally substituted with 1, 2, or 3 substituents independently selected from R^{2D}; and wherein the C₁₋₃ alkyl, C₂₋₃ alkenyl, and C₂₋₃ alkynyl forming R^{2A} are each optionally substituted with 1, 2, or 3 substituents independently selected from R^{2E};
R^{2B} is selected from H, C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{2B} are each optionally substituted with 1, 2, or 3 substituents independently selected from R^{2D}; and wherein the C₁₋₃ alkyl, C₂₋₃ alkenyl, and C₂₋₃ alkynyl forming R^{2B} are each optionally substituted with 1, 2, or 3 substituents independently selected from R^{2E};
R^{2C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{2C} are each optionally substituted with 1, 2, or 3 substituents independently selected from R^{2E};
each R^{2D} is independently selected from C₁₋₃ alkyl, C₁₋₃ haloalkyl, C₂₋₃ alkenyl, C₂₋₃ alkynyl, and R^{2E}; wherein each of the C₁₋₃ alkyl, C₂₋₃ alkenyl, and C₂₋₃ alkynyl forming R^{2D} is optionally substituted with 1, 2, or 3 substituents independently selected from R^{2E};
each R^{2E} is independently selected from D, halo, CN, OH, C₁₋₃ alkoxy, C(O)OH, C(O)C₁₋₃ alkoxy, C(O)NH₂, C(O)NH(C₁₋₃ alkyl), C(O)N(C₁₋₃ alkyl)₂, and OC(O)C₁₋₃ alkyl;
R³ is independently selected from H, halo, C₁₋₃ alkyl, C₂₋₃ alkenyl, C₂₋₄ alkynyl, C3-10 cycloalkyl, 4-10 membered heterocycloalkyl, 5-6 membered heteroaryl, CN, OR^{3A}, NR^{3B}R^{3C}, and NR^{3B}C(O)R^{3C}; wherein the C₃₋₁₀ cycloalkyl, 4-10 membered heterocycloalkyl, and 5-6 membered heteroaryl forming R³ are each optionally substituted with 1, 2, or 3 substituents independently selected from R^{3D}; and wherein the C₁₋₃ alkyl, C₂₋₃ alkenyl, and C₂₋₄ alkynyl forming R³ are each optionally substituted with 1, 2, or 3 substituents independently selected from R^{3E};
R^{3A} is selected from C₁₋₃ alkyl, C₂₋₃ alkenyl, C₂₋₃ alkynyl, C₃₋₁₀ cycloalkyl, 4-10 membered heterocycloalkyl, C₃₋₁₀ cycloalkyl-C₁₋₃ alkylene, and 4-10 membered heterocycloalkyl-C₁₋₃ alkylene; wherein the C₃₋₁₀ cycloalkyl 4-10 membered heterocycloalkyl, C₃₋₁₀ cycloalkyl-C₁₋₃ alkylene, and 4-10 membered heterocycloalkyl-C₁₋₃ alkylene forming R^{3A} are each optionally substituted with 1, 2, or 3 substituents independently selected from R^{3D}; and wherein the C₁₋₃ alkyl, C₂₋₃ alkenyl, and C₂₋₃ alkynyl forming R^{3A} are each optionally substituted with 1, 2, or 3 substituents independently selected from R^{3E};
R^{3B} is selected from H, C₁₋₃ alkyl, C₂₋₃ alkenyl, C₂₋₃ alkynyl, C₃₋₁₀ cycloalkyl, and 4-10 membered heterocycloalkyl; wherein the C₃₋₁₀ cycloalkyl and 4-10 membered heterocycloalkyl, forming R^{3B} are each optionally substituted with 1, 2, or 3 substituents independently selected from R^{3D}; and wherein the C₁₋₃ alkyl, C₂₋₃ alkenyl, and C₂₋₃ alkynyl forming R^{3B} are each optionally substituted with 1, 2, or 3 substituents independently selected from R^{3E};
R^{3C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{3C} are each optionally substituted with 1, 2, or 3 substituents independently selected from R^{3E};
each R^{3D} is independently selected from C₁₋₃ alkyl, C₁₋₃ haloalkyl, C₂₋₃ alkenyl, C₂₋₃ alkynyl, and R^{3E}; wherein each of the C₁₋₃ alkyl, C₂₋₃ alkenyl, and C₂₋₃ alkynyl forming R^{3D} is optionally substituted with 1, 2, or 3 substituents independently selected from R^{3E};
each R^{3E} is independently selected from O, C₁₋₃ alkyl, 4-6 membered heterocycloalkyl, halo, CN, OH, C₁₋₃ alkoxy, C(O)OH, C(O)C₁₋₃ alkoxy, C(O)NH₂, C(O)NH(C₁₋₃ alkyl), C(O)N(C₁₋₃ alkyl)₂, OC(O)C₁₋₃ alkyl, NH₂, NH(C₁₋₃ alkyl), and N(C₁₋₃ alkyl)₂, wherein the 4-6 membered heterocycloalkyl forming R^{3E} is optionally substituted with C₁₋₃ alkyl;
R⁴ is selected from C₁₋₃ alkyl, C₂₋₃ alkenyl, C₂₋₃ alkynyl, C₁₋₃ haloalkyl, C₃₋₅cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃alkylene, phenyl-C₁₋₃ alkylene, 5-6 membered heteroaryl-C₁₋₃ alkylene, halo, CN, OR^{a4}, C(O)R^{b4}, C(O)NR^{c4}R^{d4}, NR^{c4}R^{e4}, and NR^{c4}C(O)R^{b4}; wherein the C₃₋₅ cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃ alkylene, phenyl-C₁₋₃ alkylene, and 5-6 membered heteroaryl-C₁₋₃alkylene forming R⁴ are each optionally substituted with 1, 2, or 3 substituents independently selected from R^{4A}; and wherein the C₁₋₃ alkyl, C₂₋₃ alkenyl, and C₂₋₃ alkynyl forming R⁴ are each optionally substituted with 1, 2, or 3 substituents independently selected from R^{4B};
each R^{a4} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a4} are each optionally substituted with 1, 2, or 3 substituents independently selected from R^{4A}; and wherein the C₁₋₆ alkyl, C₂₋₆ alkenyl, and C₂₋₆ alkynyl forming R^{a4} are each optionally substituted with 1, 2, or 3 substituents independently selected from R^{4B};
each R^{b4}, R^{c4}, and R^{d4} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b4}, R^{c4}, and R^{d4} are each optionally substituted with 1, 2, or 3 substituents independently selected from R^{4A}; and wherein the C₁₋₃ alkyl, C₂₋₃ alkenyl, and C₂₋₃ alkynyl forming R^{b4}, R^{c4}, and R^{d4} are each optionally substituted with 1, 2, or 3 substituents independently selected from R^{4B}; or
any R^{c4} and R^{d4} attached to the same N atom, together with the N atom to which they are attached, form a 4-, 5-, or 6-membered heterocycloalkyl group optionally substituted with 1, 2, or 3 substituents independently selected from R^{4B};
each R^{e4} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e4} are each optionally substituted with 1, 2, or 3 substituents independently selected from R^{4A}; and wherein the C₁₋₃ alkyl, C₂₋₃ alkenyl, and C₂₋₃ alkynyl, forming R^{e4} are each optionally substituted with 1, 2, or 3 substituents independently selected from R^{4B}; or
R^{c4} and R^{e4} attached to the same N atom, together with the N atom to which they are attached, form a 4-, 5-, or 6-membered heterocycloalkyl group optionally substituted with 1, 2, or 3 substituents independently selected from R^{4B};
each R^{4A} is independently selected from C₁₋₃ alkyl, C₂₋₃ alkenyl, C₂₋₃ alkynyl, C₁₋₃ haloalkyl, and R^{4B}, wherein the C₁₋₃ alkyl, C₂₋₃ alkenyl, and C₂₋₃ alkynyl, forming R^{4A} are each optionally substituted with 1, 2 or 3 substituents independently selected from R^{4B};
each R^{4B} is independently selected from C₃₋₆cycloalkyl, 4-10 membered heterocycloalkyl, phenyl, 5-6 membered heteroaryl, halo, D, CN, OR^{a4B}, C(O)R^{b4B}, C(O)NR^{c4B}R^{d4B}, C(O)OR^{a4B}, NR^{c4B}R^{d4B}, and S(O)₂R^{b4B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{4C};
each R^{4C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a4C}, C(O)R^{b4C}, C(O)NR^{c4C}R^{d4C}, C(O)OR^{a4C}, NR^{c4C}R^{d4C}, and S(O)₂R^{b4C};
each R^{a4B}, R^{b4B}, R^{c4B} and R^{d4B} is independently selected from H, C₁₋₃ alkyl, and C₁₋₃ haloalkyl;
each R^{a4C}, R^{b4C}, R^{c4C} and R^{d4C} is independently selected from H, C₁₋₃ alkyl, and C₁₋₃ haloalkyl;
R⁵ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, CN, OR^{a5}, C(O)R^{b5}, C(O)NR^{c5}R^{d5}, NR^{c5}R^{e5}, and NR^{c5}C(O)R^{b5}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁵ are each optionally substituted with 1, 2, or 3 substituents independently selected from R^{5A}; and wherein the C₁₋₃ alkyl, C₂₋₃ alkenyl, and C₂₋₃ alkynyl forming R⁵ are each optionally substituted with 1, 2, or 3 substituents independently selected from R^{5B};
each R^{a5} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a5} are each optionally substituted with 1, 2, or 3 substituents independently selected from R^{5A}; and wherein the C₁₋₆ alkyl, C₂₋₆ alkenyl, and C₂₋₆ alkynyl forming R^{a5} are each optionally substituted with 1, 2, or 3 substituents independently selected from R^{5B};
each R^{b5}, R^{c5}, and R^{d5} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b5}, R^{c5}, and R^{d5} are each optionally substituted with 1, 2, or 3 substituents independently selected from R^{5A}; and wherein the C₁₋₃ alkyl, C₂₋₃ alkenyl, and C₂₋₃ alkynyl forming R^{b5}, R^{c5}, and R^{d5} are each optionally substituted with 1, 2, or 3 substituents independently selected from R^{5B}; or
any R^{c5} and R^{d5} attached to the same N atom, together with the N atom to which they are attached, form a 4-, 5-, or 6-membered heterocycloalkyl group optionally substituted with 1, 2, or 3 substituents independently selected from R^{5B};
each R^{e5} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e5} are each optionally substituted with 1, 2, or 3 substituents independently selected from R^{5A}; and wherein the C₁₋₃ alkyl, C₂₋₃ alkenyl, and C₂₋₃ alkynyl, forming R^{e5} are each optionally substituted with 1, 2, or 3 substituents independently selected from R^{5B}; or
R^{c5} and R^{e5} attached to the same N atom, together with the N atom to which they are attached, form a 4-, 5-, or 6-membered heterocycloalkyl group optionally substituted with 1, 2, or 3 substituents independently selected from R^{B};
each R^{5A} is independently selected from C₁₋₃ alkyl, C₂₋₃ alkenyl, C₂₋₃ alkynyl, C₁₋₃ haloalkyl, and R^{5B}, wherein the C₁₋₃ alkyl, C₂₋₃ alkenyl, and C₂₋₃ alkynyl forming R^{5A} are each optionally substituted with 1, 2 or 3 substituents independently selected from R^{5B};
each R^{5B} is independently selected from C₃₋₆cycloalkyl, 4-10 membered heterocycloalkyl, phenyl, 5-6 membered heteroaryl, halo, D, CN, OR^{a5B}, C(O)R^{b5B}, C(O)NR^{c5B}R^{d5B}, C(O)OR^{a5B}, NR^{c5B}R^{d5B}, and S(O)₂R^{b5B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{5C};
each R^{5C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a5C}, C(O)R^{b5C}, C(O)NR^{c5C}R^{d5C}, C(O)OR^{a5C}, NR^{c5C}R^{d5C}, and S(O)₂R^{b5C};
each R^{a5B}, R^{b5B}, R^{c5B} and R^{d5B} is independently selected from H, C₁₋₃ alkyl, and C₁₋₃ haloalkyl; and
each R^{a5C}, R^{b5c}, R^{c5C} and R^{d5C} is independently selected from H, C₁₋₃ alkyl, and C₁₋₃ haloalkyl;
R⁶ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, CN, OR^{a6}, C(O)R^{b6}, C(O)NR^{c6}R^{d6}, NR^{c6}R^{e6}, and NR^{c6}C(O)R^{b6}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁶ are each optionally substituted with 1, 2, or 3 substituents independently selected from R^{6A}; and wherein the C₁₋₃ alkyl, C₂₋₃ alkenyl, and C₂₋₃ alkynyl forming R⁶ are each optionally substituted with 1, 2, or 3 substituents independently selected from R^{6B};
each R^{a6} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a6} are each optionally substituted with 1, 2, or 3 substituents independently selected from R^{6A}; and wherein the C₁₋₆ alkyl, C₂₋₆ alkenyl, and C₂₋₆ alkynyl forming R^{a6} are each optionally substituted with 1, 2, or 3 substituents independently selected from R^{6B};
each R^{b6}, R^{c6}, and R^{d6} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b6}, R^{c6}, and R^{d6} are each optionally substituted with 1, 2, or 3 substituents independently selected from R^{6A}; and wherein the 1-3 alkyl, 2-3 alkenyl, and 2-3 alkynyl forming R^{b6}, R^{c6}, and R^{d6} are each optionally substituted with 1, 2, or 3 substituents independently selected from R^{6B}; or
any R^{c6} and R^{d6} attached to the same N atom, together with the N atom to which they are attached, form a 4-, 5-, or 6-membered heterocycloalkyl group optionally substituted with 1, 2, or 3 substituents independently selected from R^{6B};
each R^{e6} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e6} are each optionally substituted with 1, 2, or 3 substituents independently selected from R^{6A}; and wherein the C₁₋₃ alkyl, C₂₋₃ alkenyl, and C₂₋₃ alkynyl, forming R^{e6} are each optionally substituted with 1, 2, or 3 substituents independently selected from R^{6B}; or
R^{c6} and R^{e6} attached to the same N atom, together with the N atom to which they are attached, form a 4-, 5-, or 6-membered heterocycloalkyl group optionally substituted with 1, 2, or 3 substituents independently selected from R^{6B};
each R^{6A} is independently selected from C₁₋₃ alkyl, C₂₋₃ alkenyl, C₂₋₃ alkynyl, C₁₋₃ haloalkyl, and R^{6B}, wherein the C₁₋₃ alkyl, C₂₋₃ alkenyl, and C₂₋₃ alkynyl forming R^{6A} are each optionally substituted with 1, 2 or 3 substituents independently selected from R^{6B};
each R^{6B} is independently selected from C₃₋₆cycloalkyl, 4-10 membered heterocycloalkyl, phenyl, 5-6 membered heteroaryl, halo, D, CN, OR^{a6B}, C(O)R^{b6B}, C(O)NR^{c6B}R^{d6B}, C(O)OR^{a6B}, NR^{c6B}R^{d6B}, and S(O)₂R^{b6B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{6C};
each R^{6C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a6C}, C(O)R^{b6C}, C(O)NR^{c6C}R^{d6C}, O(O)OR^{a6C}, NR^{c6C}R^{d6C}, and S(O)₂R^{b6C};
each R^{a6B}, R^{b6B}, R^{c6B} and R^{d6B} is independently selected from H, C₁₋₃ alkyl, and C₁₋₃ haloalkyl; and
each R^{a6C}, R^{b6C}, R^{c6C} and R^{d6C} is independently selected from H, C₁₋₃ alkyl, and C₁₋₃ haloalkyl;
R⁷ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a7}, C(O)R^{b7}, C(O)NR^{c7}R^{d7}, NR^{c7}R^{e7}, and NR^{c7}C(O)R^{b7}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁷ are each optionally substituted with 1, 2, or 3 substituents independently selected from R^{7A}; and wherein the C₁₋₃ alkyl, C₂₋₃ alkenyl, and C₂₋₃ alkynyl forming R⁷ are each optionally substituted with 1, 2, or 3 substituents independently selected from R^{7B};
each R^{a7} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a7} are each optionally substituted with 1, 2, or 3 substituents independently selected from R^{7A}; and wherein the C₁₋₆ alkyl, C₂₋₆ alkenyl, and C₂₋₆ alkynyl forming R^{a7} are each optionally substituted with 1, 2, or 3 substituents independently selected from R^{7B};
each R^{b7}, R^{c7}, and R^{d7} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b7}, R^{c7}, and R^{d7} are each optionally substituted with 1, 2, or 3 substituents independently selected from R^{7A}; and wherein the 1-3 alkyl, 2-3 alkenyl, and 2-3 alkynyl forming R^{b7}, R^{c7}, and R^{d7} are each optionally substituted with 1, 2, or 3 substituents independently selected from R^{7B}; or
any R^{c7} and R^{d7} attached to the same N atom, together with the N atom to which they are attached, form a 4-, 5-, or 6-membered heterocycloalkyl group optionally substituted with 1, 2, or 3 substituents independently selected from R^{7B};
each R^{e7} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e7} are each optionally substituted with 1, 2, or 3 substituents independently selected from R^{7A}; and wherein the C₁₋₃ alkyl, C₂₋₃ alkenyl, and C₂₋₃ alkynyl, forming R^{e7} are each optionally substituted with 1, 2, or 3 substituents independently selected from R^{7B}; or
R^{c7} and R^{e7} attached to the same N atom, together with the N atom to which they are attached, form a 4-, 5-, or 6-membered heterocycloalkyl group optionally substituted with 1, 2, or 3 substituents independently selected from R^{7B};
each R^{7A} is independently selected from C₁₋₃ alkyl, C₂₋₃ alkenyl, C₂₋₃ alkynyl, C₁₋₃ haloalkyl, and R^{7B}, wherein the C₁₋₃ alkyl, C₂₋₃ alkenyl, and C₂₋₃ alkynyl forming R^{7A} are each optionally substituted with 1, 2 or 3 substituents independently selected from R^{7B};
each R^{7B} is independently selected from C₃₋₆cycloalkyl, 4-10 membered heterocycloalkyl, phenyl, 5-6 membered heteroaryl, halo, D, CN, OR^{a7B}, C(O)R^{b7B}, C(O)NR^{c7B}R^{d7B}, C(O)OR^{a7B}, NR^{c7B}R^{d7B}, and S(O)₂R^{b7B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{7C};
each R^{7C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a7C}, C(O)R^{b7C}, C(O)NR^{c7C}R^{d7C}, C(O)OR^{a7C}, NR^{c7C}R^{d7C}, and S(O)₂R^{b7C};
each R^{a7B}, R^{b7B}, R^{c7B} and R^{d7B} is independently selected from H, C₁₋₃ alkyl, and C₁₋₃ haloalkyl; and
each R^{a7C}, R^{b7C}, R^{c7C} and R^{d7C} is independently selected from H, C₁₋₃ alkyl, and C₁₋₃ haloalkyl;
R⁸ is selected from H, D, C₁₋₃ alkyl, C₂₋₃ alkenyl, C₂₋₃ alkynyl, C₁₋₃ haloalkyl, C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a8}, C(O)R^{b8}, C(O)NR^{c8}R^{d8}, NR^{c8}R^{e8}, and NR^{c8}C(O)R^{b8}; wherein the C₃₋₅ cycloalkyl, 4-6 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R⁸ are each optionally substituted with 1, 2, or 3 substituents independently selected from R^{8A}; and wherein the C₁₋₃ alkyl, C₂₋₃ alkenyl, and C₂₋₃ alkynyl forming R⁸ are each optionally substituted with 1, 2, or 3 substituents independently selected from R^{8B};
each R^{a8} is independently selected from C₁₋₆ alkyl, C₂₋₆ alkenyl, C₂₋₆ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a8} are each optionally substituted with 1, 2, or 3 substituents independently selected from R^{8A}; and wherein the C₁₋₆ alkyl, C₂₋₆ alkenyl, and C₂₋₆ alkynyl forming R^{a8} are each optionally substituted with 1, 2, or 3 substituents independently selected from R^{8B};
each R^{b8}, R^{c8}, and R^{d8} is independently selected from H, C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{b8}, R^{c8}, and R^{d8} are each optionally substituted with 1, 2, or 3 substituents independently selected from R^{8A}; and wherein the 1-3 alkyl, 2-3 alkenyl, and 2-3 alkynyl forming R^{b8}, R^{c8}, and R^{d8} are each optionally substituted with 1, 2, or 3 substituents independently selected from R^{8B}; or
any R^{c8} and R^{d8} attached to the same N atom, together with the N atom to which they are attached, form a 4-, 5-, or 6-membered heterocycloalkyl group optionally substituted with 1, 2, or 3 substituents independently selected from R^{8B};
each R^{e8} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl and 5-6 membered heteroaryl; wherein the C₃₋₆ cycloalkyl, 4-6 membered heterocycloalkyl, phenyl and 5-6 membered heteroaryl forming R^{e8} are each optionally substituted with 1, 2, or 3 substituents independently selected from R^{8A}; and wherein the C₁₋₃ alkyl, C₂₋₃ alkenyl, and C₂₋₃ alkynyl, forming R^{e8} are each optionally substituted with 1, 2, or 3 substituents independently selected from R^{8B}; or
R^{c8} and Re⁸ attached to the same N atom, together with the N atom to which they are attached, form a 4-, 5-, or 6-membered heterocycloalkyl group optionally substituted with 1, 2, or 3 substituents independently selected from R^{8B};
each R^{8A} is independently selected from C₁₋₃ alkyl, C₂₋₃ alkenyl, C₂₋₃ alkynyl, C₁₋₃ haloalkyl, and R^{8B}, wherein the C₁₋₃ alkyl, C₂₋₃ alkenyl, and C₂₋₃ alkynyl forming R^{8A} are each optionally substituted with 1, 2 or 3 substituents independently selected from R^{8B};
each R^{8B} is independently selected from C₃₋₆cycloalkyl, 4-10 membered heterocycloalkyl, phenyl, 5-6 membered heteroaryl, halo, D, CN, OR^{a8B}, C(O)R^{b8B}, C(O)NR^{c8B}R^{d8B}, C(O)OR^{a8B}, NR^{c8B}R^{d8B}, and S(O)₂R^{b8B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{8C};
each R^{8C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a8C}, C(O)R^{b8C}, C(O)NR^{c8C}R^{d8C}, (O)OR^{a8C}, NR^{c8c}R^{d8C}, and S(O)₂R^{b8C};
each R^{a8B}, R^{b8B}, R^{c8B} and R^{d8B} is independently selected from H, C₁₋₃ alkyl, and C₁₋₃ haloalkyl; and
each R^{a8C}, R^{b8C}, R^{c8C} and R^{d8C} is independently selected from H, C₁₋₃ alkyl, and C₁₋₃ haloalkyl;
wherein the ring-forming atoms of heterocycloalkyl and heteroaryl consist of at least one carbon atom and 1, 2, 3, or 4 heteroatoms independently selected from N, O, and S; and
wherein a ring-forming carbon atom of heterocycloalkyl and heteroaryl is optionally substituted by oxo to form a carbonyl group.

2. The compound of claim 1, or a pharmaceutically acceptable salt thereof, wherein
Cy is selected from C₃₋₇ cycloalkyl, 4-10 membered heterocycloalkyl, C₆₋₁₀ aryl, and 5-10 membered heteroaryl; wherein the C₃₋₇ cycloalkyl, 4-10 membered heterocycloalkyl, C₆₋₁₀ aryl, and 5-10 membered heteroaryl forming Cy is substituted with 1, 2, 3, or 4 substituents independently selected from R^{Cy};
each R^{Cy} is independently selected from D, C₁₋₃ alkyl, C₁₋₃ haloalkyl, C₂₋₃ alkenyl, C₂₋₃ alkynyl, C₃₋₆ cycloalkyl, 4-10 membered heterocycloalkyl, C₆₋₁₀ aryl, 5-10 membered heteroaryl, halo, CN, OR^{aCy21}, and NR^{cCy21}R^{dCy21}; wherein the C₃₋₆ cycloalkyl, 4-10 membered heterocycloalkyl, C₆₋₁₀ aryl, and 5-10 membered heteroaryl forming R^{Cy} are each optionally substituted by 1, 2, 3, or 4 substituents independently selected from R^{Cy2A}; and wherein the C₁₋₃ alkyl, C₂₋₃ alkenyl, and C₂₋₃ alkynyl forming R^{Cy} are each optionally substituted by 1, 2, or 3 substituents independently selected from R^{Cy2B};
each R^{Cy2A} is independently selected from C₁₋₃ alkyl, C₁₋₃ haloalkyl, C₂₋₃ alkenyl, and C₂₋₃ alkynyl; wherein the C₁₋₃ alkyl, C₂₋₃ alkenyl, and C₂₋₃ alkynyl forming R^{Cy2A} are each optionally substituted by 1, 2, or 3 substituents independently selected from R^{Cy2B},
each R^{Cy2B} is independently selected from D, halo, CN, ORa^{Cy21}, C(O)R^{bCy21}, C(O)NR^{cCy21}R^{dCy21}, C(O)OR^{aCy21}, OC(O)R^{bCy21}, OC(O)NR^{cCy21}R^{dCy21}, NR^{cCy21}R^{dCy21}, NR^{cCy21}C(O)R^{bCy21}, NR^{cCy21}C(O)NR^{cCy21}R^{dCy21}, and NR^{cCy21}C(O)OR^{aCy21};
R^{aCy21}, R^{bCy21}, R^{ccy21}, and R^{dCy21} are each independently selected from H, C₁₋₃ alkyl, C₁₋₃ haloalkyl, C₂₋₃ alkenyl, and C₂₋₃ alkynyl;
R¹ is selected from halo, C₁₋₃ alkyl, C₂₋₃ alkenyl, C₂₋₃ alkynyl, CN, OR^{1A}, NR^{1B}R^{1C}, C(O)NR^{1B}R^{1C}, and NR^{1B}C(O)R^{1C};
R^{1A} is C₁₋₃ alkyl;
R^{1B} is selected from H, C₁₋₃ alkyl, C₂₋₃ alkenyl, and C₂₋₃ alkynyl;
R^{1C} is selected from H, C₁₋₃ alkyl, C₂₋₃ alkenyl, and C₂₋₃ alkynyl;
R² is selected from halo, C₁₋₃ alkyl, C₂₋₃ alkenyl, C₂₋₃ alkynyl, OR^{2A}, NR^{2B}R^{2C}, and NR^{2B}C(O)R^{2C};
R^{2A} is C₁₋₃ alkyl;
R^{2B} is selected from H, C₁₋₃ alkyl, C₂₋₃ alkenyl, and C₂₋₃ alkynyl;
R^{2C} is selected from H, C₁₋₃ alkyl, C₂₋₃ alkenyl, and C₂₋₃ alkynyl;
R³ is selected from H, halo, C₁₋₃ alkyl, C₂₋₃ alkenyl, C₂₋₄ alkynyl, 5-6 membered heteroaryl, OR^{3A}, NR^{3B}R^{3C}, and NR^{3B}C(O)R^{3C}, wherein the C₂-4 alkynyl forming R³ is optionally substituted with 1 or 2 substituents independently selected from R^{3E};
R^{3A} is C₁₋₃ alkyl or 4-10 membered heterocycloalkyl-C₁₋₃ alkylene optionally substituted with 1, 2, or 3 substituents independently selected from R^{3D};
R^{3B} is selected from H, C₁₋₃ alkyl, C₂₋₃ alkenyl, and C₂₋₃ alkynyl;
R^{3C} is selected from H, C₁₋₃ alkyl, C₂₋₃ alkenyl, and C₂₋₃ alkynyl;
each R^{3D} is independently C₁₋₃ alkyl;
R⁴ is selected from C₁₋₃ alkyl, C₂₋₃ alkenyl, C₂₋₃ alkynyl, C₁₋₃ haloalkyl, C₃₋₅cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃alkylene, phenyl-C₁₋₃ alkylene, 5-6 membered heteroaryl-C₁₋₃ alkylene; wherein the C₃₋₅cycloalkyl, 4-10 membered heterocycloalkyl, phenyl, 5-6 membered heteroaryl, C₃₋₆ cycloalkyl-C₁₋₃ alkylene, 4-10 membered heterocycloalkyl-C₁₋₃ alkylene, phenyl-C₁₋₃ alkylene, and 5-6 membered heteroaryl-C₁₋₃ alkylene forming R⁴ are each optionally substituted with 1, 2, or 3 substituents independently selected from R^{4A}; and wherein the C₁₋₃ alkyl, C₂₋₃ alkenyl, and C₂₋₃ alkynyl forming R⁴ are each optionally substituted with 1, 2, or 3 substituents independently selected from R^{4B};
each R^{4A} is independently selected from C₁₋₃ alkyl, C₂₋₃ alkenyl, C₂₋₃ alkynyl, C₁₋₃ haloalkyl, and R^{4B};
each R^{4B} is independently selected from C₃₋₆cycloalkyl, 4-10 membered heterocycloalkyl, phenyl, 5-6 membered heteroaryl, halo, D, CN, OR^{a4B}, C(O)R^{b4B}, C(O)NR^{c4B}R^{d4B}, C(O)OR^{a4B}, and NR^{c4B}R^{d4B}; wherein the C₁₋₃ alkyl, C₃₋₆ cycloalkyl, 4-6 membered heterocycloalkyl, phenyl, and 5-6 membered heteroaryl are each optionally substituted with 1, 2, or 3 substituents independently selected from R^{4C};
each R^{4C} is independently selected from C₁₋₃ alkyl, C₂₋₃ alkenyl, C₂₋₃ alkynyl, C₁₋₃ haloalkyl, C₃₋₆ cycloalkyl, 4-6 membered heterocycloalkyl, phenyl, 5-6 membered heteroaryl, halo, D, CN, OR^{a4C}, C(O)R^{b4C}, C(O)NR^{c4C}R^{d4C}, C(O)OR^{a4C}, and NR^{c4C}R^{d4C};
each R^{a4B}, R^{b4B}, R^{c4B} and R^{d4B} is independently selected from H and C₁₋₃ alkyl;
each R^{a4C}, R^{b4C}, R^{c4C} and R^{d4C} is independently selected from H and C₁₋₃ alkyl;
R⁵ is selected from H, D, and C₁₋₃ alkyl;
R⁶ is selected from H, D, and C₁₋₃ alkyl;
R⁷ is selected from H, D, and C₁₋₃ alkyl; and
R⁸ is selected from H, D, and C₁₋₃ alkyl.

3. The compound of claim 1, or a pharmaceutically acceptable salt thereof, wherein
Cy is selected from C₆₋₁₀ aryl and 5-10 membered heteroaryl; wherein C₆₋₁₀ aryl and 5-10 membered heteroaryl forming Cy is substituted with 1, 2, 3, or 4 substituents independently selected from R^{Cy};
each R^{Cy} is independently selected from C₁₋₃ alkyl, C₁₋₃ haloalkyl, halo, CN, and NR^{cCy21}R^{dCy21};
R^{cCy21} and R^{dCy21} are each independently selected from H and C₁₋₃ alkyl;
R¹ is selected from halo, C₁₋₃ alkyl, CN, NR^{1B}R^{1C}, C(O)NR^{1B}R^{1C}, and NR^{1B}C(O)R^{1C};
R^{1B} is selected from H, C₁₋₃ alkyl, C₂₋₃ alkenyl, and C₂₋₃ alkynyl;
R^{1C} is selected from H, C₁₋₃ alkyl, C₂₋₃ alkenyl, and C₂₋₃ alkynyl;
R² is selected from halo, C₁₋₃ alkyl, NR^{2B}R^{2C}, and NR^{2B}C(O)R^{2C};
R^{2B} is selected from H, C₁₋₃ alkyl, C₂₋₃ alkenyl, and C₂₋₃ alkynyl;
R^{2C} is selected from H, C₁₋₃ alkyl, C₂₋₃ alkenyl, and C₂₋₃ alkynyl;
R³ is selected from halo, C₁₋₃ alkyl, C₂₋₄ alkynyl, 5-6 membered heteroaryl, NR^{3B}R^{3C} and NR^{3B}C(O)R^{3C}, wherein the C₂₋₄ alkynyl forming R³ is optionally substituted with 1 or 2 substituents independently selected from R^{3E};
R^{3B} is selected from H, C₁₋₃ alkyl, C₂₋₃ alkenyl, and C₂₋₃ alkynyl;
R^{3C} is selected from H, C₁₋₃ alkyl, C₂₋₃ alkenyl, and C₂₋₃ alkynyl;
R⁴ is selected from C₁₋₃ alkyl, 4-10 membered heterocycloalkyl, and 5-6 membered heteroaryl-C₁₋₃ alkylene; wherein the 4-10 membered heterocycloalkyl and 5-6 membered heteroaryl-C₁₋₃ alkylene forming R⁴ are each optionally substituted with 1, 2, or 3 substituents independently selected from R^{4A}; and wherein the C₁₋₃ alkyl forming R⁴ is optionally substituted with 1, 2, or 3 substituents independently selected from R^{4B};
each R^{4A} is independently selected from C₁₋₃ alkyl, C₁₋₃ haloalkyl, and R^{4B};
each R^{4B} is independently selected from C₃₋₆cycloalkyl, 4-10 membered heterocycloalkyl, phenyl, 5-6 membered heteroaryl, and NR^{c4B}R^{d4B}; wherein the C₃₋₆cycloalkyl, 4-6 membered heterocycloalkyl, phenyl, and 5-6 membered heteroaryl are each optionally substituted with 1, 2, or 3 substituents independently selected from R^{4C};
each R^{4C} is independently selected from C₁₋₃ alkyl, C₁₋₃ haloalkyl, halo, D, CN, OR^{a4C}, C(O)R^{b4C}, C(O)NR^{c4C}R^{d4C}, C(O)OR^{a4C}, and NR^{c4C}R^{d4C};
each R^{c4B} and R^{d4B} is independently selected from H and C₁₋₃ alkyl;
each R^{a4c}, R^{b4C}, R^{c4C} and R^{d4C} is independently selected from H and C₁₋₃ alkyl;
R⁵ is selected from H and C₁₋₃ alkyl;
R⁶ is selected from H and C₁₋₃ alkyl
R⁷ is selected from H and C₁₋₃ alkyl; and
R³ is selected from H and C₁₋₃ alkyl.

4. The compound of claim 1, wherein the compound of Formula (I) is a compound of Formula (Ia): 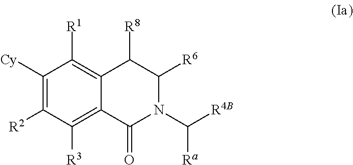
or a pharmaceutically acceptable salt thereof;
wherein Ra is C₁₋₂ alkyl; or
the compound of Formula (I) is a compound of Formula (Ib): 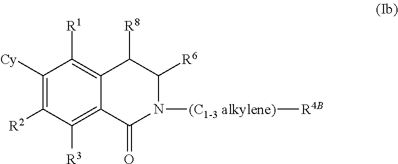
or a pharmaceutically acceptable salt thereof;
wherein
R^{4B} is 5-6 membered heteroaryl optionally substituted with 1, 2, or 3 substituents independently selected from R^{4C}.

5. (canceled)

6. The compound of claim 1, wherein the compound of Formula (I) is a compound of Formula (Ic) or Formula (Id): 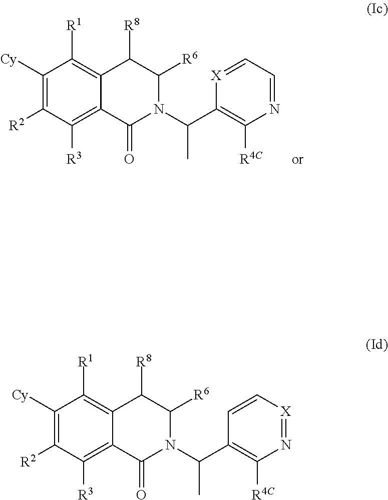
or a pharmaceutically acceptable salt thereof;
wherein
X is CH or N.

7. The compound of claim 1, or a pharmaceutically acceptable salt thereof, wherein Cy is selected from phenyl and 5-10 membered heteroaryl; wherein the phenyl and 5-10 membered heteroaryl forming Cy is substituted with 1, 2, 3, or 4 substituents independently selected from R^{Cy};
each R^{Cy} is independently selected from halo, C₁₋₃ haloalkyl, CN, and NR^{cCy21}R^{dCy21}; and
R^{ccy21} and R^{dCy21} are each independently selected from H and C₁₋₃ alkyl.

8-12. (canceled)

13. The compound of claim 1, or a pharmaceutically acceptable salt thereof, wherein R⁴ is selected from C₁₋₃ alkyl, 4-10 membered heterocycloalkyl, and 5-6 membered heteroaryl-C₁₋₃ alkylene; wherein the 4-10 membered heterocycloalkyl and 5-6 membered heteroaryl-C₁₋₃ alkylene forming R⁴ are each optionally substituted with 1 or 2 substituents independently selected from R^{4A}; and wherein the C₁₋₃ alkyl forming R⁴ is optionally substituted with 1 or 2 substituents independently selected from R^{4B};
each R^{4B} is independently selected from 5-6 membered heteroaryl and NR^{c4B}R^{d4B}; wherein the 5-6 membered heteroaryl is optionally substituted with 1 or 2 substituents independently selected from R^{4C};
each R^{4C} is independently selected from NR^{c4C}R^{d4C};
R^{c4B} and R^{d4B} are each H; and
R^{c4C} and R^{d4C} are each H.

14-18. (canceled)

19. The compound of claim 1, or a pharmaceutically acceptable salt thereof, wherein R¹ is selected from halo, C₁₋₃ alkyl, CN, and C(O)NR^{1B}R^{1C}.

20. (canceled)

21. The compound of claim 1, or a pharmaceutically acceptable salt thereof, wherein R² is selected from halo and C₁₋₃ alkyl.

22. (canceled)

23. The compound of claim 1, or a pharmaceutically acceptable salt thereof, wherein R³ is independently selected from H, halo, methyl, C₂₋₄ alkynyl, 5-6 membered heteroaryl, NR^{3B}R^{3C}, and NR^{3B}C(O)R^{3C}, wherein the C₂₋₄ alkynyl forming R³ is optionally substituted with 1 or 2 substituents independently selected from R^{3E};
R^{3B} is H; and R^{3C} is selected from H and C₁₋₃ alkyl.

24-25. (canceled)

26. The compound of claim 1, or a pharmaceutically acceptable salt thereof, wherein R⁵ is H;
R⁶ is H;
R⁷ is H; and
R⁸ is H.

27-29. (canceled)

30. A compound selected from
2-amino-4-(2-(1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-7-fluoro-5-methyl-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-5-chloro-7-fluoro-1-oxo-1,2,3,4-tetrahydroisoquinolin-6-yl)-5-fluorobenzo[b]thiophene-3-carbonitrile:
6-amino-2-(2-(1-(2-aminopyridin-3-yl)ethyl)-5-chloro-7-fluoro-1-oxo-1,2,3,4-tetrahydroisoquinolin-6-yl)-4-methyl-3-(trifluoromethyl)benzonitrile;
2-amino-4-(2-(1-(2-aminopyridin-3-yl)ethyl)-5-chloro-7-fluoro-1-oxo-1,2,3,4-tetrahydroisoquinolin-6-yl)-5-chlorobenzo[b]thiophene-3-carbonitrile;
2-amino-4-(2-(1-(3-aminopyrazin-2-yl)ethyl)-5-chloro-7-fluoro-1-oxo-1,2,3,4-tetrahydroisoquinolin-6-yl)-7-fluorobenzo[b]thiophene-3-carbonitrile;
2-amino-4-(2-((3-amino-1-methyl-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile;
2-amino-4-(2-(1-(3-aminopyridazin-4-yl)ethyl)-5-chloro-7-fluoro-1-oxo-1,2,3,4-tetrahydroisoquinolin-6-yl)-7-fluorobenzo[b]thiophene-3-carbonitrile;
4-(2-(1-(1H-1,2,4-triazol-1-yl)propan-2-yl)-5-chloro-7-fluoro-1-oxo-1,2,3,4-tetrahydroisoquinolin-6-yl)-2-amino-7-fluorobenzo[b]thiophene-3-carbonitrile;
2-amino-4-(8-amino-2-(1-(2-aminopyridin-3-yl)ethyl)-5-chloro-7-fluoro-1-oxo-1,2,3,4-tetrahydroisoquinolin-6-yl)-7-fluorobenzo[b]thiophene-3-carbonitrile;
N-(6-(2-amino-3-cyano-7-fluorobenzo[b]thiophen-4-yl)-2-(1-(2-aminopyridin-3-yl)ethyl)-5-chloro-7-fluoro-1-oxo-1,2,3,4-tetrahydroisoquinolin-8-yl)acetamide;
6-(2-amino-7-fluorobenzo[d]thiazol-4-yl)-2-(1-(2-aminopyridin-3-yl)ethyl)-5-chloro-7-fluoro-8-(1H-pyrazol-4-yl)-3,4-dihydroisoquinolin-1(2H)-one;
8-amino-6-(2-amino-7-fluorobenzo[d]thiazol-4-yl)-2-(1-(2-aminopyridin-3-yl)ethyl)-5-chloro-7-fluoro-3,4-dihydroisoquinolin-1(2H)-one;
2-amino-4-(2-(1-(2-aminopyridin-3-yl)ethyl)-7-chloro-5-fluoro-1-oxo-1,2,3,4-tetrahydroisoquinolin-6-yl)-7-fluorobenzo[b]thiophene-3-carbonitrile
2-amino-4-(2-(1-amino-6,7-dihydro-5H-cyclopenta[c]pyridin-7-yl)-5-chloro-7-fluoro-1-oxo-1,2,3,4-tetrahydroisoquinolin-6-yl)-7-fluorobenzo[b]thiophene-3-carbonitrile;
2-amino-4-(2-(1-(4-aminopyrimidin-5-yl)ethyl)-5-chloro-7-fluoro-1-oxo-1,2,3,4-tetrahydroisoquinolin-6-yl)-7-fluorobenzo[b]thiophene-3-carbonitrile;
2-amino-4-(5-chloro-7-fluoro-2-(1-methyl-2-oxopyrrolidin-3-yl)-1-oxo-1,2,3,4-tetrahydroisoquinolin-6-yl)-7-fluorobenzo[b]thiophene-3-carbonitrile;
2-amino-4-(2-((3-amino-1H-pyrazol-4-yl)methyl)-5-chloro-7-fluoro-1-oxo-1,2,3,4-tetrahydroisoquinolin-6-yl)-7-fluorobenzo[b]thiophene-3-carbonitrile:
6-(2-amino-3-cyano-7-fluorobenzo[b]thiophen-4-yl)-2-(1-(2-aminopyridin-3-yl)ethyl)-7-fluoro-1-oxo-1,2,3,4-tetrahydroisoquinoline-5-carbonitrile;
2-amino-4-(2-(1-(2-aminopyridin-3-yl)ethyl)-5-ethynyl-7-fluoro-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-7-fluoro-5-(hydroxymethyl)-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-5-(difluoromethyl)-7-fluoro-1-oxo-1,2,3,4-tetrahydroisoquinolin-6-yl)-7-fluorobenzo[b]thiophene-3-carbonitrile;
6-(2-amino-3-cyano-7-fluorobenzo[b]thiophen-4-yl)-2-(1-(2-aminopyridin-3-yl)ethyl)-7-fluoro-1-oxo-1,2,3,4-tetrahydroisoquinoline-5-carboxamide;
2-amino-4-(2-(1-(2-aminopyridin-3-yl)ethyl)-5-chloro-4,4,7-trifluoro-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-5-chloro-8-(3-(dimethylamino)prop-1-yn-1-yl)-7-fluoro-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-5-chloro-8-(4-(dimethylamino)but-1-yn-1-yl)-7-fluoro-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-5-chloro-7-fluoro-8-((1-methylpyrrolidin-2-yl)ethynyl)-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-5-chloro-7-fluoro-8-methoxy-1-oxo-1,2,3,4-tetrahydroisoquinolin-6-yl)-7-fluorobenzo[b]thiophene-3-carbonitrile;
2-amino-4-(2-(1-(2-aminopyridin-3-yl)ethyl)-5-chloro-8-(2-(dimethylamino)ethoxy)-7-fluoro-1-oxo-1,2,3,4-tetrahydroisoquinolin-6-yl)-7-fluorobenzo[b]thiophene-3-carbonitrile; and
6-(2-amino-3-cyano-7-fluorobenzo[b]thiophen-4-yl)-2-(1-(2-aminopyridin-3-yl)ethyl)-5-chloro-7-fluoro-1-oxo-1,2,3,4-tetrahydroisoquinoline-8-carbonitrile;
and pharmaceutically acceptable salts thereof.

31. A pharmaceutical composition comprising the compound or pharmaceutically acceptable salt thereof according to claim 1, and at least one pharmaceutically acceptable carrier or excipient.

32. A method of inhibiting KRAS activity, the method comprising contacting the compound or pharmaceutically acceptable salt thereof according to claim 1, with KRAS.

33. (canceled)

34. The method of claim 32, wherein KRAS is characterized as having a somatic mutation of G12C, G12D, or G12V.

35-36. (canceled)

37. A method of treating a disease or disorder associated with activity of KRAS, the method comprising administering to a patient in need thereof a therapeutically effective amount of the compound or pharmaceutically acceptable salt thereof according to claim 1.

38. A method of treating a disease or disorder associated with activity of a KRAS protein harboring a G12C, G12D, or G12V mutation, the method comprising administering to a patient in need thereof a therapeutically effective amount of the compound or pharmaceutically acceptable salt thereof according to claim 1.

39-40. (canceled)

41. A method for treating a cancer in a patient, the method comprising administering to the patient a therapeutically effective amount of the compound or pharmaceutically acceptable salt thereof according to claim 1.

42. The method of claim 41, wherein the cancer is selected from a carcinoma, a hematological cancer, a sarcoma, and glioblastoma.

43. The method of claim 42, wherein the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma.

44. The method of claim 42, wherein the cancer is a carcinoma selected from pancreatic, colorectal, lung, bladder, gastric, esophageal, breast, head and neck, cervical, skin, and thyroid cancers.

45. The method of claim 41, wherein abnormally proliferating cells of the cancer comprise KRAS having a G12C, G12D, or G12V mutation.

46-47. (canceled)

48. A method of treating an immunological or inflammatory disorder comprising administering to a patient in need thereof a therapeutically effective amount of the compound or pharmaceutically acceptable salt thereof according to claim 1.

49. The method of claim 48, wherein the immunological or inflammatory disorder is associated with activity of KRAS.

50-52. (canceled)

53. The method of claim 48, wherein the immunological or inflammatory disorder is Ras-associated lymphoproliferative disorder or juvenile myelomonocytic leukemia caused by a somatic mutation of KRAS.

54. The method of claim 53, wherein the somatic mutation of KRAS is G12C, G12D, or G12V.

55-59. (canceled)
